(19) United States
(12) Patent Application Publication
(10) Pub. No.: US 2019/0113423 A1
(43) Pub. Date: Apr. 18, 2019

(54) METHODS FOR PREPARING AND ANALYZING TUMOR TISSUE SAMPLES FOR DETECTION AND MONITORING OF CANCERS

(71) Applicant: Clearlight Diagnostics, LLC, Sunnyvale, CA (US)

(72) Inventors: Laurie Goodman, Sunnyvale, CA (US); Karl A. Deisseroth, Stanford, CA (US)

(21) Appl. No.: 15/781,082

(22) PCT Filed: Dec. 2, 2016
(86) PCT No.: PCT/US16/64751

§ 371 Date: Jun. 1, 2018

Related U.S. Application Data
(60) Provisional application No. 62/262,314, filed on Dec. 2, 2015.

(51) Int. Cl. G01N 1/36; G01N 33/483; G06T 7/00; G01N 1/30

(52) U.S. Cl. CPC G01N 1/36; G01N 33/4833; G01N 2001/305; G01N 1/30; G06T 7/0014

(57) ABSTRACT

The present invention provides methods for processing and analyzing large intact biological samples, including tumor tissue samples. The methods have a variety of uses, including for the diagnosis and monitoring of tumors and tumor metastasis.

### CROSS REFERENCE TO RELATED APPLICATIONS

This application claims priority to U.S. Provisional Application No. 62/262,314, filed on Dec. 2, 2015, which is incorporated by reference herein in its entirety.

### BACKGROUND

### Field

The present invention is directed to methods for processing and analyzing large biological tissue samples, including tumor tissue samples, e.g., for diagnostic, prognostic, and predictive clinical cancer research and standard of care practice.

### Description of the Related Art

Solid tumors are heterogeneous with multiple cell types, and recent data suggests that the surrounding microenvironment plays a key role in tumor growth, metastasis, and response or resistance to therapeutic agents. However, technologies currently utilized for preclinical as well as diagnostic, prognostic, and predictive clinical cancer research and standard of care practice are limited in their ability to analyze the tumor in the context of it microenvironment. Standard methods that utilize 2-dimensional analysis of thin tissue sections (4-10 micron) in the format of formalin fixed paraffin embedded tissue (FFPE) are limited by the inability to examine 3-dimensional features of tumors, such as changes in vascular structure and the surrounding extracellular matrix, or lymphatic or immune cell invasion. In addition, rare events, such as rare tumor cells or rare tumor marker gene expression, may be missed due to heterogeneity within a tumor or tumor microenvironment. The sample processing and preservation procedures may also destroy certain tumor characteristics. Newer technologies utilizing flow cytometry, RT-PCR, or next generation sequencing and analysis of tumors have advanced the preclinical and clinical cancer field; however, they also suffer from the ability to correlate key quantitative information while maintaining tumor and the surrounding microenvironment architecture and morphology.

Preserving spatial information, while at the same time capturing high resolution qualitative and quantitative information, leads to a better understanding of tumor microstructure, vascularization, the types of cells within the solid tumor microenvironment, and cellular interactions across a 3-dimensional structure of the tumor tissue. This will aid in characterizing tumors, determining or predicting the likelihood of tumor growth and/or metastasis, predicting and/or monitoring tumor response to therapy, and developing novel cancer treatments. In addition, it will provide a greater understanding of mechanisms of response and eventual resistance to therapy.

Clearly, there is a need in the art for improved methods and compositions for evaluating larger biological samples (e.g., 5000-10,000 microns), such as tumor tissue samples, as well as being able to visualize and quantify biomarker distribution throughout the tissue sample and its microenvironment, for diagnostic uses, prognostic uses, and for predicting response or resistance to treatment. The present invention provides such methods, which allow the examination or large, intact biological samples, including tumor tissue samples.

### BRIEF SUMMARY

The present invention provides methods and related composition useful in the analysis of tissues and organs.

In one embodiment, the present invention includes a method for analyzing a biological sample comprising: (a) processing a biological sample by: (i) fixing the sample in the presence of hydrogel subunits; (ii) polymerizing the hydrogel subunits to form a hydrogel-embedded sample; (iii) clearing the hydrogel-embedded sample; and (iv) labeling the cleared hydrogel-embedded sample with one or more first detectable marker; (b) imaging the processed sample by microscopy, optionally light microscopy, to generate at least one first image of the biological sample and/or determine an amount of the first detectable marker in the biological sample. In particular embodiments, the biological sample is a tumor tissue sample, a previously frozen biological sample, or a cell line. In particular embodiments, the biological sample is a cell line pellet, e.g., a frozen cell line pellet. In certain embodiments, the biological sample has a length of greater than 10 microns and/or a thickness of greater than 10 microns. In certain embodiments, the biological sample has a length of greater than 20 microns and/or a thickness of greater than 20 microns. In certain embodiments, the method further comprises: (c) washing the processed sample after imaging to remove the one or more first detectable label; (d) relabeling the washed sample with one or more second detectable label; and (e) imaging the relabeled sample by microscopy, optionally light microscopy, to obtain at least one second image of the biological sample and/or determine an amount of the second detectable marker in the biological sample. In certain embodiments, the biological sample is a tumor tissue sample, and at least one of the one or more first and second detectable labels is associated with a tumor characteristic.

In one embodiments, the present invention includes a method for diagnosing a tumor or determining the prognosis of a tumor in a subject comprising: (a) processing a tumor tissue sample obtained from the subject by: (i) fixing the tumor tissue sample in the presence of hydrogel subunits; (ii) polymerizing the hydrogel subunits to form a hydrogel-embedded sample; (iii) clearing the hydrogel-embedded sample; and (iv) optionally, labeling the cleared hydrogel-embedded sample with one or more first detectable marker; (b) imaging the processed sample to generate at least one tumor image of and/or determine an amount of the first detectable marker; and (c) comparing the tumor image or amount against one or more control images or amounts or predetermined images or amounts obtained from normal tissue, tumor tissue, tumor tissue associated with a good prognosis, or tumor tissue associated with a poor prognosis, thereby determining the presence of a tumor or the prognosis of a tumor.

In a related embodiments, the present invention includes a method for determining the responsiveness of a tumor in a subject to a therapeutic treatment comprising: (a) processing a first tumor tissue sample obtained from the subject at a first time point by: (i) fixing the tumor tissue sample in the presence of hydrogel subunits; (ii) polymerizing the hydrogel subunits to form a hydrogel-embedded sample; (iii) clearing the hydrogel-embedded sample; and (iv) optionally, labeling the cleared hydrogel-embedded sample with one or more first detectable marker; (b) processing a second tumor tissue sample obtained from the subject at a second time point following a therapeutic treatment by: (i) fixing the tumor tissue sample in the presence of hydrogel subunits; (ii) polymerizing the hydrogel subunits to form a hydrogel-embedded sample; (iii) clearing the hydrogel-embedded sample; and (iv) optionally, labeling the cleared hydrogel-embedded sample with one or more first detectable marker; (c) imaging the processed samples of (a) and (b) to generate a first tumor image of the processed sample of (a) and a second tumor image of the processed sample of (b) and/or determining a first amount of the one or more first detectable marker in the processed samples of (a) and a second amount of the one or more first detectable marker in the processed sample of (b); and(d) comparing the first tumor image against the second tumor image, or the first amount to the second amount of the one or more detectable marker, wherein if the second tumor image or second amount shows less tumor characteristics than the first tumor image or amount, the tumor is responding favorably to the therapeutic treatment, and wherein if the second tumor image or amount show more tumor characteristics than the first tumor image or amount, the tumor is responding poorly to the therapeutic treatment. Optionally, a tumor tissue sample obtained from the subject at a third or more time point is also processed, imaged and compared in the same manner as the first and second tumor tissue samples.

In particular embodiments of any of the methods of the present invention, the subject is a mammal, e.g., a human, or an animal model of human disease.

In certain embodiments, the imaging of to generate the first tumor image includes: placing the processed sample in an optically homogenous sample manipulation component; aligning one or more light sheets and one or more detection focal planes of a microscopic device at a plurality of locations within the processed sample; performing an imaging procedure to collect an image from each of the plurality of locations with the processed sample; and generating the first tumor image based on the collected images from each of the plurality of locations. In certain embodiments, the imaging further includes: applying the alignment parameter to each location; simultaneously illuminating the location with a light sheet and capturing an image of the location; and constructing a three-dimensional image of the sample using the image from each location.

In particular embodiments of any of the methods, fixing the tumor tissue sample comprises contacting the sample with a paraformaldehyde.

In certain embodiments of any of the methods, the hydrogel subunits comprise an acrylamide.

In certain embodiments of any of the methods, polymerizing the tumor tissue sample comprises thermal cross-linking.

In certain embodiments of any of the methods, clearing the tumor tissue sample comprises electrophoresing the sample. In particular embodiments, the electrophoresing is performed using a buffer solution that comprises an ionic surfactant, optionally sodium dodecyl sulfate. In particular embodiments, the sample is electrophoresed using a voltage ranging from about 10 to about 60 volts; and/or the sample is electrophoresed for a period of time ranging from about 15 minutes to about 10 days.

In particular embodiments of any of the methods, the method further comprises incubating the cleared sample in a mounting medium that has a refractive index that substantially matches that of the cleared sample, wherein the mounting medium increases the optical clarity of the sample, and wherein the mounting medium optionally comprises glycerol.

In particular embodiments of any of the methods, the method further comprises: (a) washing the processed sample obtained at the first and/or second time point before or after imaging to remove the one or more first detectable label; (b) relabeling the washed sample(s) with one or more second detectable label; (c) imaging the relabeled sample(s) by microscopy, optionally light microscopy, to obtain a second tumor image(s); and (d) examining the second tumor image(s) as described herein.

In particular embodiments of any of the methods, the labeling and/or relabeling comprises contacting the cleared hydrogel-embedded sample with one or more detectable markers that bind to cellular or extracellular components within the sample. In certain embodiments, the cellular or extracellular components are selected from the group consisting of: immune cell markers; cancer stem cell markers; extracellular matrix proteins; blood vessel or microvasculature markers; apoptosis markers; and/or tumor markers. In certain embodiments, the detectable marker comprises a polypeptide, nucleic acid, or small molecule. In particular embodiments, the detectable marker is detectable with or without the use of a detectable secondary agent that binds the detectable marker.

In certain embodiments, any of the methods described herein comprise fixing or embedding the biological sample in a solution of hydrogel monomer, cross-linking the embedded hydrogel monomers, clearing the cross-linked sample, staining the cleared sample with one or more detectable markers, and imaging the stained sample using COLM or another imaging process described herein. In particular embodiments, the sample is fixed in a hydrogel monomer solution comprising acrylamide and one or more cross-linkers, e.g., bis-acrylamide, and paraformaldehyde; the acrylamide is cross-linked or polymerized until the hydrogel monomer solution solidifies; the cross-linked sample is cleared using a solution comprising a detergent such as sodium dodecyl sulfate (SDS), for several hours to several weeks or months, the cleared sample is then stained using one or more detectable markers; and the stained sample is imaged via COLM or another microscopy technique. In particular embodiments, the imaging comprises microscopic analysis that includes: placing the sample in a sample chamber in an optically homogenous sample manipulation component; performing a calibration procedure to align one or more light sheets and one or more detection focal planes of a microscope device at a plurality of locations within the sample to acquire an alignment parameter for each location; performing an imaging procedure to collect an image from each of the plurality of locations within the sample; applying the alignment parameter to each location and simultaneously illuminating the location with a light sheet and capturing an image of the location; and constructing a three-dimensional image of the sample using the image from each location.

In certain embodiments of any of the methods, the method further comprises imaging one or more control biological sample, e.g., a positive or negative control to assess processing and labeling. In particular embodiments, it further comprises labeling the control biological sample and imaging the labeled sample. In particular embodiments, it comprises fixing, polymerizing, clearing, and labeling the one or more control biological sample. In particular embodiments, the control biological sample is a cell pellet, such as, e.g., a pellet of a cultured cell line. In certain embodiments, the control is a frozen or previously frozen cell pellet. In particular embodiments, the control is a previously fixed and cleared biological sample, such as, e.g., a previously fixed and cleared pell of cultured cells. In certain embodiments of methods that comprise determining an amount of a detectable marker for a test sample, the amount determined is compared to the amount of the detectable marker determined under the same conditions for a control sample, and the amount determined for the test sample. The present invention further includes cell line pellets processed as described herein. In particular embodiments, the cell line pellet has been fixed in the presence of hydrogel subunits; (ii) the hydrogel subunits were polymerized to form a hydrogel-embedded cell line pellet; (iii) and the hydrogel-embedded sample was cleared, as described herein. In addition, the cell line pellets processed as described herein may be frozen. In certain embodiments, a cell line pellet has a length of greater than 10 microns and/or a thickness of greater than 10 microns. In certain embodiments, the biological sample has a length of greater than 20 microns and/or a thickness of greater than 20 microns.

In certain embodiments, the disclosure includes a kit comprising one or more cell line pellets processed as described herein, optionally frozen pellets. In certain embodiments, the cell pellet comprises cells of a tumor cell line, e.g., MCF7 cells. In certain embodiments, the kit further comprises one or more detectable marker that bind to or identifies a biomarker (e.g., protein, gene or mRNA) associated with a tumor. In certain embodiments, the biomarker is also present in the cells of at least one of the one or more cell line pellets. In some embodiments, the kit further comprises hydrogel monomers, e.g., a fixing solution comprising hydrogel monomers, such as acrylamide, and a fixative such as paraformaldehyde. In some embodiments, the kit further comprises a clearing solution, e.g., a buffer solution that comprises an ionic surfactant, optionally sodium dodecyl sulfate.

In certain embodiments of any of the methods, the tumor is selected from the group consisting of: adrenal cortical cancer, anal cancer, aplastic anemia, bileduct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity and paranasal sinus cancer, nasopharyngeal cancer, neuroblastoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, and Wilms tumor.

In one embodiment, the tumor is a breast cancer tumor, and the one or more detectable marker binds to or identifies one or more of HER2, estrogen receptor (ER), progesterone receptor (PR), pan-cytokeratin, Ki67, CD3, CD4, CD8, CD20, CD68, or Foxp3, programmed death ligand-1 (PD-L1), programmed death-1 (PD-1), programmed death-ligand 2 (PD-L2), cytotoxic T-lymphocyte-associated protein-4 (CTLA-4), and androgen receptor (AR). In certain embodiments, the tumor is a breast cancer tumor, and the one or more detectable marker binds to or identifies human epidermal growth factor receptor-2 (HER2 or HER2/neu), estrogen receptor (ER), progesterone receptor (PR) or Ki-67. In certain embodiments, a detectable marker binds the target polypeptide, while in other embodiments, it binds a polynucleotide that encodes the target polypeptide. In certain embodiments, the detectable marker binds to a human target polypeptide. A detectable marker may be directly detectable or detectable using a secondary molecule.

In one embodiment, the tumor is a lung tumor, and the one or more detectable marker binds to or identifies one or more of epidermal growth factor receptor, including sensitizing and resistance mutations thereof, and ALK/ROS/RET rearrangements, BRAF mutations, and PD-L1, PD-1, PD-L2, CTLA4, CD4, CD8, CD20, pan-cytokeratin. In particular embodiments, PD-L1 is a biomarker associated with clinical responsiveness to treatment with pembrolizumab. In certain embodiments, the tumor is a lung tumor, e.g., a non squamous cell lung carcinoma, a head and neck tumor, e.g., a squamous cell carcinoma of the head and neck, a kidney tumor, or a melanoma, and the one or more detectable marker binds to or identifies programmed death ligand 1 (PD-L1), CD3, CD8 or forkhead box P3 (FoxP3).

In other embodiments, the tumor is a cancer listed in FIG. 19, and the one or more detectable marker binds to or identifies the associated antigen shown in FIG. 19.

In one embodiment, the tumor is a melanoma, and the one or more detectable marker binds to or identifies one or more of B-raf (BRAF), N-ras (NRAS), KIT, Guanine Nucleotide Binding Protein (G Protein), Alpha 11 (GNA11)/G Protein Subunit Alpha Q (GNAQ), cyclin dependent kinase-4 (CDK4), and mitogen-activated protein kinase (MEK) mutations and expression of one or more PD-L1, PD-1, CTLA-4, CD4, CD8, CD20, or pan-cytokeratin.

In one embodiment, the tumor is a colon cancer, and the one or more detectable marker binds to or identifies one or more of V-Ki-ras2 Kirsten rat sarcoma viral oncogene homolog (KRAS) mutation and epidermal growth factor receptor (EGFR), e.g., level of EGFR expression.

In one embodiment, the tumor is a prostate tumor, and the one or more detectable marker binds to or identifies androgen receptor (AR).

### BRIEF DESCRIPTION OF THE SEVERAL VIEWS OF THE DRAWINGS

FIG. 1 illustrates a perfusion chamber setup, according to embodiments.

FIGS. 2A-2B illustrate brain and spinal cord clearing, according to embodiments.

FIGS. 3A-3D illustrate operation of an electrophoresis device, according to embodiments.

FIGS. 4A-4J illustrate operation of an electrophoresis device, according to other embodiments.

FIGS. 5A-5B illustrate an imaging system, according to embodiments.

FIGS. 6A and 6B illustrates electronic control of the system of FIGS. 5A-5B, according to embodiments.

FIGS. 7A-7C illustrate image acquisition with the system of FIGS. 5A-5B, according to embodiments.

FIG. 8 illustrates the optical setup of the system of FIGS. 5A-5B, according to embodiments

FIG. 9 illustrates an imaging system with four detection paths, according to embodiments.

FIG. 10 illustrates a method of image acquisition and processing, according to embodiments.

FIGS. 11A-11B illustrate another imaging system, according to embodiments.

FIG. 12 illustrates a method of image acquisition and processing using the system of FIGS. 11A-11B, according to embodiments.

FIGS. 13A-13C show an MCF-7 frozen cell pellet that was HM embedded, cleared, and stainged with propidium iodide (PI) at: pre-clearing (FIG. 13A); 5 days post-clearing (FIG. 13B); and following PI staining (FIG. 13C; 1000 mm Z-stack, 10 mm step size).

FIGS. 14A-14D show mouse patient derived xenograft (PDX) at: pre-clearing (FIG. 14A); 7 days clearing (FIG. 14B); stained with anti-pan cytokeratin antibody (FIG. 14C); and stained with anti-CB11 HER2 antibody (FIG. 14D).

FIGS. 15A-15F show a human breast cancer excision biopsy specimen frozen tumor (FIG. 15A); after HM embedding (FIG. 15B); following 35 days clearing (FIG. 15C); stained with anti-HER2 affinity FITC (FIG. 15D); stained with anti-pan cytokeratin (FIG. 15E); and stained with anti-Ki67 primary antibody (FIG. 15F).

FIGS. 16A-16D show a human metastatic breast cancer lymph node as: frozen tissue (FIG. 16A); HM embedded (FIG. 16B); after passive clearing for 32 days (FIG. 16C); and following multiplex staining with Sytox blue, anti-pan cytokeratin antibody, and antiCD-31 antibody (FIG. 16D). Blood vessels, nuclei and tumor are indicated by arrows.

FIGS. 17A-D show a human lung adenocarcinoma, stage 1B tumor as: frozen tissue (FIG. 17A); HM embedded (FIG. 17B); following passive clearing for 32 days (FIG. 17C); and following multiplex staining with Sytox blue, anti-pan cytokeratin antibody, and anti-CD-31 antibody (FIG. 17D). Tumor, nuclei and blood vessels are indicated by arrows.

FIGS. 18A and 18B show hematoxylin and eosin (H&E) staining of HM embedded and cleared normal mouse kidney tissue at 10× (FIG. 18A) and 40× (FIG. 18B) magnification.

FIG. 19 provides a listing of different types of cancers and associated antigens, e.g., antigens that may be analyzed in tumor tissue samples according to methods of the present invention.

### DETAILED DESCRIPTION

In the following description, certain specific details are set forth in order to provide a thorough understanding of various embodiments of the invention. However, one skilled in the art will understand that the invention may be practiced without these details.

Definitions

Unless defined otherwise, all technical and scientific terms used herein have the same meaning as commonly understood by those of ordinary skill in the art to which the invention belongs. For the purposes of the present invention, the following terms are defined below.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A “decreased” or “reduced” or “lesser” amount is typically a “statistically significant” amount, and may include a decrease that is about 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times lower (e.g., 100, 500, 1000 times) an amount or level described herein. In particular embodiments, it indicates a decrease of at least 10%, at least 20%, at least 30%, at least 40%, at least 50%, at least 60%, at least 70%, at least 80%, or at least 90% (including all integers and decimal points in between, e.g., 15%, 26%, etc.) as compared to the reference amount.

Reference throughout this specification to “an embodiment” or “one embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times greater (e.g., 100, 500, 1000 times) (including all integers and decimal points in between and above 1, e.g., 2.1, 2.2, 2.3, 2.4, etc.) an amount or level described herein. In particular embodiments, it indicates an increase of at least 10%, at least 20%, at least 30%, at least 40%, at least 50%, at least 60%, at least 70%, at least 80%, at least 90%, at least 100%, at least 150%, at least 200%, at least 500%, or at least 1000% (including all integers and decimal points in between, e.g., 15%, 26%, etc.) as compared to the reference amount.

By “isolated” is meant material that is substantially or essentially free from components that normally accompany it in its native state. For example, an “isolated polynucleotide,” as used herein, includes a polynucleotide that has been purified from the sequences that flank it in its naturally-occurring state, e.g., a DNA fragment which has been removed from the sequences that are normally adjacent to the fragment. Alternatively, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The terms “antibody” or “antibodies” refers to immunoglobulin molecules or immunologically active portions of immunoglobulin molecules, i.e., molecules that contain an antigen binding site that specifically binds an antigen, and synthetic antibodies. The immunoglobulin molecules can be of any class (e.g., IgG, IgE, IgM, IgD or IgA) or subclass of immunoglobulin molecule. Antibodies include, but are not limited to, polyclonal, monoclonal, bispecific, synthetic, humanized and chimeric antibodies, single chain antibodies, Fab fragments and F(ab)₂ fragments, Fv or Fv′ portions, fragments produced by a Fab expression library, anti-idiotypic (anti-Id) antibodies, or epitope-binding fragments of any of the above. An antibody, or generally any molecule, “binds specifically” to an antigen (or other molecule) if the antibody binds preferentially to the antigen, and, e.g., has less than about 30%, 20%, 10%, 5% or 1% cross-reactivity with another molecule. In some embodiments, antibodies that cross react with multiple markers are used. For example, an antibody that cross reacts with related members of a cell surface protein family can be used to bind cells displaying various members of that family.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template.

By “obtained from” is meant that a sample such as, for example, a biological sample or tumor sample, is isolated from, or derived from, a particular source, such as a desired organism (e.g., subject) or a specific tissue within a desired organism. For example, a biological sample may be obtained from a subject. “Derived from” or “obtained from” can also refer to the source of a biological sample or tumor tissue sample.

The recitation “polynucleotide” or “nucleic acid” as used herein refers to polymeric forms of nucleotides, typically of at least 10 bases in length, either ribonucleotides or deoxynucleotides or a modified form of either type of nucleotide. The term includes single and double stranded forms of DNA and RNA. Polynucletoides include but are not limited to mRNA, RNA, cRNA, rRNA, cDNA, DNA and microRNA. Polynucleotides include non-coding RNA (ncRNA), which is a functional RNA molecule that is transcribed from DNA but not translated into proteins. Epigenetic related ncRNAs include miRNA, siRNA, piRNA and lncRNA. In general, ncRNAs function to regulate gene expression at the transcriptional and post-transcriptional level. Those ncRNAs that appear to be involved in epigenetic processes can be divided into two main groups; the short ncRNAs (<30 nts) and the long ncRNAs (>200 nts). The three major classes of short non-coding RNAs are microRNAs (miRNAs), short interfering RNAs (siRNAs), and piwi-interacting RNAs (piRNAs). Both major groups are shown to play a role inheterochromatin formation, histone modification, DNA methylation targeting, and gene silencing.

The terms “polypeptide” and “protein” are used interchangeably herein to refer to a polymer of amino acid residues and to variants and synthetic and naturally occurring analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers and naturally occurring chemical derivatives thereof.

A “subject,” as used herein, includes any animal that exhibits a symptom, or is at risk for exhibiting a symptom, which can be treated or diagnosed according to the invention. Also included are subjects for which it is desirable to analyze biological samples, e.g., tumor tissue samples, for diagnostic or other purposes. Suitable subjects (patients) include mammals (such as humans and non-human primates), laboratory animals (such as mouse, rat, rabbit, or guinea pig), farm animals, and domestic animals or pets (such as a cat or dog).

“Treatment” or “treating,” as used herein, includes any desirable effect on the symptoms or pathology of a disease or condition, e.g., a cancer, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Subjects in need thereof also include animal models of disease, e.g., animal models used in evaluating drug therapies. Exemplary markers of clinical improvement will be apparent to persons skilled in the art.

“Prevention” or “preventing,” as used herein, includes delaying or inhibiting the onset or progression of symptoms or pathology of a disease or condition, e.g., a cancer or tumor metastasis, and may include even minimal changes or improvements in one or more measurable markers of the disease or condition being treated. “Prevent” or “preventing” does not necessarily indicate complete prevention of the onset or progression of the disease or condition, or associated symptoms thereof.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R.I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005), Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, (2002, Cold Spring Harbor Laboratory, ISBN 0-87969-630-3).

Certain embodiments may employ conventional biology methods, software and systems for diagnostic purposes of the present invention. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

Certain embodiments may employ various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

### DETAILED DESCRIPTION

The present invention provides methods for the preparation and analysis of biological samples. In particular embodiments, the methods are advantageous for analyzing a large and/or intact biological sample or sample, such as a tumor tissue sample. These methods may be used to render tissue transparent and amenable to light microscopy techniques, thus permitting the in situ detection of biomarkers, cellular interactions, and tissue microenvironment across a 3-dimensional structure of the tissue. These methods permit 3-dimensional analysis of a biological sample, which allows analysis of various biological structures and staining patterns within the sample indicative or predictive of disease states, such as tumor presence, tumor growth, tumor metastasis, tumor prognosis, tumor development over time, and/or predicted or actual tumor response or non-response to treatment and development of resistance. The methods also allow for repeated analysis of a biological sample, e.g., through successive analysis using different probes. This permits the high-content, many-feature description of single cells in situ, as well as multi-cellular structures, using various types of probes and agents to analyze different disease markers or indicators. In addition, the detection of key biomarkers (e.g., protein, DNA and RNA) within and between cell types in the tumor microenvironment, including the immune system, in their native state allows for better tumor characterization and understanding of drug response and potential resistance. Effectively, it captures a much better snapshot of the inherent heterogeneity of tumors, without the loss of critical temporal-spatial information. Thus, the methods provide valuable information beyond that obtained by traditional pathological analysis of thin sections of tumor tissue or using multiple sections with molecular techniques that do not preserve the morphologic and spatial relationship of the tissue.

The methods of the present invention may be used for a variety of purposes, including but not limited to detecting and/or charactering disease tissue (e.g., tumor tissue), determining or predicting prognosis or metastasis of a tumor, monitoring a disease (e.g., a tumor) over time and/or in response to therapy, testing candidate therapeutic agents (e.g., antitumor agents), both in actual patients and in animal models of disease. In particular embodiments, the methods of the invention are useful in detecting a disease or condition (including pre-symptomatic early stage detecting), determining the prognosis, diagnosis, or theranosis of a disease or condition, or determining the stage or progression of a disease or condition. Characterizing a disease tissue sample can also include identifying appropriate treatments or treatment efficacy for specific diseases, conditions, disease stages and condition stages, predictions and likelihood analysis of disease progression, particularly disease recurrence, metastatic spread or disease relapse. Methods of the present invention may also be used to identify clinically distinct type or subtype of a condition or disease, such as a cancer or tumor. In further embodiments, methods of the present invention are practiced using a tissue sample that is a cell line or 3D cell line culture, which may be derived from a human or animal, and used, e.g., as a control or disease model.

The unique nucleic acid labeling features of methods of the present invention, including those that comprise CLARITY and hydrogel-based methods, are advantageous for detecting a variety of mutations associated with diseases such as tumora and cancers, including, e.g., gene mutations, including those in non-coding RNAs that do not express proteins. Non-coding RNAs comprise 98% of the mammalian transcriptome, and GWAS studies report preferential involvement of non-coding RNAs in a vast majority of human disease (as compared to protein coding genes. Even for the translated RNA population, antibodies used to detect resulting proteins will remain largely blind to certain splice variants, edited transcripts, sequence polymorphisms, and other transcriptional biomarkers. Nucleic acid labeling is also very well suited to multiplexing for each feature detection at the single-cell level in heterogeneous tumor samples.

### I. General Methods

Embodiments of the invention include methods for analyzing tissue samples, e.g., tumor tissue samples, which comprise: (i) processing the tissue sample to render it amenable to analysis by microscopy; and (ii) analyzing the processed tissue sample by microscopy. In particular embodiments, the methods further comprise: (iii) labeling the tissue sample, e.g., with a detectable label that specifically binds to a marker that may be useful in characterizing the tissue sample. The tissue sample may be labeled after it is processed, or, in certain situations, the tissue may be labeled before processing. In addition, the method may be performed using tissue samples obtained at one two or more different time point, e.g., to assess as disease's progression over time. Methods of the invention may be used for a variety of purposes, including, e.g., detecting the presence of a disease (e.g., cancer) in the tissue sample, predicting the prognosis of a disease, predicting or determining the responsiveness of a disease to a particular therapy, predicting or determining an appropriate therapy for the disease, and predicting or determining the development of resistance of the disease to a therapy.

In particular embodiments, one or more phenotypic or genotypic characteristics of the tissue sample are characterized, by analyzing a biological sample and determining the presence, level, amount, location ,or concentration of one or more biomarkers in the sample, e.g., proteins or nucleic acids. In embodiments, characterization includes determining whether the biomarkers in the sample are altered as compared to a reference, which can also be referred to a standard or a control. An alteration can include any measurable difference between the sample and the reference, including without limitation an absolute presence or absence, a quantitative level, a relative level compared to a reference, an elevated or increased level, a decreased level, overexpression, underexpression, differential expression, a mutation or other altered sequence, a modification (glycosylation, phosphorylation, epigenetic change) and the like. Methods may be used to determine a biosignature of a tissue sample, which may include specific pattern of biomarkers, e.g., patterns of biomarkers indicative of a phenotype that is desirable to detect, such as a disease phenotype. A biosignature can be used when characterizing a phenotype, such as a diagnosis, prognosis, theranosis, or prediction of responder/non-responder status. The biosignature can also be used to determine treatment efficacy, stage of a disease or condition, or progression of a disease or condition, or to monitor progression of a disease or condition or to monitor a subject's response to a treatment.

Biomarkers can be evaluated by comparing an amount of or level of biomarkers with a reference level or value. In certain embodiments, the reference value can be from the same subject from whom a sample is assessed, or the reference value can be from a representative population of samples (e.g., samples from normal subjects not exhibiting a symptom of disease; or samples from subjects with the same disease, e.g., breast cancer, that have a different set of biomarkers of prognosis or prediction). Therefore, a reference value can provide a threshold measurement which is compared to a subject sample's readout for a biosignature assayed in a given sample. Such reference values may be set according to data pooled from groups of sample corresponding to a particular cohort, including but not limited to age (e.g., newborns, infants, adolescents, young, middle-aged adults, seniors and adults of varied ages), racial/ethnic groups, normal versus diseased subjects, smoker v. non-smoker, subject receiving therapy versus untreated subject, different time points of treatment for a particular individual or group of subjects similarly diagnosed or treated or combinations thereof. Furthermore, by determining a biosignature at different timepoints of treatment for a particular individual, the individual's response to the treatment or progression of a disease or condition for which the individual is being treated for, can be monitored.

A reference value may be based on samples assessed from the same subject so to provide individualized tracking. In some embodiments, frequent testing of a biosignature in samples from a subject provides better comparisons to the reference values previously established for that subject. Such time course measurements are used to allow a physician to more accurately assess the subject's disease stage or progression, or prediction of treatment response/non-response, and therefore provide a more informed decision for treatment. In some cases, the variance of a biosignature is reduced when comparing a subject's own biosignature over time, thus allowing an individualized threshold to be defined for the subject, e.g., a threshold at which a diagnosis is made.

Reference values can be established for unaffected individuals without a particular phenotype by determining the biosignature of interest in an unaffected individual. For example, a reference value for a reference population can be used as a baseline for detection of one or more biomarkers in a test subject. If a sample from a subject has a level or value that is similar to the reference, the subject can be identified to not have the disease, or of having a low likelihood of developing a disease.

Alternatively, reference values or levels can be established for individuals with a particular phenotype by determining the amount of one or more biomarkers in an individual with the phenotype. In addition, an index of values can be generated for a particular phenotype. For example, different disease stages can have different values, such as obtained from individuals with the different disease stages. A subject's value can be compared to the index and a diagnosis or prognosis of the disease can be determined, such as the disease stage or progression wherein the subject's levels most closely correlate with the index. In other embodiments, an index of values is generated for therapeutic efficacies. For example, the level of biomarkers of individuals with a particular disease can be generated and noted what treatments were effective for the individual. The levels can be used to generate values of which is a subject's value is compared, and a treatment or therapy can be selected for the individual, e.g., by predicting from the levels whether the subject is likely to be a responder or non-responder for a treatment.

In some embodiments, a reference value is determined for individuals unaffected with a particular cancer, by isolating or detecting biomarkers with an antigen that specifically targets biomarkers for the particular cancer. As a non-limiting example, individuals with varying stages of cancer can be surveyed using the same techniques described for unaffected individuals and the levels of biomarkers for each group can be determined. In some embodiments, the levels are defined as means +/− standard deviations from at least two separate experiments, performed in at least duplicate or triplicate. Comparisons between these groups can be made using statistical tests to determine statistical significance of distinguishing biomarkers observed. In some embodiments, statistical significance is determined using a parametric statistical test. The parametric statistical test can comprise, without limitation, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than 0.05, 0.01, 0.005, 0.001, 0.0005, or 0.0001. The p-values can also be corrected for multiple comparisons, e.g., using a Bonferroni correction, a modification thereof, or other technique known to those in the art, e.g., the Hochberg correction, Holm-Bonferroni correction, \check{S}idak correction, Dunnett's correction or Tukey's multiple comparisons. In some embodiments, an ANOVA is followed by Tukey's correction for post-test comparing of the biomarkers from each population.

Reference values can also be established for disease recurrence monitoring, for therapeutic response monitoring, or for predicting responder/non-responder status, in particular embodiments.

Methods of the present invention may be practiced on a variety of different tissues types and tissues associated with a variety of different diseases, including but not limited to the various tumors and cancers described herein. Illustrative methods for processing tissue samples, labeling tissue samples, and analyzing tissue samples by microscopy are described herein. Any of the various processing methods, labeling methods, and microscopy methods may be combined in any manner to practice the present invention.

In particular embodiments, methods of the invention involve analyzing or detecting two or more different characteristics or components of the biological sample. These may be analyzed simultaneously, sequentially, or in an overlapping manner, either physically or temporally. Two or more components (e.g., presence or expression of specific cells, polypeptides, or polynucleotides) may be analyzed at the same time, e.g., using two or more differently labeled detectable probes, which distinguish between the two or more components. One advantage of the present invention is that a processed biological sample may be analyzed using by one method, e.g., using particular probes, and those probes may then be removed or washed from the biological sample, which may then be analyzed using a second method, e.g., using different probes. In particular embodiments, the method comprises analyzing or detecting a first characteristic or component, and then subsequently analyzing or detecting a second characteristic or component. In certain embodiments, the two or more characteristics or components may be analyzed using the same type of probe (e.g., antibody or nucleic acid probe), whereas in other embodiments, they are analyzed using different types of probes.

In particular embodiments, two or more tumor tissue samples are obtained from a subject at different time points and analyzed according to the present methods, e.g., to determine the effectiveness of treatment with an anti-tumor drug. These may be obtained from the same or different tumors or sites within the subject. For example, in certain embodiments, a first tumor tissue sample is obtained as excisional therapeutic removal of a primary tumor at a first time point, and a second tumor tissue sample is obtained at a later, second time point from a different tumor in the subject, which may be, e.g., a metastasis of the primary tumor or a secondary tumor. In particular embodiments, the second tumor tissue sample comprises the same type of tumor cells as the first tumor tissue sample, and is optionally derived from the first tumor as a metastasis.

Antibodies and fragments thereof, and other binding agents, e.g., peptides and aptamers, that specifically bind to the proteins and nucleic acids described herein are known and available in the art, or may be readily produced, e.g., based on the nucleic acid sequence of the target gene or mRNA. In particular embodiments, the antibody or other binding agent is detectably labeled, or is bound by a detectably labeled secondary binding agent. A variety of detectable molecules may be used, such as a radioisotopes, fluorochromes, dyes, enzymes, nanoparticles, chemiluminescent markers, biotin, quantum dots, or other monomer known in the art that can be detected directly (e.g., by light emission) or indirectly (e.g., by binding of a fluorescently-labeled antibody).

The use of detectable labels is well known in the art. Methods for conjugating polypeptides and detectable labels are well known in the art, as are methods for imaging using detectable labels. Examples of detectable labels include but are not limited to radionucleotides, enzymes, coenzymes, fluorescers, chemiluminescers, chromogens, enzyme substrates or co-factors, enzyme inhibitors, prosthetic group complexes, free radicals, particles, dyes, and the like. Several radioisotopes can be used as detectable molecules for labeling peptides including, for example, 32P, 33P, 35S, 3H, and 125I. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin, coumarin, Alexa488, Oregon green 488, rhodamine green, Alexa 532, Cy3, Bodipy 588/586, Alexa586, TAMRA, Rox, Alexa 594, Texas red, Bodipy 630/650, Cy5, Alexa647, IR Dye 680, IR Dye 680, IR Dye 700 DX, Cy5.5, Alexa 750, IR Dye 800CW, IR Dye 800, Atto 532, Atto 465; an example of a luminescent material is luminol; examples of bioluminescent materials include luciferase, luciferin, and aequorin; and examples of suitable radioactive material include 125 I, 131 I, 35 S, or 3 H. In some embodiments, the detectable labels include fluorescent proteins. Suitable fluorescent proteins include Tag13FP, mTagRFP2, Azurite, EBFP2, mKalama1, Sirius, Sapphire, T-Sapphire, ECFP, Cerulean, SCFP3A, mTurquoise, mTurquoise2, monomeric Midoriishi-cyan, TagCFP, mTFP1, GFP, EGFP, Emeral, Superfolder GFP, monomeric Azami Green, TagGFP2, mUKG, mWasabi, Clover, mNeonGreen, EYFP, YFP, Citrine, Venus, SYFP2, TagYFP, monomeric Kusabira Orange, MKOK, mKO2, mOrange, mOrange2, mRaspberry, mCherry, mStrawberry, mTangerine, tdTomato, TagRFP, TagRFP1, mApple, mRuby, mRuby2, TagRFP675, IFP1,4, iFRP, mKeima Red, LSS-mKate1, LSS-mKate2, mBeRFP, PA-GFP, PAmCherryl, PATagRFP, Kaede green, Kaede red, KikGR1 green, KikGR1 red, PS-CFP2, mEos2 green, mEos2 red, mEos3.2 green, mEos3.2 red. PSmOrange. In some embodiments of the present invention, detectable labels also include quenchers suitable for fluorescence resonance energy transfer (FRET) pairings. Examples of suitable quenchers include Dabcyl, BHQ1, BHQ2, BHQ3, CY5Q, CY7Q, Iowablack FQ, Iowablack RQ, IR Dye QC-1, QSY35, QSKY7, QXL570, QXL610, QXL680.

### II. Preparation of Biological Samples

Methods of the invention include preparing biological samples for analysis, e.g., microscopic image analysis.

A. Biological Samples

Biological samples include tissue or organ samples obtained from a subject, e.g., a mammal. In certain embodiments, the subject is diagnosed with a disease or disorder, such as a cancerous tumor, or considered at risk of having or developing the disease or disorder. Biological samples may also be obtained from healthy donors, e.g., as normal control samples. In certain embodiments, both a disease tissue (e.g., a tumor tissue) sample and a normal sample are obtained from the same subject. In particular embodiments, the biological sample is obtained from a patient, e.g., a mammal such as a human. In other embodiments, a biological sample is obtained from an animal model of disease. Various animal models of disease are known and available in the art. Particular animal models of cancer include but are not limited to xenograft, syngeneic, and PDx models, e.g., in mice or rats. Animal models may also include human cells, cancerous or otherwise, introduced into animal models wherein tumor properties, progress, and treatment may be assessed. In vitro 3D tissue arrangements, organoids, and stem or iPS-cell-derived 3D compositions are also relevant models, and these may comprise human or other animal cells, for example.

Biological tissue samples suitable for use with the methods and systems described herein generally include any type of tissue samples collected from living or dead subjects, such as, for example, tumor tissue and autopsy samples. Tissue samples may be collected and processed using the methods and systems described herein and subjected to microscopic analysis immediately following processing, or may be preserved and subjected to microscopic analysis at a future time, e.g., after storage for an extended period of time. In some embodiments, the methods described herein may be used to preserve tissue samples in a stable, accessible and fully intact form for future analysis. For example, tissue samples, such as, e.g., human tumor tissue samples, may be processed as described herein and cleared to remove a plurality of cellular components, such as, e.g., lipids, and then stored for future analysis. In some embodiments, the methods and systems described herein may be used to analyze a fresh biological sample. In some embodiments, the methods and systems described herein may be used to analyze a previously-preserved (e.g., previously fixed) or stored biological sample (e.g., tissue sample). For example, in some embodiments a previously-preserved tissue sample that has not been subjected to a sample preparation process described herein may be processed and analyzed as described herein. In particular methods, a tissue sample is frozen prior to being processed as described herein.

In certain embodiments, biological samples are tumor tissue samples. Tumor samples may contain only tumor cells, or they may contain both tumor cells and non-tumor cells. In particular embodiments, a biological sample comprises only non-tumor cells. In particular embodiments, the tumor is a solid tumor. In particular embodiments, the biological sample is obtained from or comprises an adrenal cortical cancer, anal cancer, aplastic anemia, bileduct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal or squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment, is a biological sample obtained from a subject diagnosed with or suspected of having any of these tumors or cancers.

The methods of the invention can be used to characterize a cancer or metastasis thereof, including without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer under analysis may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

Biological samples may be obtained from a subject by any means known and available in the art. In particular embodiments, a biological sample, e.g., a tumor tissue sample, is obtained from a subject by fine needle aspiration, core needle biopsy, stereotactic core needle biopsy, vacuum-assisted core biopsy, or surgical biopsy. In particular embodiments, the surgical biopsy is an incisional biopsy, which removes only part of the suspicious area. In other embodiments, the surgical biopsy is an excisional biopsy, which removes the entire diseased tissue (e.g., tumor) or abnormal area. In particular embodiments, an excisional tumor tissue sample is obtained from a tumor that has been excised with the intent to “cure” a patient in the case of early stage disease, wherein in other embodiments, the excisional tumor tissue sample is obtained from an excised bulk of primary tumor in later stage disease. Tumor tissue samples may include primary tumor tissue, metastastic tumor tissue and/or secondary tumor tissue. Tumor tissue samples may be cell cultures, e.g., cultures of tumor-derived cell lines. In certain embodiments, a biological sample is a cell line, e.g., a cell pellet of a cultured cell line, such as a tumor cell line. In particular embodiments, the cell line or cell pellet is frozen or was previously frozen. Such cell lines and pellets are useful, e.g., as positive or negative controls for imaging with various reagents. Tumor tissue samples may also be xenograft tumors, e.g., tumors obtained from animals administered with tumor cells, e.g., a human tumor cell line. The present invention includes hydrogel-embedded cell pellets, including cell pellets processed as described herein, e.g., to serve as controls. In particular embodiments, the cell pellets were fixed in the presence of hydrogel subunits and then the hydrogen subunits were polymerized to form a hydrogel-embedded cell pellet. In certain embodiments, the hydrogel-embedded cell pellel was also cleared, e.g., as described herein. In certain embodiments, the cell pellets are frozen. In certain embodiments, a first tumor tissue sample from a subject is a primary tumor tissue sample obtained during an initial surgery intended to remove the entire tumor, and a second tumor tissue sample is obtained from the same subject is a metastatic tumor tissue sample or a secondary tumor tissue sample obtained during a later surgery.

Biological samples, e.g., tumor tissue samples, may be obtained surgically or using a laparoscope. A biological sample may be a tissue sample obtained from any part of the body to examine it for disease or injury, e.g., presence of cancer tissue or cells, or the extent or characteristics thereof. In particular embodiments, the biological sample comprises abdominal tissue, bone, bone marrow, breast tissue, endometrial tissue, kidney tissue, liver tissue, lung or chest tissue, lymph node, nerve tissue, skin, testicular tissue, head or neck tissue, or thyroid tissue. In certain embodiments, the tissue is obtained from brain, breast, skin, bone, joint, skeletal muscle, smooth muscle, red bone marrow, thymus, lymphatic vessel, thoracic duct, spleen, lymph node, nasal cavity, pharynx, larynx, trachea, bronchus, lung, oral cavity, esophagus, liver, stomach, small intestine, large intestine, rectum, anus, spinal cord, nerve, pineal gland, pituitary gland, thyroid gland, thymus, adrenal gland, pancreas, ovary, testis, heart, blood vessel, kidney, uterus, urinary bladder, urethra, prostate gland, penis, prostate, testis, scrotum, ductus deferens, mammary glands, ovary, uterus, vagina, or uterine tube.

In particular embodiments, a biological sample has a size greater than sections typically examined by traditional pathology thin section or immunohistochemical analysis, which are typically in the range of 4-10 microns thick. In certain embodiments, a biological sample is greater than 20 microns, greater than 50 microns, greater than 100 microns, greater than 200 microns, greater than 500 microns, greater than 1 mm, greater than 2 mm, greater than 5 mm, greater than 10 mm or greater than 20 mm in thickness and/or length. In particular embodiments, the biological sample has a length and/or a thickness between 20 micros and 20 mm, between 20 microns and 10 mm, or between 50 microns and 1 mm. In certain embodiments, a biological sample is a cubic sample with each side greater than 10 microns, greater than 20 microns, greater than 50 microns, greater than 100 microns, greater than 200 microns, greater than 500 microns, greater than 1 mm, greater than 2 mm, greater than 5 mm, greater than 10 mm, or greater than 2 mm in thickness and/or length. In some embodiments, a biological sample is thinner, e.g., from about 4-10 or 4-20 microns in thickness.

B. Methods of Preparing Biological Samples

Methods of the invention comprise preparing a biological sample (e.g., a tumor tissue sample) for microscopic analysis. Microscopic analysis may include, without limitation, optical microscopy (e.g., bright field, oblique illumination, dark field, phase contrast, differential interference contrast, interference reflection, epifluorescence, confocal, two-photon, temporal focusing, light sheet with or without methods for extending the detection depth of field, etc., microscopy), laser microscopy, electron microscopy, and scanning probe microscopy. These methods find many uses, for example in medicine and research, e.g., to diagnose or monitor disease (e.g., cancerous tumors), to study healthy or diseased tissues (e.g., cancerous tumors), to screen candidate agents (e.g., anti-tumor agents) for toxicity and efficacy in disease modification. Also provided are reagents, devices, kits and systems thereof that find use in practicing the subject methods.

In some embodiments, a biological sample is prepared as generally described in PCT Application Publication No. WO2014025392 (“the '392 publication”) titled “METHODS AND COMPOSITIONS FOR PREPARING BIOLOGICAL SPECIMENS FOR MICROSCOPIC ANALYSIS”, filed Mar. 13, 2013, the entire disclosure of which is incorporated herein by reference. In particular embodiments, preparing a biological sample comprises fixing or preserving one or more structural features or components of the biological sample, such as, e.g., cells, blood vessels, and/or or extracellular matrix of a tumor tissue sample, and also removing one or more components of the biological sample, such as, e.g., lipids. These methods may be employed to preserve the 3-dimensional architecture or structure of the tissue or one or more components thereof, while removing one or more components that interfere with microscopic analysis of the biological sample.

In particular embodiments, a biological sample (e.g., a tumor tissue sample) is fixed in the presence of hydrogel subunits, e.g., hydrogel monomers. In some embodiments, fixing includes exposing the sample, e.g., cells of the sample, to a fixation agent such that the cellular components become crosslinked to one another. The hydrogel/hydrogel network can include any suitable network of polymer chains that are water-insoluble, sometimes found as a colloidal gel in which water is the dispersion medium. In other words, hydrogels can belong to a class of polymeric materials that can absorb large amounts of water without dissolving. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published U.S. patent application 20100055733, herein specifically incorporated by reference and as detailed below. Examples of suitable hydrogels include acrylamide. Concentrations of hydrogel subunits and modifiers that provide desired hydrogel characteristics may be readily determined by methods in the art. Hydrogel subunits or hydrogel precursors can encompass hydrophilic monomers, prepolymers, or polymers that can be crosslinked, or “polymerized”, to form a three-dimensional (3D) hydrogel network. Without being bound by scientific theory, it is believed that this fixation of the biological sample in the presence of hydrogel subunits crosslinks the components of the sample to the hydrogel subunits, thereby securing molecular components in place, preserving the tissue architecture and cell morphology.

In some embodiments, the preparation of the biological sample may include swelling or expansion of the tissue or gel-tissue composite as shown in Tomer, R. T et al., Nature Protocols, Vol. 9, No. 71682-1697 (June 2014), for higher resolution access or better permeability to probes and labels, contraction of the tissue or gel-tissue composite for better access by objectives with a given working distance or reducing the dataset size, digestion or disassembly of components such as proteins separate from bulk removal of components (e.g., as described in U.S. Patent Application Publication No. 20140030192, which is hereby incorporated by reference in its entirety), and/or any other method designed to allow appropriate access to biological information.

Any convenient fixation agent, or “fixative,” may be used in the fixative/hydrogel composition to fix the biological sample in the presence of hydrogel subunits such as, for example, formaldehyde, paraformaldehyde, glutaraldehyde or any other aldehyde, acetone, ethanol, methanol, aminoalcohols, etc.

In some embodiments, the fixative can be diluted in a buffer, e.g., saline, phosphate buffer (PB), phosphate buffered saline (PBS), citric acid buffer, potassium phosphate buffer, etc., usually at a concentration of about 1-10%, e.g. 1%, 2%, 3%, 4%, 5%, 6%, 7%, 8%, or 10%, for example, 4% paraformaldehyde/0.1 M phosphate buffer; 2% paraformaldehyde/0.2% picric acid/0.1 M phosphate buffer; 4% paraformaldehyde/0.2% periodate/1.2% lysine in 0.1 M phosphate buffer; 4% paraformaldehyde/0.05% glutaraldehyde in phosphate buffer; etc. The type of fixative used and the duration of exposure to the fixative can depend on the sensitivity of the molecules of interest in the biological sample to denaturation by the fixative, and can be readily determined using conventional histochemical or immunohistochemical techniques, for example as described in Buchwalow and Bocker. Immunohistochemistry: Basics and Methods. Springer-Verlag Berlin Heidelberg 2010.

The hydrogel composition, e.g., fixative/hydrogel solution, may include any convenient hydrogel subunits/monomers, such as, but not limited to, poly(ethylene glycol) and derivatives thereof (e.g., PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. In some embodiments, the hydrogel subunits may be modified to add specific properties to the hydrogel; for example, peptide sequences can be incorporated to induce degradation (see, e.g., West and Hubbell, 1999, Macromolecules, 32:241) or to modify cell adhesion (see, e.g. Hem and Hubbell, 1998, J. Biomed. Mater. Res., 39:266). Agents such as hydrophilic nanoparticles (e.g., poly-lactic acid (PLA), poly-glycolic acid (PLG), poly(lactic-co-glycolic acid) (PLGA), polystyrene, poly(dimethylsiloxane) (PDMS), etc.) may be used to improve the permeabilityof the hydrogel while maintaining patternability (see, e.g., U.S. patent application Ser. No. 13/065,030; Lee W. et al. 2010 Proc. Natl. Acad. Sci. 107, 20709-20714). Materials such as block copolymers of PEG, degradable PEO, poly(lactic acid) (PLA), and other similar materials can be used to add specific properties to the hydrogels (see, e.g., Huh and Bae, 1999, Polymer, 40:6147). Crosslinkers (e.g., bis-acrylamide, diazirine, etc.) and initiatiors (e.g., azobisisobutyronitrile (AIBN), riboflavin, L-arginine, etc.) may be included to promote covalent bonding between interacting macromolecules in later polymerization steps.

In some embodiments, the concentration and molecular weight of the hydrogel subunit(s) and modifying agents will depend on the selected polymer and the desired characteristics, e.g., pore size, swelling properties, conductivity, elasticity/stiffness (Young's modulus), biodegradability index, etc., of the hydrogel network into which they will be polymerized. For example, it may be desirable for the hydrogel to comprise pores of sufficient size to allow the passage of macromolecules, e.g., proteins, nucleic acids, or small molecules as described in greater detail below, into the sample. Pore size generally decreases with increasing concentration of hydrogel subunits and generally increases with an increasing ratio of hydrogel subunits to crosslinker, and will prepare a fixative/hydrogel composition that comprises a concentration of hydrogel subunits that allows the passage of such macromolecules. As another example, it may be desirable for the hydrogel to have a particular stiffness, e.g., to provide stability in handling the embedded sample, e.g., a Young's Modulus of about 2-70 kN/m², for example, about 2 kN/m², about 4 kN/m², about 7 kN/m², about 10 kN/m², about 15 kN/m², about 20 kN/m², about 40 kN/m², not more than about 70 kN/m², including all values and sub ranges in between.

The elasticity of a hydrogel network may be influenced by a variety of factors, including the branching of the polymer, the concentration of hydrogel subunits, and the degree of cross-linking, and the fixative/hydrogel composition is prepared that comprises a concentration of hydrogel subunits to provide such desired elasticity. Thus, for example, the fixative/hydrogel composition may include an acrylamide monomer at a concentration of from about 1% w/v to about 20% w/v, e.g., about 2% to about 15%, about 3% to about 10%, about 4% to about 8%, including all values and sub ranges in between; and a concentration of bis-acrylamide crosslinker in the range of about 0.01% to about 0.075%, e.g., 0.01%, 0.02%, 0.025%, 0.03%, 0.04%, 0.05%, 0.06%, or 0.075%, including all values and sub ranges in between; or, for example, the fixative/hydrogel composition may include PEG prepolymers having a molecular weight ranging from at least about 2.5K to about 50K, e.g., 2.5K or more, 3.5K or more, 5K or more, 7.5K or more, 10K or more, 15K or more, 20K or more, not more than about 50K, including all values and sub ranges in between, at a concentration in a range from about 1% w/w to about 50% w/w, e.g., 1% or more, 5% or more, 7.5% or more, 10% or more, 15% or more, 20% or more, 30% or more, 40% or more, not more than about 50%, including all values and sub ranges in between. Concentrations of hydrogel subunits and modifiers that provide desired hydrogel characteristics may be readily determined by suitable methods or as described in PCT Publication No. WO2014/025392, which is incorporate herein in its entirety. In certain embodiments, the hydrogel monomer solution includes a cross-linker, such as, e.g., a bis-acrylamide. The hydrogel monomer solution may further comprise an initiator, such as, e.g., VA-044 Initiator. The hydrogel monomer solution may further comprise paraformaldehyde. One illustrative hydrogel monomer fixing solution is described in Example 1.

The fixative/hydrogel solution may be delivered to the sample by any convenient method, e.g., perfusion, injection, instillation, absorption, application, immersion/submersion, etc. The sample will typically be fixed in the presence of the hydrogel for 15 minutes or more, for example, for 30 minutes or more, 1 hour or more, 2 hours or more, 4 hours or more, 6 hours or more, 12 hours or more, in some instances, for 16 hours or more, 20 hours or more, 24 hours or more, 48 hours or more, or 72 hours or more, including all values and sub ranges in between. In certain embodiments, the sample is embedded in the hydrogel for 24-72 hours.

In some embodiments, following fixation of the sample, the hydrogel subunits are polymerized, i.e., covalently or physically crosslinked, to form a hydrogel network. Polymerization may be by any method including, but not limited to, thermal crosslinking, chemical crosslinking, physical crosslinking, ionic crosslinking, photo-crosslinking, irradiative crosslinking (e.g., x-ray, electron beam), and the like, and may be selected based on the type of hydrogel used. For example, mixing of an un-polymerized or partially polymerized resin with specific crosslinking chemicals results in a chemical reaction that forms cross-links. Crosslinking can be induced in materials that are normally thermoplastic through exposure to a radiation source, such as electron beam exposure, gamma-radiation, or UV light; for example, electron beam processing is used to polymerize the C type of crosslinked polyethylene. Other types of crosslinked polyethylene are made by addition of peroxide during extruding (type A) or by addition of a cross-linking agent (e.g. vinylsilane) and a catalyst during extruding and then performing a post-extrusion curing. Many polymers undergo oxidative cross-linking, typically when exposed to atmospheric oxygen. In some instances, the reaction is more rapid than desired and thus polymerization reactions may involve the use of an antioxidant to slow the formation of oxidative cross-links. In other instances, e.g., when more rapid formation of cross-links by oxidation is desirable, an oxidizer such as hydrogen peroxide may be used to speed up the process. An azo iniator such as VA-044 Initiator may be included. The length of time for polymerization will depend on the type of hydrogel subunits used and the chosen polymerization method, but will typically be about 15 minutes to about 48 hours, for example, 15 minutes or more, 1 hour or more, 2 hours or more, 3 hours or more, 4 hours or more, 6 hours or more, 12 hours or more, 16 hours or more, 24 hours or more, or in some instances, 48 hours, including all values and sub ranges in between. In particular embodiments, the sample is polymerized until the hydrogel monomer solution is solidified.

In some embodiments, a sample is fixed and/or cross-linked via the SWITCH method described, e.g., in Murray E. et al., Cell 163, 1500-1514 (2015). SWITCH (system-wide control of interaction time and kinetics of chemicals) controls a range of chemical reactions in tissue processing via a set of buffers: a SWITCH-on buffer that facilitates checmic reactions between exogenous chemicals and endogenous biomolecules, and a SWITCH-OFF buffer that suppresses the reactions.

In certain embodiments, a sample is processed (e.g., fixed and cross-linked) and imaged as described in Ku, T. et al., Nature Biotechnology, published online Jul. 25, 2016. The method described therein, magnified analysis of the proteome (MAP) linearly expands tissue samples four-fold while preserving their overall architecture and three-dimensional proteome organization.

Once polymerized, the hydrogel-embedded (i.e., hydrogel-hybridized) biological sample may be cleared, i.e., one or more tissue components removed. Clearing can include ensuring that the sample is made substantially permeable to light, i.e., transparent. In other words, about 70% or more of the visual (i.e., white) light, ultraviolet light or infrared light that is used to illuminate the sample will pass through the sample and illuminate only selected cellular components therein, e.g., 75% or more of the light, 80% or more of the light, 85% or more of the light, in some instances, 90% or more of the light, 95% or more of the light, 98% or more of the light, e.g. 100%, including all values and sub ranges in between, of the light will pass through the sample. This change in the optical properties of the sample provides for the visualization of cellular and subcellular structures internal to the tissue.

Any treatment that forces one or more cellular components, e.g., lipids, from the sample, that draws cellular components, e.g., lipids, from a sample, or that causes cellular components, e.g., lipids, to break down, i.e., dissolve, within a sample may be used to clear the sample, including, without limitation, exposure to organic solvents such as xylenes, ethanol or methanol, exposure to detergents such as saponin, Triton X-100 and Tween-20, exposure to ionic surfactants, e.g., sodium dodecyl sulfate (SDS), electrophoresis (e.g., using an electrophoresis device as described herein), hydrodynamic pressure, ultrasonic vibration, solute contrasts, microwave radiation, vascular circulation, and/or the like. In some instances, clearing is performed using a solvent that does not quench fluorescent proteins. Examples of organic solvents that are known to quench fluorescent proteins include tetrahydrofuran, hexane, benzylalcohol/benzylbenzoate (BABB), and dibenzyl ether. Accordingly, in order to preserve the fluorescence of various proteins, in some embodiments clearing is conducted using solvents other than those listed above, e.g., is conducted using non-organic solvents.

In some instances, clearing is conducted using an ionic surfactant, e.g., SDS, in order to expedite the clearing process by actively transporting charged ionic micelles out of the sample that is being cleared. Clearing may be performed in any convenient buffer that is compatible with the selected clearance method, e.g., saline, phosphate buffer, phosphate buffered saline (PBS), sodium borate buffer, boric acid buffer, citric acid buffer, etc., as known in the art, and will typically take about 1-10 days per centimeter thickness of sample, i.e., usually about 1 day, in some instances 2 days, sometimes 5 days, and typically no more than 10 days per cubic centimeter. In certain emboidiments, clearing may take between two days and two or three months. In some embodiments, a clearing solution comprises a buffer, such as a borate buffer, and SDS. One particular clearing solution comprising 200 mM borate buffer, pH 8.5 and 8% SDS is described in Example 1. In some embodiments, optimal clearing time may be readily determined by visual inspection of the sample for clarity.

In certain embodiments, a tissue is cleared via the avtive clarity technique-pressure related efficient and stable transfer of macromoleculard into organs (ACT-PRESTO) procedure described in Lee, E. et al, Sci. Rep. 6 18631 (2016). This process is a modified version of CLARITY that employs a two-step fixation protocol: paraformaldehyde (PFA) fixation followed by acrylamide fusion without bis-acrylamide, prior to clearing via active clarity technique (ACT). Clearing occurs using a modified ETC chamber system with a platinum plate to generate a dense regular current in the ETC chamber.

After clearing, a sample will generally be substantially free of lipids. In some embodiments, the original amount of lipid present in the sample before clearing can be reduced by approximately 70% or more, such as by 75% or more, such as by 80% or more, such as by 85% or more, such as by 90% or more, such as by 95% or more, such as by 99% or more, such as by 100%, including all values and sub ranges in between.

In some instances, no further manipulation of the sample will be necessary for microscopic analysis. For example, the sample may comprise structures or biomolecules that can be directly visualized by microscopy. The structures or biomolecules can generally include, but are not limited to, cells, vasculature, extracellular matrix, lymphatic vessels, proteins, lipids, steroids, nucleic acids, etc. within a tissue or cell.

Additionally, or alternatively, in some embodiments, it may be desirable to contact the cells and intracellular structures of the sample with one or more macromolecules prior to microscopic analysis, as described in further detail herein. For example, macromolecules may be provided that promote the visualization of particular tissue components or structures, or cellular biomolecules, e.g., vasculature, extracellular matrix, cells (e.g., immune cells or stem cells), lymphatic vessels, proteins, lipids, steroids, nucleic acids, etc. and sub-cellular structures. In some embodiments, the macromolecules are diagnostic. In some embodiments, the macromolecules are prognostic. In some embodiments, the macromolecules are predictive of responsiveness to a therapy. In certain embodiments, the macromolecules are detectable probes that bind target tissue structures or cellular components. In some embodiments, the macromolecules are candidate agents in a screen, e.g., a screen for agents that will aid in the diagnosis and/or prognosis of disease, in the treatment of a disease, and the like.

For example, samples may be contacted with nucleic acid stains like DAPI and Hoechst, which bind the minor groove of DNA, thus labeling the nuclei of cells. Drugs or toxins that bind specific cellular structures and have been derivatized with a fluorescent reporter may be employed, e.g., fluorescently labelled-phalloidin, which is used to stain actin fibers in mammalian cells. There are many fluorescent reported molecules, called fluorophores or fluorochromes such as fluorescein, Alexa Fluors or DyLight 488, which can be chemically linked to molecules which bind the target biomolecules of interest within the sample. In certain embodiments, samples are contacted with agents, e.g., antibodies that bind various keratins.

As another example, the sample may be contacted with one or more polypeptides, e.g. antibodies, labeled peptides, and the like, that are specific for and will bind to particular cells or cellular biomolecules for either direct or indirect labeling by color or immunofluorescence (i.e. probes). Immunofluorescence can include any suitable technique that uses the highly specific binding of an antibody to its antigen or binding partner in order to label specific proteins or other molecules within the cell. A sample is treated with a primary antibody specific for the biomolecule of interest. A fluorophore can be directly conjugated to the primary antibody or peptide.

In particular embodiments, the antibodies bind to a protein associated with tumors or cancer. In certain embodiments, antibodies used to stain samples, e.g., tumor samples, are any of the antibodies listed in Table 3.

In some embodiments, a secondary antibody, conjugated to a detection moiety or fluorophore, which binds specifically to the first antibody can be used. See, for example, Buchwalow and Bocker. Immunohistochemistry: Basics and Methods, Springer-Verlag, Berlin Heidelberg 2010, and Hayat, M.A. Microscopy, Immunohistochemistry, and Antigen Retrieval Methods for Light and Electron Microscopy. Kluwar Academic Publishers, New York 2002, for examples of protocols that may be followed. Peptides that are specific for a target cellular biomolecule and that are conjugated to a fluorophore or other detection moiety may also be employed.

In particular embodiments, a probe such as a detectable probe is a fluorescent molecule or protein. Green fluorescent protein (green FP, or GFP) refers to a polypeptide that has a peak in the emission spectrum at 510 nm or about 510 nm. A variety of FPs that emit at various wavelengths are known in the art. FPs of interest include, but are not limited to, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein. As used herein, Aequorea GFP refers to GFPs from the genus Aequorea and to mutants or variants thereof. Such variants and GFPs from other species, such as Anthozoa reef coral, Anemonia sea anemone, Renilla sea pansy, Galaxea coral, Acropora brown coral, Trachyphyllia and Pectimidae stony coral and other species are well known and are available and known to those of skill in the art. Example GFP variants include, but are not limited to, BFP, CFP, YFP and OFP. Examples of florescent proteins and their variants include GFP proteins, such as Emerald (Invitrogen, Carlsbad, Calif), EGFP (Clontech, Palo Alto, Calif), Azami-Green (MBL International, Woburn, Mass.), Kaede (MBL International, Woburn, Mass.), ZsGreenl (Clontech, Palo Alto, Calif.) and CopGFP (Evrogen/Axxora, LLC, San Diego, Calif.); CFP proteins, such as Cerulean (Rizzo, Nat Biotechnol. 22(4):445-9 (2004)), mCFP (Wang et al., PNAS USA. 101 (48):16745-9 (2004)), AmCyanl (Clontech, Palo Alto, Calif), MiCy (MBL International, Woburn, Mass.), and CyPet (Nguyen and Daugherty, Nat Biotechnol. 23(3):355-60 (2005)); BFP proteins such as EBFP (Clontech, Palo Alto, Calif); YFP proteins such as EYFP (Clontech, Palo Alto, Calif.), YPet (Nguyen and Daugherty, Nat Biotechnol. 23(3):355-60 (2005)), Venus (Nagai et al., Nat. Biotechnol. 20(1):87-90 (2002)), Zs Yellow (Clontech, Palo Alto, Calif), and mCitrine (Wang et al., PNAS USA. 101 (48):16745-9 (2004)); OFP proteins such as cOFP (Strategene, La Jolla, Calif.), mKO (MBL International, Woburn, Mass.), and mOrange; and others (Shaner N C, Steinbach P A, and Tsien R Y., Nat Methods. 2(12):905-9 (2005)).

Another class of fluorescent proteins is the red fluorescent protein Discosoma RFP (DsRed) that has been isolated from the corallimorph Discosoma (Matz et al., Nature Biotechnology 17: 969-973 (1999)), and red or far-red fluorescent proteins from any other species, such as Heteractis reef coral and Actinia or Entacmaea sea anemone, as well as variants thereof RFPs include, for example, Discosoma variants, such as monomeric red fluorescent protein 1 (mRFP1), mCherry, tdTomato, mStrawberry, mTangerine (Wang et al., PNAS U S A. 101 (48) :16745-9 (2004)), DsRed2 (Clontech, Palo Alto, Calif.), and DsRed-T1 (Bevis and Glick, Nat. Biotechnol., 20: 83-87 (2002)), Anthomedusa J-Red (Evrogen) and Anemonia AsRed2 (Clontech, Palo Alto, Calif.). Far-red fluorescent proteins include, for example, Actinia AQ143 (Shkrob et al., Biochem J. 392(Pt 3):649-54 (2005)), Entacmaea eqFP61 1 (Wiedenmann et al. Proc Natl Acad Sci USA. 99(18):1 1646-51 (2002)), Discosoma variants such as mPlum and mRasberry (Wang et al., PNAS U S A. 101 (48) :16745-9 (2004)), and Heteractis HcRedl and t-HcRed (Clontech, Palo Alto, Calif).

Another example of a class of agents that may be provided as macromolecules is nucleic acids. For example, a sample may be contacted with an antisense RNA that is complementary to and specifically hybridizes to a transcript of a gene of interest, e.g., to study gene expression in cells of the sample. As another example, a sample may be contacted with a DNA that is complementary to and specifically hybridizes to genomic material of interest, e.g., to study genetic mutations, e.g., loss of heterozygosity, gene duplication, gene amplification, chromosomal inversions, and the like. The hybridizing RNA or DNA is conjugated to detection moieties, i.e. agents that may be either directly or indirectly visualized microscopically. Examples of in situ hybridization techniques may be found at, for example, Harris and Wilkinson. In situ hybridization: Application to developmental biology and medicine, Cambridge University Press 1990; and Fluorescence In Situ Hybridization (FISH) Application Guide. Liehr, T, ed., Springer-Verlag, Berlin Heidelberg 1990. Additional methods that may be used to perform in situ mutation detection in hydrogel embedded samples include, e.g., those described in Grundberg et al. (Oncotarget, Dec 2013, Vol. 4, No. 12, pp. 2407-2418) and Mignardi et al., Nucleic Acid Research, 2015, Vol. 43, No. 22, e151, pp. 1-12. In certain embodiments, in situ mutation detection of individual mRNA molecules is initiated by converting target transcripts into cDNA molecules and then detected by using padlock probes and target primed rolling circle amplification (RCA). Padlock probes are short linear oligonucleotides that become circular when the ends are brought together by hybridization to a target sequence and then joined together by a DNA ligase if perfectly matched. These padlock probes contain tag sequences that after amplification act as detection sites for for fluorescently labeled oligonucleotides. Padlock gap probes, as described in Mignardi et al, are an alternative version of padlock probes whereby the probe arms hybridize to the target with a gap of a defined number of nucleotides between the 5′ and 3′-end of the probe. In this method, cocktails of gap probes can be utilized to compete for hybridization on the target region and ligation to the padlock gap probe. Combined with RCA, the detection events can be visualized and sequenced in situ. In embodiments, the probe specifically binds to a target mutation in the target RNA (or corresponding cDNA) associated with a disease or condidtion, e.g., a cancer or tumor, including but not limited to any of those described herein. These techniques has been demonstrated in FFPE thin sections but never been demonstrated in tumor tissue prepared by CLARITY methods.

As another example, the sample may be contacted with small molecules. For example, if the sample includes β-galactosidase or alkaline phosphatase, it may be desirable to visualize cells and regions of the tissue expressing these proteins. Towards this end, a sample may be contacted with substrates for β-galactosidase (e.g. X-gal, 4-Trifluoromethylumbelliferyl-3-D-galactopyranoside (TFMU-Gal), Resorufin β-D-galactopyranoside (Res-gal), 4-Methylumbelliferyl β-D-galactopyranoside (MUG), di-3-D-galactopyranoside (FDG), Carboxyumbelliferyl β-D-galactopyranoside (CUG)) or for alkaline phosphatase (e.g. nitro-blue tetrazolium (NBT)/5-bromo-4-chloro-3′-indolyphosphate (BCIP)) and other reagents that allow for visualization of β-galactosidase or alkaline phosphatase activity. As another example, it may be desirous to visualize the dendritic arbors and spins of neurons in, e.g., a CNS sample. To do so, the sample may be exposed to chemicals used in Golgi-Cox impregnation, e.g., 3% potassium bichromate followed by a 2% silver nitrate solution.

In some instances, the biomolecules that are targeted by the provided macromolecules are endogenous to the cell. In other instances, the macromolecules may be provided to the sample to target/visualize biomolecules that were ectopically provided to the cells of the sample, e.g. agents that were introduced to the sample in vivo or ex vivo to label certain cell populations or subcellular structures. In some instances, the biomolecules are produced in genetically engineered cell lines through expression of exogenous genes that were introduced. For example, stereotactic surgery may be used to provide biomolecules such as proteins, viruses, chemicals to tissue that label, or “trace”, structures such as vasculature, neurons, cells or subsets of cells in vivo or ex vivo. In this technique, a needle comprising a labeling macromolecule is lowered into the structure or tissue at a precise location and the labeling molecule is released into the structure or tissue. The molecule will fill the structure, tissue or cells in the vicinity of the injection site and, depending on the type of macromolecule delivered, may be transported across synapses to label their targets. Examples of agents that may be used to label structures or cells are well known in the art, including, for example, nucleic acids that encode fluorescent proteins; viral tracers, e.g. Herpes simplex virus type 1 (HSV) and the Rhabdoviruses; wheat-germ agglutinin (WGA); Phaseolus vulgaris leucoagglutinin (PHA-L); horseradish peroxidase-conjugated lectins; biotinylated dextran amines (BDA); cholera toxin B; NEUROBIOTIN Tracer® (Vector labs). Samples labeled in this way may be contacted with macromolecules, e.g. polypeptides or chemicals, that promote the visualization of these ectopically provided labels.

In some instances, the macromolecules that are used to visualize the cellular biomolecules or subcellular structures are passively transported into the sample. In other words, the macromolecules diffuse into the sample. In other instances, the macromolecules are actively transported into the sample, e.g. by electroporation, hydrodynamic pressure, ultrasonic vibration, solute contrasts, microwave radiation, vascular circulation, or the like. In some embodiments, the sample is contacted with the macromolecules after the sample has been cleared. In other embodiments, the hydrogel-embedded sample may be contacted with the macromolecules prior to clearing the sample. In such embodiments, contact with the macromolecules may be facilitated by permeabilizing the sample, that is, changing the properties of the sample to improve the permeability of the sample to macromolecules. By a “permeabilized” sample it is meant that about 50% or more of the macromolecules applied to the sample will penetrate to the deepest regions of the sample, e.g. 60% or more of the macromolecules, 70% or more of the macromolecules, or 80% or more of the macromolecules, in some instances 85% or more of the macromolecules, 90% or more of the macromolecules, or 95% or more of the macromolecules, for example 98% or more of the macromolecules, e.g. 100% of the macromolecules will pass through the sample. Permeabilization of the sample, and of the cells therein, may be achieved by any of the protocols discussed above for the removal of cellular components, e.g. lipids, from the sample or as known in the art for permeabilizing cells. In particular embodiments, the sample is a tumor tissue sample.

In some embodimetns, samples are contacted with one or more macromolecule, e.g., antibodies, via stochastic electrotransport, an electrokinetic method that uses a rotational electric gield to selectively disperse highy electromobile molecules throughout a porous sample without displacing the low-electromobility molecules that constitute the sample (Kim, S.-Y. et al., PNAS, published online Nov. 2, 2015). In some embodiments, cleared samples are contacted with one or more macromolecule, e.g., antibodies, via the eTANGO method, which utilizes dynamic electric fields to move molecules such as antibodies through cleared tissue and ensure an even distribution of the label both spatially and temporally across the sample. In some embodies, the cleared samples are contacted with the macromolecule by a process including stochastic electrotransport and dynamic affinity shift. Stochastic electrotransport drives charged molecules (e.g. antibodies and RNA probes) without disrupting the surrounding charged matrix, and probe-target binding affinities may be modulated to synchronize reaction times sample-wide. Integration of these two concepts enables the endogenous molecular targets in the sample to experience the same reaction condition (time and concentration).

In certain embodiments, samples are contacted with biolecules, e.g., antibodies, via methods described in US Patent Application Publication No. US 20150285765. These methods utilize an electric field to move biolmolecules within the sample matrix.

In some embodiments, the cleared sample is contacted with a blocking solution prior to staining with one or more macromolecules used to identify or visualize cell components, e.g., structures or nucleic acid sequences. In certain embodiments, a blocking solution comprises serum, e.g., goat serum, and/or bovine serum albumin (BSA) and/or Triton-100. An illustrative blocking solution is described in Example 1.

To microscopically visualize samples prepared by the subject methods, in some embodiments, the sample is embedded in a mounting medium. Mounting medium is typically selected based on its suitability for the reagents used to visualize the cellular biomolecules, the refractive index of the sample, and the microscopic analysis to be performed. For example, for phase-contrast work, the refractive index of the mounting medium should be different from the refractive index of the sample, whereas for bright-field work the refractive indexes should be similar. As another example, for epifluorescence work, a mounting medium should be selected that reduces fading, photobleaching or quenching during microscopy or storage. In certain embodiments, a mounting medium or mounting solution may be selected to enhance or increase the optical clarity of the cleared tissue sample. Nonlimiting examples of suitable mounting media that may be used include glycerol, CC/Mount™, Fluoromount™ Fluoroshield™, ImmunHistoMount™, Vectashield™, Permount™, Acrytol™, CureMount™, FocusClear™RapidClear™, or equivalents thereof.

In some instances, the hydrogel-embedded sample is permanently mounted. In other words, once mounted in mounting medium, the hydrogel-embedded sample cannot be removed for further manipulation. In other instances, the sample is temporarily, or reversibly, mounted. In other words, the hydrogel-embedded sample may be removed from the mounting medium and re-stained after microscopy to visualize alternative/additional biomolecules or subcellular structures. In such instances, macromolecules that were previously added to the sample, e.g., to visualize certain biomolecules, may be removed after microscopic analysis by, for example, exposure to organic solvents such as xylenes, ethanol or methanol, exposure to detergents such as sodium dodecyl sulfate (SDS), saponin, Triton X-100 and Tween-20, electrophoresis, hydrodynamic pressure, ultrasonic vibration, solute contrasts, microwave radiation, vascular circulation, and the like. The hydrogel-embedded sample is then contacted with different macromolecules specific for other biomolecules or subcellular structures. As such, iterative staining may be performed on the same sample.

In particular embodiment, a sample is cleared via passive clearing, whiles in other embodiments, a sample is cleared by electrophoretic tissue clearing (ETC), or a combination of passive clearing and ETC. In particular embodiments, both methods rely on SDS micelles to diffuse into the tissue, collect fatty lipids and other unattached biomolecules, and carry them out of the sample, leaving behind a hydrogel-tissue hybrid that is visibly transparent. Passive diffusion of the micelles is a slow process, and ETC accelerates it by using ionic charge on the SDS micelles. When placed in an electric field, the negatively-charged micelles actively travel from one electrode (where they are repelled) to the other electrode (where they are attracted). Thus, applying an electric field across the hydrogel-embedded tissue sample in clearing solution stimulates active transport of the micelles in and out of the tissue.

In some embodiments, the subject methods of the invention are directed to rendering thick tissue, such as whole intact organs or tissue samples, substantially optically transparent for imaging, wherein the tissue/whole organ is cross-linked and hybridized to hydrogel subunits/monomers to stabilize biomacromolecules in the tissue. In some embodiments, the subject methods of the invention are directed to rendering thick tissue, such as whole intact organs or tissue samples, substantially optically transparent for imaging in a passive manner (e.g., using passive CLARITY, or PACT), as generally disclosed in PCT Application Publication No. WO2015041755 (“the '755 publication”) titled “METHODS FOR PHENOTYPING OF INTACT WHOLE TISSUES”, filed Jul. 30, 2014, the entire disclosure of which is incorporated herein by reference. In some embodiments, subject methods of the invention are directed to rendering thick tissue substantially optically transparent for imaging as generally disclosed in “Stochastic electrotransport selectively enhances the transport of highly electromobile molecules”, Kim et al., PNAS 2015 112 (46) E6274-E6283. The approach of Kim et al. includes stochastic electrotransport of clearing agents into a biological sample via use of a rotational electric field to selectively disperse highly electromobile molecules throughout the porous biological sample, without displacing the relatively low-electromobility molecules ofthe sample. Stochastic electrotransport can be attained by continuously rotating a sample chamber, containing the biological sample, with respect to two parallel electrodes positioned next to the sample chamber to create an external rotational electric field with respect to the sample. The sample chamber may be immersed in a circulating buffer solution that is temperature controlled to prevent Joule heating from causing thermal damage to the biological sample. Stochastic electrotransport can effectively amplify the differences in electromobilities to selectively transport highly electromobile molecules, such as clearing agents, into biological samples. In some embodiments, the clearing agent(s) used with stochastic electrotransport can include a lithium borate buffer with SDS.

In some embodiments, such subject methods can further include extracting the tissue lipids from the tissue-hydrogel matrix with detergents such as saponin, Triton X-100 and Tween-20. In some embodiments, such subject methods can further include embedding the cleared tissue/organ in a refractive index matching solution (RIMS) for imaging and/or for long-term storage. In some embodiments, such subject methods obviate the need for active clearing methods that require electrophoresis and/or other methods that may have unintended effects on the tissue such as variability in final tissue quality and tissue browning due to heating.

In some embodiments, the subject methods for rendering thick tissue to be substantially optically transparent can be as follows: 4% paraformaldehyde (PFA)-fixed tissue sections are incubated at 4° C. overnight in the hydrogel monomer solution A4P0 (4% acrylamide in PBS) supplemented with 0.25% photoinitiator 2,2′-Azobis[2-(2-imidazolin-2-yl)propane]dihydrochloride (VA-044, Wako Chemicals USA, Inc.). A4P0-infused samples are degassed with nitrogen for 1-5 minutes and then incubated for 2-3 hours at 37° C. to initiate tissue-hydrogel hybridization. After removing excess hydrogel via brief PBS washes, tissue-hydrogel matrices are transferred into 50 mL conical tubes containing 8% SDS in 0.1M PBS (pH 7.5), and depending on tissue size, were incubated for 2-5 days at 37° C. with shaking. For immunostaining, 1-3 mm thick PACT-processed samples are washed in PBS with 4-5 buffer changes over the course of a day and then transferred to buffer containing small-molecule dyes or primary antibodies followed by fluorescently-conjugated secondary antibody (e.g., 1:200-400 dilution, e.g., in PBS containing 2% normal donkey serum, 0.1% TritonX-100 and 0.01% sodium azide) for 3-7 days or with small-molecule dyes for 1-3 days. Antibody or small molecule dye solutions need to be replaced every day. Unbound antibody is removed via PBS washes, and then samples are incubated with secondary antibodies (Fab fragment secondary antibodies are preferred, 1:200-400) for 2-5 days then washed for 1 day in PBS or phosphate buffer (PB) prior to incubation in imaging media (RIMS). All staining and mounting steps are conducted at room temperature with gentle shaking.

In some embodiments, the RIMS has a refractive index (RI) that is appropriate for tissue imaging, and is about 1.2 or more, about 1.3, about 1.4, about 1.5, about 1.6, about 1.7, about 1.8, including all values and sub ranges in between. In some embodiments, the RIMS has a RI from about 1.38 to about 1.49, is biocompatible, and safe for biological use. In some embodiments, the RIMS is characterized as generally disclosed in the '755 publication.

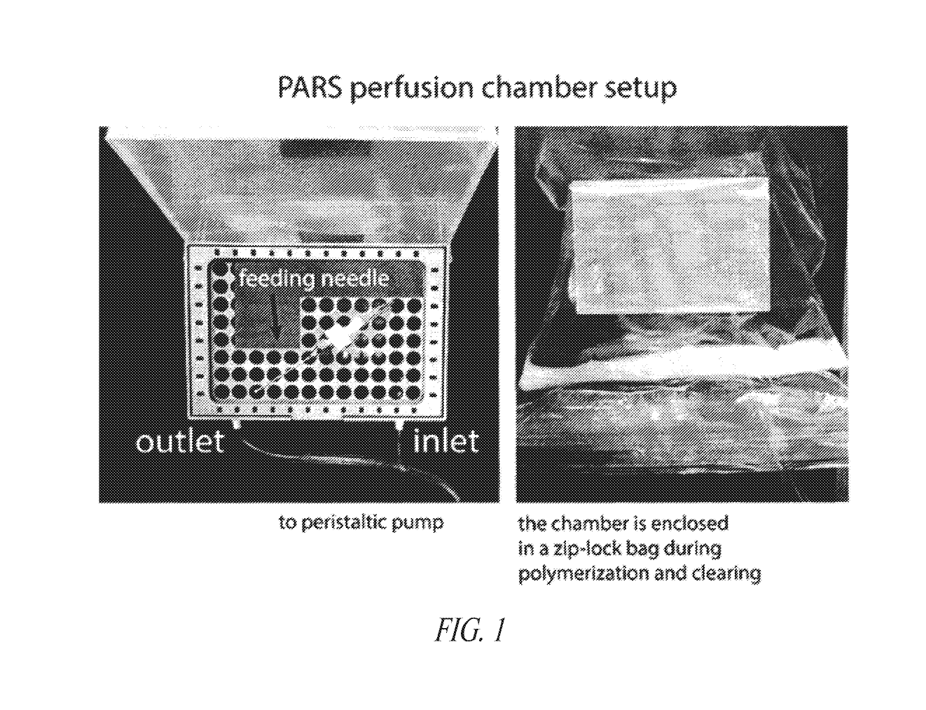

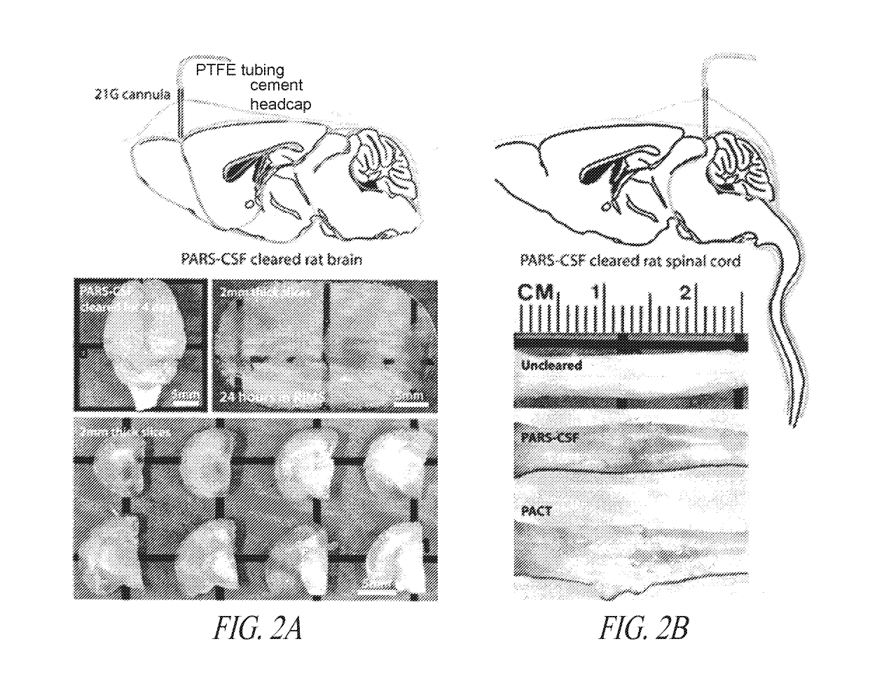

In some embodiments, the subject methods of the invention are directed to a perfusion-assisted agent release in situ (PARS) approach for whole organ/whole animal imaging. In some embodiments, the PARS approach is as generally disclosed in the '755 publication. In such embodiments, the intact vasculature of the organ/animal is exploited to infuse the hydrogel subunits/monomers and clearing solutions (e.g., an SDS solution), which then diffuse throughout the tissues of interest. In some embodiments, the subject methods for rendering the whole animal to be substantially optically transparent can be as follows: following standard cardiac perfusion with 4% PFA (in PBS, pH 7.4), the fixed animal is transferred onto a perfusion chamber (FIG. 1) which recirculates all subsequent PACT and immunolabeling reagents continuously (about 1 ml/min) through rodent vasculature via a peristaltic pump. Perfusion tubing connects the chamber to a feeding needle inserted through the left ventricle into the aorta and loosely sutured in place. The a post-fixed with 4% PFA for 1 hour and then perfusion-washed with PBS for 1 hour. A4P0 monomer is cycled through vasculature overnight, followed by a 2 hour PBS perfusion wash. Before polymerization and without disconnecting perfusion lines, the perfusion chamber is placed into a ziplock bag (FIG. 1), and the bag containing the chamber with the animal is degassed for 2 minutes under nitrogen gas. Polymerization is initiated via perfusion-recirculation of 200 mL of 0.25% VA-044 initiator in PBS at 37° C. for 2-3 hours. The whole-body of the animal can be cleared through a <2-week perfusion with 8% SDS in PBS, pH 7.5 at 37-42° C. followed by extensive PBS perfusion-washing over 2-3 days. Antibodies and small molecule dyes (similar to PACT) are then delivered via a 3-day perfusion and 1-day wash. In some embodiments, such as for brain or spinal cord clearing (FIGS. 2A-2B), transcardially-fixed animals (e.g., rodents) are decapitated and a subdural cannula is inserted above the region of interest and cemented to the skull. All PACT reagents are delivered in the same order and timeframe as PARS reagents at aboutl ml/min.

In some embodiments, the subject methods of the invention are directed to rendering thick tissue, such as whole intact organs or tissue samples, substantially optically transparent for imaging. In such embodiments, the tissue/whole organ is rendered substantially optically transparent via treatment with a clearing agent including urea and/or a urea-based derivative. In some embodiments, the clearing agent includes urea, and can be formulated as generally disclosed in “SCALE: A CHEMICAL APPROACH FOR FLUORESCENCE IMAGING AND RECONSTRUCTION OF TRANSPARENT MOUSE BRAIN”, Hama et al., Nature Neuroscience 14, 1481-1488 (2011), the entire disclosure of which is incorporated herein by reference. In some embodiments, the clearing agent includes an aqueous solution of urea, Triton X-100, and glycerol, and can be formulated as generally disclosed in U.S. patent application publication no. 2014/0178927 titled “CLARIFYING REAGENT FOR BIOLOGICAL MATERIALS AND USE THEREOF”, filed May 18, 2012, the entire disclosure of which is incorporated herein by reference. In some embodiments, the clearing agent includes an aqueous solution of urea and an aminoalcohol. For example, in some embodiments, the clearing agent includes an aqueous solution of N,N,N′,N′-tetrakis(2-hydroxypropyl)ethylenediamine, Triton X-100, and urea, and can be formulated as generally disclosed in “WHOLE-BRAIN IMAGING WITH SINGLE-CELL RESOLUTION USING CHEMICAL COCKTAILS AND COMPUTATIONAL ANALYSIS”, Susaki et al., Cell, Volume 157, Issue 3, 24 Apr. 2014, Pages 726-739, the entire disclosure of which is incorporated herein by reference. In some embodiments, the clearing agent is ScaleView-A2, as sold by Olympus Corporation.

Samples prepared using the subject methods (e.g., tumor tissue samples) may be analyzed by any of a number of different types of microscopy, for example, optical microscopy (e.g. bright field, oblique illumination, dark field, phase contrast, differential interference contrast, interference reflection, epifluorescence, confocal, two-photon, temporal focusing, light sheet with or without methods for extending the detection depth of field, etc., microscopy), laser microscopy, electron microscopy, scanning probe microscopy, and CLARITY optimized light microscopy (COLM, described below).

Also provided are reagents and kits thereof for practicing one or more of the above-described methods. Reagents and kits may include one or more of the following: fixative; hydrogel subunits; clearing reagents; detection macromolecules, e.g., labeled and or unlabeled antibodies or aptamers, nucleic acid probes (oligonucleotides, vectors, etc.), chemicals, etc.; buffers, e.g. buffer for fixing, washing, clearing, and/or staining samples; mounting medium; embedding molds; dissection tools; etc. The subject reagents and kits thereof may vary greatly. In particular embodiments, kits of the present invention may comprise any of the probes described herein for analyzing tissue samples having an associated disease.

Also provided are samples that have been prepared by the subject methods for use in, for example, studying tissue at the cellular and subcellular level. For example, fixed and polymerized samples, or samples that have been fixed, polymerized, and cleared, are provided for use in studying the expression of genes of interest, for screens to identify candidate agents that target cells and/or subcellular structures of interest, etc. Such prepared samples may also be provided as a positive control in one of the kits or systems as described herein.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Aspects of the invention can further include one or more devices for performing aspects of the subject methods described above. In some embodiments, the invention includes one or more devices as generally disclosed in the '392 publication. Example devices may include, but are not limited to, electrophoresis apparatus, ultrasounds, microwaves, needles, tubing, perfusion pumps, etc., for fixing, clearing, and/or staining samples.

In some embodiments, the one or more devices includes an electrophoresis device suitable for use in removing cellular components from a sample, e.g., cellular components that are not crosslinked to the hydrogel network. The electrophoresis device can be any device configured for application of an electric field to a sample, e.g., to a biological sample, such as a tumor biopsy sample. Electrophoresis is most commonly used to mobilize biological macromolecules, e.g., nucleic acids, proteins, in a sample to separate and analyze those macromolecules. The electrophoresis device can include one or more of capillary electrophoresis, gel electrophoresis, paper electrophoresis, and immunoelectrophoresis. In some embodiments, the electrophoresis device includes gel electrophoresis. Examples of electrophoresis devices and methods of gel electrophoresis may be found in, for example, U.S. Pat. Nos. 3,129,158, 3,208,929, 3,346,479, 3,375,187, 3,616,454, 3,616,457, 3,616,454, 3,616,457, 3,563,880, 3,576,727, 3,674,678, 3,865,712, 4,088,561 , 4,151,065, 4,292,161 , 4,339,327, 4,375,401 , 4,415,418, 4,479,861 , and 4,588,491; and Martin, Robin. Gel electrophoresis: nucleic acids. BIOS Scientific, 1996; Hames, B.D. Gel Electrophoresis of Proteins: A Practical Approach, Oxford University Press 1998; and Burmeister, M. and Ulanovsky, L. Pulsed-field Gel Electrophoresis, The Humana Press Inc. 1992, the disclosures of which are incorporated herein by reference.

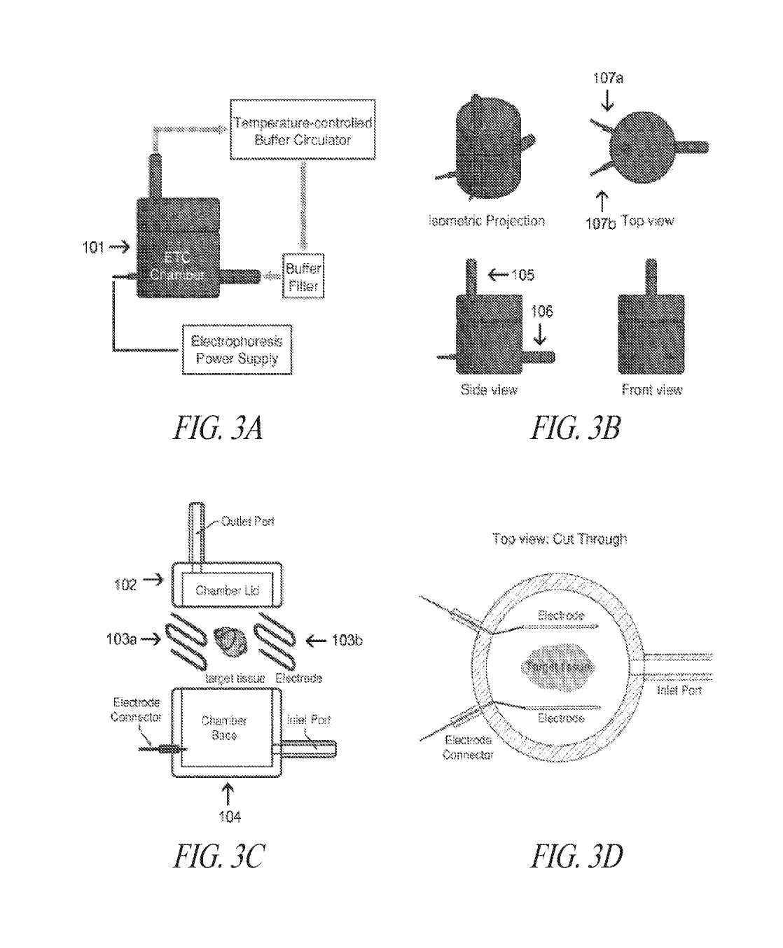

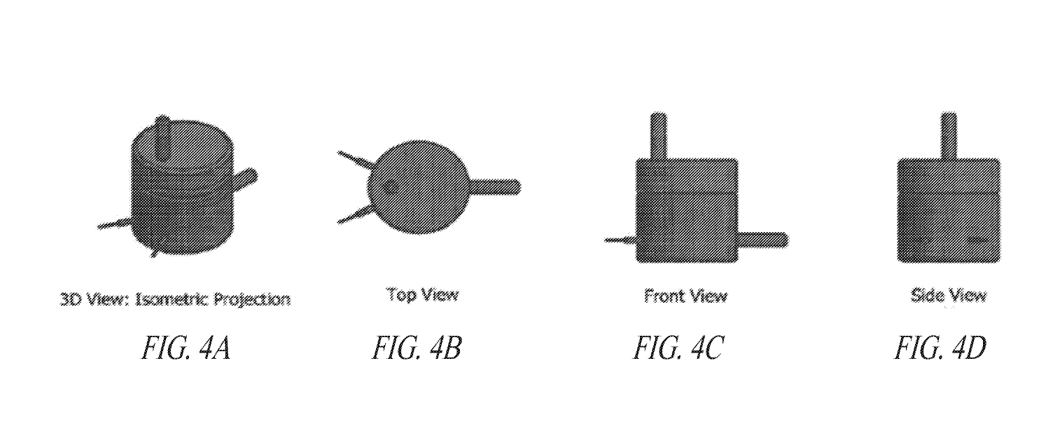

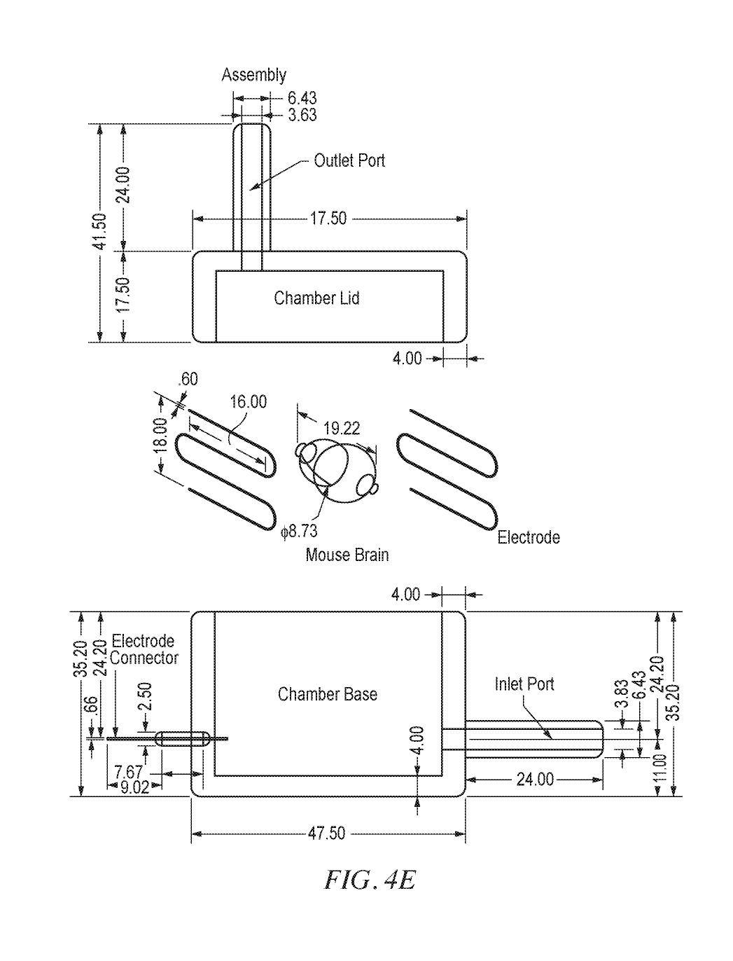

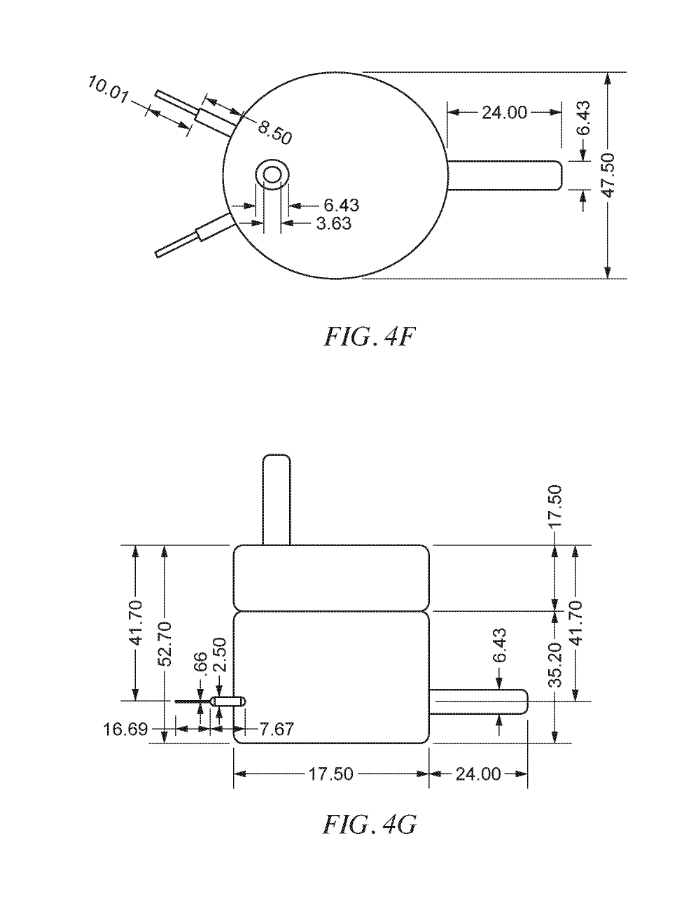

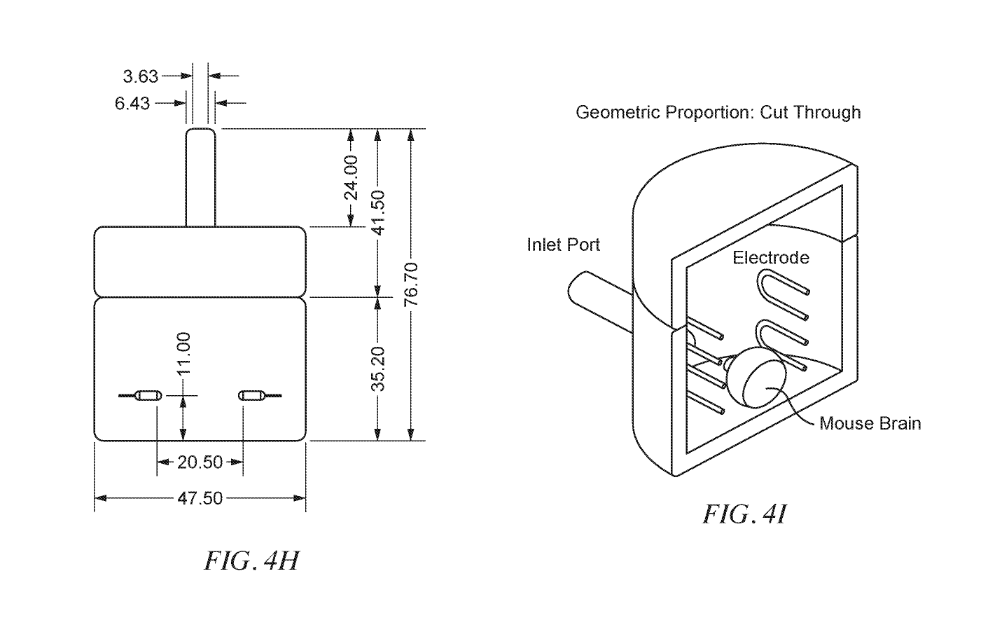

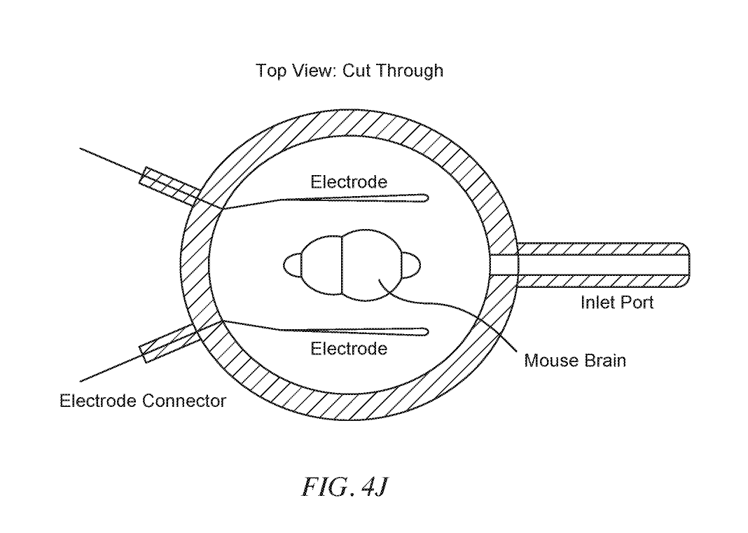

In some embodiments, the electrophoresis device(s) can include an electrophoresis chamber into which a buffer solution and the hydrogel-embedded sample may be placed. See, for example, FIGS. 3A-3D and FIGS. 4A-4J. The electrophoresis chamber may generally be any suitable size to accommodate a hydrogel-embedded sample of interest, and may be constructed of any material that will retain solution within the chamber, for example glasses and plastics, such as, for example, acrylics, polycarbonates, polystyrenes, polymethyl methacrylates, polyethylene, polyfluoroethylene, polypropylene, polyurethane, polyethylene terephthalate, polytetrafluoroethylene and the like. In some embodiments, a chamber may be molded or machined or otherwise formed from a resin or hard plastic, as appropriate for particular applications. In some embodiments, an electrophoresis chamber may further comprise a component that is configured to support a hydrogel-embedded sample, such as, e.g., a platform, within the electrophoresis chamber.

In some embodiments, an electrophoresis device may include a lid that fits over the electrophoresis chamber to close the chamber. Lids in accordance with embodiments of the invention may include a seal that forms a liquid-tight and/or air-tight seal with the body of the electrophoresis chamber when the lid is coupled to the chamber. In some embodiments, one or more sealing components may be attached to the lid, attached to the chamber, or attached to both the lid and the chamber. When the lid is coupled to the chamber, the sealing components may form a liquid and/or air-tight seal.

Electrophoresis devices in accordance with some embodiments of the invention may include two or more electrodes of opposite polarity (i.e., “anode” (negatively charged) and at least one “cathode” (positively charged)) operably associated with the electrophoresis chamber to which an electric current may be applied to create an electric field within the chamber. The electrodes may be constructed of any material that will result in an electric field being established upon the application of an electric current to the electrodes, and may be configured within the chamber and relative to the site where the sample is to be placed in any convenient way that will promote the establishment of an electric field across a sample positioned therein, for example as well known in the art of nucleic acid or protein electrophoresis. See, for example, U.S. Pat. Nos. 3,129,158, 3,208,929, 3,346,479, 3,375,187, 3,616,454, 3,616,457, 3,616,454, 3,616,457, 3,563,880, 3,576,727, 3,674,678, 3,865,712, 4,088,561 , 4,151,065, 4,292,161 , 4,339,327, 4,375,401 , 4,415,418, 4,479,861 , and 4,588,491 ; and Martin, Robin. Gel electrophoresis: nucleic acids. BIOS Scientific, 1996; Hames, B. D. Gel Electrophoresis of Proteins: A Practical Approach. Oxford University Press 1998; and Burmeister, M. and Ulanovsky, L. Pulsed-field Gel Electrophoresis. The Humana Press Inc. 1992. For example, the electrodes may be configured within the chamber so as to substantially flank the sample.

In some embodiments, one or more of the electrodes may include an extension component that is used to enlarge the size of the electric field that is generated between the electrodes. For example, in certain embodiments, an electrode may include an extension component in the form of a serpentine portion of the electrode the doubles back on itself to form a plurality of S-shaped bends. An example of an electrode including a serpentine extension component can be seen in FIG. 3, Panel c (reference numbers 103a and 103b). The extension component enlarges the size of the electric field that is generated when a voltage is applied to the electrodes so that an entire three-dimensional tissue sample can be placed inside the electric field. The length and width of the extension component can be adjusted as needed to accommodate tissue samples of various dimensions. For example, in some embodiments the length and the width of an electrode comprising an extension component are approximately equal, as depicted in FIG. 4, Panel e. In certain embodiments, an electrophoresis chamber may be partitioned by, e.g., a solid divider or by air into two distinct regions, where each region comprises one electrode in a buffer, and the sample is positioned within the buffer such that the sample spans, or straddles the two regions, such that the electric field created by the electrodes is created through the sample. In some instances, the chamber may comprise a platform or support structure, upon or into which the hydrogel-embedded sample is placed, e.g., a platform between two electrodes, a platform that spans regions of the chamber comprising the electrodes, etc.

The electrophoresis apparatus may be operably linked to a power source from which voltage may be applied to the electrodes. In some instances, the power source may be separate from the electrophoresis apparatus, i.e. the electrophoresis apparatus may be a separate module from the power source. In other instances, the power source may be integrated into the electrophoresis apparatus, i.e., the electrophoresis apparatus will comprise the power source.

In some instances, it may be desirable to replace or recirculate buffer in the electrophoresis chamber. In some embodiments, circulating or recirculating the buffer includes removing the buffer from the chamber and then returning the buffer to the chamber, for example, after passing through a cooling unit (refrigeration unit, ice bath, etc.), a heating unit, a filter, etc. In some embodiments, replacing the buffer includes removing the buffer from the chamber and adding fresh buffer in its place. For example, it may be desirable to control the temperature of the buffer inside the electrophoresis chamber (e.g., to prevent the chamber from reaching temperatures that might cause the hydrogel to depolymerize or the biomolecules in the sample to denature, e.g., 35° C. or more, 40° C. or more, or 50° C. or more, 60° C. or more, 70° C. or more, 80° C. or more, 90° C. or more, or 100° C. or more); to remove macromolecules from the buffer as they exit the sample; to vary the ionic strength of the buffer; etc. Towards this end, the electrophoresis apparatus may optionally include one or more ports through which buffer may enter and/or exit the chamber. In some instances, the chamber may comprise two or more ports, e.g. a first port through which buffer enters the chamber and a second port through which buffer exits the chamber.

Buffer may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. For example, a first tube having a first and second end may be attached to a first port and a second tube having a first and second end may be attached to a second port, where the first end of the first tube is attached to the first port and the second end of the first tube is operably linked to a receptacle, e.g. a cooling unit, heating unit, filtration unit, waste receptacle, etc.; and the first end of the second tube is attached to the second port and the second end of the second tube is operably linked to a receptacle, e.g. a cooling unit, beaker on ice, filtration unit, waste receptacle, etc.

As another example, one tube having a first and second end may be removably attached to both a first and second port, i.e., the first end of the tube is removably attached to the first port and the second end of the tube is removably attached to the second port, where the tubing is operably linked to, for example, a refrigeration unit (e.g., the tubing passes through the unit), a filter (e.g. the tubing comprises a filter), a buffer reservoir (e.g. the tubing receives replacement buffer from a reservoir via, e.g., a splitter), etc. In some instances, the tubing will also be operably connected to a pump, e.g. a peristaltic pump, an electro-osmotic pump, an oscillatory pump, a diaphragm pump etc., that will facilitate the movement of liquid through the tubing, facilitate the addition/removal/recirculation of buffer from the electrophoresis chamber, etc. In this way, the electrophoresis apparatus may be operably connected to a cooling unit, heating unit, filtration unit, buffer reservoirs/receptacles, pump, etc. In some embodiments, a refrigeration unit, heating unit, filtration unit, buffer reservoirs/receptacles, pump, etc. will be integrated into the electrophoresis apparatus. In other words, the electrophoresis apparatus may comprise the refrigeration unit, heating unit, filtration unit, buffer reservoirs/receptacles, pump, etc. In other embodiments, the refrigeration unit, heating unit, filtration unit, buffer reservoirs/receptacles, pump, etc., may be a separate module from the electrophoresis apparatus.

As illustrated in FIGS. 3A-3D, an example electrophoretic tissue clearing device 101 is shown. The example device 101 includes a lid 102, a first electrode 103a, a second electrode 103b, a base 104, an outlet port 105, an inlet port 106, a first electrode connector 107a and a second electrode connector 107b.

The present disclosure also provides systems for performing the subject methods. Systems may include one or more of the modules described herein, e.g. an electrophoresis apparatus, a power supply, a refrigeration unit, a heating unit, a pump, etc. Systems may also include any of the reagents described herein, e.g. fixative; hydrogel subunits; clearing reagents; detection macromolecules, e.g., antibodies, nucleic acid probes (oligonucleotides, vectors, etc.), chemicals, etc.; buffers, e.g., buffer for fixing, washing, clearing, and/or staining samples; mounting medium; embedding molds; etc. Systems in accordance with certain embodiments may also include a microscope and/or related imaging equipment, e.g., camera components, digital imaging components and/or image capturing equipment, computer processors configured to collect images according to one or more user inputs, and the like.

Certain embodiments of CLARITY procedures that may be used according to the present invention are described in PCT Publication No. WO2014/025392, Tomer, R. T et al., Nature Protocols, Vol. 9, No. 7, pp. 1682-1697 (June 2014), and Kim, S. Y. et al., P.N.A.S. Plus, Vol. 112, No. 46, pp. E6274-E6283 (October 2015), which are hereby incorporated by reference in their entireties.

### III. Imaging of Biological Samples

Aspects of the invention are directed to methods, devices, and systems for conducting high-speed, high-resolution imaging of tissue samples (e.g., tumor tissue samples), large organs, whole animals, etc. prepared as described above. In some embodiments, aspects of the invention are directed to methods, devices, and systems for conducting high-speed, high-resolution imaging of tissue samples as generally disclosed in PCT Application No. PCT/US2015/032951 titled “METHODS AND DEVICES FOR IMAGING LARGE INTACT TISSUE SAMPLES”, filed May 28, 2015, the entire disclosure of which is incorporated herein by reference.

In some embodiments, the subject systems include imaging devices configured for one or more of the following: fluorescence imaging, reflectance imaging, brightfield imaging, darkfield imaging, differential interference contrast (DIC) imaging, phase contrast imaging, polarization imaging, and/or the like. In some embodiments, the subject systems include imaging devices configured for one or more of the following: X-ray imaging, computed tomography (CT) imaging, magnetic resonance imaging (MRI), ultrasound imaging, and/or the like. In some embodiments, the subject systems are configured for multi-modal imaging and include two or more imaging approaches. In some embodiments, the subject systems are configured for correlation and/or co-registration of images acquired via two or more imaging approaches.

In some embodiments, the subject systems include a microscope device that includes an illumination beam path with a light source, a detection beam path with a camera, an optically homogenous sample manipulation component that includes a sample chamber, a controller, a processor, and a computer-readable medium including instructions that, when executed by the processor, (in some embodiments) cause the controller to execute a calibration procedure to acquire a plurality of alignment parameters for a sample in the sample chamber, and executes an imaging procedure that utilizes the alignment parameters to generate a three dimensional image of the sample.

In some embodiments, the illumination beam path components may include a collimator, shutter, illumination filter wheel, beam expander, two-dimensional scanner, scan lens, tube lens, one or more mirrors, and a light source, as described further below. Any of these components, any number of any of these components, or combinations or arrangements thereof, may be utilized in a suitable manner in the subject systems and devices.

In some embodiments, an illumination beam path includes a cylindrical lens that is configured to generate a static light sheet. In some embodiments, an illumination beam path includes galvanometer scanner/f-theta lens configured to create dynamic light sheets with a Gaussian or Bessel beams. In some embodiments, a system may include two illumination beam paths, wherein the illumination beam paths are configured to illuminate a sample from opposite or opposing sides of the sample.

In some embodiments, a system may be configured to generate a plurality of light sheets, such as one, two, three, four or more light sheets from a single illumination beam path. In certain embodiments, a light sheet can be independently manipulated to illuminate a desired portion of a sample. In some embodiments, two or more light sheets can be used to illuminate a portion of a sample in a coordinated manner, such that a first light sheet illuminates a first portion of the sample, and a second light sheet illuminates second portion of the sample, wherein the first portion of the sample is different from the second portion of the sample. In some embodiments, a plurality of light sheets can be used to illuminate a portion of a sample, for example, up to three or more, such as four or more, such as five or more, such as six or more, such as seven or more, such as eight or more light sheets can be used to illumination a portion of a sample in a coordinated manner. In some embodiments, each light sheet can be manipulated independently to illuminate a given portion of a sample. In some embodiments, a plurality of light sheets can be manipulated in a coordinated manner to accomplish imaging of a sample in accordance with the methods described further herein.

In some embodiments, a system may include two illumination beam paths. In certain embodiments, two illumination beam paths may be used to illuminate a sample from opposite sides, thereby allowing one half of the sample to be imaged from one side, and the other half of the sample to be imaged from another side.

Illumination beam paths in accordance with embodiments of the invention may also include various light sources configured to generate light in the visible spectrum, having a wavelength ranging from about 390 nm to about 700 nm (including all values and sub ranges in between), or in the infrared spectrum in the range from about 700 nm-about 1500 nm (including all values and sub ranges in between). In some embodiments, a light source may include a laser. In some embodiments, a light source (such as a laser light source) is configured to emit light having a wavelength of, e.g., 405 nm, 488 nm, 514 nm, 561 nm, 594 nm, or 647 nm. Any of a variety of suitable light sources may be used with the subject systems.

As summarized above, aspects of the invention include systems that include a detection beam path. Detection beam path components are well known in the art and are not described in great detail herein. In some embodiments, detection beam path components include a camera, tube lens, emission filter wheel, and a detection objective. Any of these components, or combinations or arrangements thereof may be utilized in a suitable manner in the subject systems and devices.

In some embodiments, a camera is a CCD camera or a scientific CMOS camera (sCMOS) providing extremely low noise, rapid frame rates, wide dynamic range, high quantum efficiency (QE), high resolution, and large field of view. Such cameras are commercially available from scientific technology vendors.

In some embodiments, a detection objective is configured to have a refractive index (RI) that matches the RI of a sample undergoing imaging. For example, in some embodiments, a detection objective may be a 25×, 10×, or 4× detection objective whose RI is matched with that of an immersion liquid and/or tissue sample undergoing imaging analysis.

In some embodiments, a detection objective may be a low numerical aperture detection objective. In some embodiments, the numerical aperture of the detection objective ranges from about 0.1 to about 1.4, such as about 0.6 to about 1.0, including all values and sub ranges in between.

In some embodiments, a detection objective has a working distance (WD) in a range from about 0.1 mm to about 100 mm, such as about 6-8 mm, including all values and sub ranges in between.

In some embodiments, a system may include two detection beam paths. In certain embodiments, two detection beam paths may be used to simultaneously image a sample from opposite sides, thereby allowing one half of the sample to be imaged from one side, and the other half of the sample to be imaged from another side. This embodiment provides a two-fold increase in the sample size that can be imaged, as the overall working distance of the combination of both objectives is increased by two-fold through the addition of a second objective in the second detection beam path.

Aspects of the invention include an optically homogenous sample manipulation component configured to contain a sample in an optically homogenous environment. By “optically homogenous” is meant that the refractive indices (RIs) of the various materials in the environment are matched, or are similar, such that a beam of light traveling through the optically homogenous environment will be insubstantially impacted by any changes in the RIs of the materials through which it travels.

In some embodiments, the optically homogenous sample manipulation component includes a bottom or base and an outer wall that defines a sample chamber in the shape of a box with an open top. In some embodiments, a lens from one or more illumination beam paths is disposed on or in a portion of the outer wall such that light emitted from an illumination beam path enters directly into an interior portion of the sample chamber via a transparent window. In some embodiments, the transparent window is made of a material that matches the RI of the optically homogenous environment. In some embodiments, the transparent window is made from, e.g., quartz coverslips. In some embodiments, a detection objective of a detection beam path is disposed on or in a portion of the outer wall. In some embodiments, a detection objective is positioned in an orthogonal relationship to an illumination beam path (e.g., is positioned at a 90° angle to an illumination beam path).

In some embodiments, the optically homogenous sample manipulation component includes an xyz-theta sample mount that is configured to move a sample in any of a plurality of directions, including x, y, and z directions as well as angular or 8 rotational directions. In some embodiments, the xyz-theta stage mount has a large travel range and is configured to move at least 45 mm is each of the x, y, and z directions. In some embodiments, the xyz-theta stage mount is configured to rotate the sample by a full 360° in the angular or theta direction. Such xyz-theta sample mounts are commercially available from scientific technology vendors.

In some embodiments, the optics of the system are configured to move with respect to the sample, whereas in some embodiments, the sample manipulation component is configured to move the sample with respect to the optics of the system. Movement of either the optics or the sample manipulation components can be, e.g., in a step-by-step manner or in a continuous manner. In some embodiments, the optics and the sample manipulation components can be moved in a synchronous manner, whereas in certain embodiments, the optics and the sample manipulation components can be moved in an asynchronous manner.

In some embodiments, the sample chamber of the optically homogenous sample manipulation component is filled with a solution. In some embodiments, the solution has an RI that matches the RI of the sample or the detection objective of the detection beam path. For example, in some embodiments, the solution used to fill the sample chamber is FocusClear or MountClear (both commercially available from CelExplorer Labs). In some embodiments, the solution used to fill the chamber is a liquid with a refractive index that ranges from about 1.42 up to about 1.46, such as about 1.45, including all values and sub ranges in between. In some embodiments, the solution used to fill the sample chamber is about 87% glycerol. Solutions having the desired range of refractive indices are commercially available from vendors, such as Cargille Labs.

In some embodiments, the sample chamber of the optically homogenous sample manipulation component includes a smaller inner chamber whose volume is less that the volume of the larger, outer sample chamber. For example, in some embodiments, the inner chamber is a cuvette that is configured to house a sample for analysis. In some embodiments, a cuvette made from fused quartz is used as the inner chamber. In some embodiments, the inner chamber is filled with a solution, as described above, whose RI matches the RI of the sample or the detection objective of the detection beam path. For example, in some embodiments, the solution used to fill the inner chamber is FocusClear or MountClear (both commercially available from CelExplorer Labs). In some embodiments, the solution used to fill the inner chamber is RI 1.454, commercially available from vendors such as Cargille Labs. In some embodiments, the solution used to fill the inner chamber is about 87% glycerol. In certain embodiments, a first solution may be used to fill the inner chamber, and a different solution may be used to fill the larger, outer chamber. For example, in some embodiments, the inner chamber is filled with FocusClear, while the outer chamber is filled with RI 1.454 solution, or 87% glyercol solution.

In some embodiments, aspects of the invention include a controller, processor and computer readable medium that are configured or adapted to control or operate one or more components of the subject systems. In some embodiments, a system includes a controller that is in communication with one or more components of the systems, as described herein, and is configured to control aspects of the systems and/or execute one or more operations or functions of the subject systems. In some embodiments, a system includes a processor and a computer-readable medium, which may include memory media and/or storage media. Applications and/or operating systems embodied as computer-readable instructions on computer-readable memory can be executed by the processor to provide some or all of the functionalities described herein.

In some embodiments, a system includes a user interface, such as a graphical user interface (GUI), that is adapted or configured to receive input from a user, and to execute one or more of the methods as described herein. In some embodiments, a GUI is configured to display data or information to a user.

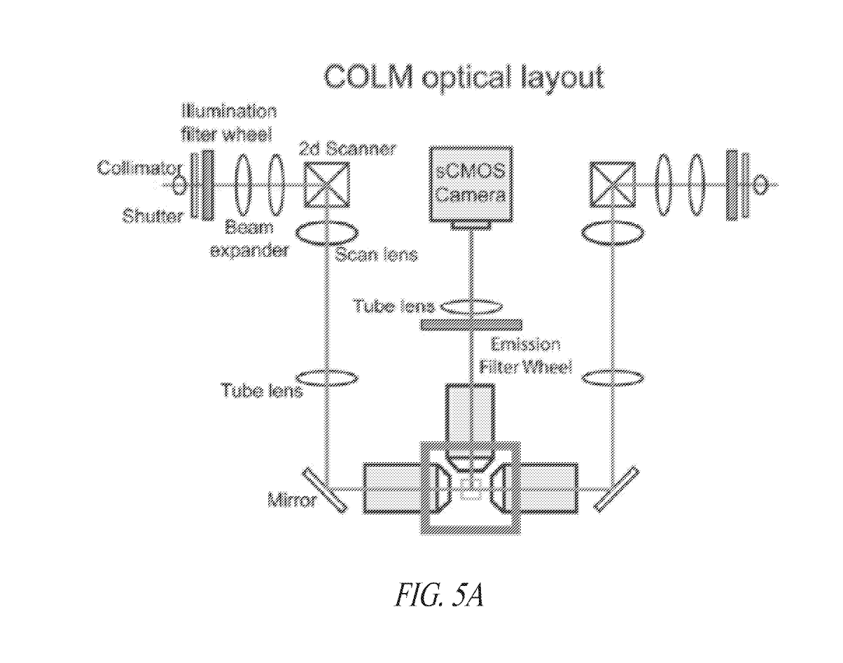

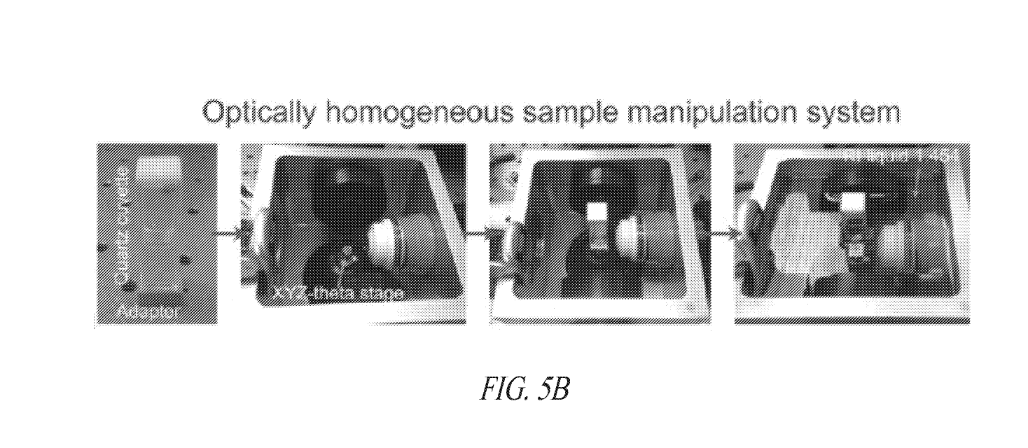

Referring now to FIG. 5A, an embodiment of a microscope device is depicted. The depicted microscope device includes a first and a second illumination beam path, wherein each illumination beam path includes a collimator, shutter, illumination filter wheel, beam expanders, 2d galvanometer scanner, scan lens (or f-theta lens), tube lens, mirror and illumination objective. The depicted microscope device also includes a detection beam path that includes an sCMOS camera, tube lens, emission filter wheel and a detection objective that is positioned within the optically homogenous sample manipulation component.

Referring now to FIG. 5B, various components of an optically homogenous sample manipulation component are depicted. FIG. 5B, panel a shows a quartz cuvette that may serve as an inner sample chamber. FIG. 5B, panel b shows an xyz-theta sample mount stage positioned on the base of the sample chamber. The detection objective of the detection beam path and the lenses of two illumination beam paths are also shown. FIG. 5B, panel c shows the cuvette placed within the sample chamber to form an inner sample chamber. FIG. 5B, panel d shows the sample chamber filled with RI 1.454 liquid.

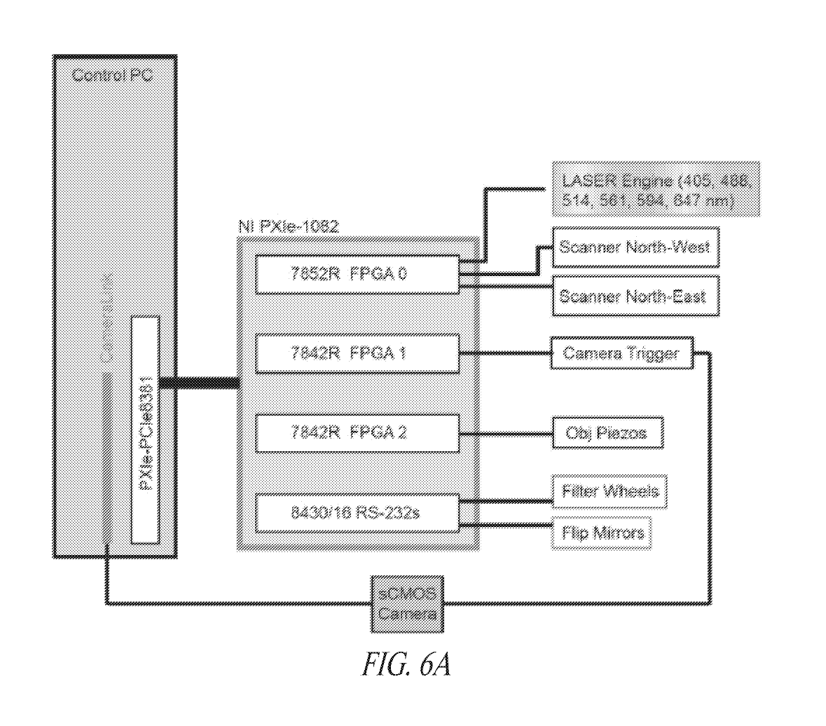

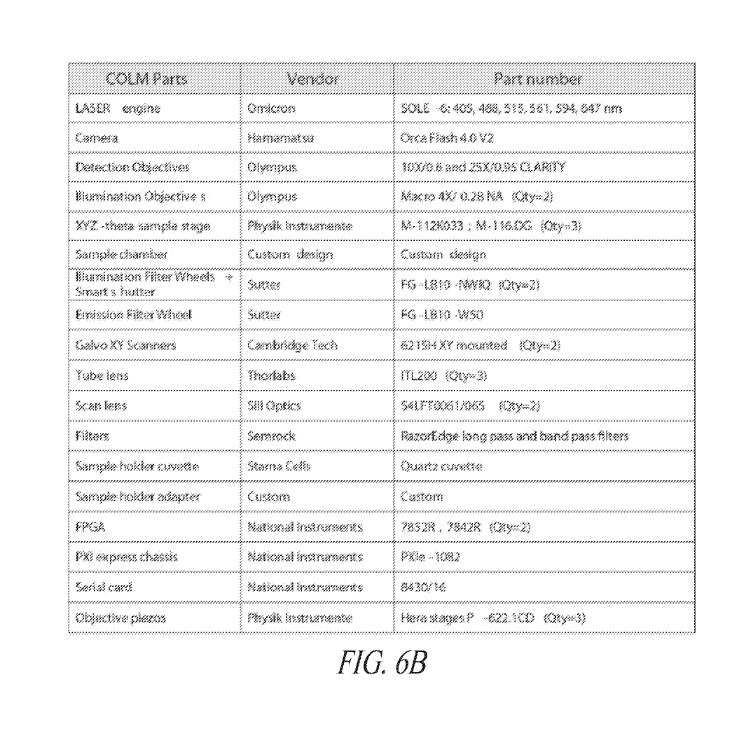

Referring now to FIG. 6, a schematic summary of the control electronics framework and COLM (CLARITY-optimized light microscopy) parts are shown. As depicted, a control computer or processor communicates with the various components of the systems, including, e.g., an sCMOS camera in the detection beam path, a laser controller in the illumination beam path, and various additional components.

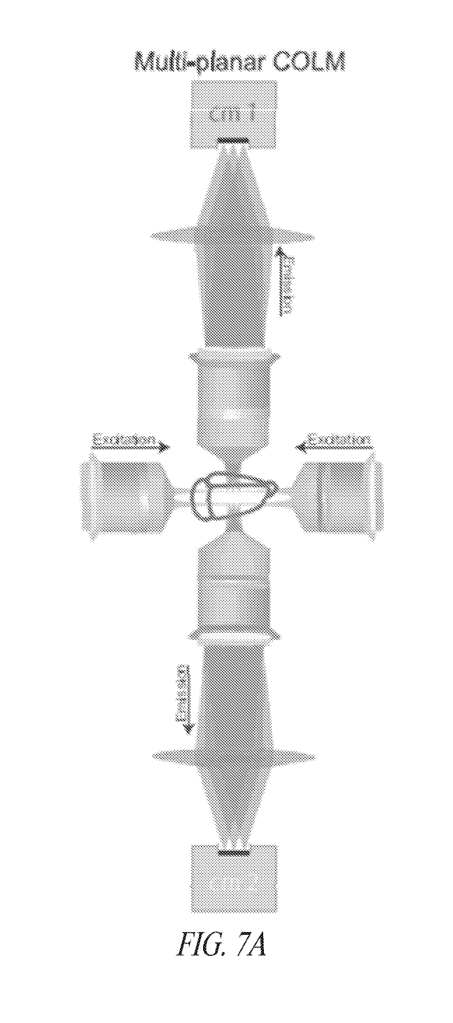

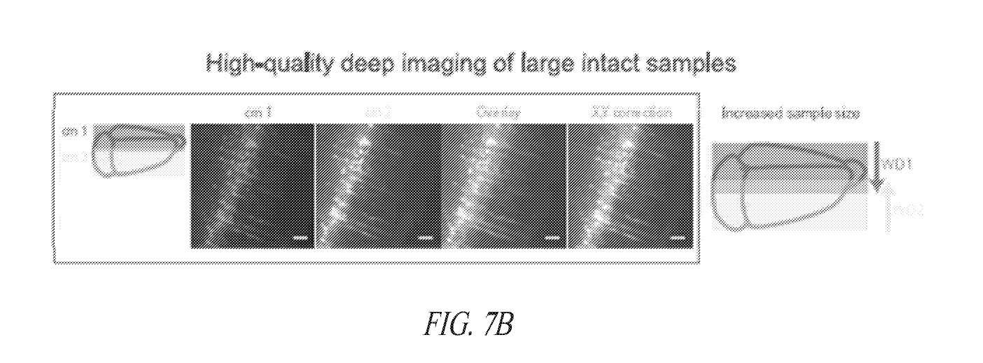

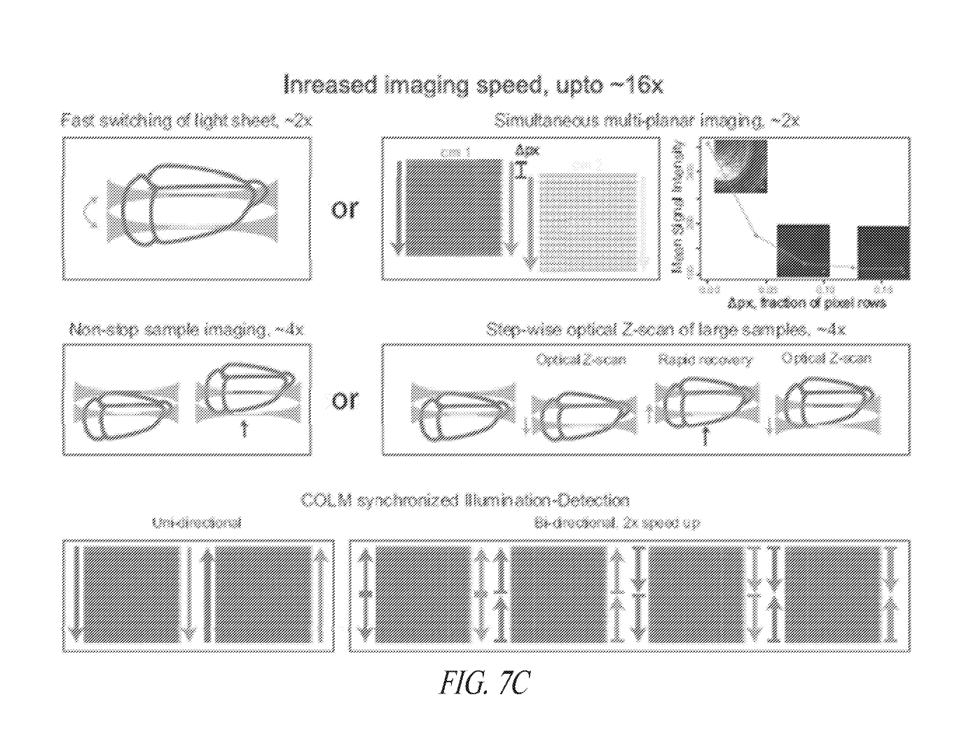

Referring now to FIG. 7, Panel a, an embodiment of a multi-planar COLM system is shown. As depicted, the system includes two illumination beam paths and two detection beam paths. The illumination beam paths are configured to illuminate a sample from opposite sides, and the detection beam paths are configured to image the sample from opposite sides. Simultaneous imaging of multiple planes is achieved by operating the two detection arms slightly shifted (about 100 microns is sufficient as shown in FIG. 7, Panel c.) from each other in the row-by-row readout direction.

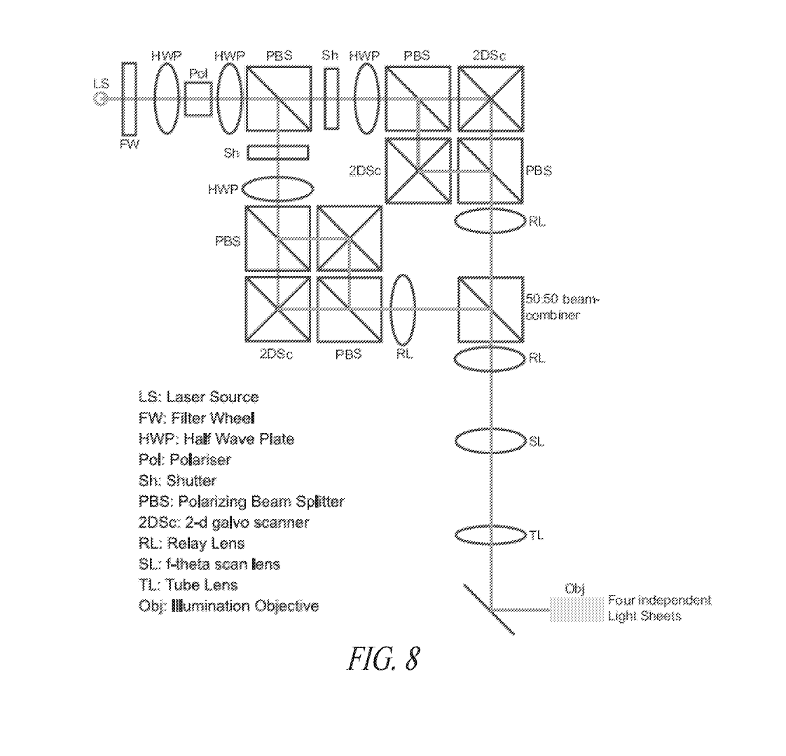

Referring now to FIG. 8, an embodiment of an illumination beam path of a multiplanar COLM system is shown. As depicted, the illumination beam path includes a variety of components that are configured to generate multiple independent light sheets. The depicted embodiment is configured to generate four independent light sheets.

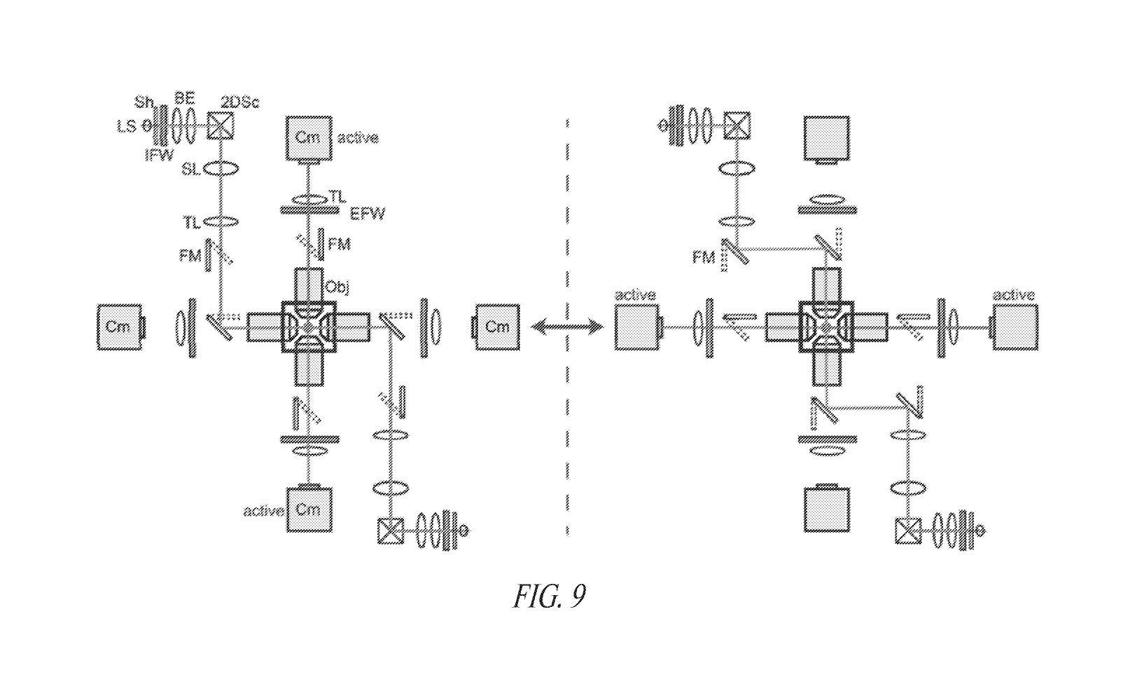

Referring now to FIG. 9, a two detection path can be extended to four detection paths by employing the use of motorized flip mirrors to redirect illumination beams to two different configurations that allow imaging by the corresponding pair of detection arms.

Aspects of the invention include methods that may be used for imaging large tissue samples, such as tumor tissue samples, rendered substantially transparent by the methods described herein. In some embodiments, the subject methods involve placing a sample in the sample chamber of an optically homogenous sample manipulation component, performing a calibration procedure to align a light sheet and a detection focal plane of a microscope device at a plurality of locations within the sample to acquire an alignment parameter for each location, performing an imaging procedure to collect an image from each of the plurality of locations within the sample, and constructing a three dimensional image of the sample using the image from each location. Aspects of the methods are now further described in greater detail below.

Aspects of the methods involve placing a sample in an optically homogenous sample manipulation component. In some embodiments, a sample is placed on a sample mount stage, such as an xyz-theta sample mount stage that is configured to move the sample in any direction. In some embodiments, the methods involve placing a sample in the sample chamber of an optically homogenous sample manipulation component, and filling the sample chamber with a solution that has a refractive index (RI) that matches that of the sample. In some embodiments, prior to performing the imaging analysis described herein, a sample may be prepared for microscopic analysis. In some embodiments, a sample is prepared by fixing the sample in the presence of hydrogel subunits, polymerizing the hydrogel subunits to form a hydrogel-embedded sample, and clearing the hydrogel-embedded sample. Preparation methods are further described in International Patent Application No. PCT/US2013/031066, the disclosure of which is herein incorporated by reference in its entirety.

Aspects of the methods involve executing a calibration procedure that is used to align a detection focal plane of the microscope device with an illumination plane of the sample that is illuminated with a sheet of light. In some embodiments, the calibration procedure involves specifying a start position and an end position for the sample in the z direction.

In certain embodiments, the calibration procedure involves specifying a z-step value that is used to divide the sample into a plurality of planes in the z-direction, where each plane represents a two-dimensional segment or portion of the sample. In some embodiments, the z-step value ranges from 0.1 μm to 1 mm, such as 1 to 5 μm.

In some embodiments, the calibration procedure involves digitally dividing a sample into a plurality of tiles. A tile can be an image of a discrete portion of a sample. When the field of view of a microscope objective is smaller than the sample itself, it may be necessary to collect multiple stacks of images in tiling arrangements, and then stitch together the tiles to generate a complete image. In some embodiments, the methods include defining the coordinates of the two opposite corners of a region that will define the number of tiles, and setting a desired z-step value that will be used to collect a stack of tile images. In some embodiments, regions are selected as tiles having an overlap with each other ranging from 10 to 50%, such as 15 to 20%.

Aspects of the calibration procedure involve acquiring an alignment parameter for each of a plurality of locations within the sample. To acquire the alignment parameter at each location, a light sheet produced by moving a beam of light in one dimension is used to illuminate a plane of the sample. The plane of the sample that is illuminated by the light sheet is referred to herein as a “sample illumination plane” or “illumination plane.” Alignment between the detection focal plane of the microscope and the sample illumination plane is accomplished by finding a maximum image quality measurement in a specified neighborhood corresponding to optimal alignment between the sample illumination plane and the detection focal plane. In some embodiments, the image quality measurement is an optical focus quality measurement that comprises a ratio of high frequency and low frequency signals in Fourier space.

The result of the calibration procedure is multiple alignment parameters that correspond to different locations within the sample. By applying an alignment parameter at a given location within the sample, optimal alignment between the detection focal plane of the microscope device and the light sheet is achieved at the location. In some embodiments, a z-step value of 1 mm is used to carry out the calibration procedure, and linear interpolation between two adjacent locations within the sample is used to determine an alignment parameter at a location between the two adjacent locations. In this way, the calibration procedure can be performed using a z-step value of 1 mm, and the results can then be applied to the entire sample to determine an alignment parameter at any location. In some embodiments, the calibration procedure is automated. In some embodiments, a processor executes instructions that cause a controller to execute the calibration procedure and acquire a plurality of alignment parameters for the sample.

Aspects of the methods include performing an imaging procedure that utilizes the alignment parameters to align a detection focal plane of the microscope device with an illumination plane of the sample. In some embodiments, the imaging procedure involves aligning a detection focal plane of the microscope device with an illumination plane of the sample, illuminating a linear portion of the illumination plane with a beam of light from a light source, and capturing a plurality of emitted light signals from the illuminated linear portion of the illumination plane with the camera. In certain embodiments, alignment of a detection focal plane of the microscope device and an illumination plane of the sample is synchronized, such that illumination of the illumination plane is carried out at the same time that the detection focal plane is aligned with the illumination plane. In this way, only the portion of the sample that is actively being imaged is illuminated, thereby reducing photo-bleaching of signals in the sample that may result from excessive illumination.

In some embodiments, the imaging procedure involves directing a beam of light from a light source to sweep across an illumination plane and thereby illuminate a plurality of linear portions of the illumination plane. In some embodiments, as the beam of light sweeps across the illumination plane, a plurality of emitted light signals from each different linear portion of the illumination plane is captured by the camera, resulting in a two dimensional image of the sample that coincides with the illumination plane. In some embodiments, the period of time during which an illumination plane of the sample is illuminated ranges from 1 millisecond (ms) to 1 second, such as 5 to 100 ms.

In some embodiments, the methods involve directing multiple independent light sheets to illuminate different portions of a sample. For example, in some embodiments, two or more light sheets are used to illuminate a sample from opposite sides of the sample. In some embodiments, a plurality of light sheets are used to illuminate the sample, such as two or more, three or more, four or more, five or more, six or more, seven or more, or up to eight or more light sheets. In some embodiments, a single light sheet is used to illuminate two or more different portions of a sample by quickly moving, or “switching” the light sheet from one position to another.

In some embodiments, the imaging procedure further involves moving the sample in the z-direction such that the light sheet will illuminate a new illumination plane, and repeating the imaging procedure to generate another two dimensional image of the sample coinciding with the new illumination plane. In some embodiments, the sample remains stationary while the light sheet and the detection objective are moved to image a new illumination plane. In some embodiments, the imaging procedure involves synchronously moving the optics (e.g., the objectives in the illumination beam path(s)) and the light sheets that are used to illuminate the sample. In some embodiments, the imaging procedure involves continuously moving the sample with the sample manipulation components at a defined rate, such that the camera in the detection beam path can continuously image various planes of the sample as they are illuminated. Any of the above-described methodology can be implemented with respect any given light sheet. As such, utilizing multiple light sheets in a COLM system can be used to increase the overall speed of the imaging procedure for a particular sample, as well as increase the size of a sample that can be imaged using the subject systems and methods.

In some embodiments, the result of the illumination procedure is a plurality of two dimensional images of the sample, each corresponding to a different illumination plane of the sample. In some embodiments, the imaging procedure is automated. In some embodiments, a processor executes instructions that cause a controller to execute the imaging procedure and acquire a plurality of two dimensional images of the sample, each corresponding to a different illumination plane of the sample.

In some embodiments, the imaging procedure further involves data processing of two dimensional images to form a three dimensional image of the sample. In some embodiments, two or more different two dimensional images of a sample may be combined, or “stitched” together to form a single two dimensional image of a plane of the sample. For example, as described above, when a sample is imaged using two or more different independent light sheets, the images obtained from each light sheet may be combined to form a single two dimensional image corresponding to a given plane of the sample.

Three dimensional reconstruction software is commercially available, and can be used to stitch together tiled images and/or reconstruct a plurality of two dimensional images into a three dimensional image. Commercially available software programs include those from Imaris, Bitplane and Amira, as well as open source software such as the stitching plugins in Fiji, XuvTools, Vaa3D plugin, and TeraStitcher. In some embodiments, manual or semi-automatic tracing of neuronal morphology in a sample comprising neuronal tissue can be performed using specific modules in commercial software, such as Imaris and Amira, or with open-source tools such as Neuromantic.

In come embodimetns, microscopic analysis imaging includes: placing the sample in a sample chamber in an optically homogenous sample manipulation component; performing a calibration procedure to align one or more light sheets and one or more detection focal planes of a microscope device at a plurality of locations within the sample to acquire an alignment parameter for each location; performing an imaging procedure to collect an image from each of the plurality of locations within the sample; applying the alignment parameter to each location and simultaneously illuminating the location with a light sheet and capturing an image of the location; and constructing a three-dimensional image of the sample using the image from each location.

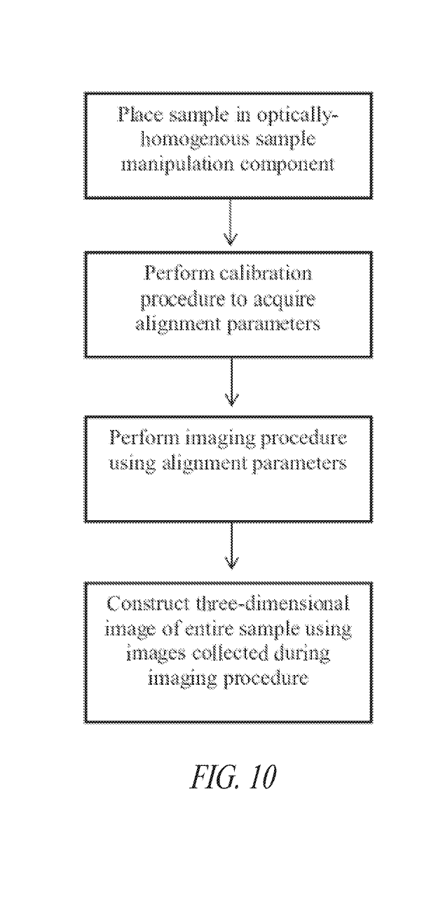

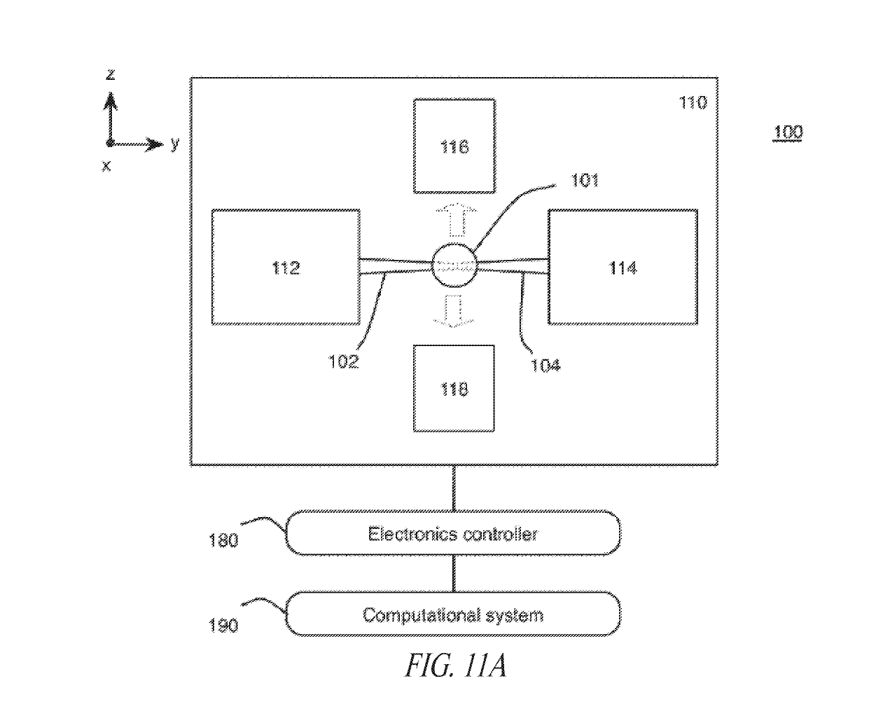

Referring now to FIG. 10, a block flow diagram of one embodiment of the methods is shown. In the depicted embodiment, the method includes placing a sample in the optically-homogenous sample manipulation component, performing a calibration procedure on the sample to acquire a plurality of alignment parameters, performing an imaging procedure on the sample using the alignment parameters, and constructing a three-dimensional image of the entire sample using the images captured during the imaging procedure.

Referring now to FIG. 7, panel b, an embodiment of a method for obtaining high-quality deep images or a large intact sample is shown. As depicted, two independent planes, illuminated with two aligned sheets of light (either one beam from each side illuminating the two different planes, or two from each side, with one from each plane side illuminating the same plane and the remaining illuminating the other plane), are imaged, either simultaneously or sequentially from opposite detection arms. A two dimensional image of the sample is generated from each light sheet. Panel b demonstrates images of the same plane imaged from two opposite detection arm. The detection arms can be aligned precisely (for example, with sub-micron accuracy). FIG. 7, panel b demonstrates images from slightly misaligned arms, and that by small corrections in the X or Y dirrection can be superimposed. Using this method, larger samples can be imaged, as the overall working distance of the combination of both objectives is increased by two-fold through the addition of the second objective in the second detection beam path.

Referring now to FIG. 7, panel c, various methods of increasing imaging speed are shown. As depicted, fast switching of a light sheet may to illuminate two independent planes be used to increase overall imaging speed. Similarly, simultaneous multi-planar imaging can also be used to increase the overall speed of the imaging process. Non-stop sample imaging, wherein multiple light sheets are continuously moved through the sample or the sample is moved continuously through multiple light sheets while continuously acquiring images, can also be used to increase the overall speed of the imaging process. In addition, step-wise optical z-scanning can be used with multiple light sheets to sequentially image different optical planes of a sample. Uni-directional and bi-directional synchronized illumination and detection can also be used to increase the overall speed of the imaging process. In bi-directional synchronized illumination and detection, two or more independent light sheets are used to illuminate and image different portions of a sample, as depicted in FIG. 7, panel c. The use of multiple light sheets moving in different directions increases the overall speed of the imaging process.

Referring now to FIG. 9, a method of increasing axial (z-resolution) is shown. By imaging a sample from four independent, orthogonally arranged, signal detection arms, a 3D image is acquired from four orthogonal views. By fusing these four views, improvement in axial resolution (z-resolution) is achieved. FIG. 9 shows two automatically switchable configurations wherein a sample is first image by a set of two detection arms, and then by an orthogonally arranged part of the detection arm. This switching is performed using fast, precise flip mirrors, which either reflect excitation light or allow it to pass through unhindered.

In some embodiments, aspects of the invention are directed to methods, devices, and systems for imaging of tissue samples (e.g., tumor biopsy samples, prepared as described above) using multiple, parallel light sheets, such as generally disclosed in U.S. Patent Application Publication No. 2015/0098126 titled “MULTIVIEW LIGHT-SHEET MICROSCOPY”, filed Oct. 8, 2014, the entire disclosure of which is incorporated herein by reference.

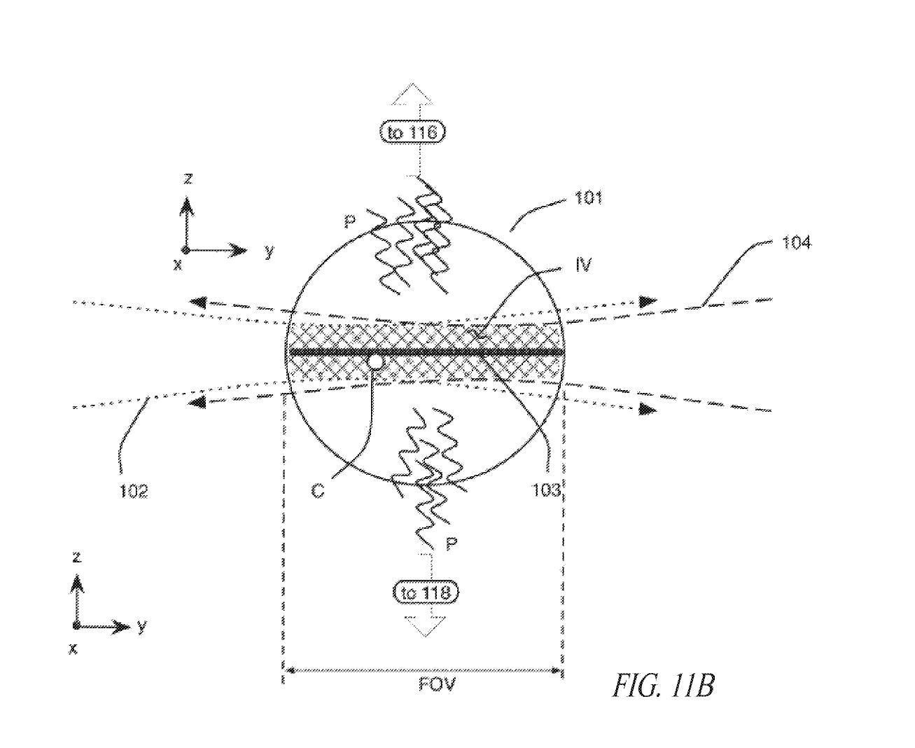

Referring to FIG. 11A, this description relates to a microscope system 100 and corresponding process for live imaging of a complex biological sample (or sample) 101, such as a developing embryo or a tissue biopsy sample, in its entirety. The microscope system 100 uses light-sheet microscopy technology that provides simultaneous multiview imaging, which eliminates or reduces spatiotemporal artifacts that can be caused by slower sequential multiview imaging. Additionally, because only a thin section (for example, on the order of a micrometer (μm) wide taken along the z axis) of the sample 101 is illuminated at a time with a scanned sheet of laser light while a detector records the part of the sample 101 that is being illuminated, damage to the sample 101 is reduced. No mechanical rotation of the sample 101 is required to perform the simultaneous multiview imaging.

In general, the optical microscope 110 is made up of light sheets (for example, light sheets 102, 104) that illuminate the sample 101 from distinct directions along respective light sheet axes, and a plurality of detection subsystems (for example, detection subsystems 116, 118) that collect the resulting fluorescence along a plurality of detection views. In the example that follows, two light sheets 102, 104 are produced in respective illumination subsystems 112, 114, which illuminate the sample 101 from opposite directions or light sheet axes; and the respective detection subsystems 116, 118 collect the resulting fluorescence along two detection views. In this particular example, the light sheet axes are parallel with an illumination axis (they axis) and the detection views are parallel with a detection axis (the z axis), which is perpendicular to the y axis.

Therefore, in this example, the microscope system 100 provides near-complete coverage with the acquisition of four complementary optical views; the first view comes from the detection system 116 detecting the fluorescence emitted due to the interaction of the light sheet 102 with the sample 101; the second view comes from the detection system 116 detecting the fluorescence emitted due to the interaction of the light sheet 104 with the sample 101; the third view comes from the detection system 118 detecting the fluorescence emitted due to the interaction of the light sheet 102 with the sample 101; and the fourth view comes from the detection system 118 detecting the fluorescence emitted due to the interaction of the light sheet 104 with the sample 101.

Referring to FIG. 11B, which is exaggerated to more clearly show the interactions between the light sheets 102, 104, and the sample 101, the light sheets 102, 104 spatially overlap and temporally overlap each other within the sample 101 along an image volume IV that extends along the y-x plane, and optically interact with the sample 101 within the image volume IV. The temporal overlap is within a time shift or difference that is less than a resolution time that corresponds to the spatial resolution limit of the microscope 110. In particular, this means the light sheets 102, 104 overlap spatially within the image volume IV of the biological sample 101 at the same time or staggered in time by the time difference that is so small that any displacement of tracked cells C within the biological sample 101 during the time difference is significantly less than (for example, an order of magnitude below) a resolution limit of the microscope 110, where the resolution limit is a time that corresponds to a spatial resolution limit of the microscope 110.

Each light sheet 102, 104 can be generated with a laser scanner that rapidly moves a thin (for example, μm-thick) beam of laser light along an illumination axis (the x axis), which is perpendicular to the y and z axes, to form a light beam that extends generally along or parallel with a plane to form the sheet 102, 104. In some embodiments, the laser beam in the form of the light sheet 102, 104 illuminates the sample 101 along they axis on opposite sides of the sample 101. Rapid scanning of a thin volume and fluorescence detection at a right angle (in this example, along the z axis) to the illumination axis provides an optically sectioned image. The light sheets 102, 104 excite fluorophores within the sample 101 into higher energy levels, which then results in the subsequent emission of a fluorescence photon P, and the fluorescence photons P are detected by the detectors within the detection subsystems 116, 118 (along the z axis). As discussed in detail below, in some implementations, the excitation is one-photon excitation, or it is multi-photon (for example, two-photon) excitation.

The fluorophores that are excited in the sample can be labels that are attached to the cells, such as, for example, genetically-encoded fluorescent proteins such as GFP or dyes such as Alexa-488. However, the fluorophores can, in some implementations that use second-harmonic generation or third-harmonic generation, be actual or native proteins within the cells that emit light of specific wavelengths upon exposure with the light sheets 102, 104.

As shown schematically in FIG. 11B, the light sheets 102, 104 pass through the sample 101 and excite the fluorophores. However, the light sheets 102, 104 are subject to light scattering and light absorption along their respective paths through the sample 101. Moreover, very large (large compared with the image volume IV or the field-of-view (FOV)) or fairly opaque samples can absorb energy from the light sheets 102, 104.

Moreover, if the light sheets 102, 104 are implemented in a two-photon excitation scheme, then only the central region 103 of the overlapping light sheets 102, 104 may have a high enough power density to efficiently trigger the two-photon process, and it is possible that only (the close) half of the sample 101 emits fluorescence photons P in response to exposure to two-photon light sheets 102, 104.

The term “spatial overlap” of the light sheets could mean that the light sheets 102, 104 are overlaid geometrically within the sample 101. The term “spatial overlap” can also encompass having the light sheets both arrive geometrically within the FOV (as shown in FIG. 11B) of the detection subsystems 116, 118 and within the sample 101. For example, to efficiently trigger two-photon excitation, each light sheet 102, 104 could cover only a part of (for example, one half) of the field-of-view of the detection subsystems 116, 118 (so that each light sheet is centered in the respective half of the field-of-view) so that the use of both of the light sheets 102, 104 leads to the full field-of-view being visible.

Because of this, each two-photon light sheet 102, 104 can be made thinner (as measured along the z axis), if the light sheet 102, 104 only needs to cover half of the field-of-view. However, if the light sheet 102, 104 is thinner (and the laser power unchanged), the same number of photons travel through a smaller cross-section of the sample 101, that is, the laser power density is higher, which leads to more efficient two-photon excitation (which is proportional to the square of the laser power density). At the same time, because the light sheets are thinner, the resolution is increased when compared to a scenario in which each light sheet 102, 104 covers the entire field-of-view.

As an embodiment, if the light sheets 102, 104 are implemented in a one-photon excitation scheme, the light sheet 102, 104 could also excite fluorophores on the area of the sample 101 outside of the central region 103 but within the image volume, and this part will appear blurrier in the resultant image. In this latter case, two images can be sequentially recorded with each of the two light sheets 102, 104, and the computational system 190 can use a calculation to adjust the images to obtain a higher quality. For example, the two images can be cropped such that the low-contrast regions are eliminated (and complementary image parts remain after this step), and then the images recorded with the two light sheets can be stitched together to obtain a final image that covers the entire field-of-view in high quality.

The light sheet 102, 104 is configured so that its minimal thickness or width (as taken along the z axis) is within the image volume IV and the FOV. When two light sheets 102, 104 are directed toward the sample 101, then the minimal thickness of the respective light sheet 102, 104 should overlap with the image volume IV. As discussed above, it can be set up so that the minimal thickness of the light sheet 102 is offset from the minimal thickness of the light sheet 104, as shown schematically in FIG. 11B. This set up provides improved or superior spatial resolution (for both one-photon and two-photon excitation schemes) and improved or superior signal rates (for two-photon excitation schemes).

For example, for a sample 101 that is a Drosophila embryo that is about 200 μm thick (taken along the z axis), the light sheet 102 can be configured to reach its minimal thickness about 50 μm from the left edge (measured as the left side of the page) of the sample 101 after it crosses into the sample 101 while the light sheet 104 can be configured to reach its minimal thickness about 50 μm from the right edge (measured as the right side on the page) of the sample 101 after it crosses into the sample 101.

There is a tradeoff between the minimal thickness of the light sheet 102, 104 and the uniformity of the light sheet 102, 104 thickness across the image volume IV. Thus, if the minimal thickness is reduced, then the light sheet 102, 104 becomes thicker at the edges of the image volume IV. The thickness of the light sheet 102, 104 is proportional to the numerical aperture of the respective illumination subsystem 112, 114; and the useable length of the light sheet 102, 104, that is, the length over which the thickness is sufficiently uniform, is inversely proportional to the square of the numerical aperture. The thickness of the light sheet 102, 104 can be estimated using any suitable metric, such as the full width of the light sheet 102, 104 along the z axis taken at half its maximum intensity (FWHM).

For example, a light sheet 102, 104 having a minimal thickness of 4 μm (using a suitable metric such as the FWHM) is a good match for an image volume IV that has a FOV of 250 μm (which means that it is 250 μm long (as taken along the y axis)). A good match means that it provides a good average resolution across the field of view. A thinner light sheet would improve resolution in the center (taken along they axis) but could degrade the resolution dramatically and unacceptably at the edges of the sample 101, and possibly lead to worse average resolution across the field-of-view. A thicker light sheet would make the light sheet more uniform across the image volume IV, but it would degrade the resolution across the entire image volume IV. As another example, a light sheet 102, 104 having a minimal thickness of 7 μm (using a suitable metric such as the FWHM) is a good match for an image volume IV that has a FOV of 700 μm (and thus it is 700 μm long (as taken along the y axis)).

In general, the thickness of the light sheet 102, 104 (as taken along the z axis) should be less than the size (and the thickness) of the sample 101 to maintain image contrast and reduce out of focus background light. In particular, the thickness of the light sheet 102, 104 should be substantially less than the size of the ssample 101 in order to improve the image contrast over a conventional illumination approach in which the entire sample 101 is illuminated. Only in this regime (light sheet thickness is substantially smaller than sample thickness), light-sheet microscopy provides a substantial advantage over conventional illumination approaches.

For example, the thickness of the light sheet 102, 104 can be less than one tenth of the width of the sample 101 as taken along the z axis. In some implementations, the thickness of the light sheet 102, 104 is on the order of one hundredth of the width or size of the sample 101 if each light sheet is used to cover only a portion (such as a half) of the field-of-view, that is, the point of minimal thickness of each light sheet is located in the center of one of the two halves of the field-of-view, as discussed above.

One consideration for setting the minimal thickness of the light sheet 102, 104 or for defining a realistic image volume IV, is the size of the structure within the sample 101 that needs to be resolved by the microscope system 100. For example, if the microscope system 100 is set up to image cell nuclei, which are about 2-10 μm in size (taken along a straight line across the nucleus; about 10 μm in human fibroblasts). To achieve reasonably good spatial sampling and resolution, the light sheet 102, 104 should have a minimal thickness that is not much thicker than half the cross-sectional size or length of these nuclei, e.g., about 5 μm. Moreover, the images of the sample 101 should be recorded in steps taken along the z axis (by either translating the sample 101, the light beams 102, 104, or both the sample 101 and the light beams 102, 104 along the z axis); and the size of the steps should be about the size of this minimal thickness.

The microscope system 100 also includes an electronics framework that includes the electronics controller 180 and the computational system 190. The electronics controller 180 provides synchronized control of all opto-mechanical components within the microscope 110 with millisecond precision over long periods of time. The computational system 190 rapidly performs the complex optical alignment on the live sample 101, and provides a robust pipeline for simultaneous high-speed image acquisition with a plurality of detectors (or cameras) within the detection subsystems 116, 118 at sustained data rates of several hundreds of megabytes per second and a high-throughput computational strategy for efficient automated image processing to register and reconstruct the terabytes of raw multiview image data arising from every experiment.

While the example provided herein describes a four-view system using two light sheets for illumination and two detectors for collecting the fluorescence, more views are possible by adding additional detectors and/or illumination systems. In this example, the illumination subsystems 112, 114 face each other such that the sample 101 can be illuminated with light sheets from two sides, as more clearly shown in the schematic drawing of FIG. 11B. Each detection subsystem 116, 118 includes a respective detector or camera 106, 108 in addition to a set of detection optical devices arranged to collect and record the fluorescence emitted from the sample 101. Each detection subsystem 116, 118 also includes a set of actuators that are coupled to one or more of the detector 106, 108 and the detection optical devices at one end and interface at the other end with the electronics controller 180.

Each combination of illumination and detection provides a different view or perspective. By capturing the four views (in this example) simultaneously, delays caused by rotating of the sample 101 repeatedly into new positions (as in the sequential imaging techniques of past) are reduced or eliminated. Thus, the microscope system 100 is designed for the simultaneous acquisition of multiple complementary views without having the rotate the sample 101.

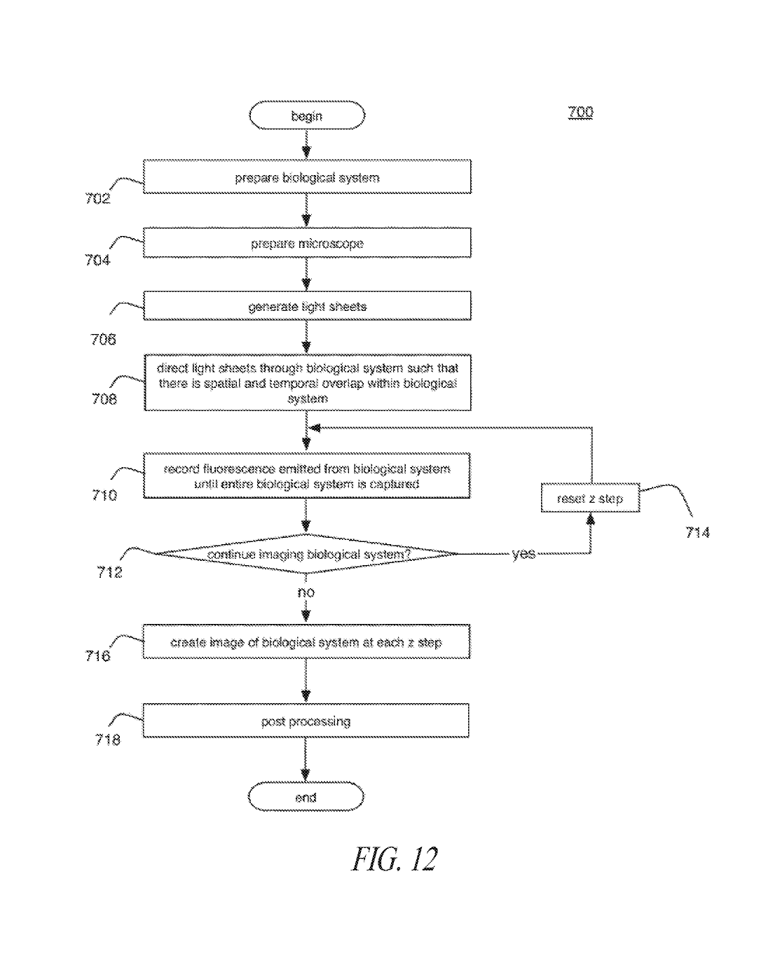

Referring to FIG. 12, a procedure 700 is performed by the microscope system 100 to image the complex biological sample 101. Initially, the biological sample 101 is prepared (702). The biological sample 101 is prepared (702) by chemically and biologically preparing the sample as described above, physically transferring or mounting the sample to the holder 166, and placing the holder 166 inside the chamber 168.

The microscope 110 is prepared (704), for example, by adjusting properties (such as the alignment) of the light sheets 102, 104. Once the microscope 110 is prepared, the light sheets 102, 104 are generated (706). The lights sheets 102, 104 are directed through the biological sample 101 such that there is spatial and temporal overlap within the sample 101 (708). The beginning of the illumination and recording of the fluorescence can start at the moment the fertilized egg is formed, to enable imaging of the biological sample 101 in its development from a fertilized egg to a complex system.

Fluorescence emitted from the biological sample 101 is recorded by the cameras 106, 108 until the entire biological sample 101 is captured (710). It is then determined if the imaging of the biological sample 101 should continue (712). For example, imaging can usually continue until the onset of strong muscle contractions in the developing embryo; at that point, imaging can be stopped because the sample 101 becomes more physically active and can be more difficult to image. However, it is possible that imaging could continue past this developmental point.

If imaging is to continue (712), then the relative alignment between the light sheets and the biological sample 101 is reset (714) and the fluorescence is once again recorded (710). The image of the biological sample 101 is created (716) and additional post processing can be performed (718).

Aspects of the invention are directed to methods, systems, and devices for measurement of fluorescence in biological samples, such as tumor biopsy samples. In some embodiments, aspects of the invention are directed to methods, systems, and devices for quantification of fluorescence in biological samples, such as tumor biopsy samples. Any suitable quantification approach can be employed including, but not limited to, fluorescence intensity, dual-wavelength ratiometric fluorescence, fluorescence lifetime imaging microscopy (FLIM), fluorescence in situ hybridization (FISH), fluorescence thresholding, Forster resonance energy transfer (FRET), fluorescence recovery after photobleaching (FRAP), fluorescence loss in photobleaching (FLIP), fluorescence localization after photobleaching (FLAP), combinations thereof (e.g., FRET-FLIM) and/or the like. According to certain embodiments, image analysis software utilized for 2-D capture and quanitification of fluorescence is used or adapted for use in the present methods. Such image analysis software may include, e.g., the Vectra®, InForm, and Mantra automated quantitative pathology imaging systems (Perkin-Elmer). For example, the Vectra® 3 automated quantitative pathology imaging system accurately detects and measures weakly expressing and overlapping biomarkers within a single H&E, IHC or IF intact FFPE tissue section or TMA, and may be adapted for use in the present invention. Vectra° and inForm° software analysis combine the power of multiplexed biomarker imaging and quantitative analysis. In certain embodiments, tissue samples or TMAs can be labeled with immunofluorescent (IF) or immunohistochemical (IHC) stains, or with conventional stains such as H&E and trichrome. When using IF or IHC stains, multiple proteins can be measured on a per tissue sample, per cell, or per cell compartment (e.g. nuclear, cytoplasmic) basis—even when signals are spectrally similar, are located in the same cellular compartment or are obscured by autofluorescence. Mantra™ quantitative pathology workstation with inForm® image analysis software enables easy visualization, quantification and phenotyping of multiple types of immune cells simultaneously in intact FFPE tissue sections for cancer immunology research, and may be adapted for use with larger tissue samples according to the present invention. Mantra™ is a compact workstation that can be used to phenotype immune cells in situ in solid tumors using multiplexed biomarkers. Unlike existing flow cytometry and next generation sequencing methods which can phenotype and quantify immune cells in homogenized samples, Mantra™ was designed to do this using images of FFPE tissue sections, while maintaining tissue architecture and morphology. The Mantra™ is an integrated workstation incorporating multispectral imaging technology, novel image acquisition and inForm analysis software. It can be used with a variety of stains including PerkinElmer's Opal™ reagent kits, any of which may be used in the methods of the present invention.

Certain embodiments of methods of analysis are described in PCT Application No. PCT/US2015/032951 and Tomer, R. T et al., Nature Protocols, Vol. 9, No. 71682-1697 (June 2014) , which are hereby incorporated by reference in their entireties.

### IV. Characterization of Biological Samples and Disease States

Methods of the present invention include determining one or more traits of a biological sample, e.g., a tumor tissue sample, for example, to diagnosis the presence of a disease (e.g., a cancer), determine the prognosis of a disease (e.g., a cancer), monitor progression of a disease over time (e.g., a cancer), determine the response of a disease (e.g., a cancer) to a therapeutic treatment, determine the likelihood of a disease becoming resistant to therapy (e.g., a cancer treatment), or detect or monitor resistance to a therapy. In addition, methods of the present invention may be used to test candidate therapeutic agents, e.g., by determining the response of a subject or disease to treatment with the candidate agent. Methods of the present invention may be practiced on biological samples obtained from patients or from animal models of disease.

The traits or characteristics examined may include phenotypic characteristics and/or genetic traits or characteristics. A variety of different characteristics of a biological sample may be determined or measured, including but not limited to: (i) the presence of, absence of, location, or distribution of specific cellular or extracellular structures; (ii) the shape, size, complexity, location or distribution of specific cellular or extracellular structures, e.g., blood vessels, lymphatic vessels or extracellular matrix components; (iii) the presence of, absence of, location of, distribution of, or the amount of, specific cell types, e.g., immune cells or stem cells; and (iv) the presence or absence of, location or distribution of, or the amount of specific disease markers, e.g., the expression levels of proteins associated with disease, such as tumor markers. In certain embodiments, the spatial distribution and/or quantity of a plurality of such characteristics may be more important than each individual characteristic for purposes of the present methods.

A variety of reagents and methods are available for examining tissue cellular structures and disease marker expression levels, including macromolecules (e.g., antibodies or fragments thereof and aptamers) that specifically bind cell surface and intracellular markers indicative of particular cell types, macromolecules that specifically bind polypeptides having expression levels indicative of a disease, and nucleic acid probes that specifically bind nucleic acids, such as mRNA, having expression levels indicative of disease, macromolecules that specifically bind mutated polypeptide associated with a disease, and polynucleotides that specifically bind nucleic acids, e.g., genes or mRNAs, comprising a mutation associated with a disease, such as a translocation, insertion, deletion, or codon substitution. In particular embodiments, macromolecules, i.e., detectable labels, include antibodies or fragments thereof, or aptamers. In particular embodiments, nucleic acid probes, i.e., detectable labels, comprises an antisense sequence as compared to the target polynucleotide biomarker being detected. In certain embodiments, nucleic acid primers are between 8 and 40 nucleotides in length. In particular embodiments, they bind both 5′ and 3′ to a site of gene translocation. Conditions suitable for the binding of polypeptides, e.g., antibodies or aptamers, and nucleic acids, e.g., primers, to target molecules are known in the art. During labeling, the tissue sample may be contacted with a detectable probe under conditions suitable for and for a time sufficient to allow the detectable label to selectively bind to or hybridize to target biomarkers in the tissue sample. In certain embodiments nucleic acid biomarkers can be detected using fluorescence in situ hybridization (FISH). Methods of using FISH to detect and localize specific DNA sequences, localize specific mRNAs within tissue samples or identify chromosomal abnormalities are described in Shaffer D R et al, Clin Cancer Res. 2007 Apr. 1; 13(7):2023-9, Cappuzo F et al, Journal of Thoracic Oncology, Volume 2, Number 5, May 2007, Moroni Met al, Lancet Oncol. 2005 May; 6(5):279-86, each of which is herein incorporated by reference in its entirety.

A disease, such as a cancer, may be diagnosed, detected or confirmed based on the results of examining one or more traits or characteristics of a biological sample, e.g., a tumor tissue sample, using methods described herein. In particular embodiments, the one or more characteristics of the biological sample are compared to those characteristics in a normal control sample, e.g., a disease-free sample obtained from the same tissue or organ. This may be performed by side-by-side analysis, or by comparing the one or more characteristics of the biological sample to pre-determined values or parameters previously obtained from one or more normal control samples or samples having the same cancer but different in phenotype or genotype.

The prognosis of a disease, such as a cancer, may be determined based on the results of examining one or more traits or characteristics of a biological sample, e.g., a tumor tissue sample, using methods described herein. In particular embodiments, the one or more characteristics of the biological sample are compared to those characteristics in biological samples of the same type of disease identified as having a “good” or “poor” prognosis. In certain situations, the presence or absence of one or more traits may be associated with an aggressive form of the disease or increased mortality, whereas the presence or absence of one or more other traits may be associated with a mild form of the disease or low mortality. This prognostic analysis may be performed by side-by-side analysis of the biological sample with biological samples known to be associated with a good or poor prognosis, or by comparing the one or more characteristics of the biological sample to pre-determined values or parameters of the characteristics known to be associated with a good or poor prognosis, e.g., values or parameters previously determined based on analysis of these characteristics in a plurality of diseased biological samples, each associated with a good or poor disease prognosis.

The predicted responsiveness of a disease, such as a cancer in a subject to a particular therapy or treatment may be determined based on the results of examining one or more traits or characteristics of a biological sample, e.g., a tumor tissue sample, at one or more different time points, using methods described herein. In certain embodiments, the biological sample is obtained from the subject prior to treatment. In particular embodiments, the one or more characteristics of the biological sample are compared to those characteristics in biological samples of the same type of disease identified as having a good response or a poor response to the particular therapy or treatment. This analysis may be performed by side-by-side analysis of the biological sample with biological samples known to be associated with a good or bad response to the particular therapy or treatment, or by comparing the one or more characteristics of the biological sample to pre-determined values or parameters of the characteristics known to be associated with a good or bad response to the particular therapy, e.g., values or parameters previously determined based on analysis of these characteristics in a plurality of diseased biological samples, each associated with a good or poor response to the particular treatment or therapy. In certain embodiments, the responsiveness to therapy or treatment is a recognized clinical endpoint, such as overall survival (OS), progression-free survival (PFS), time to progression (TTP), time to treatment failure (TTF), event-free survival (EPS), time to next treatment (TTNT), overall response rate (ORR) or duration of response (DofR). In other embodiments, the one or more characteristics include the absence of, the presence of, or an amount of a marker of resistance to a particular treatment, and the predicted responsiveness of the disease to the treatment may be determined based on the presence or absence of the treatment resistance marker. In particular embodiments, the presence of the treatment resistance marker, or a high amount of the treatment resistance marker is predictive of a poor response to the treatment, whereas the absence of the treatment resistance marker, or a low amount of the treatment resistance marker is predictive of a good response to the treatment.

In particular embodiments, methods of the present invention may be practiced to determine what particular treatment or therapy to provide to a subject diagnosed with a particular disease, such as a cancerous tumor, based on determining the predicted responsiveness of the subject's disease to one or more different treatments or therapies for the disease, e.g., as described above.

The actual responsiveness of a disease, such as a cancer, in a subject to a particular therapy or treatment may be determined based on the results of examining one or more traits or characteristics of a biological sample, e.g., a tumor tissue sample, at two or more different time points, using methods described herein. In particular embodiments, one or more characteristics are determined for a biological sample obtained at a first time point, e.g., at initial diagnosis or before a treatment, and the one or more characteristics are then determined at a second, later time point, e.g., during or after the treatment. The one or more characteristics determined at the first time point may be compared to the one or more characteristics determined at the second or further time points, and any changes that occurred between the time points identified. Where the one or more characteristics determined at the second or further time point are more similar to a normal control than those determined at the first time point, it is indicative that the therapy is treating or inhibiting progression of the disease. Where the one or more characteristics determined at the second time point are more similar to those of a known disease sample, it is indicative that the therapy is not treating or inhibiting progression of the disease. Characteristics associated with normal or non-disease or with disease may be pre-determined based on prior examination of biological samples obtained normal or diseased tissues. In certain embodiments, assessment, e.g., for later stages of cancer, comprising assessing the RESIST criteria of tumor progression, which are described, e.g., in Eisenhauer, E. A. et al., European Journal of Cancer, Vol. 45, pp. 228-247 (2009). In other embodiments, the one or more characteristics include the absence of, the presence of, or an amount of a marker of resistance to a particular treatment, and the responsiveness of the disease to the treatment may be determined based on a change in the presence, absence or amount of the marker of resistance at two different time points. For example, the initial absence of a marker of treatment resistance at a first time point, e.g., before treatment, may indicate that the treatment is efficacious, but the later presence of the marker of treatment resistance at a later time point may indicate that the treatment will no longer be efficacious. Methods of the present invention may be used to screen candidate therapeutic agents for their effectiveness in treating a disease, such as a tumor.

In certain embodiments of any of the methods described herein, the method further comprises imaging one or more control biological sample, e.g., a positive or negative control, e.g., to confirm that tissue processing and/or labeling was successful. In particular embodiments, e.g., those that comprise determining an amount of a detectable marker, the amount of the detectable marker determined for the control sample under the same conditions is used to normalize the amount detected in the test biological sample. In particular embodiments, the method further comprises labeling the control biological sample and imaging the labeled sample. In particular embodiments, it comprises fixing, polymerizing, clearing, and labeling the one or more control biological sample. In particular embodiments, the control biological sample is a cell pellet, such as, e.g., a cultured cell line. In certain embodiments, the control is a frozen or previouly frozen cell pellet. In particular embodiments, the control is a previously fixed and cleared biological sample, such as, e.g., a previously fixed and cleared pell of cultured cells. In certain embodiments of methods that comprise determining an amount of a detectable marker for a test sample, the amount determined is compared to the amount of the detectable marker determined under the same conditions for a control sample, and the amount determined for the test sample.

A. Biological Structures

In particular embodiments, 3-dimensional structures or cell locations and patterns within a biological structure are examined according to methods of the present invention. In addition, the tissue or tumor microenvironment may be examined. In certain embodiments, the present invention includes a method of examining a biological structure is a tissue sample, e.g., a tumor tissue sample, comprising processing a tissue sample obtained from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a biological structure whose characteristics are indicative of the presence or absence of disease, such as a cancer or tumor. In certain embodiments, any of the detectable markers described herein are used to detect an associated disease or tumor. In particular embodiments, the methods further comprises: (vi) comparing the image to one or more control images or predetermined images obtained from normal tissue, disease tissue, tumor tissue, tumor tissue associated with a good prognosis, or tumor tissue associated with a poor prognosis, thereby determining the presence of a tumor or the prognosis of a tumor.

As tumors grow and metastasize, they can effect on the surrounding tissue in which they grow, which is referred to as the tumor microenvironment (TME). The TME includes a variety of non-malignant cells, including blood endothelial cells and lymphatic endothelial cells, along with the extracellular matrix and the inflammatory mediators they secrete. The basal lamina normally separates the parenchyma from the stromal region of tissues, but it is typically incomplete in solid tumors. Changes in the TME may be analyzed according to methods of the present invention, in order to identify changes indicative of tumors, metastasis, prognosis, or responsiveness to treatment. Examples of particular TME features that may be examined include but are not limited to: (i) vascularization, since tumors are abnormally vascularized by disorganized and leaky vessels; (ii) tumor infiltration by innate and adaptive immune cells that can perform either or both protumor or antitumor functions; and (iii) the presence in the TME of non-hematopoietic cell types, including blood endothelial cells and lymphatic endothelial cells, as well as cells of mesenchymal origin, including mesenchymal stem cells and their differentiated progeny, cancer-associated fibroblasts, and pericytes. As compared to normal healthy tissue, the tumor microenvironment may be characterized by any of a variety of cellular and architectural changes, such as leaky blood vessels, disorganized blood vessels, increased interstitial pressure, restructure access of recruited immune cells to the tumor bed, and increased collagen and extracellular matrix deposition. These and other changes, any of which may be assessed according to the present invention are described in further detail in Turley, S. J. et al., Nature Reviews Immunology, Vol. 15, pp. 669-682 (November 2015).

In certain embodiments, blood vessels or microvasculature within the biological sample are examined. Angiogenesis, or new blood vessel formation, is associated with tumor growth and metastasis. New growth in the vascular network of tumors is important, since the proliferation and metastatic spread of cancer cells depends on an adequate supply of oxygen and nutrients and the removal of waste products. Lymphangiogenesis, or new lymphatic vessel formation, also plays a role in tumor growth and metastasis. In addition, increased microvascular density and disorganization may be indicative of tumorigenesis, tumor growth and/or a metastatic cancer. Methods of the present invention allow for the examination of microvasculature throughout a tumor sample, e.g., to determine microvasculature density and organization. In addition, the location of microvasculature within a tumor may be determined, e.g., the location of blood vessels as compared to tumor margins, which may be indicative of the likelihood that a tumor has or will metastasize. In particular embodiments, the presence of blood vessels that protrude beyond the tumor margin is predictive of or indicative of tumor metastasis.

In certain embodiments, microvasculature is examined by staining a sample using agents that specifically bind to blood vessels or cells thereof. In particular embodiments, immunostaining is performed using agents that bind platelet endothelial cell adhesion molecule 1 (PECAM-1) (a marker for endothelial cells) and/or neural/glial antigen 2 (NG2) (a marker for pericytes) to investigate the extent of vascularization. PECAM-1 is used to demonstrate the presence of endothelial cells in histological tissue sections. This can help to evaluate the degree of tumor angiogenesis, which can imply a rapidly growing tumor. Malignant endothelial cells also commonly retain the antigen, so the presence of PECAM-1 can also be used to identify both angiomas and angiosarcomas. NG2 is found both in the central nervous system (CNS) and peripheral tissues. In the central nervous system, NG2 is found on pericytes, various tumors including glioblastoma, and a population of progenitor cells known as polydendrocytes or oligodendrocyte precursor cells (OPCs). Peripherally, NG2 is found on chondroblasts, cardiomyocytes, aortic smooth muscle cells, myoblasts, and several different human tumors, including melanoma. Levels of expression of angiogenic factors reflect the aggressiveness of tumor cells. Epidermal growth factor domain-like 7 (EGFL7) is an extracellular matrix protein that supports endothelial cell adhesion, promotes cell survival under stress, and forms perivascular tracks that regulate blood vessel formation. EGFL7 is selectively expressed in nascent blood vessels in tumors and other proliferating tissues, but is absent or expressed at low levels in healthy quiescent vessels. Preclinical studies also report that EGFL7 may promote tumor escape from immunity. Increased tumor or TME vascularization is characterized by increased levels of soluble factors such as vascular endothelial growth factor A (VEGFA), VEGFC and VEGFD.

Abnormal or increased microvasculature associated with tumors and/or abnormal angiogenesis may be characterized or determined by altered or increased levels of biomarkers such as PECAM-1, NG2 and EGLF7, and/or alterations in the density, structure, branching patterns of the microvasculature as compared to normal tissue samples. Alternatively, or in addition, abnormal or increased microvasculature associated with tumors and/or abnormal angiogenesis may be determined based upon comparison with pre-determined patterns or expression levels in the same type of tumor. In certain embodiments, the predicted clinical outcome may be determined by comparing one or more characteristics of the tumor tissue sample microvasculature to those observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of those having characteristics most similar to the tumor tissue sample being tested. Expression levels of biomarkers may be determined by measuring amounts of expression of the biomarkers, and alterations may be observed by comparing these to a control or pre-determined values, or at two or more time points. Microvasculature structure may be determined by measuring characteristics such as, e.g., the number of micovessel branches, the number of capillaries connecting larger vessels, and the length of microvessels, e.g., by visual assessment, and alterations may be observed by comparing these to a control or predetermined values, at two or more time points.

In certain embodiments, the predicted responsiveness of a tumor to treatment with an anti-angiogenic drug may be determined by measuring the expression level in a tumor tissue sample of any of the following marker genes: VEGFR1, VEGFR2, VEGFR3, VEGFA, VEGFB, VEGFC, VEGFD, PDGFRA and PDGFRB, as these have been implicated as potential markers of response to anti-angiogenic drugs. For example, the expression level of any of these markers in the tumor tissue sample may be compared to its expression level in tumors of the same type either known to be responsive to anti-angiogenic or known to not be responsive to anti-angiogenic drugs, and correlating the predicted outcome based on identifying the expression level most similar to that in the tumor tissue sample.

In other embodiments, the extracellular matrix within the biological sample is examined. The local microenvironment of a cancer cell plays an important role in cancer development. A major component of the local microenvironment is the extracellular matrix (ECM), a complex extracellular network of macromolecules. The ECM serve many functions, such as providing support, segregating tissues from one another, and regulating intercellular communication. The extracellular matrix regulates a cell's dynamic behavior. In addition, it sequesters a wide range of cellular growth factors and acts as a local store for them. The ECM is commonly deregulated and disorganized in diseases such as cancer. Abnormal ECM affects cancer progression by directly promoting cellular transformation and metastasis. ECM anomalies also deregulate stromal cells, facilitate tumor-associated angiogenesis and inflammation, and thus lead to generation of a tumorigenic microenvironment.

Components of the ECM are produced intracellularly by resident cells and secreted into the ECM via exocytosis. Once secreted, they then aggregate with the existing matrix. The ECM is composed of an interlocking mesh of fibrous proteins and glycosaminoglycans GAGs). Components of the ECM include, but are not limited to, proteoglycans, heparan sulfate, chondroitin sulfate, keratan sulfate, non-proteoglycan polysaccharides, hyaluronic acid, fibrins, elastins, collagens and other proteins such as fibronectic, laminin and thrombospondin. Increased expression of thrombospondin has been associated with tumor growth and/or metastasis. One significant difference in clinical prognosis between in situ and invasive or metastatic cancer results predominantly from the presence or absence of the surrounding extracellular matrix basement membrane (BM). Normal or pre-invasive tumor epithelia are normally devoid of lymphatic ducts and blood vessels and are also physically segregated from vascular structures within the stroma by the BM. The BM consists of mainly type IV collagen, laminins, and other molecules that form a continuous sheet (more commonly called the tumor capsule), surrounding the epithelial cells.

Alterations associated with the extracellular matrix may be characterized or determined by altered levels of extracellular matrix components as compared to normal tissue samples. Alternatively, or in addition, the comparison may be made to pre-determined expression levels in the same type of tumor. In certain embodiments, the predicted clinical outcome may be determined by comparing expression levels of one or more extracellular matrix component of the tumor tissue sample to those observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of those having characteristics most similar to the tumor tissue sample being tested. Levels of or changes in expression levels of biomarkers may be determined by measuring amounts of expression of the biomarkers.

Alterations in functional structure of tissue is highly relevant to staging and treatment decision. As just one example, integrity and continuity of ductal structure is integral to the staging of prostate cancer in the Gleason system, in which 3D ductal structure visualization would be of high value in diagnosis, prognosis, and treatment decisions (as it carries crucial information regarding the degree of de-differentiation, metaplasia, neoplasia, metastatic potential, and tissue destruction. This concept may be extended to any other tissue with ductal structure (breast, pancreas, gall bladder, renal, etc.) as well as any tissue in which functional structure relates to neoplasia diagnosis, prognosis, or treatment.

Alterations associated with the extracellular matrix may also be characterized or determined by altered architecture of the extracellular matrix or altered locations or patterns of extracellular matrix components as compared to normal tissue samples, as compared to normal tissue. Alternatively, or in addition, the comparison may be made to pre-determined architecture or protein expression patterns in the same type of tumor.

B. Cell Types

In other embodiments, the presence and/or amount of particular cell types, and/or the 3D arrangement and location of particular cells types, within the biological sample is examined. In particular embodiments, the cell types include stem cells and/or immune cells, such as tumor-infiltration lymphocytes (TILs). In certain embodiments, the present invention includes a method of examining cell types is a tissue sample, e.g., a tumor tissue sample, comprising processing a tissue sample obtained from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a cell type whose presence or amount is indicative of the presence or absence of disease, such as a cancer or tumor, tumor metastasis, or tumor resistance to therapy, e.g., a cancer stem cell. In certain embodiments, any of the detectable markers described herein are used to detect an associated disease or tumor. In particular embodiments, the methods further comprises: (vi) comparing the image to one or more control images or predetermined images obtained from normal tissue, disease tissue, tumor tissue, tumor tissue associated with a good prognosis, or tumor tissue associated with a poor prognosis or metastasis, or prediction of treatment response, thereby determining the presence of a tumor, the likelihood of metastasis, or the prognosis of a tumor.

Leukocytes and their soluble mediators play important regulatory roles in all aspects of solid tumor development. Adaptive and innate immune responses play an important role in tumor immunosurveillance, and they may limit the development and growth of neoplasms. It is hypothesized that the tumor microenvironment immune balance plays a role in prognosis and response to therapy. In addition, chemotherapy may trigger an immune response, which contributes to treatment response. TILs are the main actors in the response against cancer cells, so they constitute surrogate markers of the immune balance between the host and the tumor. TILS are believed responsible for killing tumor cells, and their presence generally correlates with a better clinical outcome. For example, in breast cancer, studies addressing the issue of tumor immune cell infiltration have demonstrated that a high lymphocytic infiltration predicts a better prognosis and a better response to neoadjuvant chemotherapy (NCT), although this benefit might be restricted to some tumor subtypes. Similarly, the relationship between some subtypes of TIL and breast cancer survival is supported by some studies.

Various types of TILs include those expressing CD3, CD4, CD8, CD20, CD68, or Foxp3. CD8+cytotoxic T lymphocytes (CTLs) are directly capable of killing tumor cells. CD4+T helper lymphocytes (Th) are a heterogeneous cytokine secreting class of T lymphocytes. T helper type 1 lymphocytes (Th1) have a crucial role in activating CTLs. T helper type 2 lymphocytes stimulate humoral immunity and activate eosinophils. In terms of antitumor immunity, Th2 activation is less effective than Th1 activation. Besides the Th1 and Th2 subsets, a CD4+ regulatory T lymphocyte (Treg) subset suppresses effector T lymphocytes. FoxP3+ is a relatively selective Treg marker. In cancer, Treg preferentially traffic to tumors, as a result of chemokines produced by tumor cells and microenvironmental macrophages. In recent years, the hypothesis that ratios between different subsets are most predictive of prognosis has gained much attention. Frequently used ratios are CD8+/FoxP3+ (effector:regulatory) ratio and CD8+/CD4+ (effector:helper) ratio.

The presence of or amount of, the types of, and/or the 3D arrangement and location of particular immune cells such as TILs, and the ratios of different TIL subsets within a biological sample may be determined using probes specific for cell surface markers characteristic for different immune cells and different types of TILs. The amounts of, location of, and ratios of different immune cells, and/or the 3D arrangement and location of particular immune cells, within a biological sample, e.g., a tumor tissue may be compared to normal controls, or to pre-determined levels known to be associated with either normal or disease cells. Alternatively, or in addition, the amounts of, location of, and ratios of different immune cells, and/or the 3D arrangement and location of particular immune cells, within a biological sample, e.g., a tumor tissue sample, may be compared to pre-determined amounts of, location of, and ratios of different immune cells in the same type of tumor. In certain embodiments, the predicted clinical outcome may be determined by comparing amounts of, location of, and ratios of different immune cells, and/or the 3D arrangement and location of particular immune cells, to those observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested to that of those tumors with known clinical outcomes having amounts of, location of, and ratios of different immune cells, and/or the 3D arrangement and location of particular immune cells ,most similar to the tumor tissue sample being tested.

Cancer stem cells are cancer cells that possess characteristics associated with normal cells, specifically the ability to give rise to all cell types found in a particular cancer sample. CSCs are turmorigenic and may generate tumors through the stem cell processes of self-renewal and differentiation into multiple cell types. CSCs are hypothesized to persist in tumors as a small, distinct population and cause relapse and metastasis. The detection of CSCs and monitoring the destruction of CSCs during cancer treatment could be important in cancer diagnosis, prognosis and monitoring the effectiveness of treatment. The present invention offers advantages in identifying CSCs, since it allows more thorough investigation of the entirety of the tumor tissue sample than conventional methods.

The presence of or amount of CSCs within a biological sample may be determined using probes specific for cell surface markers characteristic for CSCs, such but not limited to, CD90, Scal, CS133, and Oct3/4. Other probes specific for CSCs and illustrative types of cancer that they have been associated with include: CD24 (heat stable antigen), breast CSCs; A1DH1, breast CDCs; CD44, breast and prostate CSCs; ALDH1, normal and cancer stem cells in a wide range of tissues; EpCAM (Epithelial-specific antigen (ESA)), breast and pancreatic CSCs; CD133 (prominin-1), gliomas and colorectal CSCs; 4-Oct (POU5F1), CSCs in a wide range of tissues; CD34, intestinal, hepatic, and pancreatic CSCs; c-Kit (CD117), intestinal, hepatic, and pancreatic CSCs; and CD10 (CALLA), head and neck squamous cell carcinomas. The amount of CSCs within a biological sample, e.g., a tumor tissue sample, may be compared to normal controls, or to pre-determined levels known to be associated with either normal or diseased cells. Alternatively, or in addition, the amount of CSCs within a biological sample, e.g., a tumor tissue sample, may be compared to pre-determined amounts of, location of, and ratios of CSCs in the same type of tumor. In certain embodiments, the predicted clinical outcome may be determined by comparing the amount of CSCs in the tumor tissue sample to the amount observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested with that of those tumors with known clinical outcomes having amounts of CSCs most similar to the tumor tissue sample being tested.

Cancer-associated fibroblasts (CAFs) are a heterogeneous cell population of multiple origin, and their accumulation in the tumor microenvironment is often correlated with poor prognosis in many tumors. Cancer-associated fibroblasts support cancer cell growth and metastasis, and may hinder antitumor immune responses. Cancer-associated fibroblasts may be identified by expression of markers such as vimentin, a-smooth muscle actin, and fibroblast activation protein, and are characterized by an activated, highly contractile, myofibroblastic phenotype. The amount of CAFs within a biological sample, e.g., a tumor tissue sample, may be compared to normal controls, or to pre-determined levels known to be associated with either normal or diseased cells. Alternatively, or in addition, the amount of CAFs within a biological sample, e.g., a tumor tissue sample, may be compared to pre-determined amounts of, location of, and ratios of CAFs in the same type of tumor. In certain embodiments, the predicted clinical outcome may be determined by comparing the amount of CAFs in the tumor tissue sample to the amount observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested with that of those tumors with known clinical outcomes having amounts of or location of CAFs most similar to the tumor tissue sample being tested.

Pericytes are recruited to tumors by platelet-derived growth factor-β (PDGF-β) gradients, and they posess characteristic cell markers including 3G5 ganglioside and chdroitin sulfate proteoglycan 4, also referred to as NG2. In addition, regulator of G protein signalling 5 (RGS5) is overexpressed in tumor pericytes. Periyte coverage is abnormal in tumor cessels. In the TME, pericytes are loosely associated with the blood endothelial cells and the basemene tmembrane, leading to increased leakiness of tumor vasculature, possibly due to increased VEGFA. The location and amount of pericytes within a biological sample, e.g., a tumor tissue sample, may be compared to normal controls, or to pre-determined levels known to be associated with either normal or diseased cells. Alternatively, or in addition, the location or amount of pericytes within a biological sample, e.g., a tumor tissue sample, may be compared to pre-determined amounts of or locations of pericytes in the same type of tumor. In certain embodiments, the predicted clinical outcome may be determined by comparing the amount of pericytes in the tumor tissue sample to the amount observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested with that of those tumors with known clinical outcomes having amounts of and/or location of pericytes most similar to the tumor tissue sample being tested.

Stromal cells in the tumor microenvironment express numerous surface and secreted molecules that impact tumor growth and the immune reponse to tumors. These molecules may be used as markers for tumors according to methods of the present invention. For example, blood endothelial cells express programmed cell death ligand 1, T cell immmunolobulin and mucin-containing domain molecule 3, and CD95L on their surface, and they secrete indoleamine 2,3-dioxygenase, interleukin-6, prostaglandin E2, and transforming growth factor β. They also express a soluble form of CD137. Pericytes secrete interleukin 6, nitric oxide, prostaglandin E2 and transforming growth factor b. Mesenchymal stem cells secrete heaptocyte growth factor, interleukin 10, IDO, nitric oxide, prostaglandin E2 and transforming growth factor β; cancer associated fibroblasts secrete transofrming growth factor β, thymic stromal lymphopoetin, and inflammatory cytokines including CXC-chemokine ligand 8, interleukin 4, and interleukin 6. M2 macrophages secrete secrete factors such as epidermal growth factor, fibroblast growth factor, platelet-derived growth factor β and transforming growth factor β, which support CAF survival and activation. Tumor-associated macrophages and other immune cells secrete matrix metalloproteinase 9, and soluble mediators, such as cyclooxyenase 2, fibroblast growth factor 2, platelet derived growth factor β and vascular endothelial growth factors, such as vascular endothelial growth factor C and vascular endothelial growth factor D. These may all be used as markers to for detection, prognosis, and following response to therapy of cancer.

C. Gene or Protein Expression Levels and Mutations

Various methods of the present invention include comparing the expression level or expression pattern in the tissue sample, e.g., tumor tissue sample, of one or more markers having altered expression levels in disease cell, e.g., tumor cells, to the expression level or expression pattern in control normal cells or control disease cells, including control disease cells with a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), or control disease cells known to be responsive or not to one or more particular therapies.

These methods may include measuring an amount of expression of the one or more markers and/or measuring the expression patterns throughout the biological sample as compared to the expression pattern in normal control or diseased tissue. In certain embodiments, the marker has an increased level of expression in diseased tissue or cells, while in other embodiments, the marker has a reduced level of expression in diseased tissue or cells. In particular, embodiments, the marker is a polypeptide or a nucleic acid, e.g., an mRNA. The predicted clinical outcome of the tumor tissue sample being tested may be determined based on correlation with that of those tumors with known clinical outcomes having expression levels most similar to the tumor tissue sample being tested.

In certain embodiments, the present invention includes a method of examining gene expression levels (e.g., expressed mRNA or encoded polypeptide) in a tissue sample, e.g., a tumor tissue sample, comprising processing a tissue sample obtained from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a gene expression product (e.g., mRNA or encoded polypepide) having a characteristic (e.g., expression levels or expression patterns) that is indicative of the presence or absence of disease, such as a cancer or tumor. In certain embodiments, any of the detectable markers described herein are used to detect an associated disease or tumor. In particular embodiments, the methods further comprises: (vi) comparing the image to one or more control images or predetermined images obtained from normal tissue, disease tissue, tumor tissue, tumor tissue associated with a good prognosis, or tumor tissue associated with a poor prognosis, or tumor tissue before and after or during treatment(s), thereby determining the presence of a tumor or the prognosis of a tumor. In particular embodiments of any of the aspects of the invention described herein, the imaging comprises quantifying an amount of one or more detectable markers.

A number of genes exhibiting altered levels of expression, e.g., mRNA or encoded protein expression, are known in the art. A number of databases describing such genes and their cancer-related expression patterns are known and available in the art. These include, GENT, a web-accessible database which provides gene expression patterns across diverse human cancer and normal tissues, and the Cancer Genome Atlas (TCGA), which provides information of the expression levels of various genes in a large number of different tumor types, as well as tumor-associated mutations.

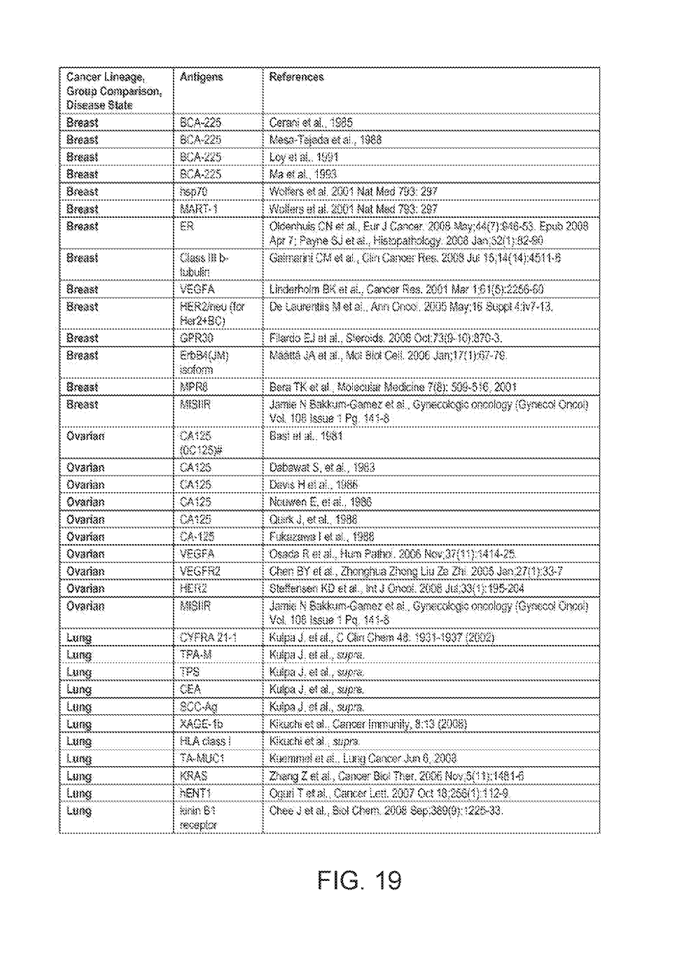

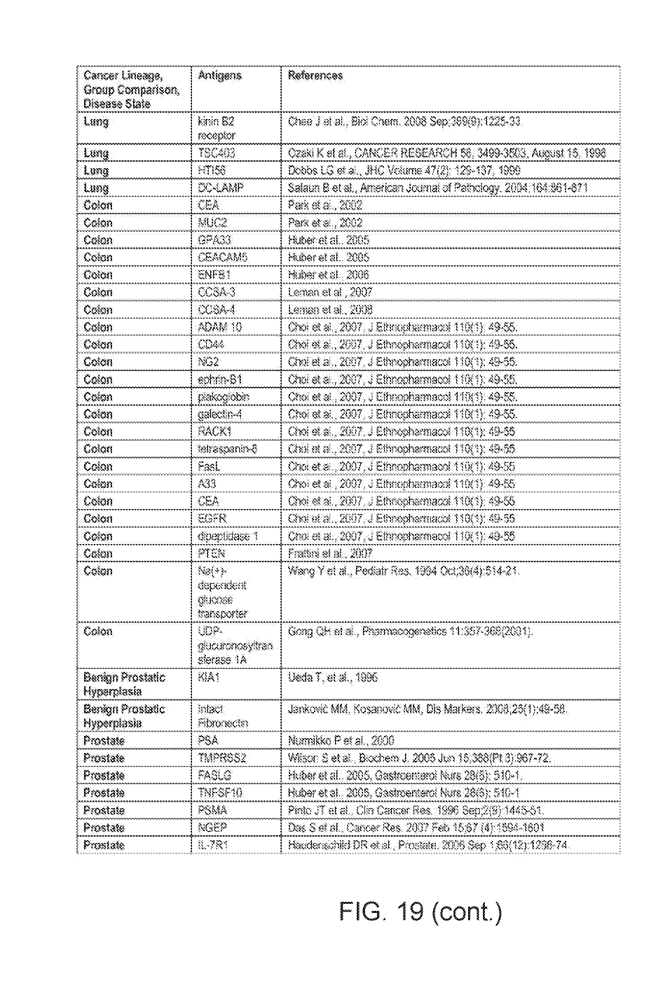

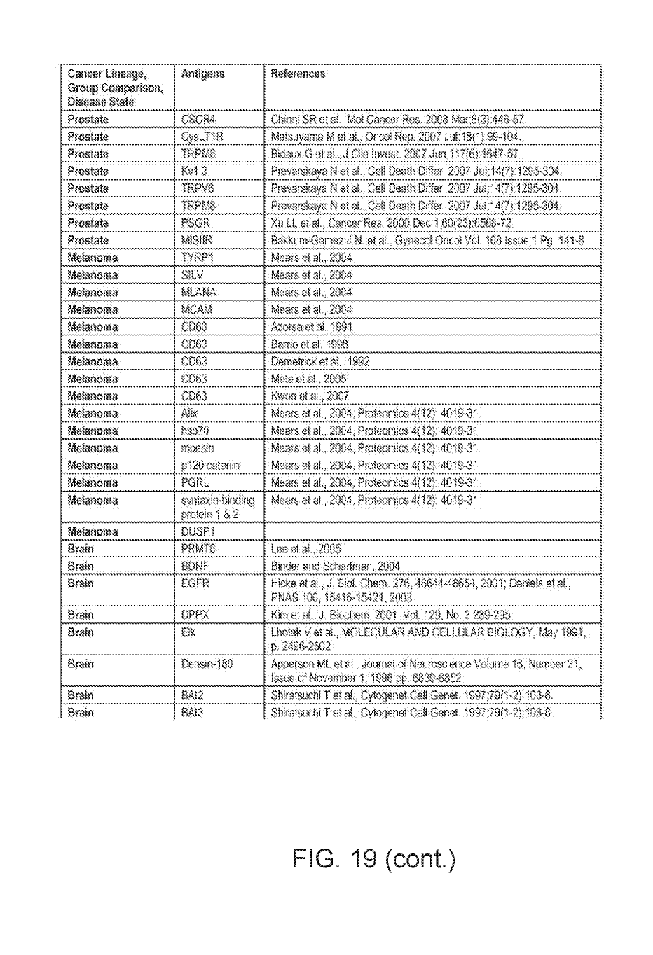

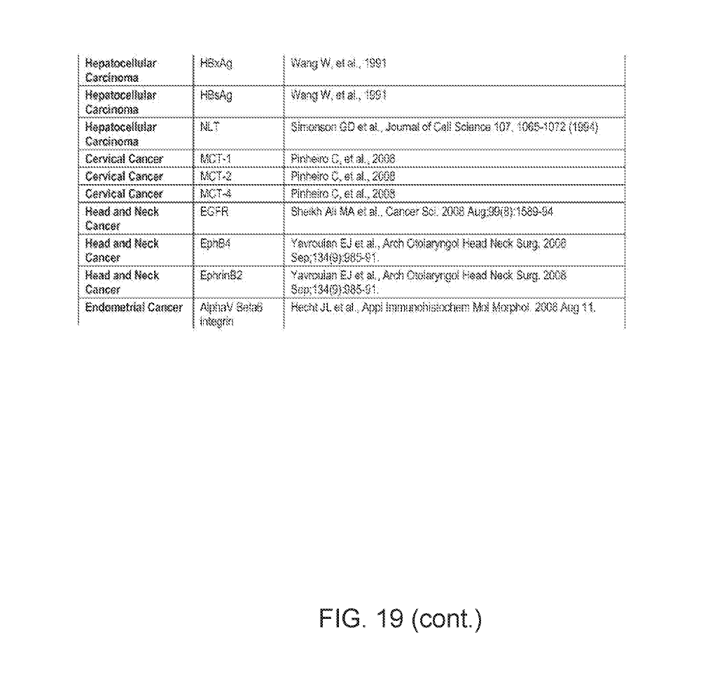

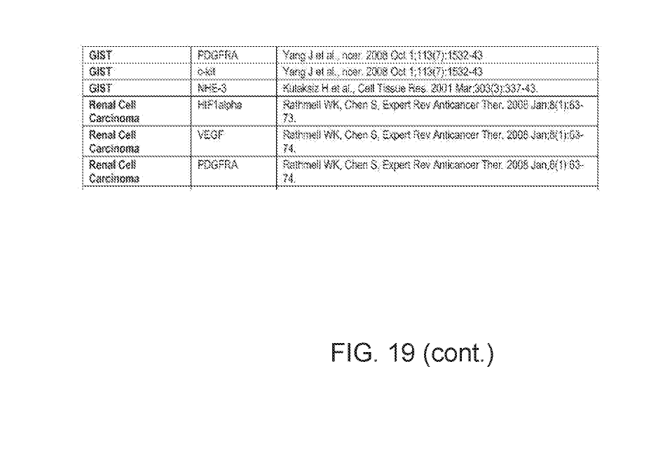

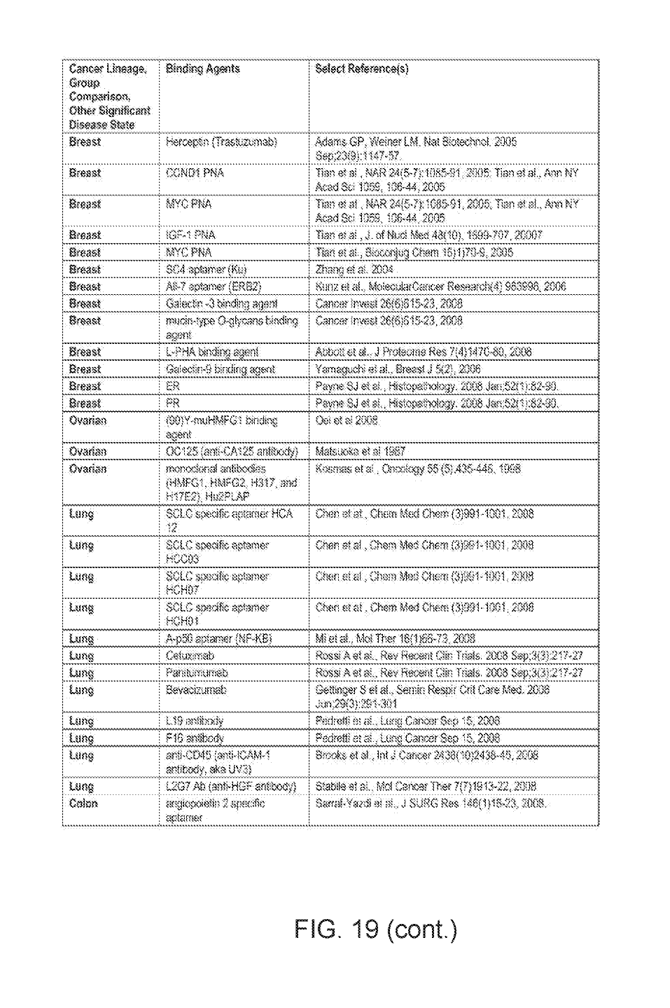

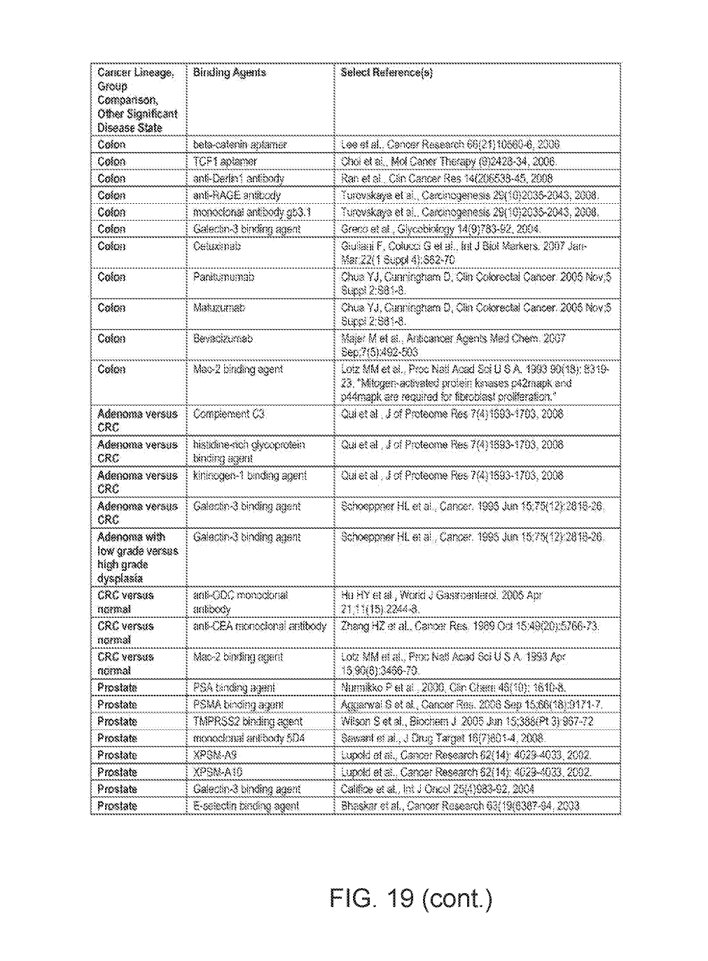

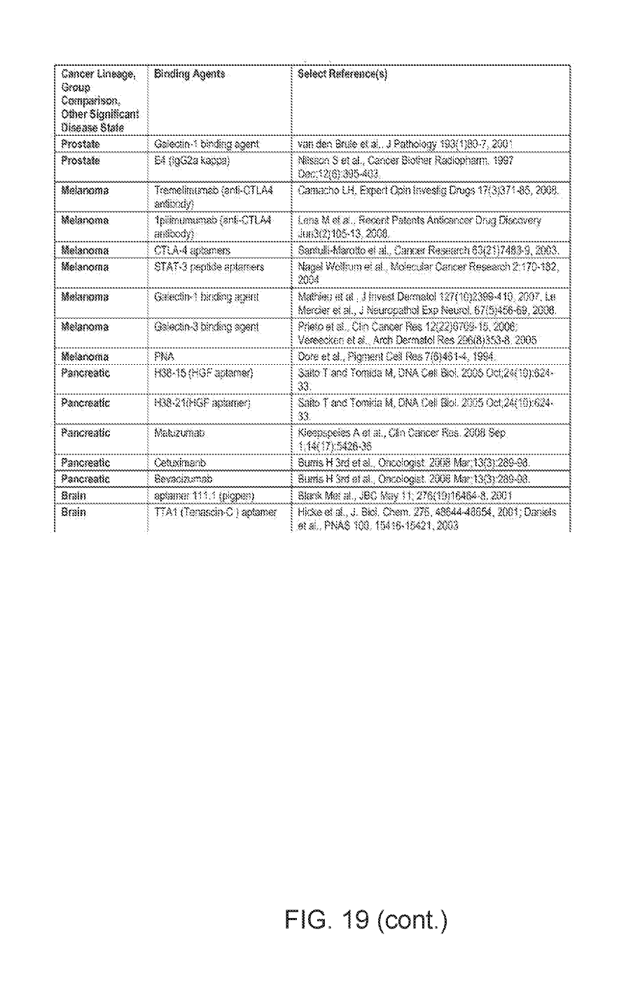

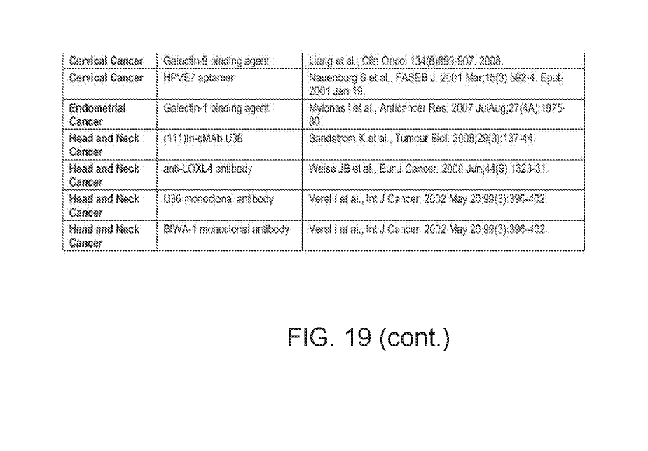

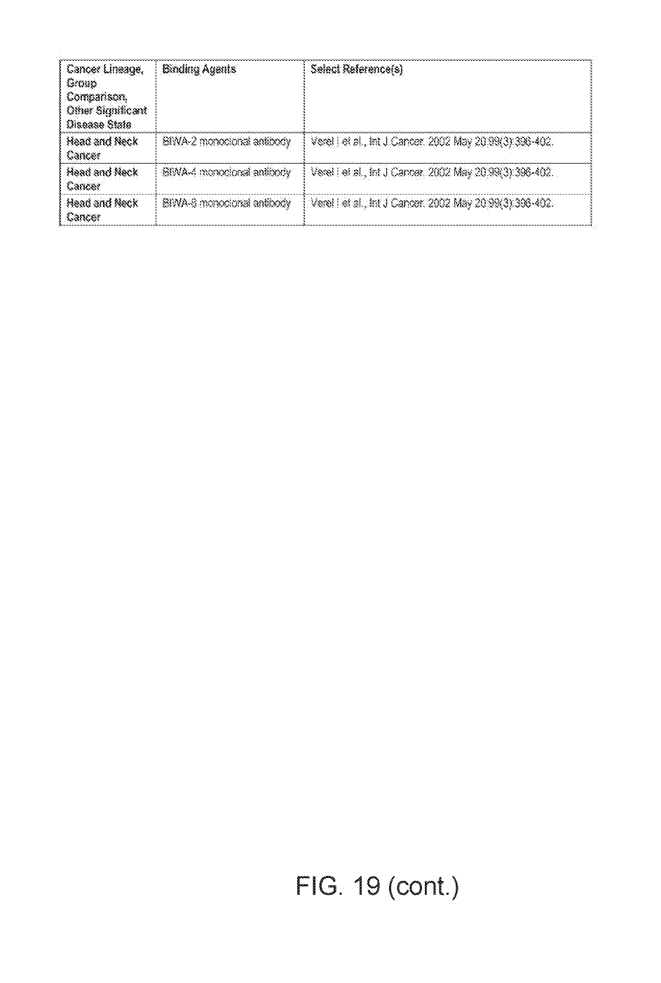

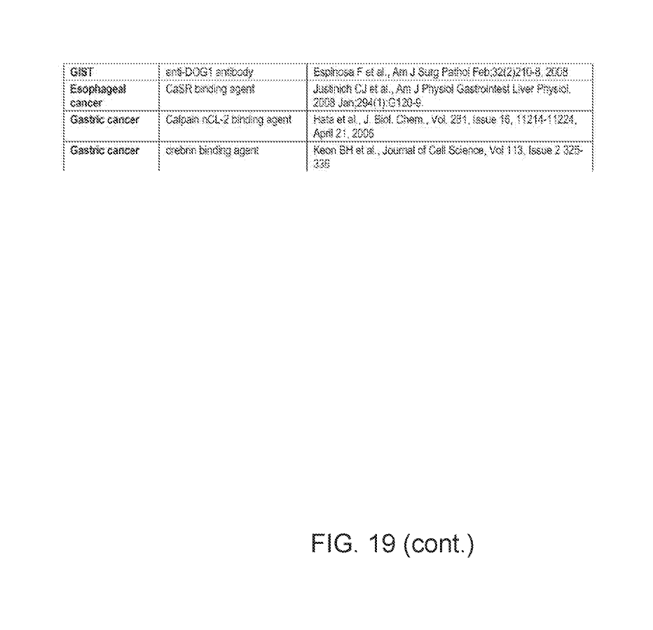

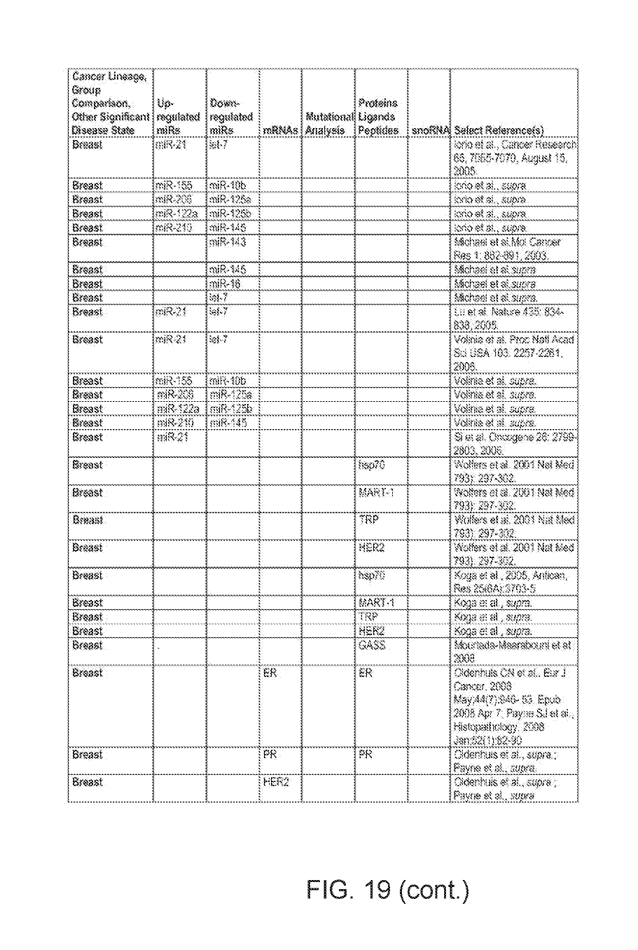

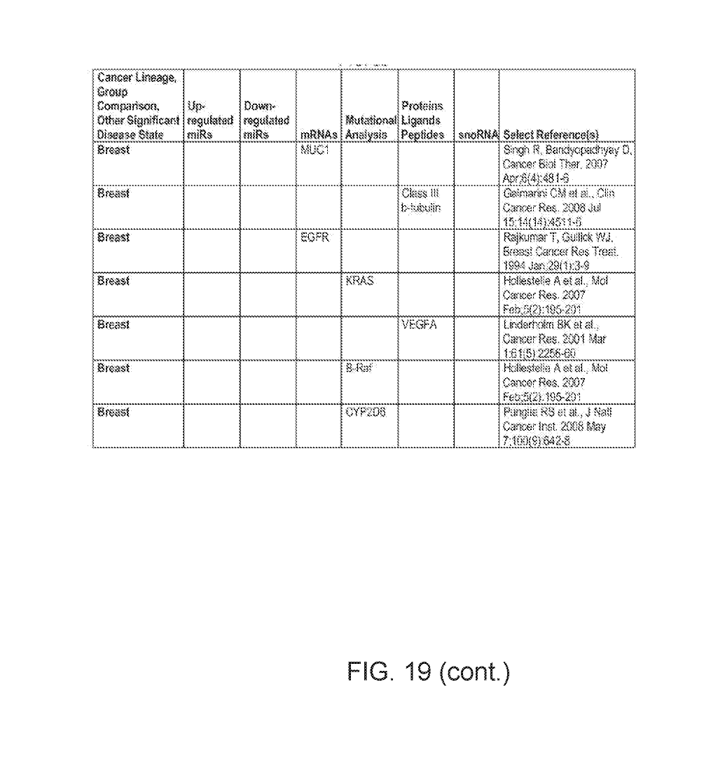

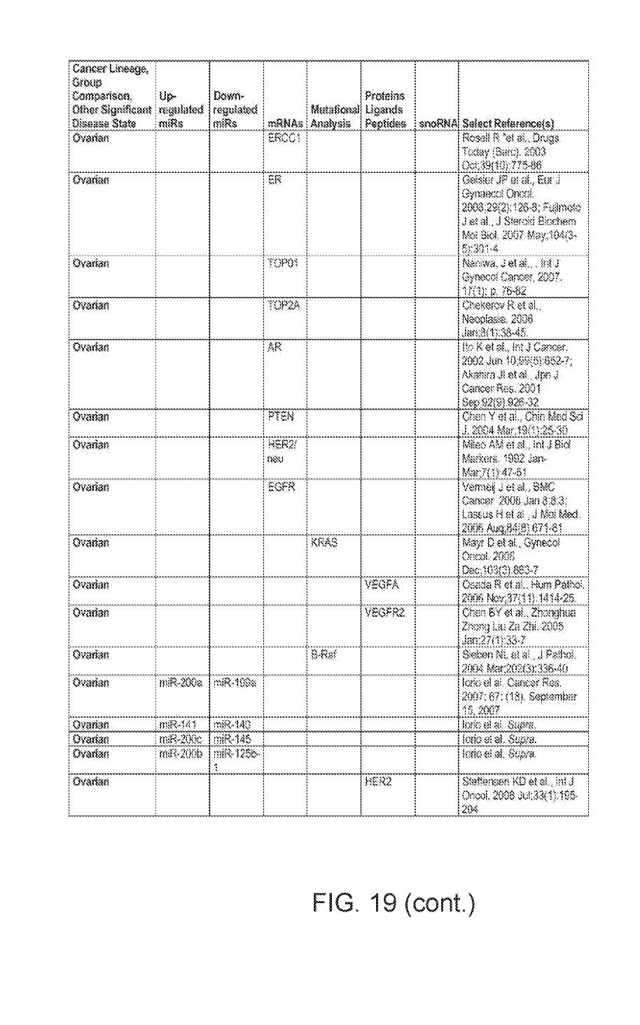

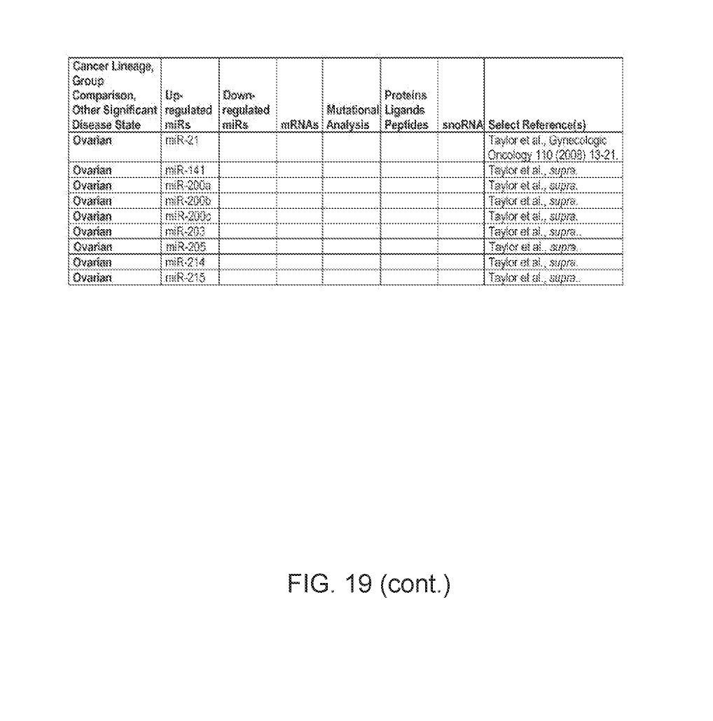

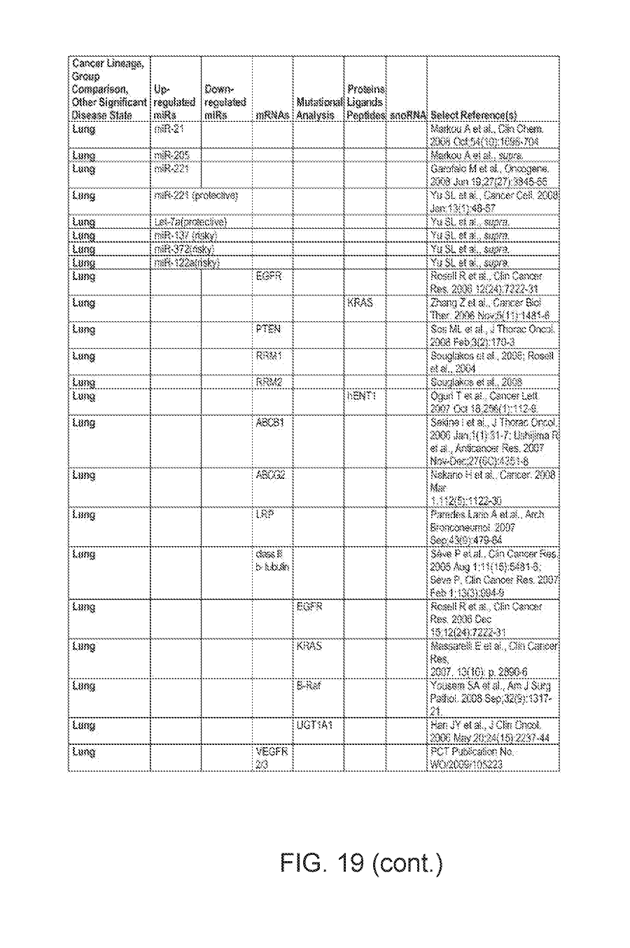

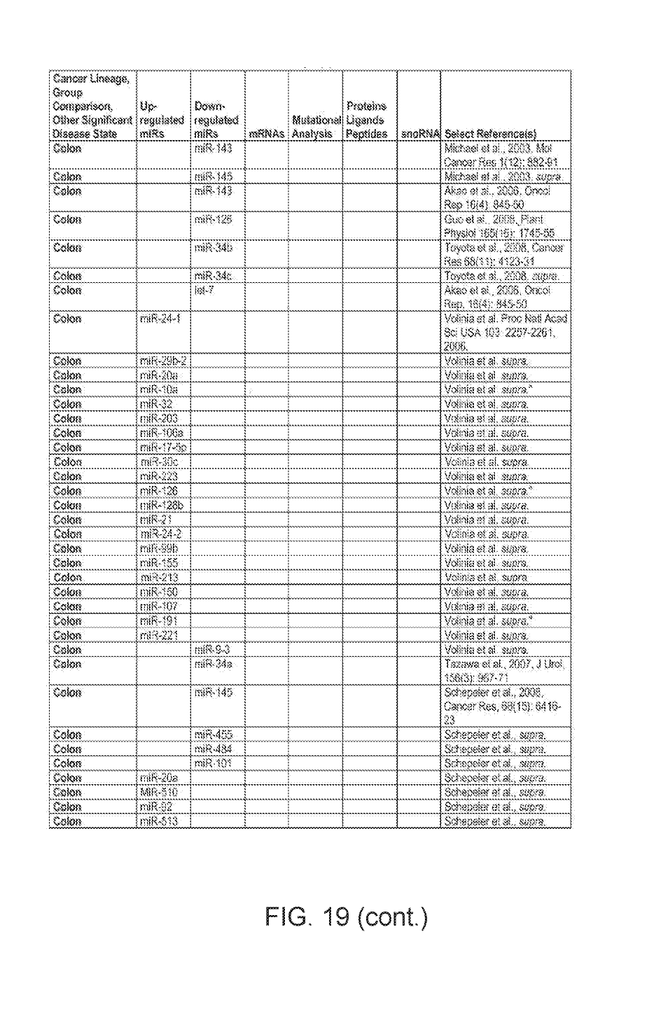

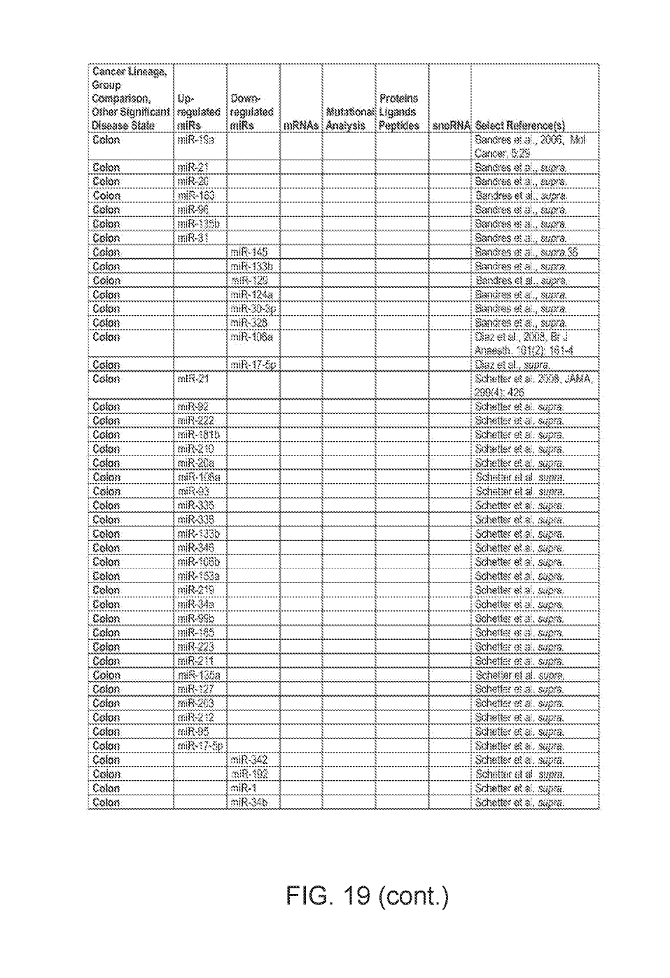

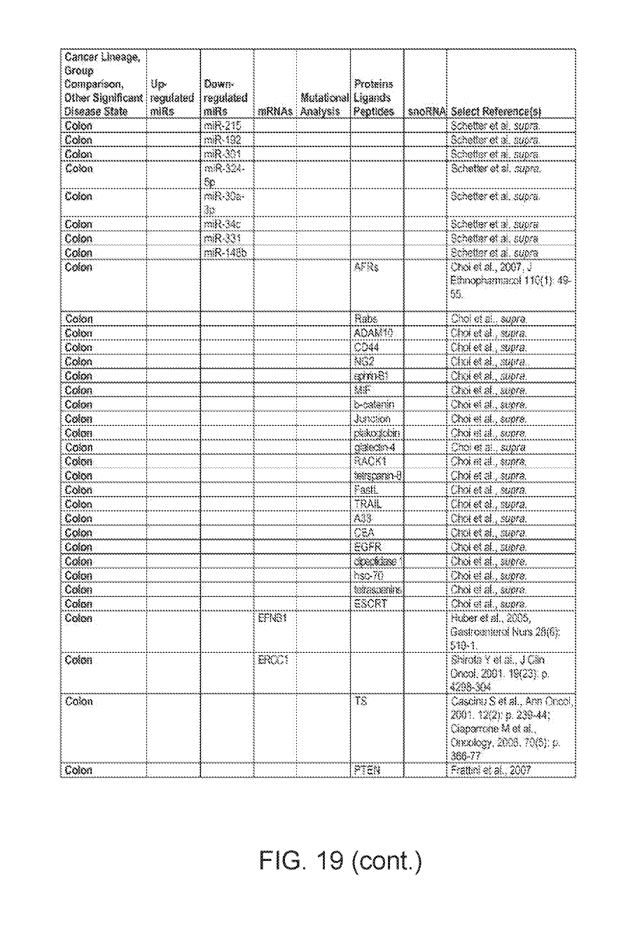

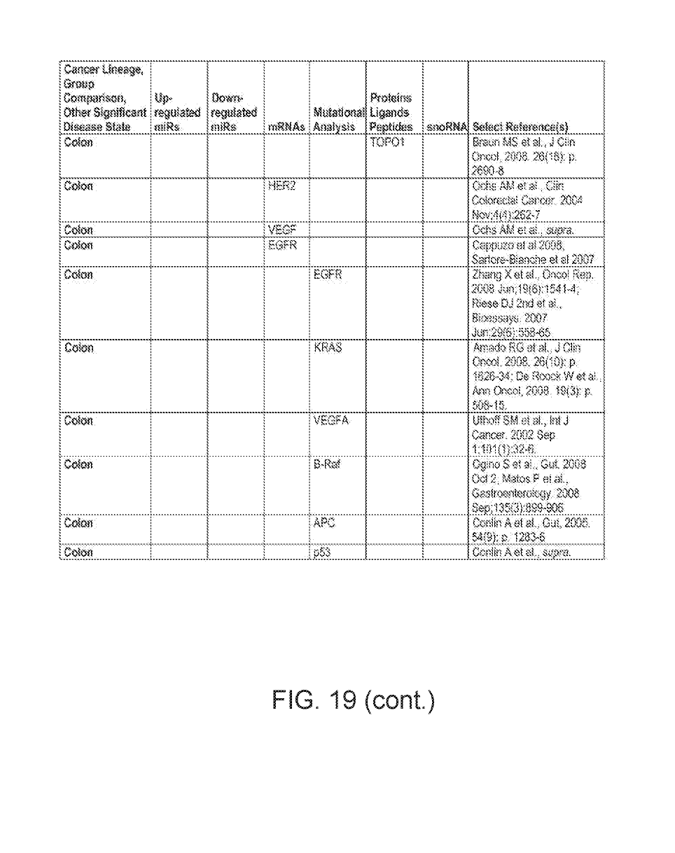

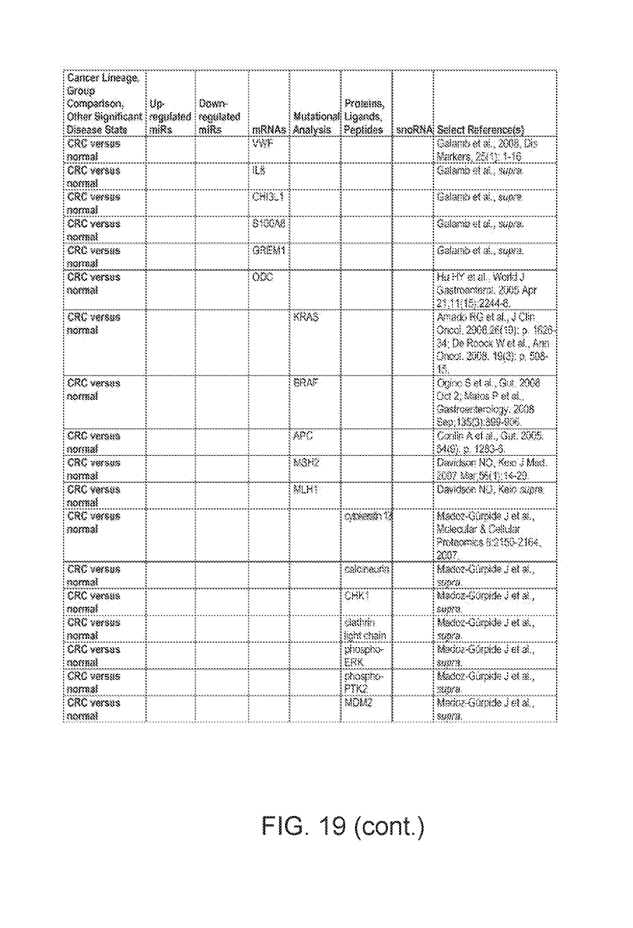

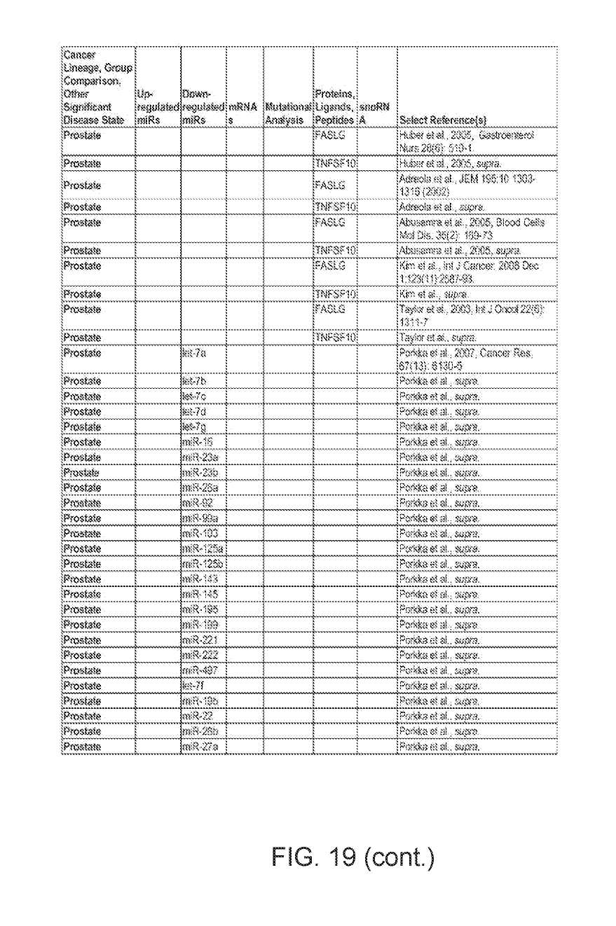

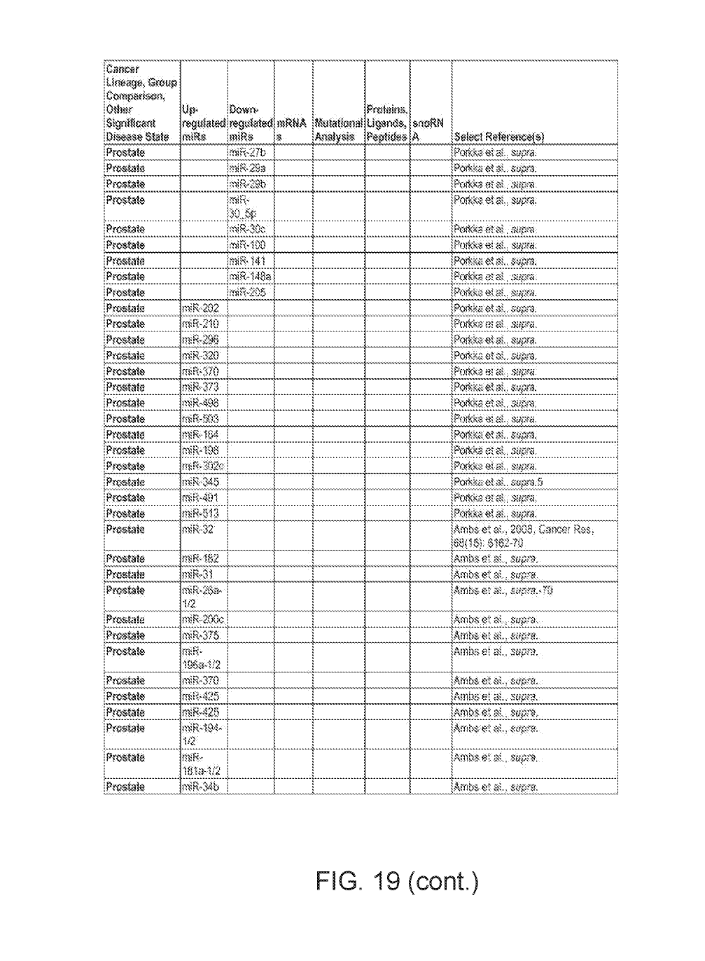

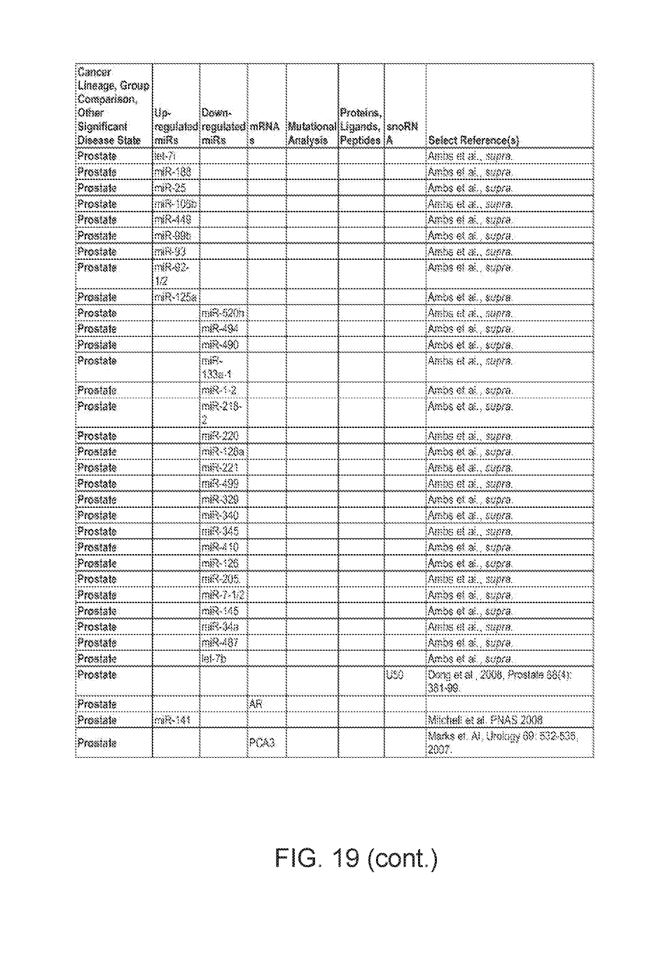

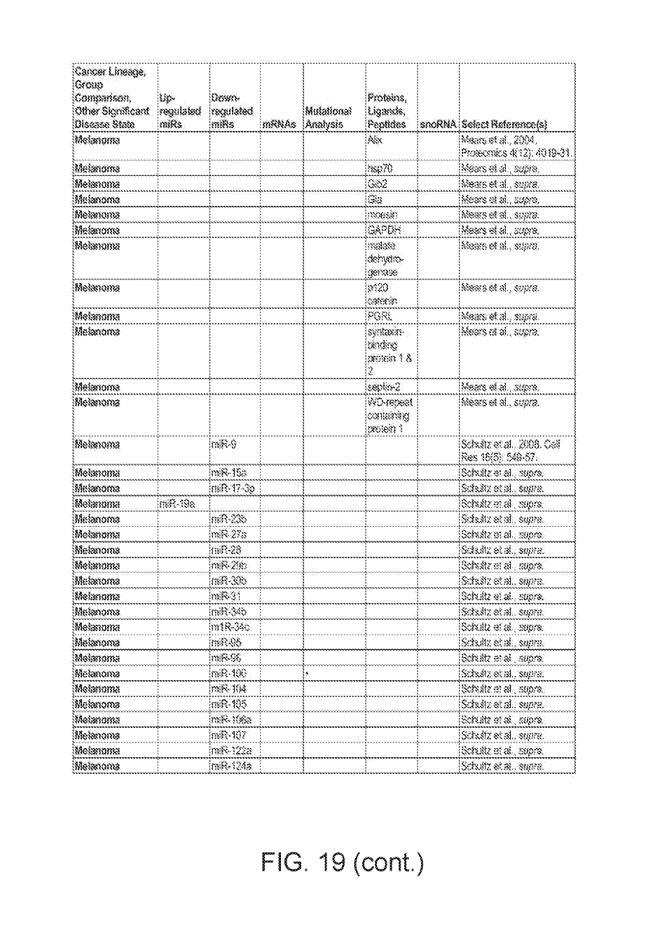

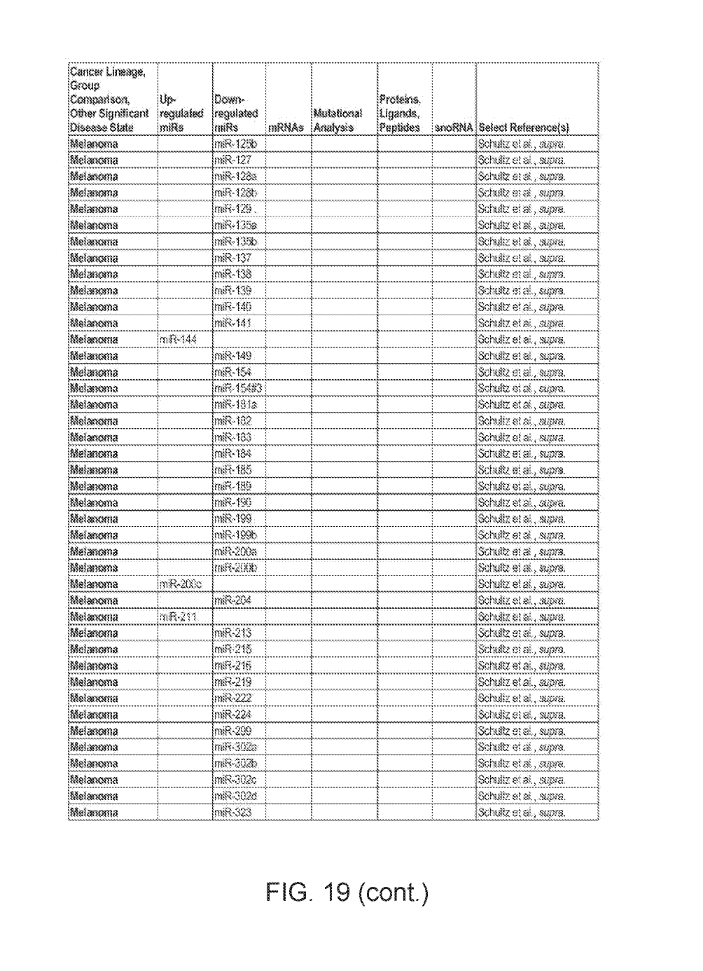

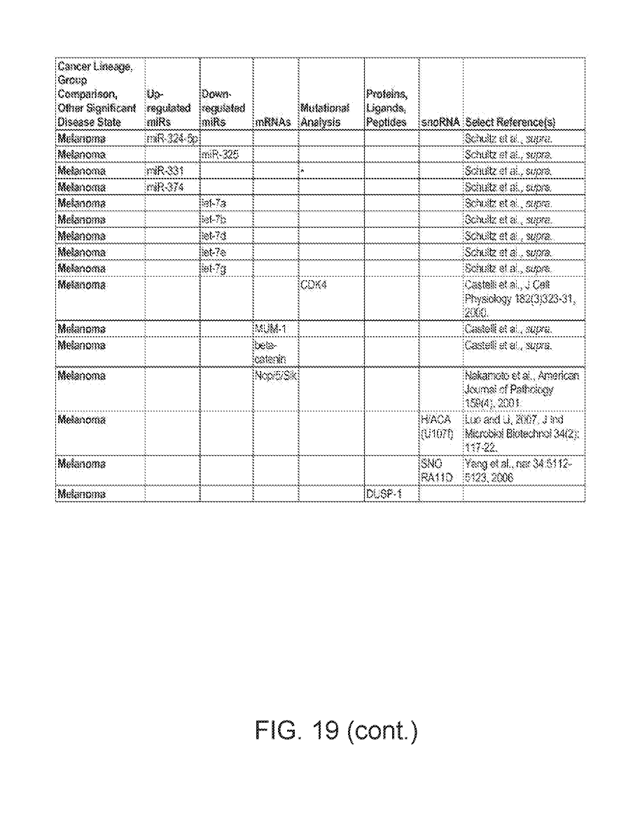

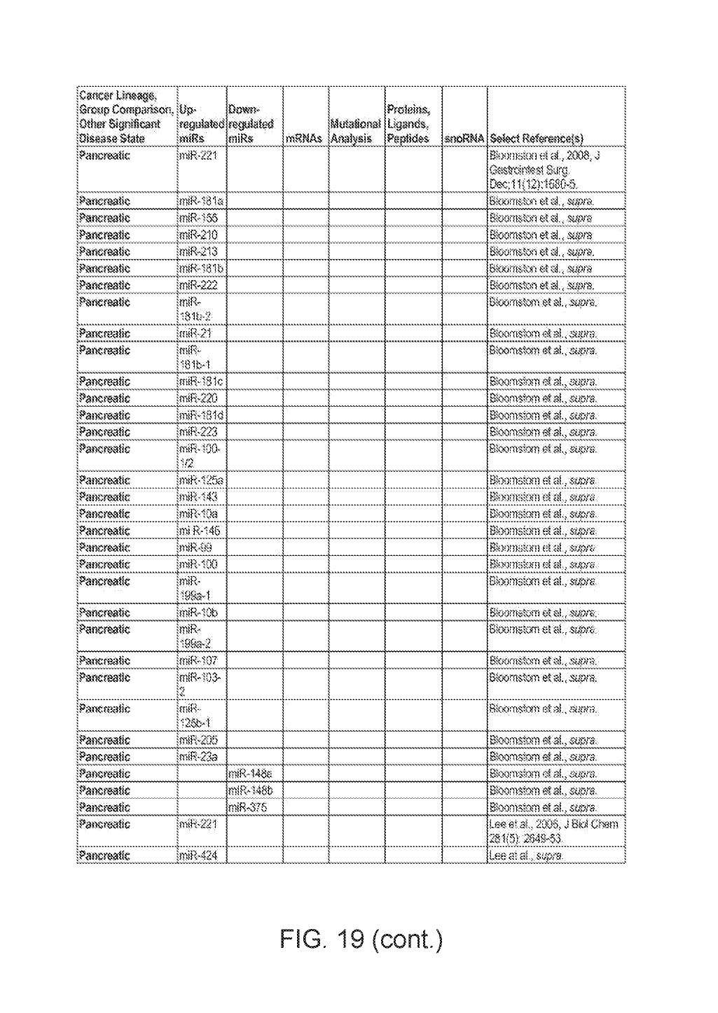

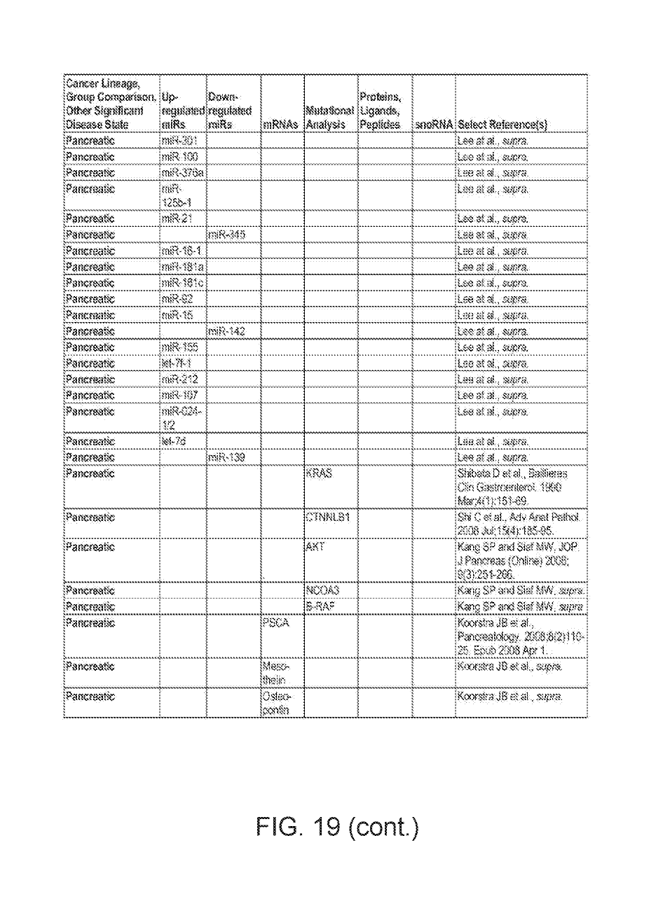

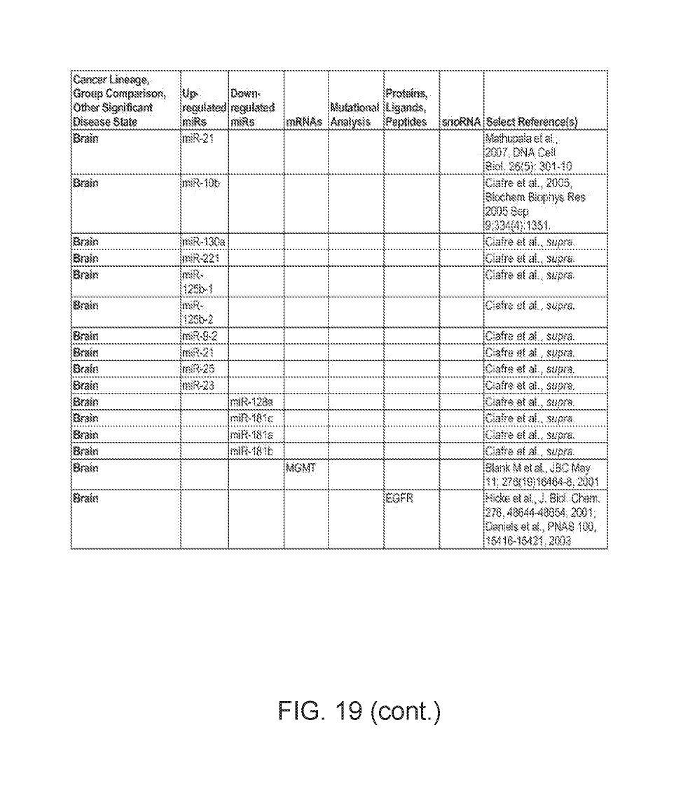

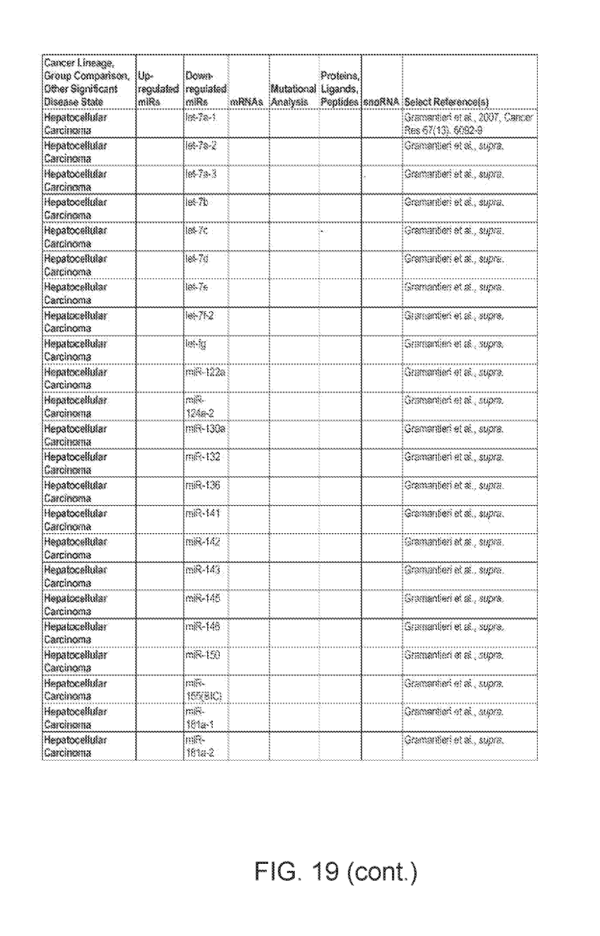

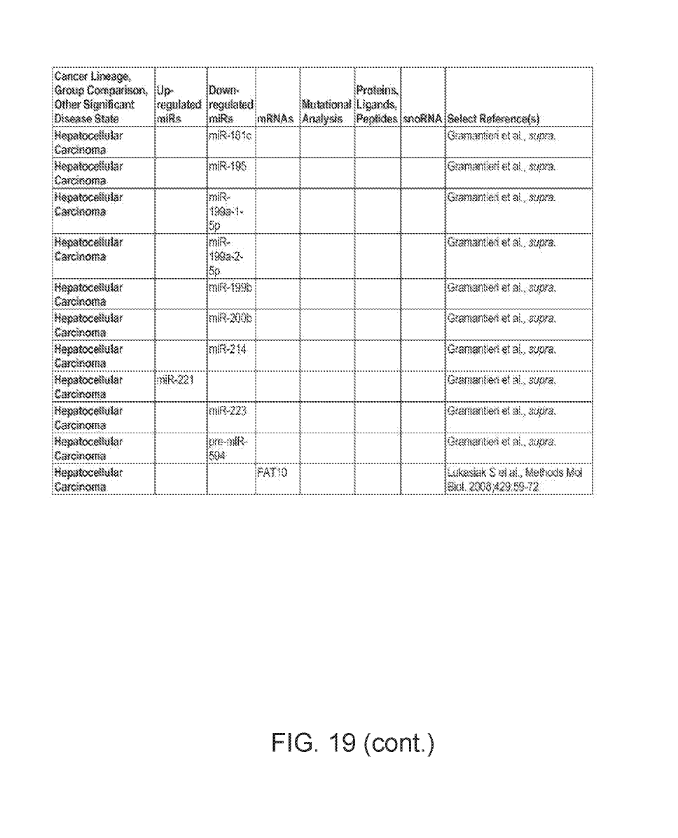

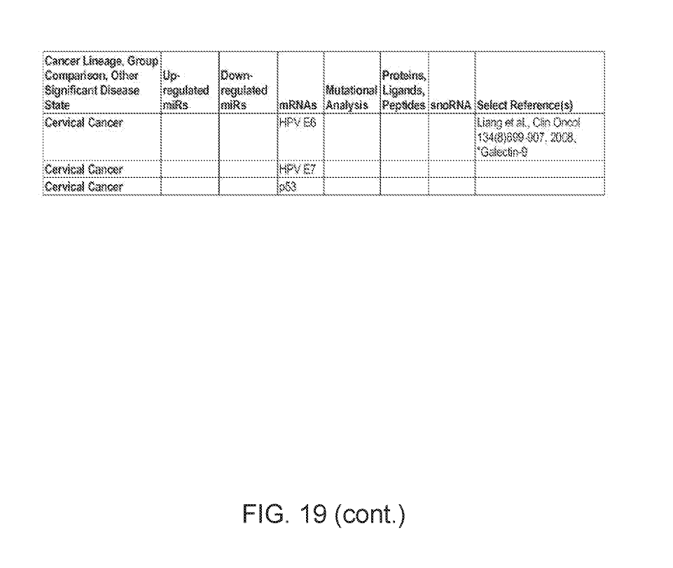

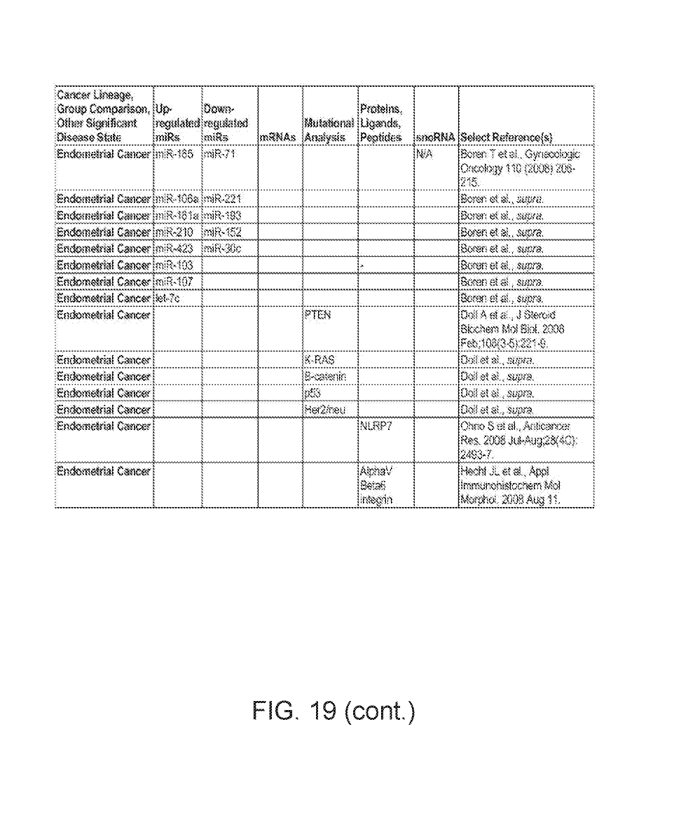

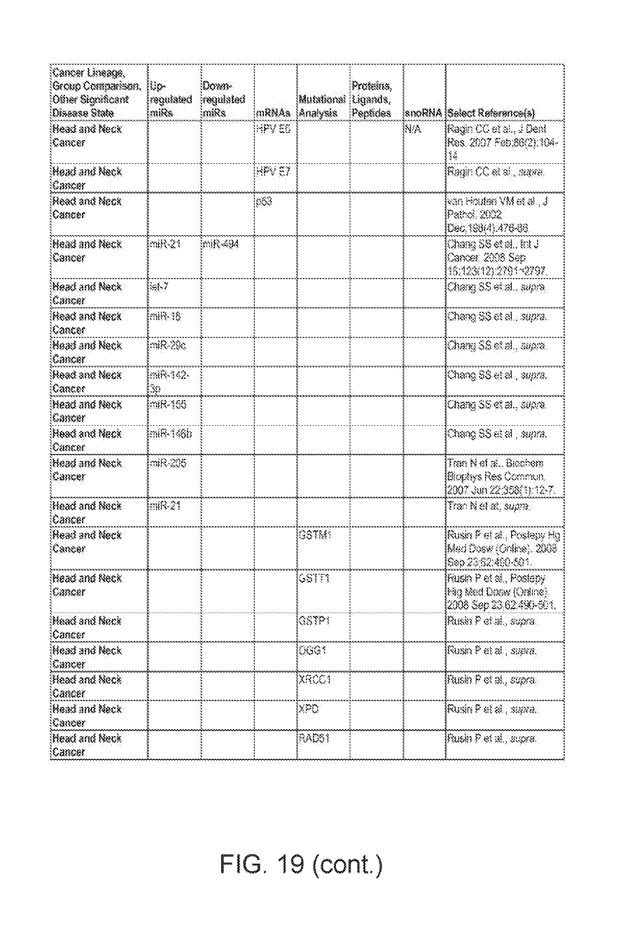

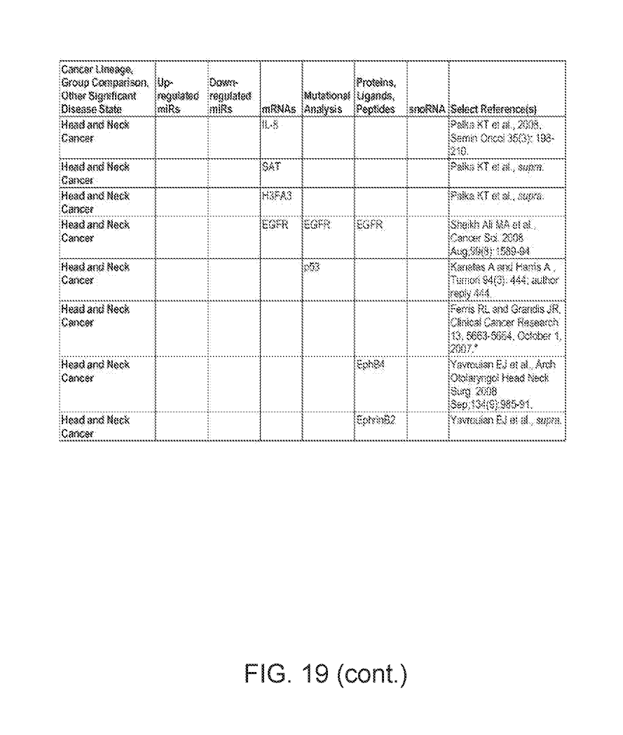

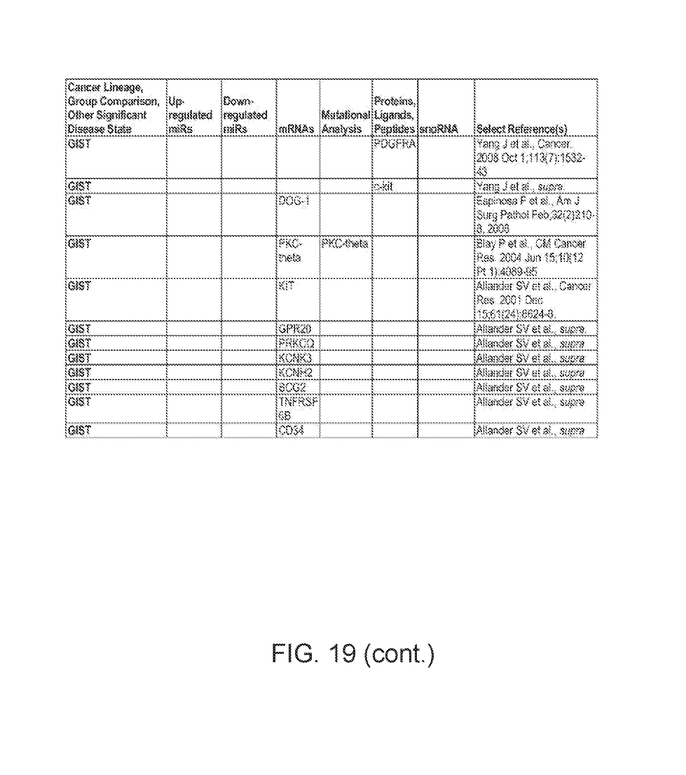

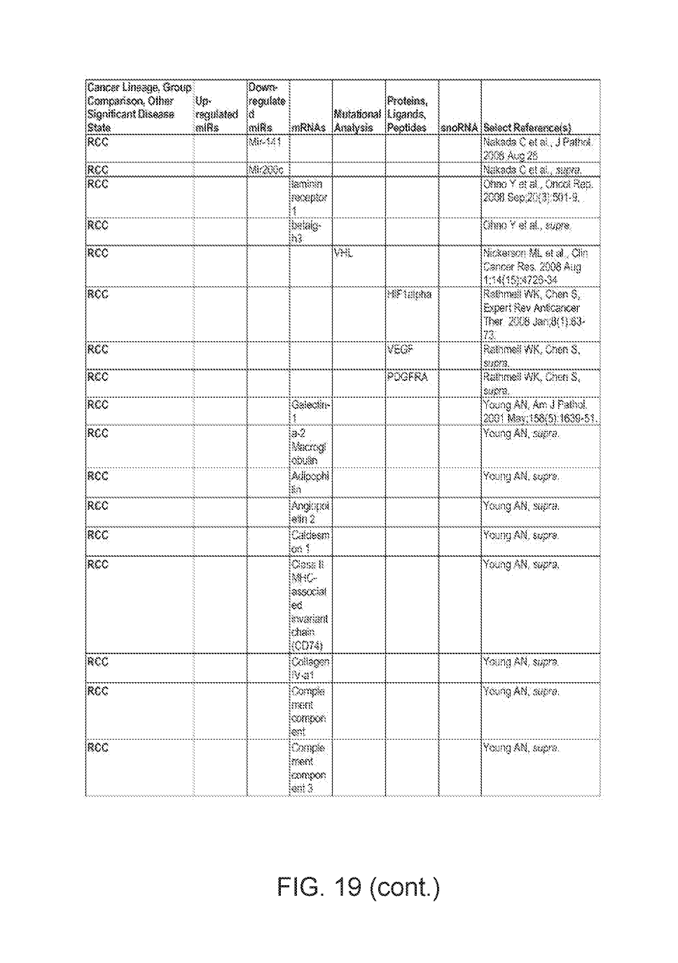

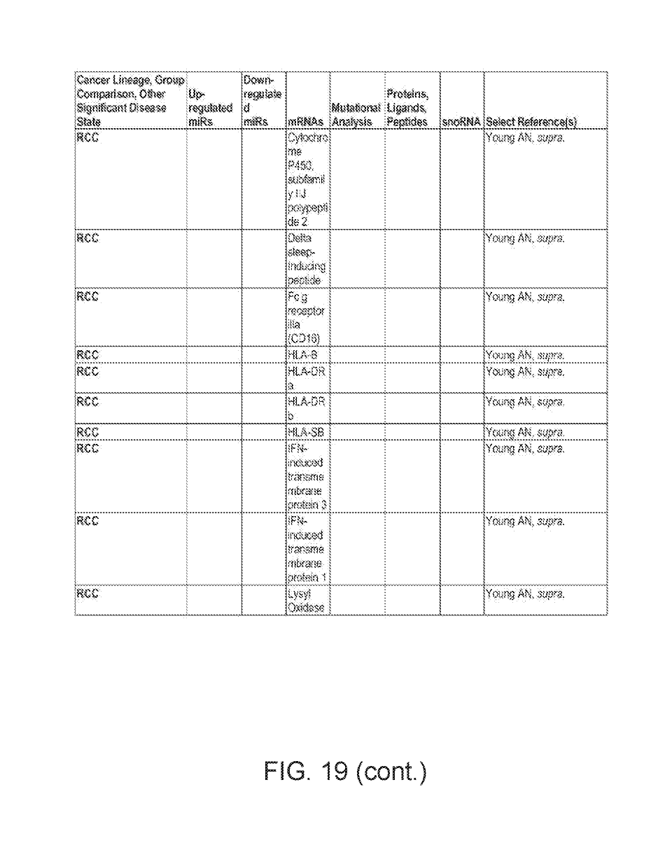

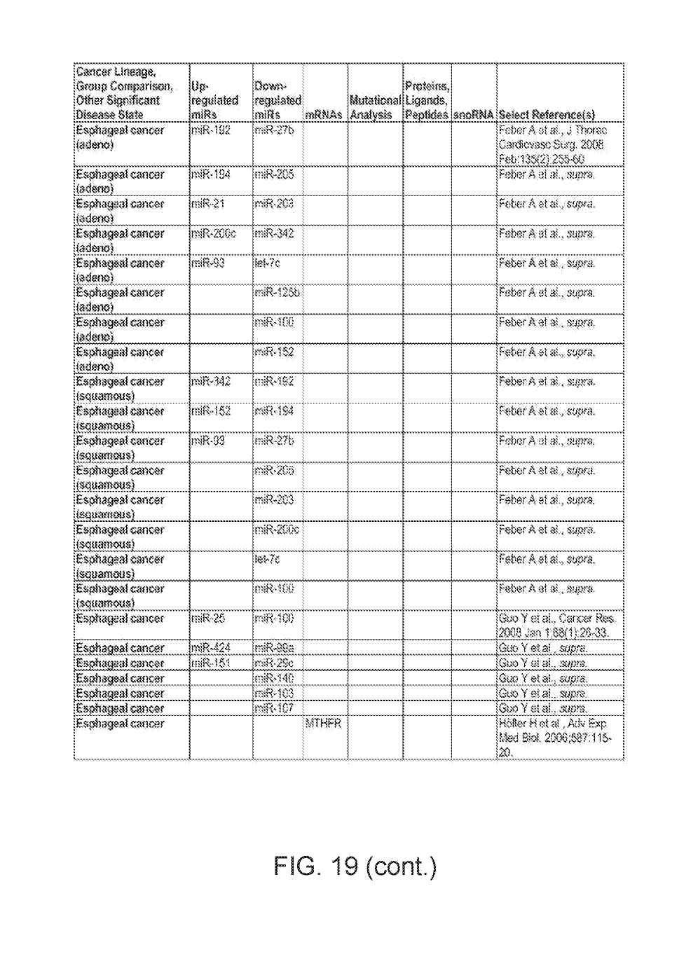

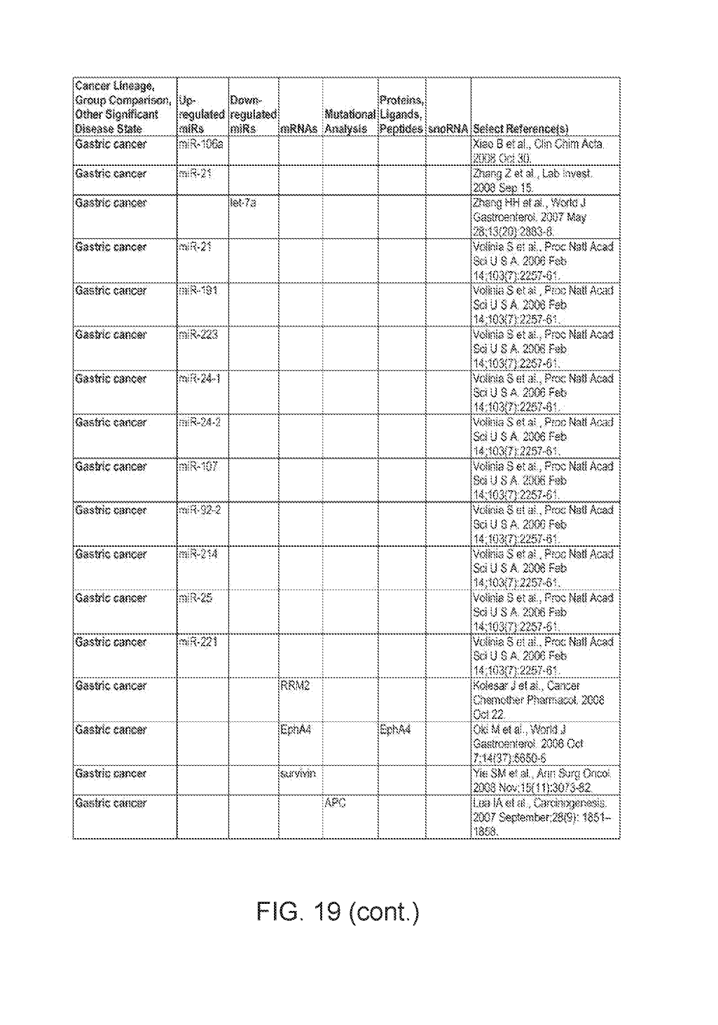

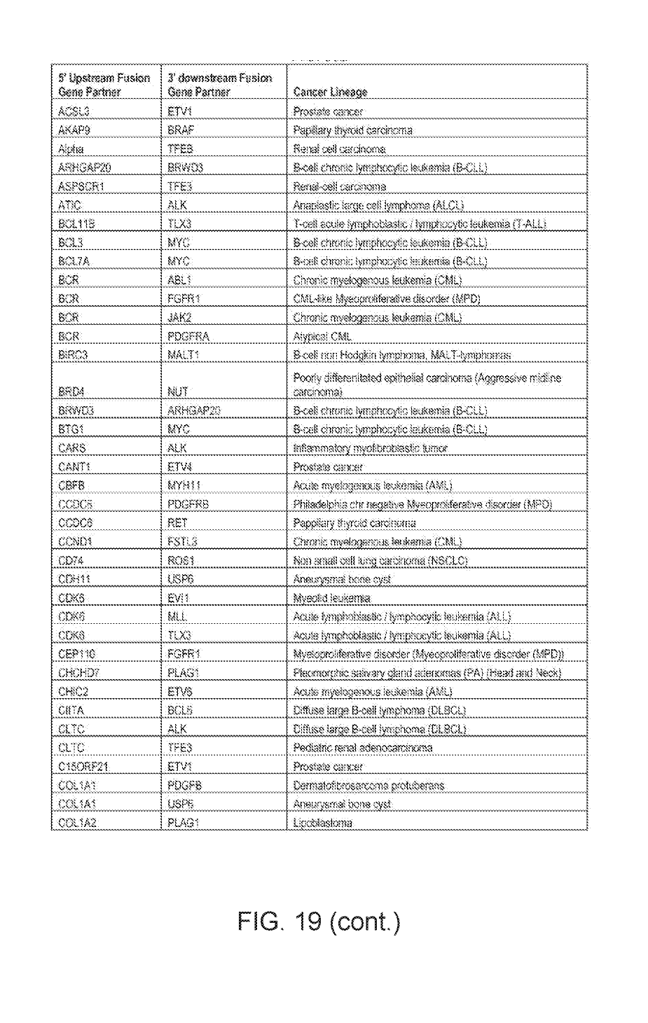

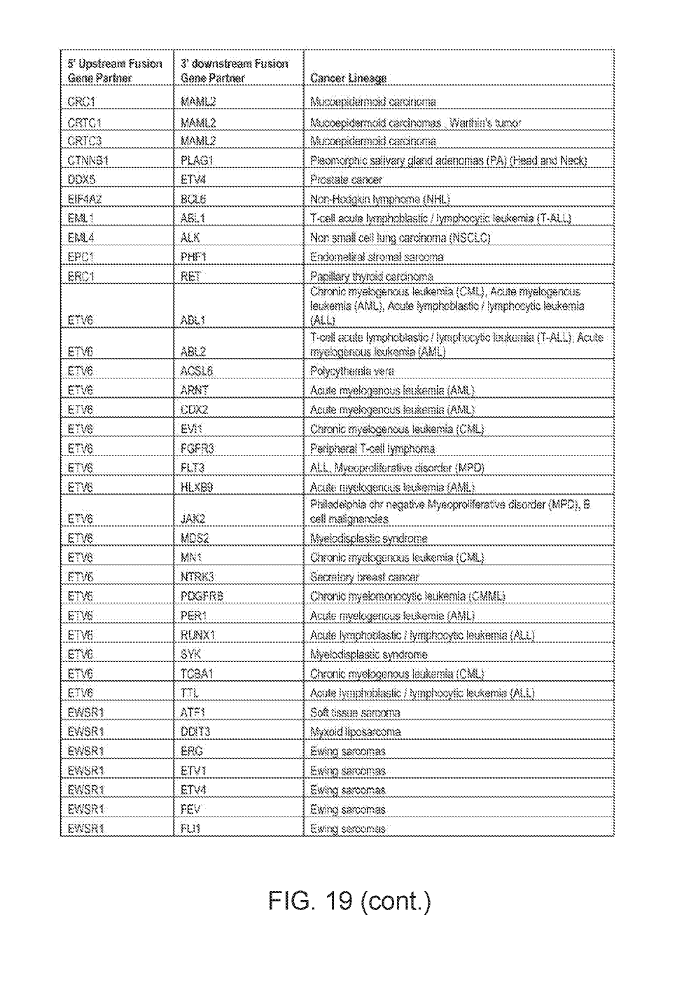

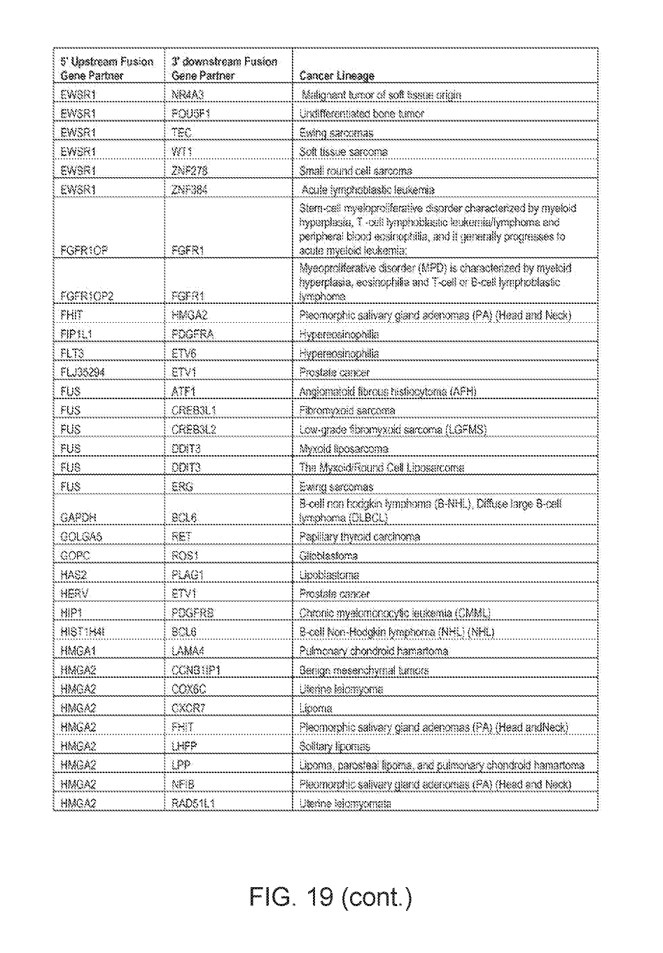

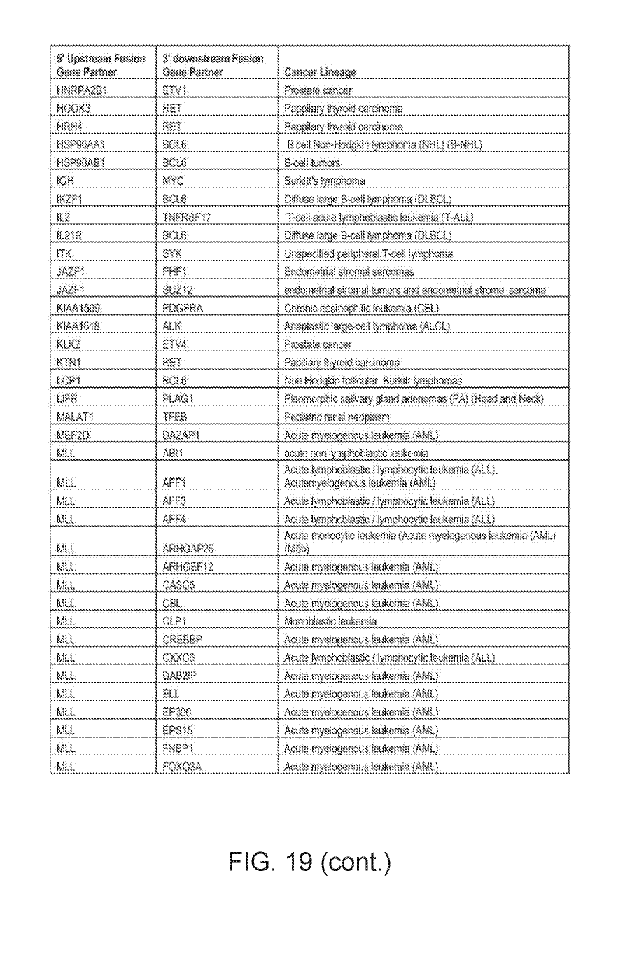

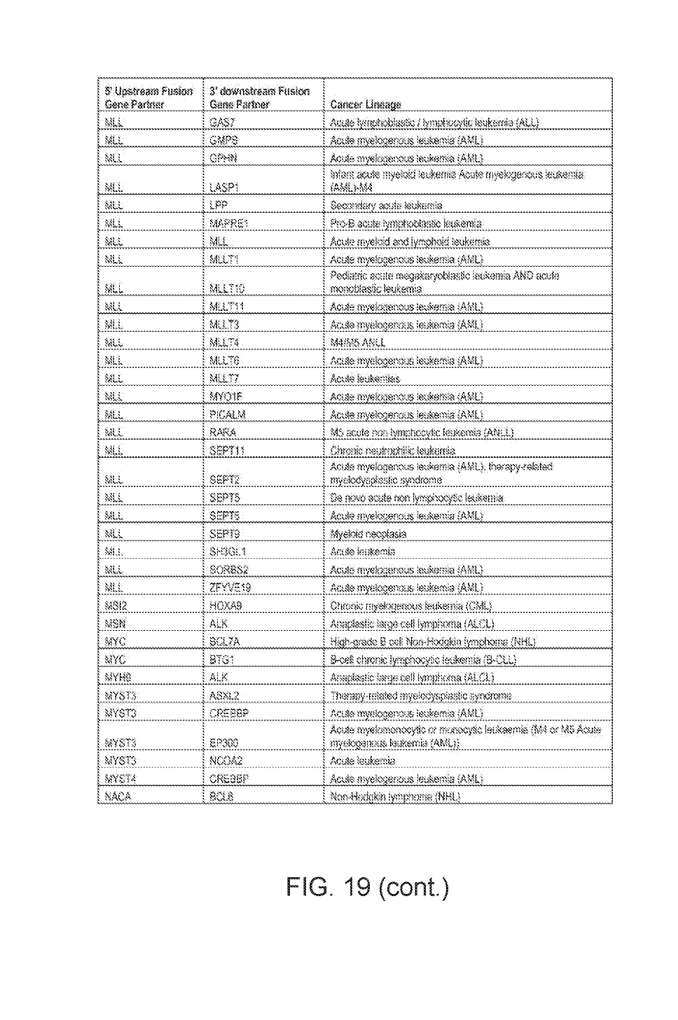

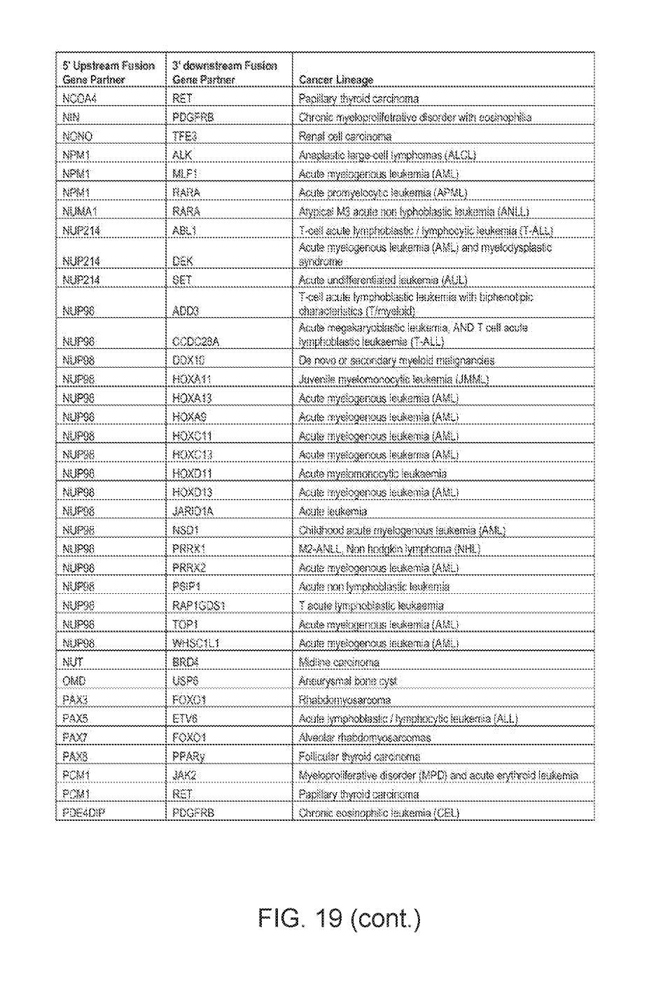

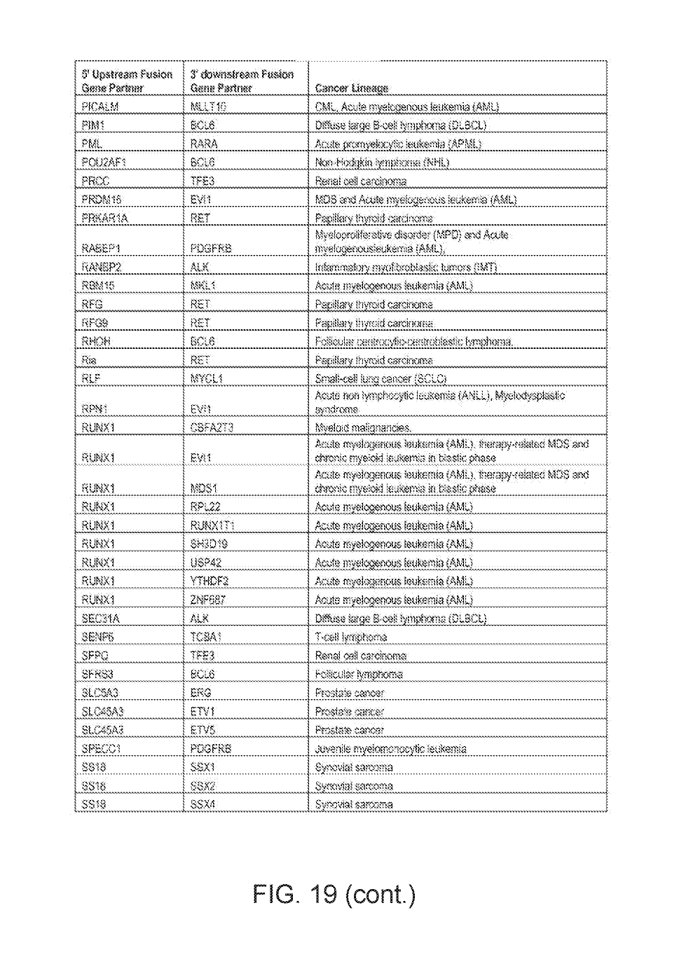

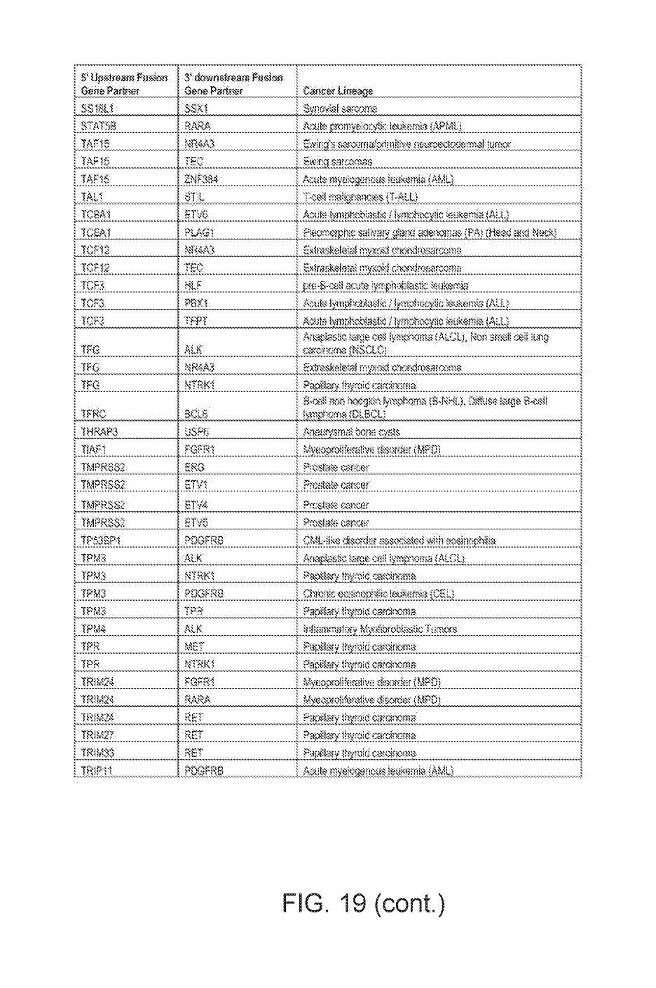

A number of cancer or tumor-associated biomarkers that may be used include those described in US2015/0301055, and those associated with cancer diagnostics, prognostics and theranostics as disclosed in U.S. Pat. Nos. 6,692,916, 6,960,439, 6,964,850, 7,074,586; U.S. patent application Ser. Nos. 11/159,376, 11/804,175, 12/594,128, 12/514,686, 12/514,775, 12/594,675, 12/594,911, 12/594,679, 12/741,787, 12/312,390; and International PCT Patent Application Nos. PCT/US2009/049935, PCT/US2009/063138, PCT/US2010/000037; each of which patent or application is incorporated herein by reference in their entirety. Further examples of biomarkers of diease, such as cancer, and agents that may be used to detect them, as well as translocations associated with cancers, are provided in FIG. 19. Any of these biomarkers may be detected, and/or quantitiated according to methods of the present invention. Additionaly, certain markers that may be used are described in Example 1. In certain embodiments, the breast cancer is an infiltrating ducal carcinoma, a non-infiltrating comedocarcinoma of the breast. Markers for these types of breast cancer may include ER, PR and/or HER2/neu. In certain embodiments, the sample may be a lymph node of a patient diagnosed with a cancer, e.g., a breast cancer, and in some embodiments, the marker is CD-31.

Breast cancer specific biomarkers can include one or more (for example, 2, 3, 4, 5, 6, 7, 8, or more) overexpressed miRs, underexpressed miRs, mRNA, genetic mutations, proteins, ligands, peptides, snoRNA, or any combination thereof. miR biomarkers overexpressed in breast cancer include, but are not limited to, miR-21, miR-155, miR-206, miR-122a, miR-210, miR-21, miR-155, miR-206, miR-122a, miR-210, or miR-21, or any combination thereof. miR biomarkers underexpressed in breast cancer include, but are not limited to, let-7, miR-10b, miR-125a, miR-125b, miR-145, miR-143, miR-145, miR-16, or any combination thereof mRN biomarkers of breast cancer that may be analyzed can include, but are not limited to, ER, PR, HER2, MUC1, or EGFR, or any combination thereof. Mutations including, but not limited to, those related to KRAS, B-Raf, or CYP2D6, or any combination thereof can also be used as specific biomarkers for breast cancer. In addition, a protein, ligand, or peptide biomarker specific to breast cancer can include, but is not limited to, hsp70, MART-1, TRP, HER2, hsp70, MART-1, TRP, HER2, estrogen receptor (ER), progesterone receptor (PR), Class III b-tubulin, or VEGFA, or any combination thereof. Furthermore, snoRNA that can be used as biomarkers for breast cancer include, but are not limited to, GAS5. The gene fusion ETV6-NTRK3 can also be used a biomarker for breast cancer. Another breast cancer specific biomarker is ETV6-NTRK3.

Biomarkers that are used in methods of the invention to assess breast cancer include without limitation BCA-225, hsp70, MARTI, ER, VEGFA, Class III b-tubulin, HER2/neu (e.g., for Her2+ breast cancer), GPR30, ErbB4 (JM) isoform, MPR8, MISIIR, CD9, EphA2, EGFR, B7H3, PSM, PCSA, CD63, STEAP, CD81, ICAM1, A33, DR3, CD66e, MFG-E8, TROP-2, Mammaglobin, Hepsin, NPGP/NPFF2, PSCA, 5T4, NGAL, EpCam, neurokinin receptor-1 (NK-1 or NK-1R), NK-2, Pai-1, CD45, CD10, HER2/ERBB2, AGTR1, NPY1R, MUC1, ESA, CD133, GPR30, BCA225, CD24, CA15.3 (MUC1 secreted), CA27.29 (MUC1 secreted), NMDAR1, NMDAR2, MAGEA, CTAG1B, NY-ESO-1, SPB, SPC, NSE, PGP9.5, a progesterone receptor (PR) or its isoform (PR(A) or PR(B)), P2RX7, NDUFB7, NSE, GAL3, osteopontin, CHI3L1, IC3b, mesothelin, SPA, AQP5, GPCR, hCEA-CAM, PTP IA-2, CABYR, TMEM211, ADAM28, UNC93A, MUC17, MUC2, IL10R-beta, BCMA, HVEM/TNFRSF14, Trappin-2, Elafin, ST2/IL1 R4, TNFRF14, CEACAM1, TPA1, LAMP, WF, WH1000, PECAM, BSA, TNFR, or any combination thereof. One or more antigens CD9, MIS Rii, ER, CD63, MUC1, HER3, STAT3, VEGFA, BCA, CA125, CD24, EPCAM, and ERB B4 can be used to assess vesicles derived from breast cancer cells. In particular embodiments, the marker is estrogen receptor (ER), progesterone receptor (PR) or HER2/neu. Antibodies that bind markers are commercially available or may be produced using routine methods practiced in the art. Illustrative antibodies are described in Table 3.

Ovarian cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, methods of the present invention may be used to detect one or more ovarian cancer overexpressed miRs, such as, but not limited to, miR-200a, miR-141, miR-200c, miR-200b, miR-21, miR-141, miR-200a, miR-200b, miR-200c, miR-203, miR-205, miR-214, miR-1 99*, or miR-215, or any combination thereof. The biosignature can also comprise one or more ovarian cancer underexpressed miRs such as, but not limited to, miR-199a, miR-140, miR-145, miR-100, miR-let-7 cluster, or miR-125b-1, or any combination thereof. The one or more mRNAs that may be analyzed can include without limitation ERCC1, ER, TOPO1, TOP2A, AR, PTEN, HER2/neu, CD24 or EGFR, or any combination thereof.

Other biomarkers of ovarian cancer that may be used include, but are not limnited to, a mutation of KRAS, mutation of B-Raf, BRCA1 and BRCA2, or any combination of mutations specific for ovarian cancer. The protein, ligand, or peptide that can be assessed can include, but is not limited to, VEGFA, VEGFR2, or HER2, or any combination thereof. Another ovarian cancer specific biomarker is CD24.

Lung cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. Markers can comprise one or more lung cancer overexpressed miRs, such as, but not limited to, miR-21, miR-205, miR-221 (protective), let-7a (protective), miR-137 (risky), miR-372 (risky), or miR-122a (risky), or any combination thereof. They can comprise one or more lung cancer upregulated or overexpressed miRNAs, such as miR-17-92, miR-19a, miR-21, miR-92, miR-155, miR-191, miR-205 or miR-210; one or more lung cancer downregulated or underexpressed miRNAs, such as miR-let-7, or any combination thereof. The one or more biomarker may be miR-92a-2*, miR-147, miR-574-5p, such as for small cell lung cancer. One or more mRNA biomarkers of lung cancer that may be analyzed include, but are not limited to, EGFR, PTEN, RRM1, RRM2, ABCB1, ABCG2, LRP, VEGFR2, VEGFR3, class III b-tubulin, or any combination thereof. In certain embodiments, the lung cancer is a lung adenocarcinoma, and the marker may be CD-31.

Biomarker mutations for lung cancer that can be assessed include, but are not limited to, a mutation of EGFR, KRAS, B-Raf, UGT1A1, or rearrangements in ALK, ROS or RET genes, or any combination of mutations specific for lung cancer. The protein, ligand, or peptide that can be assessed can include, but is not limited to, KRAS, hENT1, or any combination thereof. The lung cancer biomarker can also be midkine (MK or MDK). In some embodiments, the lung cancer biomarker comprises one or more of SPB, SPC, PSP9.5, NDUFB7, gal3-b2c10, iC3b, MUC1, TS, ERCC1, GPCR, CABYR and muc17, which can be overexpressed in lung cancer samples compared to normals. Other lung cancer specific biomarkers, such as RLF-MYCL1, TGF-ALK, or CD74-ROS1, may be used.

Colon cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 6, and can be used to create a colon cancer specific biosignature. For example, they can comprise one or more colon cancer overexpressed miRs, such as, but not limited to, miR-24-1, miR-29b-2, miR-20a, miR-10a, miR-32, miR-203, miR-106a, miR-17-5p, miR-30c, miR-223, miR-126, miR-128b, miR-21, miR-24-2, miR-99b, miR-155, miR-213, miR-150, miR-107, miR-191, miR-221, miR-20a, miR-510, miR-92, miR-513, miR-19a, miR-21, miR-20, miR-183, miR-96, miR-135b, miR-31, miR-21, miR-92, miR-222, miR-181b, miR-210, miR-20a, miR-106a, miR-93, miR-335, miR-338, miR-133b, miR-346, miR-106b, miR-153a, miR-219, miR-34a, miR-99b, miR-185, miR-223, miR-211, miR-135a, miR-127, miR-203, miR-212, miR-95, or miR-17-5p, or any combination thereof. The biosignature can also comprise one or more colon cancer underexpressed miRs such as miR-143, miR-145, miR-143, miR-126, miR-34b, miR-34c, let-7, miR-9-3, miR-34a, miR-145, miR-455, miR-484, miR-101, miR-145, miR-133b, miR-129, miR-124a, miR-30-3p, miR-328, miR-106a, miR-17-5p, miR-342, miR-192, miR-1, miR-34b, miR-215, miR-192, miR-301, miR-324-5p, miR-30a-3p, miR-34c, miR-331, miR-548c-5p, miR-362-3p, miR-422a, or miR-148b, or any combination thereof. The one or more colon cancer biomarker can be an upregulated or overexpressed miRNA, such as miR-20a, miR-21, miR-106a, miR-181 b or miR-203, for characterizing a colon adenocarcinoma. The one or more biomarker can be used to characterize a colorectal cancer, such as an upregulated or overexpressed miRNA selected from the group consisting of: miR-19a, miR-21, miR-127, miR-31, miR-96, miR-135b and miR-183, a downregulated or underexpressed miRNA, such as miR-30c, miR-133a, mir143, miR-133b or miR-145, or any combination thereof. The one or more biomarker can be used to characterize a colorectal cancer, such as an upregulated or overexpressed miRNA selected from the group consisting of: miR-548c-5p, miR-362-3p, miR-422a, miR-597, miR-429, miR-200a, and miR-200b, or any combination thereof.

The one or more colon cancer mRNAs that may be analyzed can include, but are not limited to, EFNB1, ERCC1, HER2, VEGF, or EGFR, or any combination thereof. A biomarker mutation for colon cancer that can be assessed includes, but is not limited to, a mutation of EGFR, KRAS, VEGFA, B-Raf, APC, or p53, or any combination of mutations specific for colon cancer. The protein, ligand, or peptide that can be assessedcan include, but is not limited to, AFRs, Rabs, ADAM10, CD44, NG2, ephrin-B1, MIF, b-catenin, Junction, plakoglobin, glalectin-4, RACK1, tetrspanin-8, FasL, TRAIL, A33, CEA, EGFR, dipeptidase 1, hsc-70, tetraspanins, ESCRT, TS, PTEN, or TOPO1, or any combination thereof.

Biomarkers for bladder cancer can be used to assess a bladder cancer according to the methods of the invention. The biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. Biomarkers for bladder cancer include without limitation one or more of miR-223, miR-26b, miR-221, miR-103-1, miR-185, miR-23b, miR-203, miR-17-5p, miR-23a, miR-205 or any combination thereof. Further biomarkers for bladder cancer include FGFR3, EGFR, pRB (retinoblastoma protein), 5T4, p53, Ki-67, VEGF, CK20, COX2, p21, Cyclin D1, p14, p15, p16, Her-2, MAPK (mitogen-activated protein kinase), Bax/Bcl-2, PI3K (phosphoinositide-3-kinase), CDKs (cyclin-dependent kinases), CD40, TSP-1, HA-ase, telomerase, survivin, NMP22, TNF, Cyclin E1, p27, caspase, survivin, NMP22 (Nuclear matrix protein 22), BCLA-4, Cytokeratins (8, 18, 19 and 20), CYFRA 21-1, IL-2, and complement factor H-related protein. In an embodiment, non-receptor tyrosine kinase ETK/BMX and/or Carbonic Anhydrase IX is used as a marker of bladder cancer for diagnostic, prognostic and therapeutic purposes.

Prostate cancer specific biomarkers from a vesicle can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, biomarkers for prostate cancer can comprise miR-9, miR-21, miR-141, miR-370, miR-200b, miR-210, miR-155, or miR-196a. In some embodiments, the biomarker is one or more prostate cancer overexpressed miRs, such as, but not limited to, miR-202, miR-210, miR-296, miR-320, miR-370, miR-373, miR-498, miR-503, miR-184, miR-198, miR-302c, miR-345, miR-491, miR-513, miR-32, miR-182, miR-31, miR-26a-1/2, miR-200c, miR-375, miR-196a-1/2, miR-370, miR-425, miR-425, miR-194-1/2, miR-181a-1/2, miR-34b, let-7i, miR-188, miR-25, miR-106b, miR-449, miR-99b, miR-93, miR-92-1/2, miR-125a, miR-141, miR-29a, miR-145 or any combination thereof. In some embodiments, the biomarkers comprises one or more miRs overexpressed in prostate cancer including miR-29a and/or miR-145. In some embodiments, the biomarkers comprise one or more miRs overexpressed in prostate cancer including hsa-miR-1974, hsa-miR-27b, hsa-miR-103, hsa-miR-146a, hsa-miR-22, hsa-miR-382, hsa-miR-23a, hsa-miR-376c, hsa-miR-335, hsa-miR-142-5p, hsa-miR-221, hsa-miR-142-3p, hsa-miR-151-3p and hsa-miR-21, or miR-141, or any combination thereof. Prostate cancer biomarkers can also comprise one or more underexpressed miRs such as, but not limited to, let-7a, let-7b, let-7c, let-7d, let-7g, miR-16, miR-23a, miR-23b, miR-26a, miR-92, miR-99a, miR-103, miR-125a, miR-125b, miR-143, miR-145, miR-195, miR-199, miR-221, miR-222, miR-497, let-7f, miR-19b, miR-22, miR-26b, miR-27a, miR-27b, miR-29a, miR-29b, miR-305p, miR-30c, miR-100, miR-141, miR-148a, miR-205, miR-520h, miR-494, miR-490, miR-133a-1, miR-1-2, miR-218-2, miR-220, miR-128a, miR-221, miR-499, miR-329, miR-340, miR-345, miR-410, miR-126, miR-205, miR-7-1/2, miR-145, miR-34a, miR-487, or let-7b, or any combination thereof. The prostate cancer biomarkers can comprise upregulated or overexpressed miR-21, downregulated or underexpressed miR-15a, miR-16-1, miR-143 or miR-145, or any combination thereof.

The one or more prostate cancer biomarker mRNAs that may be analyzed can include, but are not limited to, AR, PCA3, or any combination thereof and can be used as specific biomarkers for prostate cancer.

The protein, ligand, or peptide that can be assessed as prostate cancer biomarkers can include, but is not limited to, FASLG or HSP60, PSMA, PC SA, androgen receptor (AR), TNF SF10 or any combination thereof. Furthermore, the snoRNA that can be used as a biomarker for prostate cancer can include, but is not limited to, U50.

Additional prostate cancer specific biomarkers include AC SL3-ETV1, C15ORF21-ETV1, F1135294-ETV1, HERV-ETV1,TMPRS S2-ERG, TMPRSS2-ETV1/4/5, TMPRSS2-ETV4/5, SLC5A3-ERG, SLC5A3-ETV1, SLC5A3-ETV5 or KLK2-ETV

Melanoma specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 19, and can be used to create a melanoma specific biosignature. For example, biomarkers can comprise one or more overexpressed miRs, such as, but not limited to, miR-19a, miR-144, miR-200c, miR-211, miR-324-5p, miR-331, or miR-374, or any combination thereof. The biomarkers can also comprise one or more underexpressed miRs such as, but not limited to, miR-9, miR-15a, miR-17-3p, miR-23b, miR-27a, miR-28, miR-29b, miR-30b, miR-31, miR-34b, miR-34c, miR-95, miR-96, miR-100, miR-104, miR-105, miR-106a, miR-107, miR-122a, miR-124a, miR-125b, miR-127, miR-128a, miR-128b, miR-129, miR-135a, miR-135b, miR-137, miR-138, miR-139, miR-140, miR-141, miR-145, miR-149, miR-154, miR-154#3, miR-181a, miR-182, miR-183, miR-184, miR-185, miR-189, miR-190, miR-199, miR-199b, miR-200a, miR-200b, miR-204, miR-213, miR-215, miR-216, miR-219, miR-222, miR-224, miR-299, miR-302a, miR-302b, miR-302c, miR-302d, miR-323, miR-325, let-7a, let-7b, let-7d, let-7e, or let-7g, or any combination thereof.

The one or more mRNAs that may be analyzed can include, but are not limited to, MUM-1, beta-catenin, or Nop/5/Sik, or any combination thereof and can be used as specific biomarkers for melanoma.

A biomarker mutation for melanoma that can be assessed includes, but is not limited to, a mutation of CDK4 or any combination of mutations specific for melanoma.

The protein, ligand, or peptide biomarker for melanoma that can be assessed can include, but is not limited to, DUSP-1, Alix, hsp70, Gib2, Gia, moesin, GAPDH, malate dehydrogenase, p120 catenin, PGRL, syntaxin-binding protein 1 & 2, septin-2, or WD-repeat containing protein 1, or any combination thereof. The snoRNA that can be used as a biomarker for melanoma include, but are not limited to, H/ACA (U107f), SNORA11D, or any combination thereof.

Biomarkers associated with melanoma also include HSPA8, CD63, ACTB, GAPDH, ANXA2, CD81, ENO1, PDCD6IP, SDCBP, EZR, MSN, YWHAE, ACTG1, ANXA6, LAMP2, TPI1, ANXA5, GDI2, GSTP1, HSPA1A, HSPA1B, LDHB, LAMP 1, EEF2, RAB5B, RDX, GNB1, KRT10, MDH1, STXBP2, RAN, ACLY, CAPZB, GNA11, IGSF8, WDR1, CAV1, CTNND1, PGAM1, AKR1B1, EGFR, MLANA, MCAM, PPP1CA, STXBP1, TGFB1, SEPT2, and TSNAXIP1. One or more of these markers can be assessed to characterize a melanoma.

Pancreatic cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to, miR-221, miR-181a, miR-155, miR-210, miR-213, miR-181b, miR-222, miR-181b -2, miR-21, miR-181b -1, miR-220, miR-181d, miR-223, miR-100-1/2, miR-125 a, miR-143, miR-10a, miR-146, miR-99, miR-100, miR-199.alpha.-1, miR-10b, miR-199a-2, miR-221, miR-181a, miR-155, miR-210, miR-213, miR-181b, miR-222, miR-181b -2, miR-21, miR-181b -1, miR-181c, miR-220, miR-181d, miR-223, miR-100-1/2, miR-125 a, miR-143, miR-10a, miR-146, miR-99, miR-100, miR-199. alpha. -1, miR-10b, miR-199a-2, miR-107, miR-103, miR-103-2, miR-125b-1, miR-205, miR-23a, miR-221, miR-424, miR-301, miR-100, miR-376a, miR-125b -1, miR-21, miR-16-1, miR-181a, miR-181c, miR-92, miR-15, miR-155, let-7f-1, miR-212, miR-107, miR-024-1/2, miR-18a, miR-31, miR-93, miR-224, or let-7d, or any combination thereof.

The biomarker can also comprise one or more underexpressed miRs such as, but not limited to, miR-148a, miR-148b, miR-375, miR-345, miR-142, miR-133a, miR-216, miR-217 or miR-139, or any combination thereof. The one or more mRNAs that may be analyzed can include, but are not limited to, PSCA, Mesothelin, or Osteopontin, or any combination thereof and can be used as specific biomarkers for pancreatic cancer.

A biomarker mutation for pancreatic cancer that can be assessed includes, but is not limited to, a mutation of KRAS, CTNNLB1, AKT, NCOA3, B-RAF, or EGFR, or any combination of mutations specific for pancreatic cancer. The biomarker can also be BRCA2, PALB2, or p16.

Brain cancer (including, but not limited to, gliomas, glioblastomas, meinigiomas, acoustic neuroma/schwannomas, medulloblastoma) specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to miR-21, miR-10b, miR-130a, miR-221, miR-125b-1, miR-125b-2, miR-9-2, miR-21, miR-25, or miR-123, or any combination thereof. The biomarkers can also comprise one or more underexpressed miRs such as, but not limited to, miR-128a, miR-181c, miR-181a, or miR-181b, or any combination thereof.

The one or more mRNAs that may be analyzed include, but are not limited to, MGMT, which can be used as specific biomarker for brain cancer. The protein, ligand, or peptide that can be assessed in a vesicle can include, but is not limited to, EGFR.

Hepatocellular carcinoma specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to, miR-221. The biosignature can also comprise one or more underexpressed miRs such as, but not limited to, let-7a-1, let-7a-2, let-7a-3, let-7b, let-7c, let-7d, let-7e, let-7f-2, let-fg, miR-122a, miR-124a-2, miR-130a, miR-132, miR-136, miR-141, miR-142, miR-143, miR-145, miR-146, miR-150, miR-155(BIC), miR-181a-1, miR-181a-2, miR-181c, miR-195, miR-199a-1-5p, miR-199a-2-5p, miR-199b, miR-200b, miR-214, miR-223, or pre-miR-594, or any combination thereof. The one or more mRNAs that may be analyzed can include, but are not limited to, FAT10.

The one or more biomarkers can also be used to characterize hepatitis C virus-associated hepatocellular carcinoma. The one or more biomarkers can be a miRNA, such as an overexpressed or underexpressed miRNA. For example, the upregulated or overexpressed miRNA can be miR-122, miR-100, or miR-l0a and the downregulated miRNA can be miR-198 or miR-145.

Cervical cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the one or more mRNAs that may be analyzed can include, but are not limited to, HPV E6, HPV E7, or p53, or any combination thereof and can be used as specific biomarkers from a vesicle for cervical cancer.

Endometrial cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to, miR-185, miR-106a, miR-181a, miR-210, miR-423, miR-103, miR-107, or let-7c, or any combination thereof. The biosignature can also comprise one or more underexpressed miRs such as, but not limited to, miR-7i, miR-221, miR-193, miR-152, or miR-30c, or any combination thereof.

A biomarker mutation for endometrial cancer that can be assessed includes, but is not limited to, a mutation of PTEN, K-RAS, B-catenin, p53, Her2/neu, or any combination of mutations specific for endometrial cancer. The protein, ligand, or peptide that can be assessed can include, but is not limited to, NLRP7, AlphaV Beta6 integrin, or any combination thereof.

Head and neck cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to, miR-21, let-7, miR-18, miR-29c, miR-142-3p, miR-155, miR-146b, miR-205, or miR-21, or any combination thereof. The biosignature can also comprise one or more underexpressed miRs such as, but not limited to, miR-494. The one or more mRNAs that may be analyzed include, but are not limited to, HPV E6, HPV E7, p53, IL-8, SAT, H3FA3, or EGFR, or any combination thereof and can be used as specific biomarkers from a vesicle for head and neck cancer.

A biomarker mutation for head and neck cancer that can be assessed includes, but is not limited to, a mutation of GSTM1, GSTT1, GSTP1, OGG1, XRCC1, XPD, RAD51, EGFR, p53, or any combination of mutations specific for head and neck cancer. The protein, ligand, or peptide that can be assessed can include, but is not limited to, EGFR, EphB4, or EphB2, or any combination thereof. Other head and neck cancer specific biomarkers, such as CHCHD7-PLAG1, CTNNB1-PLAG1, FHIT-HMGA2, HMGA2-NFIB, LIFR-PLAG1, or TCEA1-PLAG1 may be used.

GIST specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the one or more mRNAs that may be analyzed can include, but are not limited to, DOG-1, PKC-theta, KIT, GPR20, PRKCQ, KCNK3, KCNH2, SCG2, TNFRSF6B, or CD34, or any combination thereof and can be used as specific biomarkers for GIST.

A biomarker mutation for GIST that can be assessed, but is not limited to, a mutation of PKC-theta or any combination of mutations specific for GIST. The protein, ligand, or peptide that can be assessed can include, but is not limited to, PDGFRA, c-kit, or any combination thereof.

Renal cell carcinoma (RC) specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can also comprise one or more underexpressed miRs such as, but not limited to, miR-141, miR-200c, or any combination thereof. The one or more upregulated or overexpressed miRNA can be miR-28, miR-185, miR-27, miR-let-7f-2, or any combination thereof.

One or more mRNAs that may be analyzed can include, but are not limited to, laminin receptor 1, betaig-h3, Galectin-1, a-2 Macroglobulin, Adipophilin, Angiopoietin 2, Caldesmon 1, Class II MHC-associated invariant chain (CD74), Collagen IV-al, Complement component, Complement component 3, Cytochrome P450, subfamily Ill polypeptide 2, Delta sleep-inducing peptide, Fc g receptor Ma (CD 16), HLA-B, HLA-DRa, HLA-DRb, HLA-SB, IFN-induced transmembrane protein 3, IFN-induced transmembrane protein 1, or Lysyl Oxidase, or any combination thereof and can be used as specific biomarkers for renal cell carcinoma.

A biomarker mutation for renal cell carcinoma that can be assessed includes, but is not limited to, a mutation of VHL or any combination of mutations specific renal cell carcinoma.

The protein, ligand, or peptide biomarker of renal cell carcinoma that can be assessed can include, but is not limited to, IF1alpha, VEGF, PDGFRA, or any combination thereof. Other RCC specific biomarkers include ALPHA-TFEB, NONO-TFE3, PRCC-TFE3, SFPQ-TFE3, CLTC-TFE3, or MALAT1-TFEB.

Esophageal cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biomarkers can comprise one or more overexpressed miRs, such as, but not limited to, miR-192, miR-194, miR-21, miR-200c, miR-93, miR-342, miR-152, miR-93, miR-25, miR-424, or miR-151, or any combination thereof. The biosignature can also comprise one or more underexpressed miRs such as, but not limited to, miR-27b, miR-205, miR-203, miR-342, let-7c, miR-125b, miR-100, miR-152, miR-192, miR-194, miR-27b, miR-205, miR-203, miR-200c, miR-99a, miR-29c, miR-140, miR-103, or miR-107, or any combination thereof. The one or more mRNAs that may be analyzed include, but are not limited to, MTHFR and can be used as specific biomarkers for esophageal cancer.

Gastric cancer specific biomarkers can include one or more (for example, 2, 3, 4, 5, 6, 7, 8, or more) overexpressed miRs, underexpressed miRs, mRNAs, genetic mutations, proteins, ligands, peptides, snoRNA, or any combination thereof. For example, the biosignature can comprise one or more overexpressed miRs, such as, but not limited to, miR-106a, miR-21, miR-191, miR-223, miR-24-1, miR-24-2, miR-107, miR-92-2, miR-214, miR-25, or miR-221, or any combination thereof. The biosignature can also comprise one or more underexpressed miRs such as, but not limited to, let-7a.

The one or more mRNAs that may be analyzed include, but are not limited to, RRM2, EphA4, or survivin, or any combination thereof and can be used as specific biomarkers from a vesicle for gastric cancer. A biomarker mutation for gastric cancer that can be assessed includes, but is not limited to, a mutation of APC or any combination of mutations specific for gastric cancer. The protein, ligand, or peptide that can be assessed can include, but is not limited to EphA4.

Thyroid cancer specific biomarkers include, but are not limited to, AKAP9-BRAF, CCDC6-RET, ERC1-RETM, GOLGA5-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET, characteristic of papillary thyroid carcinoma; or PAX8-PPARy, characteristic of follicular thyroid cancer.

In certain methods, the presence of a mutation in a gene associated with a disease, e.g., a tumor, is determined. In particular embodiments, the mutation is a translocation, rearrangement, or a gene amplification. In other embodiments, the mutation results in a splice mutant, or a truncated or modified encoded polypeptide gene product. In particular embodiments, the mutation comprises at least one nucleotide insertion, deletion or substitution. In particular embodiments, the encoded polypeptide gene product comprises an amino acid insertion, deletion or substitution. Mutations that may be determined include, but are not limited to, any of those described herein. Additional examples of mutations or genomic alterations associated with solid tumors that may be detected according to methods of the present invention include, but are not limited to, mutations in ABL1, AKT1, AKT2, AKT3, ALK, APC, AR, ARAF, BAP1, BCL2L11, BRAF, BRCA1, BRCA2, BTK, CBL, CCND1, CCNE1, CDH1, CDK2, CDK4, CDK6, CDKN1B, CDKN2A, CSF 1R, CTCF, CTNNB1, DDR2, DDX3 X, EGFR, EPHA2, ERBB2, ERBB3, ERBB4, ESR1, ETV6, FBXW7, FGFR1, FGFR2, FGFR3, FGFR4, FLT 1 (VEGFR1), FLT3, FOXO1, GATA3, GNAll, GNAQ, GNAS, HIF1A,HNF 1A, HRAS, IDH1, IDH2, IGF 1R, JAK 1, JAK2, JAK3, KDR, KEAP1, KIT, KMT2A/MLL, KRAS, MAP2K 1, MAP2K2, MAP2K4, MAP3K1, MAP3K9, MAPK1, MCL1, MDM2, MED12, MET, MITF, MTOR, MYC, NF1, NKX2- 1 , NO TCH1 , NO T CH2, NRAS, NTRK 1 , PALB2, PBRM 1 , PDGFRa, PDGFRb,PIK3 CA, PIK3 CD, PIK3R1, PTCH1, PTEN, PTPN11, RB1, RET, RHEB, RHOA, RICTOR, RIT1, ROS 1, RUNX1, SMAD4, SMARCB1, SMO, SPEN, SPOP, SRC, STK11, SYK, TP53, TSC1, TSC2, and VHL. Mutated genes associated with melanoma include, but are not limited to, BRAF, CTNNB1, GNAll, GNAQ, KIT, MEK1, NF1 and NRAS. Mutated or altered (e.g., rearranged or amplified) genes associated with non small cell lung carcinoma include, but are not limited to, AKT1 (mutation), ALK (rearrangement), BRAF (mutation), DDR2 (mutation), EGFR (mutation), FGFR1 (amplification), HER2 (mutation), KRAS (mutation), MEK1 (mutation), METa (amplification), NRAS (mutation), PIK3CA (mutation), PTEN (mutation), RET (rearrangement) and ROS1a (rearrangement). Mutated or altered (e.g., rearranged or amplified) genes associated with ovarian cancer include, but are not limited to, BRAF, KRAS, PIK3CA, and PTEN. Mutated or altered (e.g., rearranged or amplified) genes associated with colorectal cancer include, but are not limited to, AKT1, BRAF, KRAS, NRAS, PIK3CA, PTEN and SMAD4.

In certain embodiments, the present invention includes a method of determining the presence of a mutation in a gene or allele in a tissue sample from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a gene mutation whose presence or amount is indicative of the presence or absence of disease, such as a cancer or tumor, tumor metastasis, or tumor resistance to therapy, e.g., a cancer stem cell. In certain embodiments, any of the the mutations described herein are detected to identify an associated disease or tumor. In particular embodiments, the methods further comprises: (vi) comparing the image to one or more control images or predetermined images, or presence or absence of the gene mutation, obtained from normal tissue, disease tissue, tumor tissue, tumor tissue associated with a good prognosis, or tumor tissue associated with a poor prognosis or metastasis, or prediction of treatment response, thereby determining the presence of a tumor, the liklihood of metastasis, or the prognosis of a tumor.

Aspects of the invention maybe used to detect the presence of a genetic abnormality in any one or more loci of interest that may be associated with a disease. For example, one or more different loci associated with a tumor, a cancer, a precancer or any other disease or disorder may be assayed according to methods of the invention. Examples of target nucleic acids include, but are not limited to, one or more oncogenes, tumor suppressor genes, genomic regions containing nucleic acid repeats (e.g., different forms of satellite DNA such as micro or mini satellite DNA), other genetic loci (coding or non-coding genetic loci), or combinations thereof. Examples of genes, miRNAs etc. showing differential expression, translocation, rearrangement, etc. are provided in FIG. 19.

In certain embodiments of the invention the presence or absence of mutations can be indicative of risk associated with developing cancer, early detection of cancer, and finally prognosis and treatment of cancer. These mutations include, but are not limited to, the following genes: MSH2, MSH6, MLH1, PMS2, BUB1, BUBR1, MRE11, CDC4, APC, beta-catenin, TGF-beta, SMAD4, p21 Waft, 14-3-3 sigma, PUMA, BAX, PRL-3, and PIK3CA. It is appreciated that these genes are involved at different stages of the neoplastic process and therefore can be selectively used to detect or monitor tumor origination, progression or recurrence. For example, mutations in the APC/beta-catenin pathway initiate the neoplastic process. Detection of mutations in these genes is very useful for cancer risk assessment. A patient with identified mutations in the APC/beta-catenin genes is at an elevated risk for developing tumors. Most often, mutations in the APC/beta-catenin genes result in adenomas (small benign tumors). These tumor progress, becoming larger and more dangerous, as mutations in other growth-controlling pathway genes accumulate. Growth-controlling pathway genes include K-Ras, B-RAF, PIK3CA, or p53. Detection of mutations in these genes could lead to early detection of the development of tumors. If undetected and untreated (in some circumstances) the neoplastic process can be accelerated by mutations in stability genes, such as PIKSCA/PTEN, PUMA, p53/BAX, p21Waf1, 14-3-3 sigma, or PRL-3. Many of these gene products function to block cell birth, cell cycle and/or to activate cell death and apoptosis. Others like PRL-3 and PIK3CA are involved in regulating metastasis. Detection of mutations in these genes is significant for determining the clinical prognosis of a particular cancer, the treatment efficacy of a particular cancer treatment and for monitoring the progression or recurrence of a given tumor.

Examples of breast cancer specific fusions that may be detected according to methods of the present invention include, but not limited to, ETV6-NTRK3. Examples of lung cancer specific fusions, include, but not limited to, RLF-MYCL1, TGF-ALK, or CD74-ROS1. Examples of prostate cancer specific fusions, include, but are not limited to, ACSL3-ETV1, C15ORF21-ETV1, F1135294-ETV1, HERV-ETV1,TMPRSS2-ERG, TMPRSS2-ETV1/4/5, TMPRSS2-ETV4/5, SLC5A3-ERG, SLC5A3-ETV1, SLC5A3-ETV5 or KLK2-ETV4. Examples of brain cancer specific fusions, include, but are not limited to, GOPC-ROS1. Examples of head and neck cancer specific fusions, include, but are not limited to, CHCHD7-PLAG1, CTNNB1-PLAG1, FHIT-HMGA2, HMGA2-NFIB, LIFR-PLAG1, or TCEA1-PLAG1. Examples of RCC specific fusions, include, but are not limited to, ALPHA-TFEB, NONO-TFE3, PRCC-TFE3, SFPQ-TFE3, CLTC-TFE3, or MALAT1-TFEB. Examples of thyroid cancer specific fusions, include, but are not limited to, AKAP9-BRAF, CCDC6-RET, ERC1-RETM, GOLGA5-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET, characteristic of papillary thyroid carcinoma; or PAX8-PPARy, characteristic of follicular thyroid cancer.

D. Cell Death or Apoptosis

In cancer, apoptotic processes occur both spontaneously and induced by antitumor therapies. The balance between the cell death and cell proliferation also plays a role in the progression of malignancy. When cells are determined to undergo apoptosis, several pro-apoptotic proteins are activated, including the Bcl-2/BAX family genes, which include, among others, BAX, BAK, BOKk, Bcl-XS, etc. Truncated forms of BAX, such as the BH3-only members, are also known to be pro-apoptotic, and these include BID, BAD, BIK, BIM, NOXA, PUMA (p53 upregulated modulator of apoptosis), etc. These mRNAs are all associated with apoptotic responses in one or more tumor types. GADD153 (growth arrest and DNA damage-inducible gene) has been shown to mediate apoptosis. The Apaf-1 protein interacts with cytochrome C released by the mitochondria and dATP to form apoptosome complexes, which can activate caspase 9 and lead to apoptosis. Apaf-1 silencing or downregulation has been implicated in melanoma and glioblastoma. SUMO-1 (small ubiquitin-related modifier) is structurally related to ubiquitin, but when proteins are modified by SUMO (sumoylated) they are protected from ubiquitin-mediated degradation. Bfl-1 is a member of the Bcl-2 family that suppresses p53-mediated apoptosis. Elevated levels of Bfl-1 have been found in cancers. BCL-W is another member of the Bcl-2 family that has antiapoptotic effects. The Bcl-2 gene has antiapoptotic effects. It forms complexes with caspase-9 and Apaf-1, which prevent these proteins from forming the apoptosome and initiating the protease cascade leading to apoptosis. It is implicated in a variety of cancers. PUMA (p53-upregulated modulator of apoptosis, also known as BBC3) induces apoptosis via the mitochondrial apoptotic pathway. NOXA (also known as PMAIP1 or ARP) has been found to be highly expressed in adult T-cell leukemia and has a proapoptotic function in response to cellular damage, participating in the activation of caspase-9 and ensuing apoptosis. Hrk is an activator of apoptosis that interacts with Bcl-2. It has been implicated in astrocytic tumors and central nervous system lymphomas. Bim shares the short BH3 motif with most Bcl-2 homologues; it provokes apoptosis. It has been implicated in the development of mantle cell lymphoma. BINP3 is a proapoptotic Bcl-2 family protein that has been linked to malignant glioma. The Bik (Bcl-2-interacting killer) protein is another proapoptotic Bcl-2 family member; it has been implicated in B-cell lymphomas and breast cancer, and expression has also been observed in epithelial and lung cells. Bid (BH3 interacting domain death agonist) is a proapoptotic protein that is implicated in osteosarcoma and gastric cancers. Bad is another Bcl-2 proapoptotic family member that has been linked to B-cell lymphomas and colon cancer. Bcl-XS is another proapoptotic member of this family that has been linked to hepatocellular carcinoma, breast cancer, and ovarian cancer. Bok (Bcl-2 related ovarian killer) is another Bcl-2 proapoptotic family member that has been linked to ovarian cancer. Bak, another Bcl-2 homolog, is a strong promoter of apoptosis, and has been implicated in gastric and colorectal cancers. Bax (Bcl-2 associated X protein) is a proapoptotic protein that is implicated in numerous cancers, including acute and chronic lymphocytic leukemias, gastric and colorectal cancer, breast cancer, and pancreatic cancer. Expression of LRP (lung resistance protein) has been found in human fibrosarcomas, hepatocellular carcinoma, and acute myeloid leukemia. The MRP gene has been shown to be expressed in hepatocellular carcinoma and colorectal carcinoma.

In certain embodiments, the present invention includes a method of examining apoptosis or cell death, or markers thereof, in a tissue sample, e.g., a tumor tissue sample, comprising processing a tissue sample obtained from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a marker of apoptosis or cells death whose characteristics may be indicative of the presence or absence of disease, such as a cancer or tumor. In certain embodiments, any of the detectable markers of apoptosis or cell death described herein are used to detect an associated disease or tumor. In particular embodiments, the methods further comprises: (vi) comparing the image to one or more control images or predetermined images obtained from normal tissue, disease tissue, tumor tissue, tumor tissue associated with a good prognosis, or tumor tissue associated with a poor prognosis, thereby determining the presence of a tumor or the prognosis of a tumor, or the presence of or amount of apoptosis or cell death.

The expression levels, activities, and patterns of these and other markers of apoptosis may be determined according to the present invention. In particular embodiments, p21, GADD, Apaf-1, SUMO, Bfl-1, BCL-W, BCL-2, PUMA, NOXA, Hrk, Bim, BINP3, Bik, Bid, Bad, Bcl-XS, Bok, Bak, Bax, LRP, and MRP are markers detected according to methods of the present invention. In particular embodiments, markers that may be used to characterize an increased or reduced level of apoptosis include, but are not limited to, bad, bax, bcl-2, bcl-w, BID, BIM, Caspase 3, Caspase 8, CD40, CD40 Ligand, Cytochrome-C, DR6, Fas, Fas Ligand, HSP27, HSP60, HSP70, HTRA, IGF-1, IGF-2, IGFBP-1, IGFBP-2, IGFBP-3, IGFBP-4, IGFBP-5, IGFBP-6, IGF-1sR, livin, p21, p27, p53, SMAC, Survivin, sTNFRI, sTNFRII, TNF alpha, TNF beta, TRAIL R1, TRAIL R2, TRAIL R3, TRAIL R4, and XIAP.

The expression level of various polypeptides (or polynucleotides) involved in the apoptotic pathway may be determined. In general, an increased amount of pro-apoptotic markers is associated with tumor death or effective treatment, while an increased amount of anti-apoptotic markers is associated with tumor growth. Protein or nucleotide expression levels, or protein activity levels, within a biological sample, e.g., a tumor tissue sample, may be compared to normal controls, or to pre-determined levels known to be associated with either normal or diseased cells. Alternatively, or in addition, the amount of apoptotic markers within a biological sample, e.g., a tumor tissue sample, may be compared to pre-determined amounts of, location of, and ratios of apoptotic markers in the same type of tumor. In certain embodiments, the predicted clinical outcome may be determined by comparing the amount of apoptotic markers in the tumor tissue sample to the amount observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested with that of those tumors with known clinical outcomes having amounts of or location of apoptotic markers most similar to the tumor tissue sample being tested.

E. Activation of Signalling Pathways in Tumors

Aberrant signal transduction pathway activation is a hallmark of cancer. These signaling pathways that regulate normal cellular processes may also contribute to malignant cell proliferation. Key pathways involved in growth and proliferation include, but are not limited to, PI3K/Akt/mTor, Ras/Raf/MEK, and the Wnt/β-catenin, or the Hedgehog/patched signaling pathways. In addition to regulating cancer proliferation and growth, collectively, these pathways modulate multiple physiological processes including, apoptosis, cell cycle regulation, DNA maintenance, gene transcription, survival, metabolism, angiogenesis, would healing, cell migration, and differentiation. Stimulation of these pathways can be via cytokines, survival factors, chemokines, death factors, hormones transmitters, growth factors, extracellular matrix, WNT or Hedgehog. Activation of these pathways can be via cytokine receptors, receptor tyrosine kinases, G protein coupled receptors and integrins. Activation of these pathways could be determined by indirectly measuring the expression and/or phosphorylation of various upsteam and downstream proteins including IL-6, IL2, IGF1, TGFalpha, EGF, heregulin, EGFR, ERBB2, ERBB3, ERBB4, cMET, IGF1R, FGFR, ER, PR, Integrin beta, Frizzled, Patched, WNT, Hedgehog, FASR, PI3K, AKT, PKC, PLC, NFkB, JAK, STAT3&5, BCL-XL, Caspase 9, Caspase, 8, FADD, RAS, RAF, MEK, MEKK, MAPK, MKK, FAK, APC, beta-catenin, ARF, ERK, JNK, JUN, FOS, CDK2, p53, p21, p27, p15, p16, SMAD, CDC42. In addition, the measurement and activation of potential homodimers/heterodimers of the HER family receptors such as HER2 homodimers, HER2/HER3 heterodimers, HER1/HER2 heterodimers may be diagnostic, prognostic or predictive of treatment in patients with various cancers.

In certain embodiments, the present invention includes a method of determining the presecnce of aberrant signal transduction pathways, in a tissue sample, e.g., a tumor tissue sample, comprising processing a tissue sample obtained from the subject by: (i) fixing the tissue sample in the presence of hydrogel subunits; (ii) polymerizing the hydrogel subunits to form a hydrogel-embedded sample; (iii) clearing the hydrogel-embedded sample; (iv) imaging the processed sample to generate at least one image of the processed sample. In particular embodiments, the method further comprises: (v) labeling the cleared hydrogel-embedded sample with one or more first detectable marker. In particular embodiments, the tissue sample is a tumor tissue sample. In particular embodiments, the detectable marker binds a component of the PI3K/Akt/mTor, Ras/Raf/MEK, and the Wnt/β-catenin, or the Hedgehog/patched signaling pathways. In particular embodiments, the detectable marker binds IL-6, IL2, IGF1, TGFalpha, EGF, heregulin, EGFR, ERBB2, ERBB3, ERBB4, cMET, IGF1R, FGFR, ER, PR, Integrin beta, Frizzled, Patched, WNT, Hedgehog, FASR, PI3K, AKT, PKC, PLC, NFkB, JAK, STAT3&5, BCL-XL, Caspase 9, Caspase, 8, FADD, RAS, RAF, MEK, MEKK, MAPK, MKK, FAK, APC, beta-catenin, ARF, ERK, JNK, JUN, FOS, CDK2, p53, p21, p27, p15, p16, SMAD, or CDC42. In certain embodiments, the detectable marker binds homodimers/heterodimers of the HER family receptors such as HER2 homodimers, HER2/HER3 heterodimers, HER1/HER2 heterodimers that may be diagnostic, prognostic or predictive of treatment in patients with various cancers may be indicative of the presence or absence of disease, such as a cancer or tumor. In certain embodiments, any of the signal transucation pathways or markers described herein are used to detect an associated disease or tumor. In particular embodiments, the methods further comprises: (vi) comparing the image to one or more control images or predetermined images obtained from normal tissue, disease tissue, tumor tissue, tumor tissue associated with a good prognosis, or tumor tissue associated with a poor prognosis, thereby determining the presence of a tumor or the prognosis of a tumor, or the presence of or amount of apoptosis or cell death. Protein or nucleotide expression levels, or protein activity levels, within a biological sample, e.g., a tumor tissue sample, may be compared to normal controls, or to pre-determined levels known to be associated with either normal or diseased cells. Alternatively, or in addition, the amount of signal transducation markers within a biological sample, e.g., a tumor tissue sample, may be compared to pre-determined amounts of, location of, and ratios of signal trasnduction markers in the same type of tumor. In certain embodiments, the predicted clinical outcome may be determined by comparing the amount of signal trasnduction markers in the tumor tissue sample to the amount observed in tumors of the same type having a known clinical outcome, e.g., as measured by overall survival (OS), progression-free survival (PFS), time to progression (TTP), time to treatment failure (TTF), event-free survival (EPS), time to next treatment (TTNT), overall response rate (ORR) or duration of response (DofR), and correlating the predicted clinical outcome of the tumor tissue sample being tested with that of those tumors with known clinical outcomes having amounts of or location of signal transduction markers most similar to the tumor tissue sample being tested.

F. Evaluation of Biomarkers of Response to Targeted Therapies in Breast Cancer

In one embodiment, the present invention provides methods for evaluating the response of a breast cancer to a therapy, e.g, a current FDA-approved targeted therapy. There are significant limitations to measuring the co-expression of key biomarkers of both drug response and resistance, as well as the inherent subjectivity, in the current methodologies using single-protein/DNA measurements on formalin fixed paraffin-embedded tissue. The clinical success of trastuzumab in the treatment of breast cancer demonstrated that tumor evaluation is essential to correctly identify patients that would be candidates for targeted therapies. The two assays that are routinely utilized to select patients for trastuzumab therapy are HER2 protein expression by immunohistochemistry (IHC) and gene amplification by fluorescence in situ hybridization (FISH). Unfortunately, both assays only partially predict for response to the drug. In the case of epidermal growth factor receptor (EGFR), the link between response and EGFR tyrosine kinase inhibitors could not be established, despite high levels of expression of this protein. Although these tests have become the benchmarks for defining tumors as HER-2 positive, considerable controversy exists regarding the accuracy and reproducibility of these methods. It is estimated that up to 20% of HER-2 testing in the community may be inaccurate when validated against central laboratory testing due to the subjective nature of these studies. Furthermore, studies have shown that the expression and activation of other pathways, such as those involving HER3 expression and the truncated form of HER2 (p95HER2), can contribute to resistance to standard trastuzumab therapy. These kinds of questions can be answered in a limited way by methods that disaggregate or homogenize tissue, or that utilize conventional single-stain IHC on Formalin Fixed Paraffin-embedded Tissue (FFPE). The present invention provides alternative methods to prepare and image tissue specimens in order to be able to reconstruct the true biology and potential activation of resistance pathways.

The present invention provides a method for predicting whether a subject having breast cancer will be responsive to treatment with trastuzumab. In particular embodiments, a breast cancer tissue sample obtained from the subject is processed as described herein to form a fixed, cross-linked sample, cleared of lipids, and then stained with one or more detectable markers of HER2 protein expression, HER2 gene amplification, and/or p95HER2 expression. In other embodiments, the sample is stained with one or more detectable markers that bind to HER2, estrogen receptor (ER), progesterone receptor (PR), HER1, HER3, heregulin, or ki67 protein or mRNA. The presence of and/or amount of the markers detect (either protein or nucleic acid) is determined using microscopy techniques described herein, and the results are compared to compared to control standards, such as breast tumor samples previously characterized for HER2 and ER/PR expression status and known to respond favorably or not to trastuzumab treatment. Where the sample being tested shares characteristics in common to previously characterized samples that respond favorably, it is indicated to treat the subject with trastuzumab, but when the sample being tested shares characteristics more in common to previously characterized samples that respond poorly, it is indicate to not treat the subject with trastuzumab.

G. Evaluation of Immune Cells and Checkpoint Proteins

Methods of the present invention may also be used in tumor types relevant to therapies with immune checkpoint inhibitors, since it allows an understanding of the biological landscape of potential immune modulators in models of cancer, such as advanced melanoma and non-small cell lung cancer (NSCLC). Existing methods for the measurement of PD-Ll protein expression using formalin fixed paraffin-embedded (FFPE) tissue are not robust and reproducible. Patients that lack PD-L 1 expression by immunohistochemistry respond to PD-1 inhibitors, indicating that the potential interplay between the tumor, the microenvironment, as well as the host is crucial, and suggesting that single analyte measurements of PD-L1 are not be the optimal way to determine clinical efficacy.

Blocking the interaction between the programmed cell death (PD)-1protein and one of its ligands, PD-L 1, has been reported to have impressive anti-tumor responses in many tumor types, including advanced melanoma and NSCLC. Tumor expression of PD-LI is currently a suggestive, but an inadequate, biomarker to predict response to immune-checkpoint inhibitors. Tumor heterogeneity is a significant issue in many tumors, making smaller biopsy samples less reliable for tissue-based biomarkers, such as PD-L 1 tumor expression. The interaction between host cells, the invading tumor cells, and the immune system is quite complex and the measurement of single protein on tissue sections or by flow cytometry has significant limitations.

Immune checkpoint therapies, that target regulatory pathways in T cells to enhance antitumor immune responses, has led to important clinical advances in the area of cancer treatment. These therapies have demonstrated durable clinical responses and, in a fraction of patients, long-term outcomes where patients exhibit no clinical signs of cancer for many years. To fully understand the mechanisms and predictive biomarkers for this class of novel agents lies in the ability to understand human immune responses within the context of the tumor microenvironment. This will provide valuable de novo and dynamic information regarding the immune response and regulation of additional pathways that will need to be targeted through combination therapies to provide survival benefit for greater numbers of patients. Biomarkers associated with responsiveness to immune checkpoint therapies include, but are not limited to, those shown in Table 6.

TABLE 6

| | |
|---|---|
| 4-1BB | CD80 |
| B7H4 | VISTA |
| CD70 | CD40 |
| CTLA-4 | GITR |
| HVEM | LAG-3 |
| OX40 | PD-L1 |
| Ligand | |
| SIRP alpha | TIM-3 |
| B7-H2 | CD160 |
| 4-1BB | CD86 |
| Ligand | |
| BTLA | CD27 |
| CD28 | CD40 Ligand |
| DNAM-1 | GITR Ligand |
| ICOS | OX40 |
| PD-1 | PD-L2 |
| TIGIT | 2B4 |
| CD155 | CD47 |

In particular embodiments, the present invention provides a method for predicting whether a subject having a cancer such as advanced melanoma or NSCLC will be responsive to treatment with an agent that inhibits PD-1 protein or its ligand, PD-L1, or blocks their interaction with each other. In particular embodiments, a cancer tissue sample obtained from the subject is processed as described herein to form a fixed, cross-linked sample, cleared of lipids, and then stained with one or more detectable markers of PD-L1 protein expression. In other embodiments, the sample is stained with one or more detectable immune markers, such as CD4, CD8, CD20, cytokeratin, or PD-L1, or any of those shown in Table 6. The presence of and/or amount of the markers detected (either protein or nucleic acid) is determined using microscopy techniques described herein, and the results are compared to compared to control standards, such as tumor samples previously characterized for expression status of any of these markers and known to respond favorably or not to treatment with a PD-1 inhibitor. Where the sample being tested shares characteristics in common to previously characterized samples that respond favorably, it is indicated to treat the subject with a PD-1 inhibitor, but when the sample being tested shares characteristics more in common to previously characterized samples that respond poorly, it is indicate to not treat the subject with a PD-1 inhibitor.

H. Cancer Stem Cells

Identifying and accurately quantifying the presence and clinical implications of cancer stem cells within intact biological systems is important in predicting cancer therapy resistance. For example, breast cancer is a heterogeneous disease and several well-documented breast cancer subtypes exist that differ in their prognostic and predictive characteristics. In recent years, basic science and translational studies have demonstrated that cancer stem cells contribute to the heterogeneous histological and functional characteristics of breast cancer. Even more recently, the ability of breast epithelial cells to undergo an epithelial to mesenchymal (EMT) transition has been linked to the acquisition of stem cells properties, and enhanced tumor invasion, metastasis, and resistance to available treatments. For this reason, stem cells and cells undergoing EMT are attractive targets for potential therapy. Their identification in breast tissue samples would provide important information in determining breast cancer prognosis and treatment, and also enable pathologists to discover and validate prognostic and predictive markers, as well as identify markers of increased risk for breast cancer. From a clinical perspective, the importance of identifying and targeting stem cells is based on their reported chemo- and radio-resistance, which may be responsible for the failure of current treatment modalities to cure breast cancer. Furthermore, a number of studies have demonstrated that the presence and abundance of breast cancer stem cells in tissue samples have prognostic significance. In recent years, basic science studies have provided strong evidence that the EMT and stem cell programs are closely interrelated and are a cause of breast cancer heterogeneity.

The present invention provides methods for the identification of cancer stem cells (CSCs) and epithelial to mesenchymal transition (EMT) in a tumor tissue sample, e.g., a triple negative breast cancer model. In particular embodiments, a cancer tissue sample obtained from the subject is processed as described herein to form a fixed, cross-linked sample, cleared of lipids, and then stained with one or more detectable immune markers, such as ALDH1, CD24, CD44, CD133, E-cadherin, and vimentin. In addition, HER2 and/or ER/PR status may be determined. The presence of and/or amount of the markers detected (either protein or nucleic acid) is determined using microscopy techniques described herein, and the results are compared to compared to control standards, such as tumor samples previously characterized for expression status of any of these markers and known to develop resistance or not. Where the sample being tested shares characteristics in common to previously characterized samples that develop resistance to therapy, it is predicted that the subject will also develop resistance to therapy, but when the sample being tested shares characteristics more in common to previously characterized samples that does not develop resistance, it is indicated that the subject will not develop resistance.

I. Intact Visualization of Solid Tumors with Heterogeneous Tumor Foci

Local therapy fails in up to 30-40% of prostate cancer patients despite the presence of homogeneous clinical risk parameters (the same NCCN (National Comprehensive Cancer Network) risk category based on similar TNM stages, Gleason scores, and pretreatment PSA (prostate-specific antigen) values). Biomarkers based on mRNA abundance in primary tumor or blood samples have not yet reached full clinical utility contributing to a further lack of understanding of inherent intra-glandular and multifocal heterogeneity. Although the majority of prostate cancer is diagnosed as organ-confined disease, cancers with similar Gleason scores (for example, Gleason scores 7-10) show substantial interpatient heterogeneity and differential prostate cancer-specific mortality rates. A further complexity lies in intra-glandular biological heterogeneity between individual cancer foci as 80% of prostate cancers contain >1 disease focus. Individual prostate cancer foci are believed to be clonal, but their molecular nature within a given patient remained largely uncharacterized at whole-genome resolution until the recent publication by Boutras et al 2015. Overall, the findings reported by Boutros et al highlight the limitations of relying on one tumor section or biopsy for determining the molecular status of prostate cancer for clinical decision-making. This study provided biological rationale for future trials dedicated towards understanding the extent to which these issues influence survival outcomes in patients. This may require the extensive characterization of matched primary and metastatic tumors in patients who develop lethal disease.

The present invention provides new technologies to capture molecular heterogeneity including those involving liquid biopsies and/or molecular imaging important in translating the findings of such studies into the clinic, and providing an understanding of the complexity of prostate cancer heterogeneity using intact imaging as compared to molecular characterization of tumor tissue as described in Boutros et al. In particular embodiments, a cancer tissue sample obtained from the subject is processed as described herein to form a fixed, cross-linked sample, cleared of lipids, and then stained with one or more detectable markers, including any of those described herein. The presence of and/or amount of the markers detected (either protein or nucleic acid) is determined using microscopy techniques described herein, and the results are compared to compared to control standards, such as tumor samples previously characterized for expression status of any of these markers and known to develop resistance or not. Where the sample being tested shares characteristics in common to previously characterized samples that develop resistance to therapy, it is predicted that the subject will also develop resistance to therapy, but when the sample being tested shares characteristics more in common to previously characterized samples that does not develop resistance, it is indicated that the subject will not develop resistance.

J. Microenvironment and Response to Cancer Therapies in Solid Tumors

It is believed that the failure of most of the drugs currently available for pancreatic cancer is due to lack of understanding of the biology of the tumor microenvironment and the inability of many cancer drugs to penetrate the complex stromal component of pancreatic cancer. Pancreatic cancer is the fourth leading cause of cancer death in the United States and is typically diagnosed in later stages of the disease when cure rates are quite low. The microenvironment of pancreatic cancer (80-90% of the tumor volume) appears to be the one of the best examples of an environment perfectly suited to support cancer growth. It is very heterogeneous and contains many cellular and acellular components, such as fibroblasts, myofibroblasts, pancreatic stellate cells, immune cells, blood vessels, extracellular matrix (ECM), and soluble proteins such as cytokines and growth factors. The resulting microenvironment supports tumor initiation, progression, invasion, and metastasis. Moreover, stromal cells express multiple proteins and growth factors associated with treatment resistance. Recent efforts have been aimed at modifying or targeting structural proteins in the microenvironment including, hyaluronan (HA) (in primary and metastases), collagen, and SPARC (secreted protein, acidic, and rich in cysteine). Success in targeting this aspect of the cancer has produced a regimen that improves survival for patients with advanced pancreatic cancer (nab-paclitaxel+gemcitabine), however, better treatments and understanding of the biology of this cancer are needed. To this end, there is a large discrepancy between the effectiveness of some therapies, as reported in preclinical assays (cell lines and xenograft models) and subsequent failure in human clinical trials.

Methods of the present invention may be used to examine and understand the complexity of the tumor microenvironment in various cancers, such as pancreatic cancers, e.g., for determining cancer prognosis, predicting or monitoring response to therapy, and predicting or determining resistance to therapy. In particular embodiments, a cancer tissue sample (e.g., pancreatic tumor tissue sample) obtained from the subject is processed as described herein to form a fixed, cross-linked sample, cleared of lipids, and then stained with one or more detectable immune markers of the tumor microenvironment, e.g., markers that specifically bind to microenvironment components, such as cells or extracellular matrix components of the tumor microenvironment, including any of those described herein, e.g., SPARC, hyaluronan, and/or collagen. The presence of and/or amount of the markers detected (either protein or nucleic acid) is determined using microscopy techniques described herein, and the results are compared to compared to control standards, such as tumor samples previously characterized for expression status of any of these markers and known to develop resistance or not. Where the sample being tested shares characteristics in common to previously characterized samples that develop resistance to therapy, it is predicted that the subject will also develop resistance to therapy, but when the sample being tested shares characteristics more in common to previously characterized samples that does not develop resistance, it is indicated that the subject will not develop resistance. Where the sample being tested shares characteristics in common to previously characterized samples that respond well to a therapy, it is predicted that the subject will also respond well to the therapy, but when the sample being tested shares characteristics more in common to previously characterized samples that respond poorly to a therapy, it is indicated that the subject will also respond poorly to the therapy.

### V. Methods for Screening Drugs

Methods of the present invention may be readily adapted for screening candidate therapeutic agents to determine their effectiveness in treating diseases and disorders, including cancers. In particular embodiments, these methods are performed using animal models of disease. A variety of animal models of cancer and tumorigenesis are known and available in the art. For example, mouse cancer models are well known, and xenograft and chemically or genetically induced mouse cancers are commonly used rodent cancer models. In certain embodiments, these methods are practiced using human tissue sample, e.g., tumor tissue samples, obtained from a patient treated with a drug candidate, for example.

In some embodiments, a first disease tissue sample (e.g., a tumor tissue sample) is obtained from a subject, e.g., an animal model of disease (e.g., cancer), at a first time point, e.g., before treatment with a candidate drug and examined according to methods of the present invention. The subject is then treated with the candidate drug, and a second disease tissue sample is obtained from the subject at a second time point during or following treatment. The first and second disease tissue samples are processed and analyzed as described herein, optionally after being stained with agents that allow visualization of tissue structures and/or components (e.g., cells, proteins, nucleic acids), such as tumor markers, associated with the disease or disorder, e.g., tumor. If the second disease tissue sample has reduced amounts of structures or markers of disease than the first disease tissue sample, it is indicative that the candidate drug is effective in treating the disease or disorder. If the second disease tissue sample has increased amounts of structures or markers of disease than the first disease tissue sample, it is indicative that the candidate drug is not effective in treating the disease or disorders.

In particular embodiments, these methods may be practiced using any of the methodologies and biomarkers or structures described herein.

### VI. Methods of Treatment

The present invention also provides methods of treating diseases and disorders. The methods described herein may be used to determine the therapeutic agent to use to treat the disease or disorder, or they may be used to monitor the response of the subject to treatment. In particular embodiments, these methods comprise obtaining a disease tissue sample from a subject diagnosed with or suspected of having a disease or disorder, processing and imaging the disease tissue sample as described herein, determining the presence of, an amount of, or characteristics associated with the disease or disorder, and then treating the subject with a therapeutic agent known to be effective in treating a disease or disorder having a similar amount of or characteristics as determined in the disease tissue sample. In particular embodiments, these methods further comprise obtaining a second disease tissue sample from the subject following treatment, processing and imaging the second disease tissue sample as described herein, and determining the presence of, an amount of, or characteristics associated with the disease or disorder, to determine if the treatment is effective in treating the disease or disorder. In particular embodiments, the disease or disorder is a tumor.

In particular embodiments, the use of a particular drug to treat a cancer is indicated where the tumor displays a biomarker characteristic predictive of response to the drug. In certain embodiments, methods of the present invention may be used to ascertain the status of one or more biomarkers in a tumor to determine the drug to use to treat the subject from whom the tumor tissue sample was obtained. In particular embodiments, the status of a biomarker and referenced subgroup shown in Table 1 is determined according to a method of the present invention, and the subject is treated with the indicated drug when the corresponding phenotype is determined.

TABLE 1

| | | Referenced |
|---|---|---|
| Drug | Biomarker† | Subgroup |
| Ado-Trastuzumab | ERBB2 | HER2 protein |
| Emtansine | | overexpression |
| | | or gene |
| | | amplification |
| | | positive |
| Afatinib | EGFR | EGFR exon 19 |
| | | deletion or |
| | | exon 21 |
| | | substitution |
| | | (L858R) positive |
| Anastrozole | ESR1, PGR | Hormone |
| | | receptor- |
| | | positive |
| Ceritinib | ALK | ALK gene |
| | | rearrangement |
| | | positive |
| Cetuximab (1) | EGFR | EGFR protein |
| | | expression |
| | | positive |
| Cetuximab (2) | KRAS | KRAS codon |
| | | 12 and 13 |
| | | mutation |
| | | negative |
| Crizotinib | ALK | ALK gene |
| | | rearrangement |
| | | positive |
| Dabrafenib (1) | BRAF | BRAF V600E/K |
| | | mutation |
| | | positive |
| Dabrafenib (2) | G6PD | G6PD deficient |
| Erlotinib (1) | EGFR | EGFR protein |
| | | expression |
| | | positive |
| Erlotinib (2) | EGFR | EGFR exon 19 |
| | | deletion or |
| | | exon 21 |
| | | substitution |
| | | (L858R) |
| | | positive |
| Everolimus (1) | ERBB2 | HER2 protein |
| | | overexpression |
| | | negative |
| Everolimus (2) | ESR1 | Estrogen |
| | | receptor |
| | | positive |
| Exemestane (1) | ESR1 | Estrogen |
| | | receptor |
| | | positive |
| Exemestane (2) | PGR | Progesterone |
| | | receptor |
| | | positive |
| Fulvestrant | ESR1, PGR | Hormone |
| | | receptor |
| | | positive |
| Lapatinib (1) | ERBB2 | HER2 protein |
| | | overexpression |
| | | positive |
| Letrozole | ESR1, PGR | Hormone |
| | | receptor |
| | | positive |
| Panitumumab (1) | EGFR | EGFR protein |
| | | expression |
| | | positive |
| Panitumumab (2) | KRAS | KRAS codon |
| | | 12 and 13 |
| | | mutation |
| | | negative |
| pembrolizumab | PD-L1 | PD-L1 |
| | | overexpression |
| | | in tumors |
| Pertuzumab | ERBB2 | HER2 protein |
| | | overexpression |
| | | positive |
| Tamoxifen (1) | ESR1, PGR | Hormone |
| | | receptor |
| | | positive |
| Trametinib | BRAF | BRAF V600E/K |
| | | mutation |
| | | positive |
| Trastuzumab | ERBB2 | HER2 protein |
| | | overexpression |
| | | positive |
| Vemurafenib | BRAF | BRAF V600E |
| | | mutation |
| | | positive |

### EXAMPLES

### Example 1

### CLARITY Analysis of Tumor Cells, Mouse Xenograft Tumors, and Human Tumors

Tumor cell lines, xenografts and tumor tissues were processed and analyzed according to methods described herein. These studies demonstrate that the claimed methods may be used to successfully cross-link, clear and label tumor samples using various probes to examine structural features and expression of tumor markers within the samples.

### Methods

Cell Culture, Frozen Pellet Generation, Preparation of Cells for Mouse Xenograft Inoculations

Reagents and chemicals utilized in this set of experiments are shown in Table 2.

TABLE 2
Reagents and Chemicals Used

| Reagents | Vendor | Catalog # |
|---|---|---|
| Borate Buffer 1M | VWR | PI28341 |
| Sodium Docecal Sulfate | VWR | 89167-708 |
| SDS 20% solution | | |
| 16% Paraformadehyde | Electron Microscopy Sciences | 15710-S |
| (PFA) | | |
| Triton-X 100 | Sigma Aldrich | X100-1L |
| VA-044 | Wako Chemicals | 1119365 |
| 40% acrylamide | Bio-Rad | 1610140 |
| 2% bis-acrylamide | Bio-Rad | 1610142 |
| 1X PBS | Lonza | 17-516q |
| 10X PBS | Lonza | 17-517q |
| RapiClear mounting | SunjinLabs | RCCS002 |
| solution | | |
| RapiClear mounting gel | SunjinLabs | RCCS005 |
| Pen/Strep (100X) | Thermofisher scientific | 15460-055 |
| Trypsin/EDTA (1X) | Thermofisher scientific | R-001-100 |
| 1x Trypsin in Hepes | | |
| Glutamax | Thermofisher scientific | 35050-061 |
| PBS (1X) | Thermofisher scientific | 10010-023 |
| FBS | Thermofisher scientific | 26140079 |
| RPMI | Thermofisher scientific | A1049101 |
| McCoy's 5A | Thermofisher scientific | 16600082 |
| MEMa | Thermofisher scientific | 12561056 |
| DMEM | Thermofisher scientific | 11885084 |
| EGM-2 Bulletkit | Lonza | CC-3162 |
| vectashield mounting media | Vector Laboratories | H-1200 |
| Fluoroshield | Sigma Aldrich | F6182-20ML |
| Fluoro-gel | Electron Microscopy Sciences | 17985-10 |
| Propidium iodide | Thermofisher scientific | P3566 |
| DAPI (4′,6-Diamidino- | Thermofisher scientific | D1306 |
| 2-Phenylindole, | | |
| Dihydrochloride) | | |
| Normal Goat Serum | Thermofisher Scientific | PCN5000 |
| 10% Bovine serum | Thermofisher Scientific | 37520 |
| albumin (BSA) | | |
| Sytox Blue | Thermofisher scientific | S11348 |
| Clearing Agent, | VWR | 97060-934 |
| Histochoice | | |
| Harris haematoxylin (H) | SigmaAldrich | HHS128-4L |
| Eosin Y € | SigmaAldrich | HT110316 |
| Differentiation stain | SigmaAldrich | A3179-1L |
| Alcohol 100% | VWR | 89370-084 |
| Alcohol 95% | VWR | 89370-082 |
| Alcohol 80% | VWR | 89370-080 |
| Alcohol 70% | VWR | 89370-078 |
| Neutral buffered Formalin | VWR | 16004-128 |
| Xylene | VWR | 89370-088 |
| Scotts water | SigmaAldrich | S5134 |

SKBR3 were maintained in McCoys 5A media, MCF-7 cells in Minimal Essential Medium Alpha (MEMa) media, MDA-MB-231 cells in Dulbecco's Modified Eagle Medium (DMEM), HUVEC in EGM-2 Bulletkit, SKOV3-HER2-GFP, EGFR-RFP in McCoy's 5A. SUPT1, SU-DHL-1, NCI-H1703, NCI-H2087, NCI-H647 were all maintained in RPMI-1640. All media was supplemented with 10% Fetal Bovine Serum (BSA), 10 mM penicillin-streptomycin, and 10 mM Glutamax. All cell lines were cultured in a 37° C. humidified atmosphere containing 95% air and 5% CO₂ and were split and media replenished according to manufactures recommendations. After the cultures reached the appropriate confluence the cells were washed 1× with PBS and then dislodged in 1× Trypsin/EDTA, and the resulting solution centrifuged and washed 2× with PBS. The final centrifuged pellet was flash frozen in liquid nitrogen for ˜1-5 minutes and stored at −80 C until hydrogel monomer (HM) embedding.

For xenograft inoculations, MCF-7 cells were cultured and then harvested while still in the linear phase of growth. Prior to inoculation, cells were harvested in 1×0 PBS, containing no calcium or magnesium, by scraping the cells from a 150 cm² tissue culture dish. Following a cell count, the cell pellet was treated with 0.5 mL of 1× trypsin (in HEPES balanced salt solution) for 5 min to disrupt cells and generate a single cell suspension. The harvested population was then counted and adjusted to a concentration of 10⁷ cells per 100 uL in MEMa.

Frozen Cell Pellet/Human Tumor/Mouse Fresh Tissue HM Embedding, and Lipid Clearing

HM embedding. HM embedding was performed according to previously published methods (Chung 2013) with some modifications. In brief, frozen cell line pellets or frozen human tumor tissue or freshly harvested mouse tissue were incubated in 5-50 mL of HM solution (4% acrylamide, 0.05% BIS-Acrylamide, 0.25% VA-044 Initiator, 4% Paraformaldehyde, 1× PBS, pH 7.4) at 4° C. for ˜24-72 hours and then degassed under vacuum for 10-30 minutes, and polymerized at 37° C. for 3 hours until the HM solution solidified. The excess polymerized gel was carefully removed from and the pellet washed 2× in PBST (1× PBS, pH 7.4, 0.1% triton x-100) prior to placing in 25 mL clearing solution (200 mM Borate buffer, pH 8.5 and 8% SDS). The tissues or cell lines were cleared at temperatures ranging from 37° C-55° C. with shaking at 100 rpm and with buffer changes every ˜3-7 days and until the pellets appeared visually clear by eye, anywhere from a few days to 2 months.

Hematoxylin and Eosin (H&E) staining. HM-embedded samples underwent an abbreviated staining protocol that eliminated the Histochoice clearing and dehydrating graded ethanol series described below. In brief, the tissue sections were cleared using 3 exchanges of Histochoice for 5 minutes each. The samples were then dehydrated in a graded ethanol series (2×100%, 2×95%, 1×80%, 1×70% for 3 minutes each). The sections were then rinsed in distilled water for 3 minutes. Hematoxylin was added to each section for 1 minute, rinsed quickly in tap water, followed by differentiation solution—3 minutes, Scott's Tap Water—30-45 seconds, distilled water—1 minute, and stained with eosin for 1 minute. The excess eosin was removed and rehydrated through a graded ethanol series (2×70%, 2×80%, 2×95%, 2×100%, 2 minutes each), cleared with 3 exchanges of Histochoice at 2 minutes each, and mounted with HistoChoice mounting media using a Leica Galen III microscope with a plan 4×0.1 objective and Aptina CMOS sensor digital camera (5.1 MP 1/2.5″ color USB 2.0) and the Toupview 3.7 digital camera software.

Immunohistochemistry. Double paraffin and HM-embedded samples underwent a classic immunofluorescence staining. Tissue sections were cleared using 3 exchanges of Histochoice for 5 minutes each. The samples were then dehydrated in a graded ethanol series (2×100%, 2×95%, 1×80%, 1×70% for 3 minutes each). The serial sections were rinsed twice in ddH₂O for 5 minutes each and then exposed to Histo/zyme for antigen retrival at room temperature for 15 minutes. After a 5 min PBS wash, the sections were covered with Protein Block Serum-Free (Dako) and allowed to incubate at room temperature for one hour. The samples were then stained with a primary antibody (10 μg/m1 or 1:50) in a humidified chamber from one hour at RT to overnight at 4° C., washed 3× with TB ST, followed by staining with a secondary goat-anti-rabbit (or mouse) antibody for one hour at RT. The secondary antibody was removed, and the sections were washed 3× with TBST, 5 minutes each. Finally, the samples were counterstained with nuclear stain DAPI (1 μg/ml, 5 min) or Sytox Blue (1 μM, 15 min) at room temperature before the tissue slides were mounted with Fluorosheild and a coverslip prior to imaging using a Leica Galen III microscope with a plan 4×0.1 objective and Aptina CMOS sensor digital camera (5.1 MP ½.5″ color USB 2.0) and the Toupview 3.7 digital camera software.

Immunohistochemistry: HM embedded cell lines and tissues. HM-embedded samples were blocked using 10% normal goat serum, 3% BSA, and 0.1% Triton X-100 in PBS (blocking buffer). Samples were placed in the blocking buffer from 1-3 hours depending on sample thickness. The samples were transferred to the appropriate primary antibody solution (10 μg/ml or 1:50) diluted in the blocking buffer and allowed to incubate 12-144 hours at 4° C., depending on sample thickness. The samples were washed in PB ST (1× PBS plus 0.1% Triton X-100) for a minimum of 4-72 hours at room temperature. The appropriate species-specific secondary antibody (10 μg/ml or 1:200) was prepared in blocking buffer and allowed to incubate on the sample for 3-144 hours at room temperature before a final 1-2 PB ST washes at room temperature for at least 4-48 hours. The samples are then counterstained with DAPI diluted in reagent grade water (1 μg/ml, 5 min) or Sytox Blue diluted in reagent grade water (1 μM, 15′-24 hours) at room temperature before the tissue sections are placed in RapiClear CS (SunJin Lab Co.) mounting reagent to equilibrate the refractive index of the tissue sample for imaging.

Imaging and 3D image processing of HM stained cell lines and tissues. One of 3 different imaging modalities was used to acquire fluorescent images of stained HM-embedded tissue. The first method was primarily for thin section tissues (>100 μM) and involved the use of an IRIS digital image analysis system with LED filter cubes. The iRiS™ Digital Cell Imaging System is a multicolor fluorescence imaging system integrated with image analysis software and an onboard computer (Logos Biosystems). The following objective and LED filter cubes were used (TC planFluor 10×, 20×, Plan Apochromat fluor oil 40×, TC plan acro 4× Ph, DAPI DAPI (Ex375/28, Em460/50), mCherry (Ex580/25, Em645/75), Cy5 (Ex620/60, Em700/75) for imaging). The second method was primarily for sections that were in the 100-250 μM range, and this was an inverted LSM780 multiphoton laser scanning confocal microscope (Zeiss) using the following objectives (EC Plan Neo 40× oil, Plan Apo 20×, Zeiss), and thirdly an Ultramicroscope II Light sheet microscope (LaVision Biotec) was used for tissues ranging in size from 500 μM-10 mm range, using a standard magnification in the range of 0.63-6.3× in combination with a 2× objective (Mv PLAPO2VC, Olympus). All images were analyzed and processed utilizing the IMARIS scientific 3D/4D processing and analysis software, version 8.2.

Generation of tumor xenografts. On day 0, each of 6 animals were injected orthotopically with a cell suspension (1×10⁷ cell s/100 mL) subcutaneously into the mammary fat pad under the right second nipple into female Rag-2gamma mice (age 6-8 weeks, 20-25 g. Animals were implanted subcutaneously with a intracapsular 60-day slow release 17 B-estradiol pellet without anesthesia. The tumors grew until they reached at least 250 cm³ and no more than 500 cm3 (measured with calipers). General animal conditions were controlled daily and tumor volume and body weight was assessed 2×/week. All experiments were approved by the Molecular Medicine Research Institute (MMRI) IACUC committee, protocol 16-002.

Harvesting of organs from PDX mice, ND4 mice, and MCF-7 xenograft mice. Four PDX tumor model or ND4, Swiss Webster model mice (Table x) were euthanized and subjected to transcardial perfusion with 20 mL of ice cold 1× PBS at a rate of 10 mL/minute. Animals were then perfused with 20 mL of ice cold 4% Hydrogel Monomer (HM) solution at a rate of 10 mL/minute. Following perfusion, the PDX tumor tissue was excised and placed into 50 mL conical tubes containing HM solution.

Results

### Overall Experimental Summary: Normal Mouse Tissue, Mouse Cell Line and Patient Derived Xenografts, Human Cancer Cell Lines, and Human Excision Biopsy Samples

Antibodies identified as suitable for use in HM embedded samples are provided in Table 3. Antibodies determined to work in HM embedded tissue were determined by initially testing for antigen binding using the cell line controls listed in Table 4. Table 4 also includes the mouse (normal and cancer xenograft tissues) and human cancer tissue utilized in these studies. 2-3 different antibodies were tested using cell line monolayers as positive controls for the antigen of interest. Typically, the antigen of interest was expressed in fairly high concentrations according to the literature and was the rationale for using a particular model cell line. An antibody graduated to testing in HM embedded cell line controls, if the staining in monolayer cultures was specific with respect to isotype controls and displayed a good signal/noise ratio.

TABLE 3
Antibodies for HM embedded samples.

| Antibody | Species | Clonality | Clone | Isotype | Vendor | Cat. No. |
|---|---|---|---|---|---|---|
| Alpha smooth | Rabbit | Polyclonal | N/A | IgG | Abeam | ab5694 |
| muscle Actin | | | | | | |
| CD31 | Mouse | Monoclonal | 89C2 | Ig | Cell | 3528 |
| | | | | | Signaling | |
| Texas Red | Tomato | N/A | [GlcNac]1-3, | N/A | Vector | DL-1177 |
| conjugated | | | N-Acetylglucosamine | | Laboratories | |
| Lycopersicon | | | | | | |
| Esulentum, | | | | | | |
| tomato lection | | | | | | |
| CD3 | Rabbit | Monoclonal | Sp7 | IgG | fisher | 9107-S1 |
| CD8 | Rabbit | Polyclonal | N/A | IgG | Abcam | ab4055 |
| EGFR [EP38Y] | Rabbit | Monoclonal | EP38Y | IgG | Abcam | ab52894 |
| ErbB 2/HER2 | Mouse | Monoclonal | 3B5 | IgG1 | Abcam | ab16901 |
| [3B5] | | | | | | |
| ErbB2/HER2 | Mouse | Monoglonal | CB11 | IgG1 | Abcam | ab8054 |
| (CB11) | | | | | | |
| ErbB 2/HER2 | N/A | Monoclonal | N/A | | Abcam | ab31891 |
| Affibody-FITC | | | | | | |
| Estrogen | Rabbit | Polyclonal | N/A | IgG | Abcam | ab3575 |
| Receptor alpha | | | | | | |
| Estrogen | Mouse | Monoclonal | [C-542] | IgG1 | Abcam | ab66102 |
| Receptor alpha | | | | | | |
| [C-542] | | | | | | |
| FOXP3 | Rabbit | Polyclonal | N/A | IgG | Abcam | ab54501 |
| FoxP3 | Mouse | Monoclonal | N/A | IgG1 | Abcam | ab22510 |
| Ki67 | Rabbit | Polyclonal | N/A | IgG | Abcam | ab15580 |
| NAPSIN A | Mouse | Monoclonal | KCG1.1 | IgG1 | Abcam | ab73021 |
| [KCG1.1] | | | | | | |
| pan | Mouse | Monoclonal | [AE1/AE3 + | IgG1 | Abcam | ab86734 |
| Cytokeratin | | | D3] | | | |
| [AE1/AE3 + D3] | | | | | | |
| Cytokeratin 8 + | Rabbit | Monoclonal | EP1628Y | IgG | Abcam | ab192468 |
| 18 (Alexa Fluor | | | | | | |
| 647) | | | | | | |
| PD-L1 [28-8] | Rabbit | Monoclonal | 28-8 | IgG | Abcam | ab205921 |
| TTF1 | Rabbit | Monoclonal | EP1584Y | IgG | Abcam | ab76013 |
| [EP1584Y] | | | | | | |
| Goat Anti- | Goat | Polyclonal | N/A | IgG | Abcam | ab150117 |
| Mouse-AF488 | | | | | | |
| Goat Anti- | Goat | Polyclonal | N/A | IgG | Abcam | ab175701 |
| Mouse-AF568 | | | | | | |
| Goat Anti- | Goat | Polyclonal | N/A | IgG | Abcam | ab150115 |
| Mouse-AF647 | | | | | | |
| Goat Anti- | Goat | Polyclonal | N/A | IgG | Abcam | ab175741 |
| Mouse-AF750 | | | | | | |
| Goat Anti- | Goat | Polyclonal | N/A | IgG | Abcam | ab150081 |
| Rabbit-AF488 | | | | | | |
| Goat Anti- | Goat | Polyclonal | N/A | IgG | Abcam | ab175695 |
| Rabbit-AF568 | | | | | | |
| Goat Anti- | Goat | Polyclonal | N/A | IgG | Abcam | ab150079 |
| Rabbit-AF647 | | | | | | |
| Goat-Anti- | Goat | Polyclonal | N/A | IgG | Thermo | A-21038 |
| Rabbit AF700 | | | | | fisher | |
| Goat Anti- | Goat | Polyclonal | N/A | IgG | Abcam | ab175733 |
| Rabbit-AF750 | | | | | | |

AF = Alexa Fluor ®

TABLE 4
Cell Lines and Tissues

| Description of Cell or Tissue | Vendor | Catalog # |
|---|---|---|
| MCF-7 Human breast adenocarcinoma cell line; luminal A | ATCC | HTB-22 |
| classification | | |
| MDA-MB-231 Human Breast adenocarcinoma cell line; Triple | ATCC | HTB-26 |
| negative classification | | |
| SKBR3 Human Breast adenocarcinoma cell line; HER2 positive | ATCC | HTB-30 |
| classification | | |
| HUVEC Human Umbilical Vein Endotheial Cells | LONZA | CC-2935 |
| | | (single |
| | | donor) |
| SKOV3-HER2-GFP, EGFR-RFP | Sigma- | CLL1143 |
| | Aldrich | |
| SUP-T1 Human Lymphoblastic Lymphoma | ATCC | CRL-1942 |
| SU-DHL-1 Human Large Cell Lymphoma | ATCC | CRL-2955 |
| NCI-H1703 Human lung squamous carcinoma | ATCC | CRL-5888 |
| NCI-H2087 Human Lung adenocarcinoma | ATCC | CRL-5922 |
| NCI-H647 Human Lung adenosquamous carcinoma | ATCC | CRL-5834 |
| Fresh frozen human breast tumor, comedocarcinoma, non-infiltrating, | Asterand | N/A |
| ER−/PR−/HER2+ | | |
| Fresh frozen human lymph node; infiltrating ductal carcinoma | Asterand | N/A |
| metastatic breast; stage IIIA; poorly differentiated; ER+/PR+/HER2+ | | |
| Fresh frozen human breast tumor; infiltrating ductal carcinoma | Asterand | N/A |
| metastatic breast; stage IIIA; poorly differentiated; ER+/PR+/HER2+ | | |
| Fresh Frozen human lung adenocarcinoma stage 1A; EGFR exon 19 | Asterand | N/A |
| pos and KRAS null | | |
| Fresh Frozen Human adenosquamous lymph node stage 2A | Asterand | N/A |
| Fresh FrozenHuman Lung Squamous stage 1B | Asterand | N/A |
| Fresh Frozen Human lung adenocarcinoma stage 1B; negative EGFR | Asterand | N/A |
| and KRAS null | | |
| Fresh Frozen adenosquamous lymph node stage 2A | Asterand | N/A |
| MCF7 mouse xenograft tumors; human breast adenocarcinoma cell | In house | Rag2- |
| line; luminal A classification | prepared | gammaC |
| | xenograft; | mice |
| | mice from | |
| | Charles | |
| | Rivers; | |
| | MCF-7 from | |
| | ATCC | |
| Mouse patient derived xenograft (PDX) breast tumor; ER−/PR−/ | Jackson | TM00129 |
| HER2+ | Laboratories | |
| Mouse Kidney | Charles | ND4- |
| | Rivers | swiss |
| | | webster |

The monolayer cell culture immunofluorescence data was generated using standard assay techniques (data not shown). Table 5 summarizes the results of the testing of antibodies in HM embedded cell lines and mouse xenograft or human cancer tissue, and the relative staining of these antibodies is indicated by the following designations: 0=no staining detected, 1+=low staining, 2+=moderate staining; 3+=strong staining. These findings do not necessarily correlate with what has been published in the literature for the known targets, potentially due to a number of reasons including the concentration of the primary antibody or the secondary antibody used in a singleplex or a multiplex reaction. The primary antibody concentrations utilized were 0.38-20 mg/mL, or at 1:100, if no concentration information was available from the vendor. For fluorescent visualization, there were two methods utilized and indicated as indirect or direct and the type of secondary antibody (indirect) or fluorophore conjugate (direct) indicated in column 7. Secondary antibodies were all utilized at a 1:100 dilution. Also, listed here is the embedding method, which was exclusively standard HM embedding, for 48 hours, as well as the clearing time/temperature and the size of the tissue. The tissue sizes ranged from 7 μM-10 mm³. Anything above 5mm³ was labeled as “thick” for the purposes of this document. The clearing time for the tissues was from 5-32 days and depending on the tissue type (cancer versus normal tissue or cell line), and the clearing temperature 37°-55° C.

TABLE 5
Summary of Staining Results

| | | Embedding | Clearing | | Multiplex | Nuclear | |
|---|---|---|---|---|---|---|---|
| | Sample | Method/ | Time/ | ~Tissue | or | Stain/Antibody/ | Imaging |
| Sample | Type | time | Temperature | Size | SinglePlex | Intensity | Method |
| MCF-7 | Cell Line | HM/48 | 5 days/55 C. | 3 mm³ | Singleplex | pan-Cytokeratin | Ultramicroscope |
| Frozen Pellet | | hours | | | | (Alexa 488): 3 + | II Light sheet |
| | | | | | | Cytokeratin 8/18 | |
| | | | | | | (alexa 647 | |
| | | | | | | direct): 3 + | |
| | | | | | | Ki-67 (Alexa | |
| | | | | | | 488) 3 + | |
| | | | | | | Propidium | |
| | | | | | | Iodide: 3 + | |
| | | | | | | Sytox Blue: 3+ | |
| MCF-7 | Cell Line | HM/48 | 7 days/37 C. | 50 mM | Multiplex | ERα (Alexa | iRiS ™ |
| Frozen Pellet | | hours | | | | 488): 2 + | Digital Cell |
| | | | | | | Foxp3[mAb | Imaging |
| | | | | | | 22510] (Alexa | System |
| | | | | | | 647): 1 + | |
| | | | | | | Ki-67 (Alexa | |
| | | | | | | 488): 2 + | |
| | | | | | | pan-cytokeratin | |
| | | | | | | (Alexa 647): 2 + | |
| | | | | | | ERα[C-542] | |
| | | | | | | (Alexa488): 1 + | |
| | | | | | | Foxp3 (Alexa | |
| | | | | | | 647): 1 + | |
| | | | | | | α-SMA (Alexa | |
| | | | | | | 488): 2 + | |
| | | | | | | HER2[3B5] | |
| | | | | | | (Alexa 647): 2 + | |
| | | | | | | DAPI: 3+ | |
| NCI-647 | Cell Line | HM/48 | 5 days/37 C. | 2 mm³ | Singleplex | Ki-67 (Alexa | Ultramicroscope |
| Frozen Pellet | | hours | | | | 488): 2 + | II Light sheet |
| | | | | | | EGFR (Alexa | |
| | | | | | | 488): 0 | |
| | | | | | | Napsin A (Alexa | |
| | | | | | | 488): 2 + | |
| | | | | | | TTF1 (Alexa | |
| | | | | | | 488): 1+ | |
| NCI-1703 | Cell Line | HM/48 | 5 days/37 C. | 2 mm³ | Singleplex | Ki-67 (Alexa | Ultramicroscope |
| Frozen Pellet | | hours | | | | 488): 1 + | II Light sheet |
| | | | | | | EGFR (Alexa | |
| | | | | | | 488): 2 + | |
| | | | | | | Napsin A (Alexa | |
| | | | | | | 488): 1 + | |
| | | | | | | TTF1 (Alexa | |
| | | | | | | 488): 2+ | |
| NCI-2087 | Cell Line | HM/48 | 5 days/37 C. | 2 mm³ | Singleplex | Ki-67 (Alexa | Ultramicroscope |
| Frozen Pellet | | hours | | | | 488): 2 + | II Light sheet |
| | | | | | | EGFR (Alexa | |
| | | | | | | 488): 2 + | |
| | | | | | | Napsin A (Alexa | |
| | | | | | | 488): 1 + | |
| | | | | | | TTF1 (Alexa | |
| | | | | | | 488): 2+ | |
| Multi-cell | Cell Line | HM/48 | 10 days/45 C. | 2-3 mm³ | In | N/A | N/A |
| frozen pellet | | hours | | | progress | | |
| (SKBR3, MCF-7, | | | | | | | |
| MDA-MB-231, | | | | | | | |
| SUPT1, SUD-HL, | | | | | | | |
| Huvec) | | | | | | | |
| MCF-7 | Mouse #3 | Perfusion/ | 5 days/45 C. | 50-100 | multiplex | Foxp3 (Alexa | iRiS ™ |
| Xenograft perfused | MCF-7 | HM/48 | | mM | | 568): 1 + | Digital Cell |
| Tissue | Xenograft | hours | | | | ERα[C- | Imaging |
| | | | | | | 542]Alexa647: | System |
| | | | | | | 1 + | |
| | | | | | | DAPI: 3+ | |
| MCF-7 | Mouse #3 | Perfusion/ | 5 days/45 C. | 50-100 | multiplex | CD-8 (Alexa | Confocal |
| Xenograft perfused | MCF-7 | HM/48 | | mM | | 700): 1 + | LSM780 |
| Tissue | Xenograft | hours | | | | Lectin-Texas | |
| | | | | | | Red direct: 3 + | |
| | | | | | | Cytokeratin | |
| | | | | | | 8/18-Alexa 647 | |
| | | | | | | direct: 3 + | |
| | | | | | | PD-Ll [28-8]- | |
| | | | | | | Alexa 647 | |
| | | | | | | direct: 2 + | |
| | | | | | | Sytox Blue: 3+ | |
| Human Breast | T2N0M0 | HM/48 | 33 days/49 C. | Thick | Singleplex | Ki-67 (Alexa | Ultramicroscope |
| Comedocarcinoma, | HER2+, | hours | | | | 488)3 + | II Light sheet |
| non-infiltrating | ER/PR | | | | | Pan-cytokeratin | |
| | negative | | | | | (Alexa 488): 3 + | |
| | | | | | | HER2 Affibody- | |
| | | | | | | FITC direct: 3+ | |
| Human Lung | Stage IB, | HM/48 | 32 days/55 C. | 100 mM | Multiplex | CD-3 (Alexa | Confocal |
| Adenocarcinoma | EGFR, | hours | | (sectioned | | 700): 1 + | L5M780 |
| | KRAS | | | following | | CD-8 (Alexa | |
| | negative | | | clearing) | | 700): 1 + | |
| | | | | | | CD-31 (Alexa | |
| | | | | | | 568) 1 + | |
| | | | | | | Cytokeratin | |
| | | | | | | 8/18-Alexa 647 | |
| | | | | | | direct: 1 + | |
| | | | | | | PD-L1 [28-8]- | |
| | | | | | | Alexa 647 | |
| | | | | | | direct: 0 | |
| | | | | | | Sytox Blue: 3+ | |
| Human Metastatic | Infiltrating | HM/48 | 32 days/55 C. | 100 mM | Multiplex | CD-3 (Alexa | Confocal |
| Breast Cancer | ductal | hours | | (sectioned | | 700): 2 + | L5M780 |
| Lymph Node | carcinoma, | | | following | | CD-8 (Alexa | |
| | stage IIIA; | | | clearing) | | 700): 2 + | |
| | ER+/PR+/ | | | | | CD-31 (Alexa | |
| | HER+ | | | | | 568) 1 + | |
| | | | | | | Cytokeratin | |
| | | | | | | 8/18-Alexa 647 | |
| | | | | | | direct: 1 + | |
| | | | | | | Sytox Blue: 3+ | |
| Human Metastatic | Fresh frozen | HM/48 | 32 days/55 C. | Thick | In | N/A | N/A |
| Breast Cancer | human breast | hours | | | progress | | |
| | tumor; | | | | | | |
| | infiltrating | | | | | | |
| | ductal | | | | | | |
| | carcinoma | | | | | | |
| | metastatic | | | | | | |
| | breast; | | | | | | |
| | stage IIIA; | | | | | | |
| | poorly | | | | | | |
| | differentiated; | | | | | | |
| | ER+/PR+/ | | | | | | |
| | HER2+ | | | | | | |
| Human Lung | Fresh Frozen | HM/48 | 28 days/55 C. | Thick | In | N/A | N/A |
| Adenocarcinoma | human lung | hours | | | progress | | |
| | adenocarcinoma | | | | | | |
| | stage 1A; | | | | | | |
| | EGFR exon | | | | | | |
| | 19 pos and | | | | | | |
| | KRAS null | | | | | | |
| Human | Fresh Frozen | HM/48 | 28 days/55 C. | Thick | In | N/A | N/A |
| adenosquamous | Human | hours | | | progress | | |
| | adenosquamous | | | | | | |
| | lymph node | | | | | | |
| | stage 2A | | | | | | |
| Human Lung | Fresh Frozen | HM/48 | 28 days/55 C. | Thick | In | N/A | N/A |
| Squamous | Human Lung | hours | | | progress | | |
| | Squamous | | | | | | |
| | stage 1B | | | | | | |
| Human | Fresh Frozen | HM/48 | 28 days/55 C. | Thick | In | N/A | N/A |
| adenosquamous | adenosquamous | hours | | | progress | | |
| lymph node | lymph node | | | | | | |
| | stage 2A | | | | | | |
| Mouse perfused | Normal Kidney | Hm/48 | 7 days/37 C. | 50 μm | N/A | N/A; H & E: | Leica Gallen II |
| tissue | | hours | | | | good | |
| | | | | | | morphology | |
| Mouse perfused | Normal | Hm/48 | 7 days/37 C. | 50 & | Multiplex | Ki-67 (Alexa | iRiS ™ |
| tissue | Kidney | hours | | 100 mM | | 488): 2 + | Digital Cell |
| | | | | | | pan-cytokeratin | Imaging |
| | | | | | | (Cy3): 1 + | System |
| | | | | | | CD-31 (Alexa | |
| | | | | | | 488): 2 + | |
| | | | | | | pan-cytokeratin | |
| | | | | | | (Alexa 647 | |
| | | | | | | direct): 1 + | |
| | | | | | | α-SMA-FITC: | |
| | | | | | | 2 + | |
| | | | | | | DAPI: 3+ | |

Thick = greater than 2 mm in any dimension
Direct = fluorophore directly conjugated to antibody

These data demonstrate that it is possible to clear a variety of cancer tissues, from mouse cell line and PDX xenograft tumor tissues, as well as various histological subtypes and stages of human breast and lung cancer tissue and normal lymph node tissue. It also illustrates that HM-embedded cleared tissue that has been stained with a variety of commercially available antibodies, can be imaged with a variety of imaging modalities, depending on the thickness of the sample. To note, both frozen human tumor tissue as well as frozen pellets can be successfully HM-embedded, lipid-cleared, and successfully stained without disrupting the morphology of the tissue. To our knowledge, this has not been reported for the CLARITY method with normal or diseased tissue, including cancer tissue. Furthermore, the use of frozen cell line pellets has not been reported in conjunction with the CLARITY method. This method provides an excellent way to control for multiple aspects of the process and have a renewable and reproducible reagent for the research and development of the technology. Furthermore, mixtures of cell lines containing antigens of interest in varying concentrations can be utilized to understand sensitivity and specificity of priority stains. This development strategy can be applied to the field of oncology or any other disease state that has cell line models with nucleic acid or protein targets of interest.

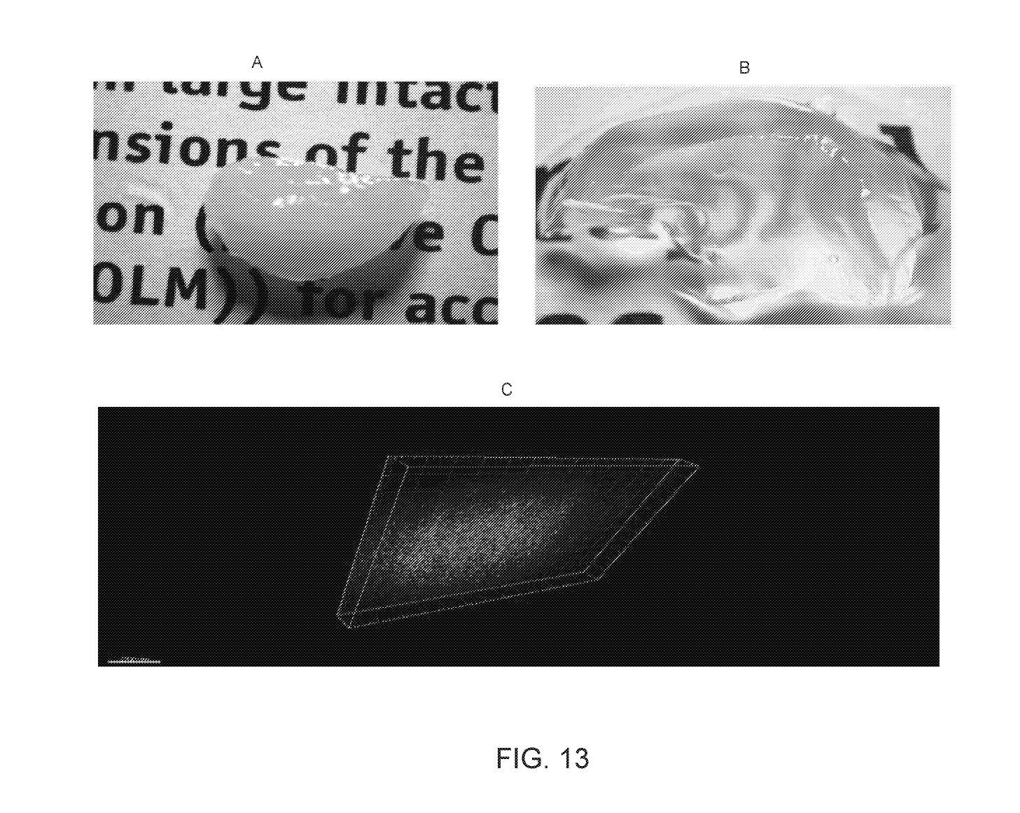

Frozen cell line control. A flash frozen cell line control (MCF-7) was HM-embedded (FIG. 13A), then cleared for 5 days at 55° C. (FIG. 13B), then incubated with propidium iodide to stain the cell nuclei, and then imaged with light sheet microscopy by creating a 1 mm Z-stack with a 10 μM step size (FIG. 13C). Image processing was done by utilizing the IMARIS scientific 3D/4D processing and analysis software, version 8.2. The before and after images (FIG. 13A versus FIG. 13B) illustrates the opaque nature of the frozen HM embedded sample versus the transparent pellet following clearing. The propidium iodide penetrated deeply into the tissue and allowed uniform imaging through 1 mm of the sample.

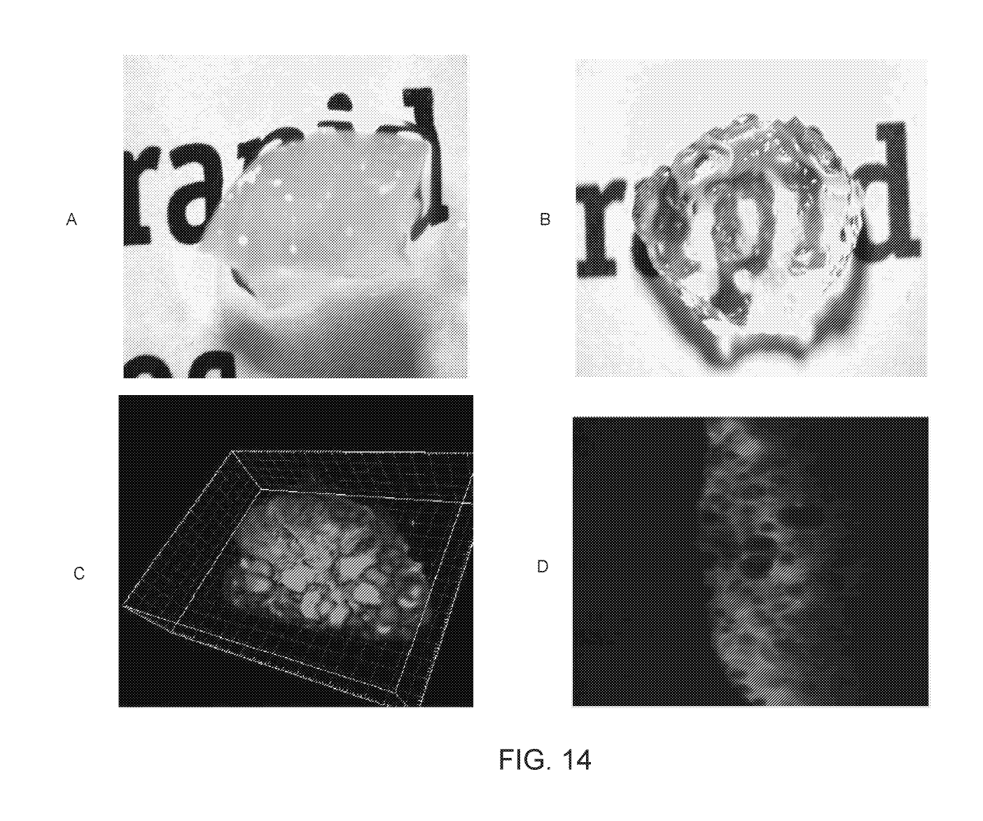

Mouse patient derived xenograft (PDX). An example of a mouse PDX human sample is illustrated in FIG. 14. This sample was from a patient with an infiltrating ductal carcinoma that was previously classified as Estrogen Receptor (ER) negative and Progesterone receptor (PR) negative and HER2 positive. The sample was prepared as described in the methods section and the HM embedded sample (FIG. 14A), cleared for 7 days at 55° C. (FIG. 2B), and stained with either anti-pan cytokeratin (FIG. 14C) or anti-HER2 (clone CB11) primary antibodies (FIG. 14D) and an Alexa Fluor® 488 secondary antibody, and imaged with light sheet microscopy and analyzed with IMARIS version 8.2 software. Refer to Table 3 for details on antibodies. The before and after images (FIG. 14A versus FIG. 14B) illustrate the opaque nature of the mouse perfused HM embedded sample versus the transparent pellet following 7 days of clearing at 55° C. The pan-cytokeratin imaging (1.2mm Z-stack with 10 μM step size) illustrates a fairly uniform 3+staining across the entire tissue with great preservation of morphology. Strong 3+ HER2 staining could be detected in this sample using the CB11 antibody clone.

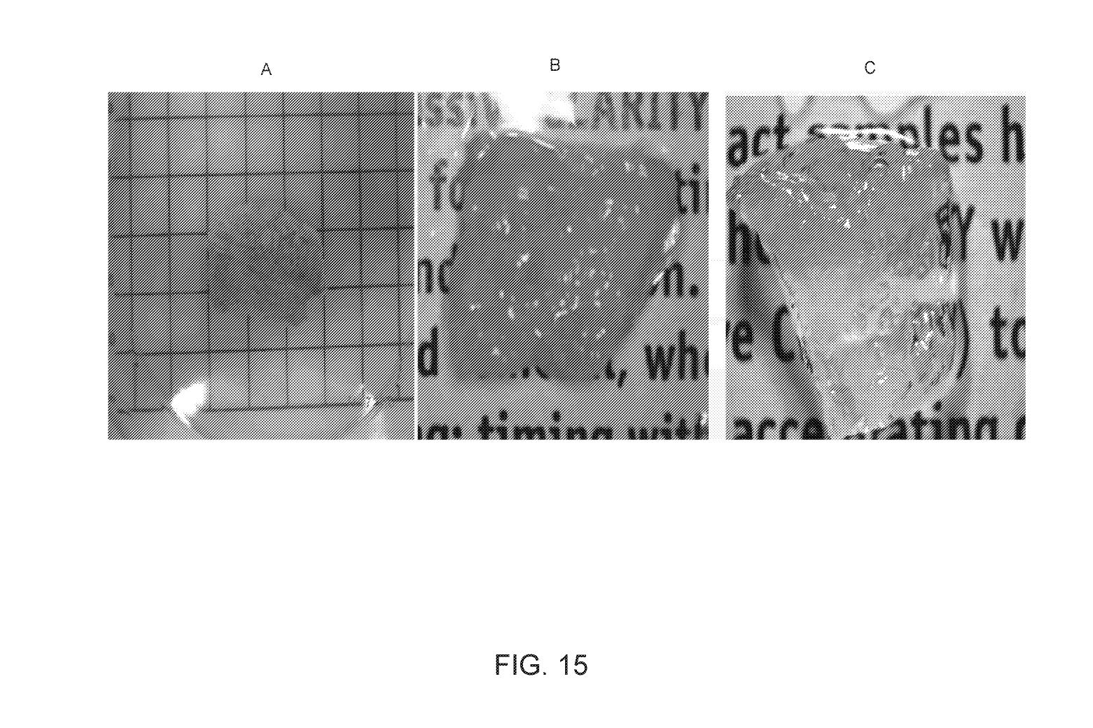

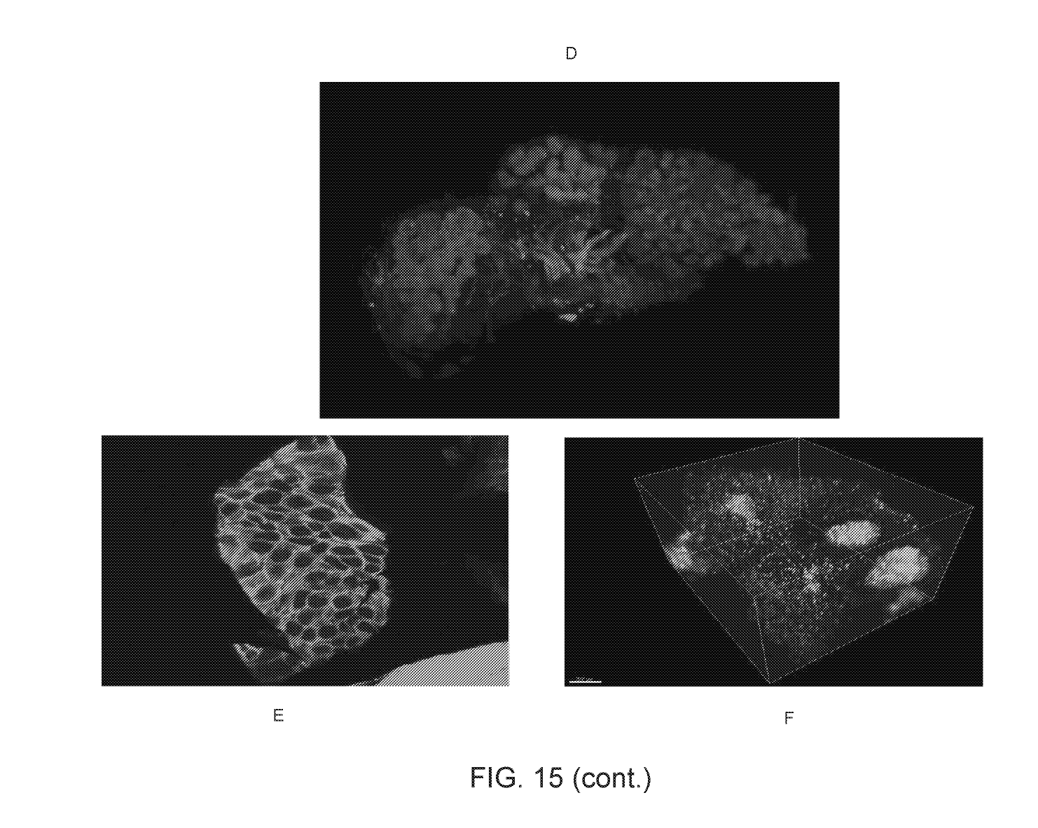

Human breast cancer excision biopsy specimen. An example of a human breast cancer excision biopsy specimen sample is illustrated in FIG. 15. This sample is from a patient with a non-infiltrating comedocarcinoma of the breast that was previously characterized, in a separate part of the tumor to be ER negative, PR negative and HER2 positive. This commercially available frozen human breast cancer excision specimen (FIG. 15A) was HM embedded (FIG. 15B) and cleared (FIG. 15C) as indicated in Table 5 for ˜35 days to clear. The sample was stained with antibodies against HER2 (ErbB 2/HER2 Affibody-FITC conjugated) (FIG. 15D), pan-cytokeratin (FIG. 15E), or Ki-67 (FIG. 15F) and imaged with either confocal or light sheet microscopy and analyzed with IMARIS version 8.2 software. From the sample that was stained with HER2, the analysis of 600 μM Z-stack with 10 μM step size from light sheet, and revealed a very ordered tree-like 3D structure that was not able to be seen with a 5 μM H & E stained slide (data not shown, Asterand). This demonstrated the ability to take a frozen human excision specimen and HM embed, clear and stain the tissue without the loss of critical architecture of the tumor. This work demonstrates that frozen specimen samples, as opposed to fresh excision samples, may be used according to the methods described herein. Strong 3+ staining of the tumor epithelial cells was seen with an anti-pan cytokeratin antibody (FIG. 15E) as well as 3+ staining of tumor nuclei with an anti-Ki-67 antibody (FIG. 15F). Interestingly, there was heterogeneity of the Ki-67 staining, indicating that there may be differential proliferation across the tumor depending on the position within the tumor. This demonstrates that the methods descrined herein may have implications for treatment response, as compared to depending on which part of the tumor is analyzed if performed with conventional procedures using 2D thin formalin fixed paraffin embedded sections.

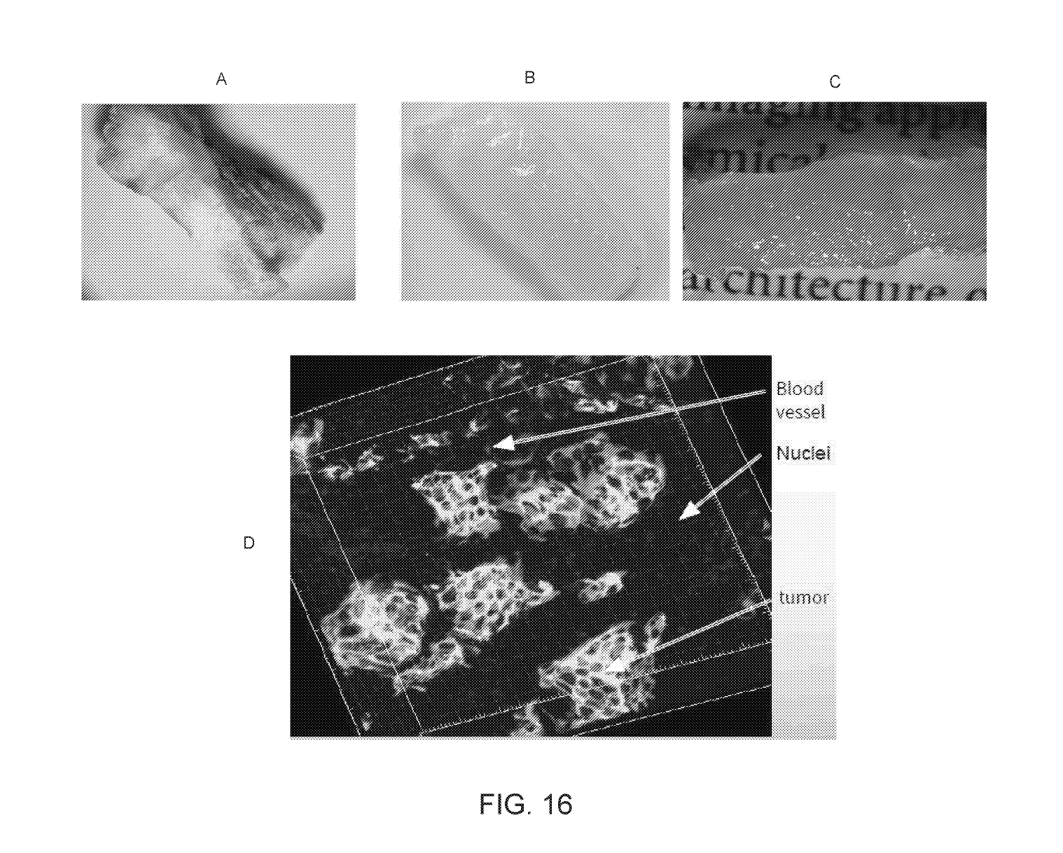

Human metastatic breast cancer lymph node. A human metastatic breast cancer lymph node sample from a patient with a primary tumor that was previously diagnosed as an infiltrating ductal carcinoma, stage IIIA; poorly differentiated; ER, PR, and HER2 positive based on thin section 2D FFPE analysis was analyzed. This commercially available frozen human lymph node from a patient with metastatic breast cancer (FIG. 16A) was HM embedded (FIG. 16B) and passively cleared (FIG. 16C) as indicated in Table 5. The sample was stained in a multiplex reaction, with a sytox blue (nuclei), an antibody against Cytokeratin 8/18 directly conjugated to Alexa Fluor® 647 (tumor cells), and an anti-CD-31 antibody, with a secondary antibody conjugated to Alexa Fluor®568 (blood vessels) (FIG. 16D). Arrows indicate the differential staining of the tumor features and illustrates the ability to embed frozen human cancer tissue, clear and image with 3 different markers in a multiplex. 100 μM sections were imaged with a confocal microscope and analyzed with IMARIS version 8.2 software (see methods section). Interestingly, there was quite a bit of variability across the 100 μM lymph node (5 μM step size) in that there were some Z-stack images that contained only nuclei and no visible tumor cells (data not shown). This suggests the methods described herein may be advantageous in situations where a patient may have micro-metastasis in a lymph node and may be staged improperly due to conventional 2D thin section FFPE testing being performed.

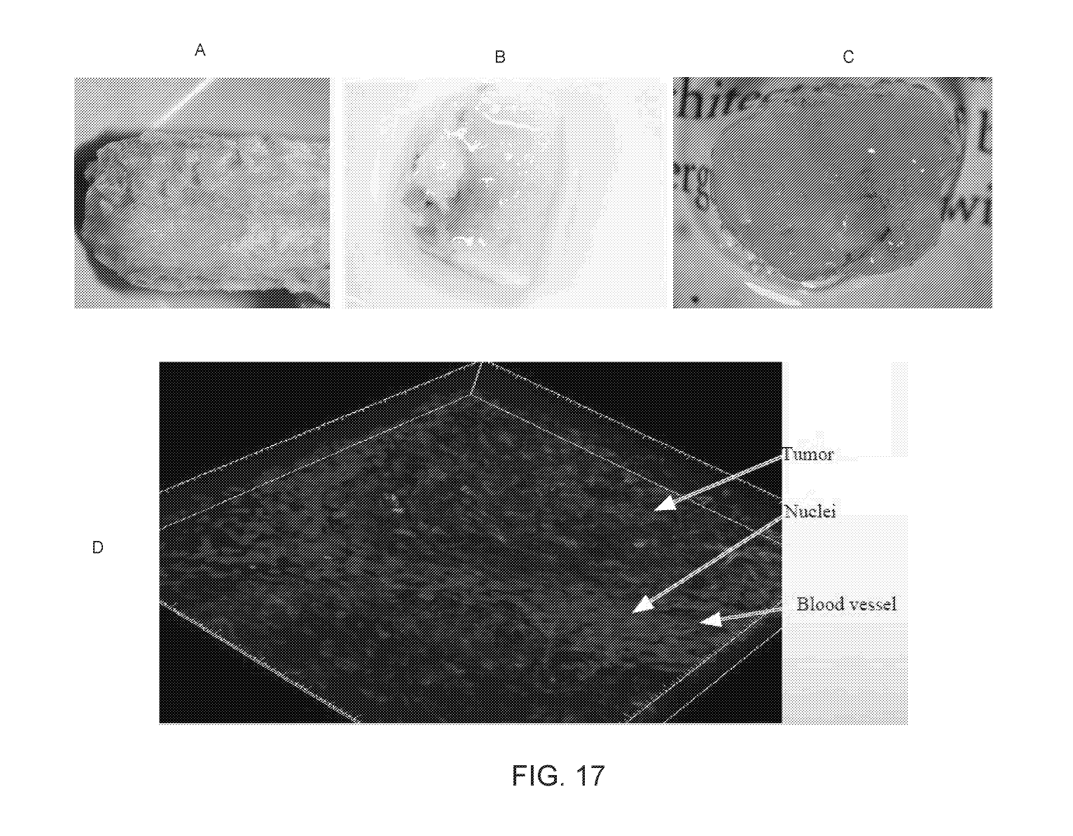

Human Lung Adenocarcinoma, stage 1B. A human lung adenocarcinoma from a patient with a stage 1B that was previously characterized to be negative for sensitizing mutations in EGFR and for KRAS by 2D thin section FFPE analysis was analyzed. This commercially available frozen human excision tumor sample (FIG. 17A) was embedded in HM (Table 5) (FIG. 17B) and then passively cleared (FIG. 17C). The sample was stained in a multiplex reaction, with a sytox blue (nuclei), an antibody against Cytokeratin 8/18 directly conjugated to Alexa Fluor® 647 (tumor cells), and an anti-CD-31 antibody, with a secondary antibody conjugated to Alexa Fluor®568 (blood vessels) (FIG. 17D). Arrows indicate the differential staining of the tumor features and illustrates the ability to embed frozen human cancer tissue, clear and image with 3 different markers in a multiplex. 100 μM sections were imaged with a confocal microscope and analyzed with IMARIS version 8.2 software (see methods section). Due to a lack of having the most optimal anti-pan cytokeratin antibody conjugated to Alexa Fluor® 647, the experiment was done with an antibody against the keratins 8/18. Lung adenocarcinoma highly expresses CK 7. This represented a good negative control for the staining of this tumor. Experiments are also performed using the standard pan cytokeratin antibody conjugated to Alexa Fluor® 647.

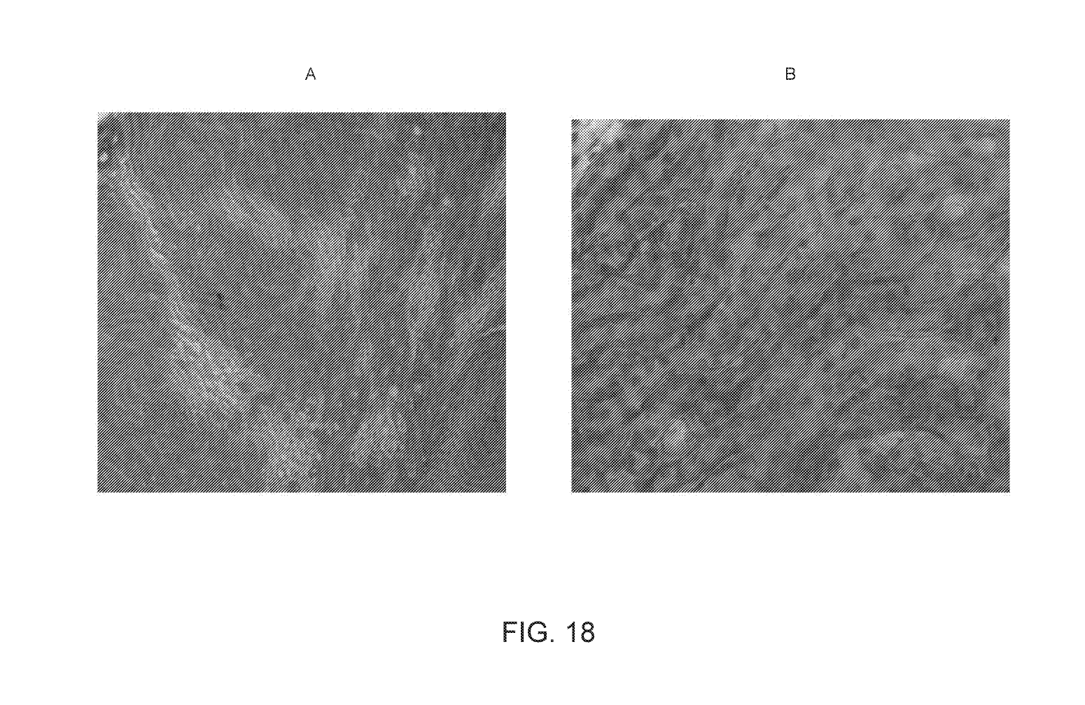

H & E staining of HM-embedded normal mouse kidney. Normal mouse kidney samples were HM embedded, lipid cleared and sectioned using a vibratome. A 50 μM section was subjected to H & E staining, with modifications indicated in the methods section. Brightfield images were taken using a Leica Gallen III microscope with an Aptina CMOS sensor digital camera (5.1 MP ½.5″ color USB 2.0) attached to one eyepiece and visualized using the Toupview 3.7 digital camera software. FIG. 18 illustrates a 10× and a 40× image of the tissue following the staining where very good morphological preservation of the mouse kidney structure following HM embedding and clearing was seen. This success of this type of methodology with HM embedded diseased tissue allows the detection of normal versus cancerous tissue as well as an understanding of the morphology of the sample.

### REFERENCES

1. Chung, K., Wallace, J., Kim, S-Y. et al. Structural and Molecular Interrogation of Intact Biological Systems. Nature. 2013; 497:332-337.

2. Yang, B., Treweek, J. B., Kulkarni, R. P. et al. Single-Cell Phenotyping within Transparent Intact Tissue Through Whole-Body Clearing. Cell. 2014; 158: 1-14.

3. Mittendorf, E. Phillips, A. Meric-Bernstam, F. et al. PD-L1 Expression in Triple Negative Breast Cancer. Cancer Immunol. Res. 2014; April 2(4): 361-370.

4. Subik, K. Lee, J-F., Baxter, L. et al. Breast Cancer: Basic and Clinical Research. 2010: 4, 35-41.

5. http://www.dot.ihcworld.com/_protocols/special_stains/h&e_ellis.dot.htm

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A method for analyzing a biological sample comprising:
processing a biological sample by:
fixing the sample in the presence of hydrogel subunits;
polymerizing the hydrogel subunits to form a hydrogel-embedded sample;
clearing the hydrogel-embedded sample; and
labeling the cleared hydrogel-embedded sample with one or more first detectable marker;
imaging the processed sample by microscopy, optionally light microscopy, to generate at least one first image of the biological sample and/or determine an amount of the first detectable marker in the biological sample,
wherein the biological sample is a tumor tissue sample, a previously frozen biological sample, or a cell line.

2. The method of claim 1, wherein the biological sample has a length of greater than 20 microns and a thickness of greater than 20 microns.

3. A method for diagnosing a tumor or determining the prognosis of a tumor in a subject comprising:
processing a tumor tissue sample obtained from the subject by:
fixing the tumor tissue sample in the presence of hydrogel subunits;
polymerizing the hydrogel subunits to form a hydrogel-embedded sample;
clearing the hydrogel-embedded sample; and
labeling the cleared hydrogel-embedded sample with one or more first detectable marker;
imaging the processed sample to generate at least one tumor image of and/or determine an amount of the first detectable marker; and
comparing the tumor image or amount against one or more control images or amounts or predetermined images or amounts obtained from normal tissue, tumor tissue, tumor tissue associated with a good prognosis, or tumor tissue associated with a poor prognosis,
thereby determining the presence of a tumor or the prognosis of a tumor.

4. A method for determining the responsiveness of a tumor in a subject to a therapeutic treatment comprising:
processing a first tumor tissue sample obtained from the subject at a first time point by:
fixing the tumor tissue sample in the presence of hydrogel subunits;
polymerizing the hydrogel subunits to form a hydrogel-embedded sample;
clearing the hydrogel-embedded sample; and
labeling the cleared hydrogel-embedded sample with one or more first detectable marker;
processing a second tumor tissue sample obtained from the subject at a second time point following a therapeutic treatment by:
fixing the tumor tissue sample in the presence of hydrogel subunits;
polymerizing the hydrogel subunits to form a hydrogel-embedded sample;
clearing the hydrogel-embedded sample; and
labeling the cleared hydrogel-embedded sample with one or more first detectable marker;
imaging the processed samples of (a) and (b) to generate a first tumor image of the processed sample of (a) and a second tumor image of the processed sample of (b) and/or determining a first amount of the one or more first detectable marker in the processed samples of (a) and a second amount of the one or more first detectable marker in the processed sample of (b); and
comparing the first tumor image against the second tumor image, or the first amount to the second amount of the one or more detectable marker, wherein if the second tumor image or second amount shows less tumor characteristics than the first tumor image or amount, the tumor is responding favorably to the therapeutic treatment, and wherein if the second tumor image or amount show more tumor characteristics than the first tumor image or amount, the tumor is responding poorly to the therapeutic treatment.

5.-7. (canceled)

8. The method of claim 1, wherein the imaging includes:
placing the processed sample of (a) in an optically homogenous sample manipulation component;
aligning one or more light sheets and one or more detection focal planes of a microscopic device at a plurality of locations within the processed sample of (a);
performing an imaging procedure to collect an image from each of the plurality of locations with the processed sample of (a); and
generating the first tumor image based on the collected images from each of the plurality of locations.

9. The method of claim 8, wherein the imaging further includes:
applying the alignment parameter to each location;
simultaneously illuminating the location with a light sheet and capturing an image of the location; and
constructing a three-dimensional image of the sample using the image from each location.

10. The method of claim 1, wherein fixing the sample comprises contacting the sample with a paraformaldehyde.

11. The method of claim 1, wherein the hydrogel subunits comprise an acrylamide.

12. The method of claim 1, wherein polymerizing the sample comprises thermal cross-linking.

13. The method of claim 1, wherein clearing the sample comprises electrophoresing the sample.

14. The method of claim 13, wherein the electrophoresing is performed using a buffer solution that comprises an ionic surfactant, optionally sodium dodecyl sulfate.

15. The method of claim 13, wherein:
the sample is electrophoresed using a voltage ranging from about 10 to about 60 volts; and/or
the sample is electrophoresed for a period of time ranging from about 15 minutes to about 10 days.

16. The method of claim 1, further comprising incubating the cleared sample in a mounting medium that has a refractive index that substantially matches that of the cleared sample, wherein the mounting medium increases the optical clarity of the sample, and wherein the mounting medium optionally comprises glycerol.

17. The method of claim 1, further comprising:
washing the processed sample obtained after imaging to remove the one or more first detectable label;
relabeling the washed sample(s) with one or more second detectable label;
imaging the relabeled sample(s) by microscopy, optionally light microscopy, to obtain a second image(s); and
optionally, examining the second image(s) as set forth in step (c) of claim 3 or step (d) of claim 4.

18. The method of claim 1, wherein the labeling and/or relabeling comprises contacting the cleared hydrogel-embedded sample with one or more detectable markers that bind to cellular or extracellular components within the sample.

19. The method of claim 18, wherein the cellular or extracellular components are selected from the group consisting of:
immune cell markers;
cancer stem cell markers;
extracellular matrix proteins;
blood vessel markers;
apoptosis markers; and
tumor markers.

20. The method of claim 1, wherein the detectable marker comprises a polypeptide, nucleic acid, or small molecule.

21. The method of claim 20, wherein the detectable marker is detectable with or without the use of a detectable secondary agent that binds the detectable marker.

22. The method of claim 1, wherein the tumor is selected from the group consisting of: adrenal cortical cancer, anal cancer, aplastic anemia, bileduct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity and paranasal sinus cancer, nasopharyngeal cancer, neuroblastoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, and Wilms tumor.

23. The method of claim 22, wherein:
(a) the tumor is a breast cancer tumor, and the one or more detectable marker binds to or identifies one or more of HER2, estrogen receptor, progesterone receptor, pan-cytokeratin, Ki67, CD3, CD4, CD8, CD20, CD68, or Foxp3, PD-L1, PD-1, PD-L2, CTLA-4, and androgen receptor;
(b) the tumor is a lung tumor, and the one or more detectable market binds to or identifies one or more of epidermal growth factor receptor, including sensitizing and resistance mutations thereof, ALK/ROS/RET rearrangements, BRAF mutations, and PD-L1, PD-1, PD-L2, CTLA4, CD4, CD8, CD20, pan-cytokeratin;
(c) the tumor is a melanoma, and the one or more detectable marker binds to or identifies one or more of BRAF, NRAS, KIT, GNA11/GNAQ, CDK4, and MEK mutations or expression of one or more of PD-L1, PD-1, CTLA-4 CD4, CD8, CD20, and pan-cytokeratin;
(d) the tumor is a colon cancer, and the one or more detectable market binds to or identifies one or more of KRAS mutation and epidermal growth factor receptor;
(e) the tumor is a prostate tumor, and the one or more detectable marker binds to or identifies androgen receptor;
(f) the tumor is a breast cancer tumor, and the one or more detectable marker binds to or identifies HER2/neu;
(g) the tumor is a breast cancer tumor, and the one or more detectable marker binds ki-67; or
(h) the tumor is a lung cancer, and the one or more detectable marker binds CD-31.

24.-30. (canceled)
